(19) United States
(12) Patent Application Publication
(10) Pub. No.: US 2025/0027078 A1
(43) Pub. Date: Jan. 23, 2025

(54) DNA-CONTAINING POLYNUCLEOTIDES AND GUIDES FOR CRISPR TYPE V SYSTEMS, AND METHODS OF MAKING AND USING THE SAME

(71) Applicant: Caribou Biosciences, Inc., Berkeley, CA (US)

(72) Inventor: Paul Daniel Donohoue, San Francisco, CA (US)

(73) Assignee: Caribou Biosciences, Inc., Berkeley, CA (US)

(21) Appl. No.: 18/134,406

(22) PCT Filed: Oct. 18, 2021
(86) PCT No.: PCT/US2021/055394

§ 371 Date: Apr. 13, 2023

Related U.S. Application Data
(60) Provisional application No. 63/093,459, filed on Oct. 19, 2020.
(60) Provisional application No. 63/127,648, filed on Dec. 18, 2020.
(60) Provisional application No. 63/229,870, filed on Aug. 5, 2021.

(51) Int. Cl. C12N 15/11; C07K 16/28; C12N 5/0783; C12N 9/22; C12N 15/90

(52) U.S. Cl. CPC C12N 15/11; C07K 16/2878; C12N 5/0636; C12N 9/22; C12N 15/907; C07K 2317/73; C12N 2310/20; C12N 2510/00

(57) ABSTRACT

The present disclosure provides guides for use in Type V CRISPR systems wherein such guides contain ribonucleotide bases and at least one deoxyribonucleotide base. CRISPR Cas12 guides that contain at least one deoxyribonucleotide base, as well as nucleoprotein complexes of Type V CRISPR-Cas12 proteins and such guides, are also described. Also disclosed are methods for making and using the deoxyribonucleotide-containing polynucleotides and guides, and for making and using the nucleoprotein complexes. Also disclosed are methods for engineering cells using Cas12 chRDNA guide/nucleoprotein complexes to produce CAR-expressing cells; and the use of such CAR-expressing cells in adaptive cell therapy.

### CROSS-REFERENCE TO RELATED APPLICATIONS

This application is a U.S. national stage of the International Application Ser. No. PCT/US2021/55394 filed on Oct. 18, 2021, which claims priority to the U.S. Provisional Application Ser. No. 63/229,870 filed 5 Aug. 2021, the U.S. Provisional Application Ser. No. 63/127,648 filed 18 Dec. 2020, and the U.S. Provisional Application Ser. No. 63/093,459 filed 19 Oct. 2020, all incorporated herein by reference.

### STATEMENT REGARDING FEDERALLY SPONSORED RESEARCH OR DEVELOPMENT

Not applicable.

### SEQUENCE LISTING

The instant application contains a Sequence Listing which has been submitted electronically in XML format and is hereby incorporated by reference in its entirety. Said XML copy, created on Sep. 16, 2024, is named CBI039_30_generic_SL.xml and is 1,150,262 bytes in size.

### TECHNICAL FIELD

The present disclosure relates generally to Clustered Regularly Interspaced Short Palindromic Repeats (CRISPR) systems. In particular, the disclosure relates to CRISPR polynucleotides and guides for use in CRISPR-Cas12 systems, wherein the CRISPR polynucleotides and Cas12 guides are designed to include ribonucleotide bases and one or more deoxyribonucleotide bases. The present disclosure further relates to Cas12 guide/nucleoprotein complexes comprising a designed CRISPR Cas12 guide and a CRISPR-Cas12 protein, and to the production of modified cells using such Cas12 guide/nucleoprotein complexes. The disclosure further relates to compositions containing, and to methods for making and using, the CRISPR polynucleotides, Cas12 guides, and Cas12 guide/nucleoprotein complexes. Further still, the present disclosure relates to the production and therapeutic use of cells modified using the Cas12 guide/nucleoprotein complexes of the present disclosure, and for instance, in the generation of chimeric antigen receptor (CAR)-expressing cells for the treatment of cancer.

### BACKGROUND

The Clustered Regularly Interspaced Short Palindromic Repeats (CRISPR) and CRISPR-associated (Cas) protein systems are found in the genomes of many prokaryotes (bacteria and archaea). These systems provide adaptive immunity against foreign invaders (e.g., viruses, bacteriophages) in prokaryotes. In this way, the CRISPR system functions as a type of immune system to help defend prokaryotes against foreign invaders. See, e.g., Barrangou et al. (Science, 2007, 315:1709-1712); Makarova et al. (Nature Reviews Microbiology, 2011, 9:467-477); Garneau et al. (Nature, 2010, 468:67-71); Sapranauskas et al. (Nucleic Acids Research, 2011, 39:9275-9282); Koonin et al. (Curr. Opin. Microbiol., 2017, 37:67-78); Shmakov et al. (Nat. Rev. Microbiol., 2017, 15 (3): 169-182); Makarova et al. (Nat. Rev. Microbiol., 2020, 18:67-83).

There are three main stages in CRISPR-Cas immune systems: (1) acquisition, (2) expression, and (3) interference. Acquisition involves cleaving the genome of invading viruses and plasmids and integrating segments (termed protospacers) of the genomic DNA into the CRISPR locus of the host organism. The segments that are integrated into the host genome are known as spacers, which mediate protection from subsequent attack by the same (or sufficiently related) virus or plasmid. Expression involves transcription of the CRISPR locus and subsequent enzymatic processing to produce short mature CRISPR RNAs, each containing a single spacer sequence. Interference is induced after the CRISPR RNAs associate with Cas proteins to form effector complexes, which are then targeted to complementary protospacers in foreign genetic elements to induce nucleic acid degradation.

Various CRISPR-Cas systems in their native hosts are capable of DNA targeting (Class 1 Type I; Class 2 Types II and V), RNA targeting (Class 2 Type VI), and joint DNA and RNA targeting (Class 1 Type III). See, e.g., Makarova et al. (Nat. Rev. Microbiol., 2015, 13:722-736); Shmakov et al. (Nat. Rev. Microbiol., 2017, 15:169-182); Abudayyeh et al. (Science, 2016, 353:1-17); and Makarova et al. (The CRISPR Journal, 2018, 1:325-336).

Type V systems are classified into several different subtypes, including, e.g., V-A, V-B, V-C, V-D, V-E, V-F, V-G, V-H, V-I, V-J, V-K and V-U. See, e.g., Makarova et al. (Nat. Rev. Microbiol., 2020, 18:67-83) and Pausch et al. (Science, 2020, 369 (6501): 333-337). The V-A subtype encodes the Cas12a protein (formerly known as Cpf1). Cas12a has a RuvC-like nuclease domain that is homologous to the respective domain of Cas9, but lacks the HNH nuclease domain that is present in Cas9 proteins.

Type V systems have been identified in several bacteria, including Parcubacteria bacterium GWC2011_GWC2_44_17 (PbCpf1), Lachnospiraceae bacterium MC2017 (Lb3 Cpf1), Butyrivibrio proteoclasticus (BpCpf1), Peregrinibacteria bacterium GW2011_GWA_33_10 (PeCpf1), Acidaminococcus sp. BV3L6 (AsCpf1), Porphyromonas macacae (PmCpf1), Lachnospiraceae bacterium ND2006 (LbCpf1), Porphyromonas crevioricanis (PcCpf1), Prevotella disiens (PdCpf1), Moraxella bovoculi 237 (MbCpf1), Smithella sp. SC_K08D17 (SsCpf1), Leptospira inadai (LiCpf1), Lachnospiraceae bacterium MA2020 (Lb2Cpf1), Franciscella novicida U112 (FnCpf1), Candidatus methanoplasma termitum (CMtCpf1), and Eubacterium eligens (EeCpf1).

CRISPR-Cas systems provide powerful tools for site-directed genome editing by deleting, inserting, mutating, or substituting specific nucleic acid sequences. The alteration can be gene- or location-specific. Genome editing can use site-directed nucleases, such as Cas proteins and their cognate polynucleotides, to cut a target nucleic acid, thereby generating a site for alteration. In certain cases, the cleavage can introduce a double-strand break (DSB) in a target DNA sequence. DSBs can be repaired, e.g., by non-homologous end joining (NHEJ), microhomology-mediated end joining (MMEJ), or homology-directed repair (HDR). HDR relies on the presence of a template for repair. In some examples of this genome editing, a donor polynucleotide or portion thereof can be inserted into the break.

### SUMMARY OF THE INVENTION

The present disclosure is based on the discovery of new polynucleotides and guides for use in Type V CRISPR-Cas systems, the polynucleotides and guides comprising ribonucleotide bases and one or more deoxyribonucleotide bases. The disclosed guides, when complexed with a Type V CRISPR-Cas protein, such as Cas12a, are capable of robust on-target editing and reduced off-target genome editing.

This genome editing process is particularly useful for generating genetically-modified cells useful in therapeutic applications. For instance, through this genome editing process, immune cells (such as T-cells) can be genetically modified to express a CAR. Such CAR-expressing cells are useful, for instance, in adoptive immunotherapy—where CAR-expressing immune cells, such as T-cells (CAR-T cells), can be infused into patients to target cells expressing a target antigen recognized by the CAR (e.g., a foreign antigen, or a cancer-associated antigen).

Non-limiting embodiments of the disclosure include as follows below.

[1] A CRISPR guide molecule, comprising: a targeting region capable of binding a target nucleic acid sequence; and an activating region capable of forming a nucleoprotein complex with a Cas12 protein, wherein said CRISPR guide molecule comprises ribonucleotide bases and at least one deoxyribonucleotide base.

[2] The CRISPR guide molecule of [1], wherein the CRISPR guide molecule comprises at least one deoxyribonucleotide base in the activating region, the targeting region, or both.

[3] The CRISPR guide molecule of [1], wherein the CRISPR guide molecule further comprises one or more base analogs selected from the group consisting of inosine, deoxy-inosine, deoxy-uracil, xanthosine, C3 spacer, 5-methyl dC, 5-hydroxybutynl-2′-deoxyuridine, 5-nitroindole, 5-methyl iso-deoxycytosine, iso deoxyguanosine, deoxyuridine, and iso deoxycytidine.

[4] The CRISPR guide molecule of [1], wherein the CRISPR guide molecule further comprises one or more abasic sites.

[5] The CRISPR guide molecule of [1], wherein the CRISPR guide molecule is selected from: a CRISPR guide molecule comprising the RNA sequence UAAUUUCUACUCUUGUAGAUGAGUCUCUCAGCUGGUACAC, wherein at least one of the bases in the sequence is replaced with a corresponding deoxyribonucleotide base, and optionally, at least one of the bases in the sequence is replaced with a base analog or abasic site; and a CRISPR guide molecule comprising the RNA sequence UAAUUUCUACUCUUGUAGAUAGUGGGGGUGAAUUCAGUGU, wherein at least one of the bases in the sequence is replaced with a corresponding deoxyribonucleotide base, and optionally, at least one of the bases in the sequence is replaced with a base analog or abasic site.

[6] The CRISPR guide molecule of [5], wherein the amount of deoxyribonucleotide bases in the CRISPR guide molecule, as a percentage of the total size of the CRISPR guide molecule, is 50% or less.

[7] The CRISPR guide molecule of [6], wherein the amount of deoxyribonucleotide bases in the CRISPR guide molecule, as a percentage of the total size of the guide molecule, is 25% or less.

[8] The CRISPR guide molecule of [5], wherein the amount of deoxyribonucleotide bases in the targeting region, as a percentage of the total size of the targeting region, is 25% or less.

[9] The CRISPR guide molecule of [8], wherein the amount of deoxyribonucleotide bases in the targeting region, as a percentage of the total size of the targeting region, is 5% or less.

[10] The CRISPR guide molecule of [5], wherein the amount of deoxyribonucleotide bases in the activating region, as a percentage of the total size of the activating region, is 50% or less.

[11] The CRISPR guide molecule of [10], wherein the amount of deoxyribonucleotide bases in the activating region, as a percentage of the total size of the activating region, is 25% or less.

[12] The CRISPR guide molecule of [5], wherein the CRISPR guide molecule has reduced off-target activity as compared to an RNA-only CRISPR guide molecule that binds to the target nucleic acid sequence and which is capable of forming a nucleoprotein complex with the Cas12 protein.

[13] The CRISPR guide molecule of [5], wherein one or more of positions 1, 3, 7, 10, 12, 14, 15, 16, 17, 18, 19, 21, 28, 29, 30, 31, 32, 34, 35, 36, 37, 38, 39, and 40 in the sequence comprise a deoxyribonucleotide base.

[14] The CRISPR guide molecule of [13], wherein fifteen or less of positions 1, 3, 7, 10, 12, 14, 15, 16, 17, 18, 19, 21, 28, 29, 30, 31, 32, 34, 35, 36, 37, 38, 39, and 40 in the sequence comprise deoxyribonucleotide bases.

[15] The CRISPR guide molecule of [14], wherein twelve or less of positions 1, 3, 7, 10, 12, 14, 15, 16, 17, 18, 19, 21, 28, 29, 30, 31, 32, 34, 35, 36, 37, 38, 39, and 40 in the sequence comprise deoxyribonucleotide bases.

[16] The CRISPR guide molecule of [2], wherein the CRISPR guide molecule contains an activating region comprising the RNA sequence UAAUUUCUACUCUUGUAGAU, wherein at least one of the bases in the sequence is replaced with a corresponding deoxyribonucleotide base, and optionally, at least one of the bases in the sequence is replaced with a base analog or abasic site.

[17] The CRISPR guide molecule of [16], wherein one or more of positions 1, 3, 7, 10, 12, 14, 15, 16, 17, 18, and 19 in the sequence comprise a deoxyribonucleotide base.

[18] The CRISPR guide molecule of [17], wherein ten or less of positions 1, 3, 7, 10, 12, 14, 15, 16, 17, 18, and 19 in the sequence comprise deoxyribonucleotide bases.

[19] The CRISPR guide molecule of [18], wherein eight or less of positions 1, 3, 7, 10, 12, 14, 15, 16, 17, 18, and 19 in the sequence comprise deoxyribonucleotide bases.

[20] The CRISPR guide molecule of [2], wherein the CRISPR guide molecule contains a targeting region comprising the RNA sequence GAGUCUCUCAGCUGGUACAC, wherein at least one of the bases in the sequence is replaced with a corresponding deoxyribonucleotide base, and optionally, at least one of the bases in the sequence is replaced with a base analog or abasic site.

[21] The CRISPR guide molecule of [20], wherein one or more of positions 1, 8, 9, 10, 11, 12, 14, 15, 16, 17, 18, 19, and 20 in the sequence comprise a deoxyribonucleotide base.

[22] The CRISPR guide molecule of [21], wherein five or less of positions 1, 8, 9, 10, 11, 12, 14, 15, 16, 17, 18, 19, and 20 in the sequence comprise deoxyribonucleotide bases.

[23] The CRISPR guide molecule of [22], wherein three or less of positions 1, 8, 9, 10, 11, 12, 14, 15, 16, 17, 18, 19, and 20 in the sequence comprise deoxyribonucleotide bases.

[24] The CRISPR guide molecule of [2], wherein the CRISPR guide molecule contains a targeting region comprising the RNA sequence AGUGGGGGUGAAUUCAGUGU, wherein at least one of the bases in the sequence is replaced with a corresponding deoxyribonucleotide base, and optionally, at least one of the bases in the sequence is replaced with a base analog or abasic site.

[25] The CRISPR guide molecule of [24], wherein one or more of positions 1, 8, 9, 10, 11, 12, 14, 15, 16, 17, 18, 19, and 20 in the sequence comprise a deoxyribonucleotide base.

[26] The CRISPR guide molecule of [25], wherein five or less of positions 1, 8, 9, 10, 11, 12, 14, 15, 16, 17, 18, 19, and 20 in the sequence comprise deoxyribonucleotide bases.

[27] The CRISPR guide molecule of [26], wherein three or less of positions 1, 8, 9, 10, 11, 12, 14, 15, 16, 17, 18, 19, and 20 in the sequence comprise deoxyribonucleotide bases.

[28] The CRISPR guide molecule of [5], wherein the CRISPR guide molecule comprises the sequence TAAUUUCUACUCUTGUAGAUGAGUCUCUCAGCUGGUACAC, wherein positions 2, 4, 5, 6, 8, 9, 11, 13, 16, 17, 18, 20, 22, 23, 24, 25, 26, 27, 28, 29, 30, 32, 33, 34, 35, 36, 37, 38, and 39 in the sequence comprise ribonucleotide bases, and wherein positions 1, 3, 7, 10, 12, 14, 15, 19, 21, 31, and 40 in the sequence comprise deoxyribonucleotide bases.

[29] The CRISPR guide molecule of [5], wherein the CRISPR guide molecule comprises the sequence TAAUUUCUACUCUTGUAGAUAGUGGGGGUGAAUUCAGUGT, wherein positions 2, 4, 5, 6, 8, 9, 11, 13, 16, 17, 18, 20, 22, 23, 24, 25, 26, 27, 28, 29, 30, 32, 33, 34, 35, 36, 37, 38, and 39 in the sequence comprise ribonucleotide bases, and wherein positions 1, 3, 7, 10, 12, 14, 15, 19, 21, 31, and 40 in the sequence comprise deoxyribonucleotide bases.

[30] The CRISPR guide molecule of [2], wherein the activating region is 20 bases in length, and wherein one or more of positions 1, 3, 7, 10, 12, 14, 15, 16, 17, 18, and 19 in the 20-nucleotide activating region sequence comprise a deoxyribonucleotide base.

[31] The CRISPR guide molecule of [30], wherein ten or less of positions 1, 3, 7, 10, 12, 14, 15, 16, 17, 18, and 19 in the sequence comprise deoxyribonucleotide bases.

[32] The CRISPR guide molecule of [31], wherein eight or less of positions 1, 3, 7, 10, 12, 14, 15, 16, 17, 18, and 19 in the sequence comprise deoxyribonucleotide bases.

[33] The CRISPR guide molecule of [2], wherein the targeting region is 20 bases in length, and wherein one or more of positions 1, 8, 9, 10, 11, 12, 14, 15, 16, 17, 18, 19, and 20 in the 20-nucleotide targeting region sequence comprises a deoxyribonucleotide base.

[34] The CRISPR guide molecule of [33], wherein five or less of positions 1, 8, 9, 10, 11, 12, 14, 15, 16, 17, 18, 19, and 20 in the sequence comprise deoxyribonucleotide bases.

[35] The CRISPR guide molecule of [34], wherein three or less of positions 1, 8, 9, 10, 11, 12, 14, 15, 16, 17, 18, 19, and 20 in the sequence comprise deoxyribonucleotide bases.

[36] A CRISPR nucleic acid/protein composition, comprising: the CRISPR guide molecule of any one of [1]-[35]; and a Cas12 protein.

[37] The CRISPR nucleic acid/protein composition of [36], wherein the CRISPR guide molecule is in a complex with the Cas12 protein.

[38] The CRISPR nucleic acid/protein composition of [36], wherein the Cas12 protein is a Cas12a protein.

[39] The CRISPR nucleic acid/protein composition of any one of [36]-[38], wherein the Cas12 protein comprises, at the C-terminus, a linker- and NLS-containing sequence having at least 80% sequence identity to an amino acid sequence selected from the group consisting of SEQ ID NOs:479-490.

[40] The CRISPR nucleic acid/protein composition of [39], wherein the linker- and NLS-containing sequence comprises an amino acid sequence selected from the group consisting of SEQ ID NOs:483, 485, 487 and 489.

[41] A cell, comprising the CRISPR guide molecule of any one of [1]-[35].

[42] The cell of [41], further comprising a Cas12 protein.

[43] The cell of [42], wherein the Cas12 protein is a Cas12a protein.

[44] The cell of [42] or [43], wherein the Cas12 protein comprises, at the C-terminus, a linker- and NLS-containing sequence having at least 80% sequence identity to an amino acid sequence selected from the group consisting of SEQ ID NOs:479-490.

[45] The cell of [44], wherein the linker- and NLS-containing sequence comprises an amino acid sequence selected from the group consisting of SEQ ID NOs:483, 485, 487 and 489.

[46] The cell of any one of [42]-[45], wherein the CRISPR guide molecule is in a complex with the Cas12 protein.

[47] The cell of any one of [41]-[46], wherein the cell is a prokaryotic cell or a eukaryotic cell.

[48] The cell of [47], wherein the cell is a eukaryotic cell selected from the group consisting of a single-cell eukaryotic organism, a cell of a eukaryotic organism, a protozoal cell, a cell from a plant, an algal cell, a fungal cell, an animal cell, a cell from an invertebrate animal, a cell from a vertebrate animal, a cell from a mammal, a stem cell, and a progenitor cell.

[49] The cell of [48], wherein the cell is a lymphocyte, a chimeric antigen receptor (CAR) T cell, a T cell receptor (TCR) cell, a TCR-engineered CAR-T cell, a tumor infiltrating lymphocyte (TIL), a CAR TIL, a dendritic cell (DC), a CAR-DC, a macrophage, a CAR-macrophage (CAR-M), a natural killer (NK) cell, or a CAR-NK cell.

[50] The cell of [49], wherein the cell is a CAR-T cell.

[51] The cell of any one of [41]-[50], further comprising a donor polynucleotide.

[52] A method of cleaving a target nucleic acid sequence, the method comprising: contacting a first target nucleic acid sequence with a nucleoprotein complex comprising a catalytically active Cas12 protein and a first CRISPR guide molecule, wherein the first CRISPR guide molecule comprises a CRISPR guide molecule of any one of [1]-[35], wherein the targeting region of the first CRISPR guide molecule is capable of hybridizing to the first target nucleic acid sequence, and the nucleoprotein complex is capable of cleaving the first target nucleic acid sequence.

[53] The method of [52], further comprising providing a donor polynucleotide.

[54] The method of [53], wherein the target nucleic acid sequence is cleaved to provide a cleavage site, and the method further comprises modifying the target nucleic acid sequence.

[55] The method of [54], wherein the modifying comprises inserting at least a portion of the donor polynucleotide at the cleavage site.

[56] The method of [54], wherein the modifying comprises deleting one or more nucleotides at the cleavage site.

[57] The method of [55], wherein the target nucleic acid sequence is in a cell.

[58] The method of [57], wherein the cell comprises a eukaryotic cell.

[59] The method of [58], wherein the donor polynucleotide comprises a CAR expression vector.

[60] The method of [59], further comprising introducing the CAR expression vector into the cell using a viral vector.

[61] The method of [60], wherein said introducing comprises transduction.

[62] The method of any one of [57]-[61], wherein the resulting cell comprises a lymphocyte, a CAR-T cell, a TCR cell, a TCR-engineered CAR-T cell, a TIL, a CAR TIL, a dendritic cell, a CAR-DC, a macrophage, a CAR-M, an NK cell, or a CAR-NK cell.

[63] The method of any one of [52]-[62], wherein the first target nucleic acid sequence is within a target gene encoding a protein selected from the group consisting of a TRAC; a TRBV; a beta-2 microglobulin (B2M); a PD1; a PD-L1; a CTLA-4; a LAG-3; a TIGIT; a TIM3; a HLA-E; a HLA-A; a HLA-B; a HLA-C; a HLA-DRA; a ADAM17; a BTLA; a CD160; a SIGLEC10; a 2B4; a LAIR1; a CD52; a CD96; a VSIR; a VISTA; a KIR2DL1; a KIR2DL2; a KIR2DL3; a CEACAM1; a CBLB; a CISH; a IL-1R8; a AHR; a Adenosine 2A receptor; a GMCSF; a VISTA; a CII2A; and a NKG2A.

[64] The method of any one of [59]-[61], wherein the CAR expression vector encodes a CAR comprising an extracellular ligand-binding domain.

[65] The method of [64], wherein the CAR expression vector further encodes a hinge region, a transmembrane region, and one or more intracellular signaling regions.

[66] The method of [64] or [65], wherein the extracellular ligand-binding domain comprises an immunoglobulin single-chain variable fragment (scFv).

[67] The method of [66], wherein the scFv is capable of binding to a cellular target selected from the group consisting of a CD37, a CD38, a CD47, a CD73, a CD4, a CS1, a PD-L1, a NGFR, a ENPP3, a PSCA, a CD79B, a TACI, a VEGFR2, a B7-H3, a B7-H6, a B-cell maturation antigen (BCMA), a CD123, a CD138, a CD171/L1CAM, a CD19, a CD20, a CD22, a CD30, a CD33, a CD70, a CD371, a CEA, a Claudin 18.1, a Claudin 18.2, a CSPG4, a EFGRvIII, a EpCAM, a EphA2, a Epidermal growth factor receptor, a ErbB, a ErbB2 (HER2), a FAP, a FRα, a GD2, a GD3, a Glypican 3, a IL-11Rα, a IL-13Rα2, a IL13 receptor alpha, a LewisY/LeY, a Mesothelin, a MUC1, a MUC16, a NKG2D ligands, a PD1, a PSMA, a ROR-1, a SLAMF7, a TAG72, a ULBP and a MICA/B proteins, a VEGF2, and a WT1.

[68] The method of [67], wherein the scFv is capable of binding to a cellular target selected from the group consisting of a BCMA, a CD19, a CD20, a CD22, a CD47, a CD371, a ROR-1, a EphA2, a MUC16, a Glypican 3, a PSCA, and a Claudin 18.2.

[69] The method of [68], wherein the scFv is capable of binding to a BCMA.

[70] The method of [68], wherein the scFv is capable of binding to a CD371.

[71] The method of any one of [57]-[62] and [64]-[70], wherein said method further comprises contacting a second target nucleic acid sequence in the cell with a nucleoprotein complex comprising a catalytically active Cas12 protein and a second CRISPR guide molecule, wherein the second CRISPR guide molecule comprises a CRISPR guide molecule of any one of [1]-[35] that is capable of binding to a different target nucleic acid sequence than the first CRISPR guide molecule, wherein the targeting region of the second CRISPR guide molecule is capable of hybridizing to the second target nucleic acid sequence, and the nucleoprotein complex is capable of cleaving the second target nucleic acid sequence.

[72] The method of [71], wherein said first and second target nucleic acid sequences are each independently within a target gene encoding a protein selected from the group consisting of a TRAC; a TRBV protein; a beta-2 microglobulin (B2M); a PD1; a PD-L1; a CTLA-4; a LAG-3; a TIGIT; a TIM3; a HLA-E; a HLA-A; a HLA-B; a HLA-C; a HLA-DRA; an ADAM17; a BTLA; a CD160; a SIGLEC10; a 2B4; a LAIR1; a CD52; a CD96; a VSIR; a VISTA; a KIR2DL1; a KIR2DL2; a KIR2DL3; a CEACAM1; a CBLB; a CISH; an IL-1R8; an AHR; an Adenosine 2A receptor; a GMCSF; a VISTA; a CII2A; and a NKG2A.

[73] The method of [71] or [72], wherein the donor polynucleotide comprises a CAR expression vector, wherein the CAR comprises an extracellular ligand-binding domain, and wherein the extracellular ligand-binding domain comprises an scFv.

[74] The method of [73], wherein the scFv is capable of binding to a BCMA.

[75] The method of [73], wherein the scFv is capable of binding to a CD371.

[76] The method of [72], wherein said first target nucleic acid sequence is within a gene encoding a TRAC protein, and wherein said second target nucleic acid sequence is within a gene encoding a PD1 protein.

[77] The method of [72], wherein said first target nucleic acid sequence is within a gene encoding a TRAC protein, and wherein said second target nucleic acid sequence is within a gene encoding a B2M protein.

[78] The method of [77], further comprising providing a second donor polynucleotide comprising a B2M-HLA-E fusion construct to the cell, wherein at least a portion of the second donor polynucleotide comprising the B2M-HLA-E fusion construct is inserted at the cleavage site of the second target nucleic acid sequence; and the B2M-HLA-E fusion construct encodes a fusion protein comprising, from the N- to C-terminus, a B2M secretion signal, a HLA-G peptide signal sequence, a first linker sequence, a B2M sequence, a second linker sequence, and a HLA-E sequence.

[79] The method of [69] or [74], wherein the anti-BCMA scFv comprises a heavy chain variable region (VH) comprising the amino acid sequence of SEQ ID NO: 474; and a light chain variable region (VL) comprising the amino acid sequence of SEQ ID NO: 475.

[80] The method of [79], wherein the scFv further comprises a linker between the VH and the VL.

[81] The method of [80], wherein the linker comprises the amino acid sequence of SEQ ID NO: 476.

[82] The method of [81], wherein said scFv comprises the amino acid sequence of SEQ ID NO: 477.

[83] The method of [64] or [73], wherein the CAR comprises: an scFv comprising a VH and a VL; a transmembrane domain; a co-stimulatory domain; and an activating domain.

[84] The method of [83], wherein the transmembrane domain is a transmembrane domain derived from a T cell receptor α chain, a T cell receptor β chain, a CD3ζ chain, a CD28, a CD3E, a CD45, a CD4, a CD5, a CD8, a CD9, a CD16, a CD22, a CD33, a CD37, a CD64, a CD80, a CD86, a CD134, a CD137, an ICOS, a CD154, or a GITR.

[85] The method of [84], wherein the transmembrane domain comprises a transmembrane domain derived from a CD8.

[86] The method of [83], wherein the co-stimulatory domain is a co-stimulatory domain derived from a CD28, a 4-1BB, a GITR, an ICOS-1, a CD27, an OX-40, or a DAP10.

[87] The method of [86], wherein the co-stimulatory domain comprises a 4-1BB co-stimulatory domain.

[88] The method of [83], wherein the activating domain comprises a CD3ζ activating domain.

[89] The method of [83], wherein the transmembrane domain comprises a transmembrane domain derived from a CD8, the co-stimulatory domain comprises a 4-1BB co-stimulatory domain, and the activating domain comprises a CD3ζ activating domain.

[90] The method of [83], wherein the VH comprises the amino acid sequence of SEQ ID NO: 474, and the VL comprises the amino acid sequence of SEQ ID NO: 475.

[91] The method of any one of [59]-[62], [64]-[70], [73]-[75] and [78]-[90], wherein the polynucleotide sequence encoding the CAR in said CAR expression vector has a leader sequence at the 5′ end.

[92] The method of [91], wherein the leader sequence comprises the nucleic acid sequence of SEQ ID NO: 478.

[93] The method of [91], wherein the CAR expression vector comprises a promoter.

[94] The method of [93], wherein the promoter comprises an MND promoter.

[95] The method of any one of [52]-[94], wherein the Cas12 protein comprises, at the C-terminus, a linker- and NLS-containing sequence having at least 80% sequence identity to an amino acid sequence selected from the group consisting of SEQ ID NOs:479-490.

[96] The method of [95], wherein the linker- and NLS-containing sequence comprises an amino acid sequence selected from the group consisting of SEQ ID NOs:483, 485, 487, and 489.

[97] A cell produced by the method of any one of [57]-[96].

[98] A CAR-T cell produced by the method of any one of [59]-[96].

[99] The CAR-T cell of [98], wherein said CAR-T cell is an allogeneic CAR-T cell.

[100] The CAR-T cell of [98], wherein said CAR-T cell is an autologous CAR-T cell.

[101] A method of producing a CAR-T cell, comprising performing the method of any one of [59]-[96] using a T-lymphocyte as the cell.

[102] A method of adoptive cell therapy, comprising administering to a subject in need thereof a cell produced by the method of any one of [57]-[96].

[103] A method of adoptive cell therapy, comprising administering to a subject in need thereof a CAR-T cell produced by the method of any one of [59]-[96].

[104] A method of killing BCMA-positive cancer cells, wherein said method comprises contacting BCMA-positive cancer cells with a CAR-T cell produced by the method of any one of [69], [74] and [90].

[105] The method of [104], wherein the BCMA-positive cancer cells comprise multiple myeloma cancer cells.

[106] The method of [105], wherein the multiple myeloma cancer cells comprise human cells.

[107] The method of [104], wherein the contacting is intra-tumoral.

[108] A method for producing a CAR-expressing cell, said method comprising: contacting a first target nucleic acid sequence in a cell with a nucleoprotein complex comprising a catalytically active Cas12 protein and a first CRISPR guide molecule, wherein the first CRISPR guide molecule comprises a CRISPR guide molecule of any one of [1]-[35], wherein the targeting region of the first CRISPR guide molecule is capable of hybridizing to the first target nucleic acid sequence, and the nucleoprotein complex is capable of cleaving the first target nucleic acid sequence; contacting a second target nucleic acid sequence in the cell with a nucleoprotein complex comprising a catalytically active Cas12 protein and a second CRISPR guide molecule, wherein the second CRISPR guide molecule comprises a CRISPR guide molecule of any one of [1]-[35] that is capable of binding to a different target nucleic acid sequence than the first CRISPR guide molecule, wherein the targeting region of the second CRISPR guide molecule is capable of hybridizing to the second target nucleic acid sequence, and the nucleoprotein complex is capable of cleaving the second target nucleic acid sequence; and providing a donor polynucleotide comprising a CAR expression vector to said cell, wherein at least a portion of the donor polynucleotide containing said CAR expression vector is capable of being inserted at the cleavage site in said first target nucleic acid sequence, and wherein the CAR comprises an extracellular ligand-binding domain.

[109] The method of [108], wherein the donor polynucleotide comprising the CAR expression vector is introduced into the cell using a viral vector.

[110] The method of [108], wherein the CAR expression vector further encodes a hinge region, a transmembrane region, and one or more intracellular signaling regions.

[111] The method of any one of [108]-[110], wherein said first target nucleic acid sequence is within a gene encoding a TRAC protein, and wherein said second target nucleic acid sequence is within a gene encoding a PD1 protein.

[112] The method of any one of [108]-[110], wherein said first target nucleic acid sequence is within a gene encoding a TRAC protein, and wherein said second target nucleic acid sequence is within a gene encoding a B2M protein.

[113] The method of any of [108]-[112], wherein the extracellular ligand-binding domain comprises an immunoglobulin single-chain variable fragment (scFv).

[114] The method of [113], wherein the scFv is capable of binding a BCMA.

[115] The method of [113], wherein the scFv is capable of binding a CD371.

[116] The method of [114], wherein the anti-BCMA scFv comprises a heavy chain variable region (VH) comprising the amino acid sequence of SEQ ID NO: 474, and a light chain variable region (VL) comprising the amino acid sequence of SEQ ID NO: 475.

[117] The method of [116], wherein the scFv further comprises a linker between the VH and the VL.

[118] The method of [117], wherein the linker comprises the amino acid sequence of SEQ ID NO: 476.

[119] The method of [114], wherein said scFv comprises the amino acid sequence of SEQ ID NO: 477.

[120] The method of any one of [108]-[119], further comprising providing a second donor polynucleotide comprising a B2M-HLA-E fusion construct to said cell, wherein at least a portion of the second donor polynucleotide comprising the B2M-HLA-E fusion construct is capable of being inserted at the cleavage site of the second target nucleic acid sequence; and the B2M-HLA-E fusion construct encodes a fusion protein comprising, from the N- to C-terminus, a B2M secretion signal, a HLA-G peptide signal sequence, a first linker sequence, a B2M sequence, a second linker sequence, and a HLA-E sequence.

[121] The method of any one of [108]-[120], wherein the CAR comprises: an scFv comprising a VH and a VL; a transmembrane domain; a co-stimulatory domain; and an activating domain.

[122] The method of [121], wherein the transmembrane domain comprises a transmembrane domain derived from a T cell receptor α chain, a T cell receptor R chain, a CD3ζ chain, a CD28, a CD3E, a CD45, a CD4, a CD5, a CD8, a CD9, a CD16, a CD22, a CD33, a CD37, a CD64, a CD80, a CD86, a CD134, a CD137, an ICOS, a CD154, or aG ITR.

[123] The method of [122], wherein the transmembrane domain comprises a transmembrane domain derived from a CD8.

[124] The method of [121], wherein the co-stimulatory domain comprises a co-stimulatory domain derived from a CD28, a 4-1BB, a GITR, a ICOS-1, a CD27, a OX-40, or a DAP10.

[125] The method of [124], wherein the co-stimulatory domain comprises a 4-1BB co-stimulatory domain.

[126] The method of [121], wherein the activating domain comprises a CD3ζ activating domain.

[127] The method of [121], wherein the transmembrane domain comprises a transmembrane domain derived from a CD8, the co-stimulatory domain comprises a 4-1BB co-stimulatory domain, and the activating domain comprises a CD3ζ activating domain.

[128] The method of any one of [108]-[127], wherein the CAR-expressing cell is a CAR-T cell.

[129] The method of [128], wherein the CAR-T cell is an allogeneic CAR-T cell.

[130] The method of [128], wherein the CAR-T cell is an autologous CAR-T cell.

[131] The method of any one of [108]-[130], wherein the Cas12 protein complexed with the first CRISPR guide molecule and/or the Cas12 protein complexed with the second CRISPR guide molecule comprises, at the C-terminus, a linker- and NLS-containing sequence having at least 80% sequence identity to an amino acid sequence selected from the group consisting of SEQ ID NOs:479-490.

[132] The method of [131], wherein the Cas12 protein complexed with the first CRISPR guide molecule and/or the Cas12 protein complexed with the second CRISPR guide molecule comprises an amino acid sequence selected from the group consisting of SEQ ID NOs:483, 485, 487, and 489.

[133] The method of [78], wherein the second donor polynucleotide further comprises a P2A sequence at the N-terminus of the B2M-HLA-E fusion construct.

[134] The method of [120], wherein the second donor polynucleotide further comprises a P2A sequence at the N-terminus of the B2M-HLA-E fusion construct sequence.

In some embodiments, the invention is a CRISPR guide molecule, comprising a targeting region capable of binding a target nucleic acid sequence and an activating region comprising the RNA sequence UAAUUUCUACUCUUGUAGAU including at least one deoxyribonucleotide in place of a ribonucleotide, wherein the activating region is capable of forming a nucleoprotein complex with a Cas12 protein. In some embodiments, one or more (e.g., ten or less) of positions 1, 3, 7, 10, 12, 14, 15, 16, 17, 18, and 19 in the activating region comprise a deoxyribonucleotide base. In some embodiments, the molecule comprises one or more chemical modifications selected from the group consisting of base modifications including inosine, deoxy-inosine, deoxy-uracil, xanthosine, C3 spacer, 5-methyl dC, 5-hydroxybutynl-2′-deoxyuridine, 5-nitroindole, 5-methyl iso-deoxycytosine, iso deoxyguanosine, deoxyuridine, iso-deoxycytidine, and an abasic site, and backbone modification, including a phosphorothioate modification.

In some embodiments, the targeting region of the CRISPR guide targets the B2M gene and comprises the RNA sequence AGUGGGGGUGAAUUCAGUGU, wherein optionally, at least one of the bases in the sequence is replaced with a base analog or an abasic site. In some embodiments, one or more (e.g., five or less) of positions 1, 8, 9, 10, 11, 12, 14, 15, 16, 17, 18, 19, and 20 in the targeting region comprise a deoxyribonucleotide base. In some embodiments, the targeting region is capable of hybridizing to a sequence selected from SEQ ID NOs: 51-133. In some embodiments, the CRISPR guide comprises a sequence selected from SEQ ID NOs: 212-231, 275-315, and 331-350. In some embodiments, the CRISPR guide comprises the sequence of SEQ ID NO: 416.

In some embodiments, the targeting region of the CRISPR guide targets the TRAC gene and comprises the RNA sequence GAGUCUCUCAGCUGGUACAC, wherein optionally, at least one of the bases in the sequence is replaced with a base analog or an abasic site. In some embodiments, one or more (e.g., five or less) of positions 1, 8, 9, 10, 11, 12, 14, 15, 16, 17, 18, 19, and 20 in the targeting region comprise a deoxyribonucleotide base. In some embodiments, the targeting region is capable of hybridizing to a sequence selected from SEQ ID NOs: 15-20. In some embodiments, the CRISPR guide comprises a sequence selected from SEQ ID NOs: 233-252, 317-329, 491-492, and 508. In some embodiments, the CRISPR guide molecule further comprises a chemical modification and comprises a sequence selected from SEQ ID Nos: 512-517. In some embodiments, the CRISPR guide molecule comprises the sequence of SEQ ID NO: 415.

In some embodiments, the targeting region targets the CISH gene and is capable of hybridizing to a sequence selected from SEQ ID NOs: 157-165. In some embodiments, the CRISPR guide comprises the sequence selected from SEQ ID NO: 509, and 519-529.

In some embodiments, the targeting region targets the PDCD1 gene and is capable of hybridizing to a sequence selected from SEQ ID NOs: 135-155.

In some embodiments, the targeting region targets the CBLB gene and is capable of hybridizing to a sequence selected from SEQ ID NOs: 167-189. In some embodiments, the CRISPR guide comprises the sequence of SEQ ID NO: 510.

In some embodiments, the invention is a CRISPR nucleic acid/protein composition, comprising the CRISPR guide molecule described above a Cas12 protein. In some embodiments, the Cas12 protein is a Cas12a protein comprising at the C-terminus, a linker- and a nuclear localization signal (NLS)-containing sequence having at least 80% sequence identity to an amino acid sequence selected from the group consisting of SEQ ID NOs: 479-490.

In some embodiments, the invention is a cell, comprising the CRISPR nucleic acid/protein composition described above, wherein the cell is a lymphocyte, a chimeric antigen receptor (CAR) T cell, a T cell receptor (TCR) cell, a TCR-engineered CAR-T cell, a tumor infiltrating lymphocyte (TIL), a CAR TIL, a dendritic cell (DC), a CAR-DC, a macrophage, a CAR-macrophage (CAR-M), a natural killer (NK) cell, an induced pluripotent stem cell (iPSC), a cell differentiated from an iPSC cell, or a CAR-NK cell.

In some embodiments, the invention is a method for producing a chimeric antigen receptor (CAR)-expressing cell, said method comprising contacting a first target nucleic acid comprising a TRAC sequence in a cell with a nucleoprotein complex comprising a catalytically active Cas12 protein and a first CRISPR guide molecule having a targeting region capable of binding the first target nucleic acid sequence; and an activating region capable of forming a nucleoprotein complex with the Cas12 protein, wherein said CRISPR guide molecule comprises ribonucleotide bases and at least one deoxyribonucleotide base in the activating region, the targeting region, or both, and the nucleoprotein complex is capable of cleaving the first target nucleic acid sequence; contacting a second target nucleic acid sequence comprising a B2M sequence in the same cell with a nucleoprotein complex comprising a catalytically active Cas12 protein and a second CRISPR guide molecule having a targeting region capable of binding the second target nucleic acid sequence; and an activating region capable of forming a nucleoprotein complex with the Cas12 protein, wherein said CRISPR guide molecule comprises ribonucleotide bases and at least one deoxyribonucleotide base in the activating region, the targeting region, or both, and the nucleoprotein complex is capable of cleaving the second target nucleic acid sequence; providing a first donor polynucleotide encoding a CAR comprising an scFv, a transmembrane domain, a co-stimulatory domain, and an activating domain, wherein the CAR is capable of being inserted into the cleavage site in the first target nucleic acid sequence; providing a second donor polynucleotide encoding a B2M-HLA-E fusion construct comprising a B2M secretion signal, a HLA-G peptide signal sequence, a first linker sequence, a B2M sequence, a second linker sequence, and a HLA-E sequence, wherein the B2M-HLA-E fusion construct is capable of being inserted into the cleavage site in the second target nucleic acid sequence; cleaving the first target nucleic acid sequence and inserting at least a portion of the first donor polynucleotide into the cleavage site; and cleaving the second target nucleic acid sequence and inserting at least a portion of the second donor polynucleotide into the cleavage site. In some embodiments, the second donor polynucleotide further comprises a P2A sequence at 5′-end of the B2M-HLA-E fusion construct. In some embodiments, the first donor polynucleotide comprises the SEQ ID NO: 413. In some embodiments, the second donor polynucleotide comprises the SEQ ID NO: 414.

In some embodiments, the scFv in the CAR is capable of binding to a cellular target selected from the group consisting of a CD37, a CD38, a CD47, a CD73, a CD4, a CS1, a PD-L1, a NGFR, a ENPP3, a PSCA, a CD79B, a TACI, a VEGFR2, a B7-H3, a B7-H6, a B-cell maturation antigen (BCMA), a CD123, a CD138, a CD171/LICAM, a CD19, a CD20, a CD22, a CD30, a CD33, a CD70, a CD371, a CEA, a Claudin 18.1, a Claudin 18.2, a CSPG4, a EFGRvIII, a EpCAM, a EphA2, a Epidermal growth factor receptor, a ErbB, a ErbB2 (HER2), a FAP, a FRα, a GD2, a GD3, a Glypican 3, a IL-11Rα, a IL-13Rα2, a IL13 receptor alpha, a Lewis Y/LeY, a Mesothelin, a MUC1, a MUC16, a NKG2D ligands, a PD1, a PSMA, a ROR-1, a SLAMF7, a TAG72, a ULBP and a MICA/B proteins, a VEGF2, and a WT1. In some embodiments, the scFv is capable of binding BCMA and comprises a first variable region comprising the amino acid sequence of SEQ ID NO: 474, a second variable region comprising the amino acid sequence of SEQ ID NO: 475, and a linker between the first and second variable regions comprising the amino acid sequence of SEQ ID NO: 476. In some embodiments, the scFv comprises the amino acid sequence of SEQ ID NO: 477.

In some embodiments, the transmembrane domain of the CAR is derived from a T cell receptor α chain, a T cell receptor β chain, a CD35 chain, a CD28, a CD38, a CD45, a CD4, a CD5, a CD8, a CD9, a CD16, a CD22, a CD33, a CD37, a CD64, a CD80, a CD86, a CD134, a CD137, an ICOS, a CD154, or a GITR. In some embodiments, the co-stimulatory domain of the CAR is derived from a CD28, a 4-1BB, a GITR, an ICOS-1, a CD27, an OX-40, or a DAP10. In some embodiments, the CAR comprises a transmembrane domain derived from a CD8, a 4-1BB co-stimulatory domain, and a CD3 activating domain. In some embodiments, the vector containing the CAR sequence comprises a leader sequence having the nucleic acid sequence of SEQ ID NO: 478.

In some embodiments, the catalytically active Cas12 protein used in the method comprises, at the C-terminus, a linker, and a nuclear localization signal (NLS)-containing sequence having at least 80% sequence identity to an amino acid sequence selected from the group consisting of SEQ ID NOs: 479-490.

In some embodiments, the method further comprises contacting a third target nucleic acid sequence in the same cell with a nucleoprotein complex comprising a catalytically active Cas12 protein and a third CRISPR guide molecule having a targeting region capable of binding the third target nucleic acid sequence; and an activating region capable of forming a nucleoprotein complex with the Cas12 protein, wherein said CRISPR guide molecule comprises ribonucleotide bases and at least one deoxyribonucleotide base in the activating region, the targeting region, or both, and the nucleoprotein complex is capable of cleaving the third target nucleic acid sequence; cleaving the third target nucleic acid sequence and deleting one or more nucleotides from the third target nucleic acid sequence at the cleavage site, wherein the third target nucleic acid sequence is selected from a PDCD gene, a CISH gene, and a CBLB gene.

In some embodiments, the CAR-expressing cell is an allogeneic or autologous CAR-T cell produced from a T-lymphocyte.

In some embodiments, the invention is a CAR-expressing cell produced by the method described above, wherein the cell is selected from a lymphocyte, a CAR-T cell, a TCR cell, a TCR-engineered CAR-T cell, a TIL, a CAR TIL, a dendritic cell, a CAR-DC, a macrophage, a CAR-M, an iPSC cell, a cell differentiated from an iPSC cell, an NK cell, or a CAR-NK cell.

In some embodiments, the invention is a method of adaptive cell therapy, comprising administering to a subject in need thereof the CAR-expressing cell described above. In some embodiments, the adaptive cell therapy comprises killing BCMA-positive cancer cells, e.g., multiple myeloma cancer cells.

### INCORPORATION BY REFERENCE

All patents, publications, and patent applications cited in the present specification are herein incorporated by reference as if each individual patent, publication, or patent application was specifically and individually indicated to be incorporated by reference in its entirety for all purposes.

### BRIEF DESCRIPTION OF THE FIGURES

The features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying figures. The figures are not proportionally rendered, nor are they to scale. The locations of indicators are approximate.

FIG. 1A, FIG. 1B, and FIG. 1C illustrate examples of Type V CRISPR-Cas12a guide RNAs.

FIG. 2 illustrates a Cas12a chRDNA guide/nucleoprotein complex cleavage of a target polynucleotide.

FIG. 3A-FIG. 3I illustrate various canonical and non-canonical nucleotides for use in Cas12 chRDNA guides.

FIG. 4 illustrates a Cas12a chRDNA guide/nucleoprotein complex cleavage of a target polynucleotide.

FIG. 5 illustrates a Cas12a crRNA guide.

FIG. 6 illustrates a Cas12a chRDNA guide comprising DNA bases in the activating region and target binding sequence.

FIG. 7 illustrates a Cas12a chRDNA guide comprising DNA bases and chemically modified nucleic acids in the activating region and target binding sequence.

FIG. 8 illustrates the formation of a Cas12 chRDNA guide/nucleoprotein complex and binding of a target polynucleotide.

FIG. 9 illustrates the generation of insertion or deletions (indels) in a target polynucleotide by a Cas12 chRDNA guide/nucleoprotein complex.

FIG. 10 illustrates the insertion of a donor polynucleotide sequence in a target polynucleotide by a Cas12 chRDNA guide/nucleoprotein complex.

FIG. 11 illustrates nicking of a target polynucleotide by a Cas12 chRDNA guide/nucleoprotein complex.

FIG. 12 illustrates the tandem nicking of a target polynucleotide with two Cas12 chRDNA guide/nucleoprotein complexes and insertion of a donor polynucleotide sequence in a target polynucleotide.

FIG. 13 illustrates the average normalized editing rates of Cas12a chRDNA guide/nucleoprotein complexes with an individual DNA base in the target binding sequence.

FIG. 14 illustrates the normalized editing rates of Cas12a chRDNA guide/nucleoprotein complexes with an individual DNA base in the activating region.

FIG. 15A and FIG. 15B illustrate the phenotypic and cytotoxic profile of CAR-T cells generated using Cas12a chRDNA guide/nucleoprotein complexes.

FIG. 16A and FIG. 16B illustrate the editing activity of a Cas12a guide/nucleoprotein complex with different polypeptide linker and nuclear localization sequence (NLS) configurations.

FIG. 17 illustrates the editing activity of a Cas12a chRDNA guide/nucleoprotein complex when targeting multiple genes simultaneously with different polypeptide linker and nuclear localization sequence (NLS) configurations.

### DETAILED DESCRIPTION OF THE INVENTION

It is to be understood that the terminology used herein is for the purpose of describing particular embodiments only and is not intended to be limiting. As used in the present specification and the appended claims, the singular forms “a,” “an,” and “the” include plural referents unless the context clearly dictates otherwise. Thus, for example, reference to “a polynucleotide” includes one or more polynucleotides, and reference to “a vector” includes one or more vectors.

Unless defined otherwise, all technical and scientific terms used herein have the same meaning as commonly understood by one of ordinary skill in the art to which the disclosure pertains. Although other methods and materials similar, or equivalent, to those described herein can be useful in the present disclosure, preferred materials and methods are described herein.

The terms “SITE-Seq®” and “SITE-Seq® assay” refer to a biochemical method of identifying the sequence of cut sites within genomic DNA generated using Cas9 programmed with single-guide RNAs (sgRNAs). The assay is fully described in Cameron. P., et al., (2017). Mapping the genomic landscape of CRISPR-Cas9 cleavage. Nature Methods, 14 (6), 600-606.

In view of the teachings of the present specification, one of ordinary skill in the art can apply conventional techniques of immunology, biochemistry, chemistry, molecular biology, microbiology, cell biology, genomics, and recombinant polynucleotides, as taught, for example, by the following standard texts: Abbas et al. (Cellular and Molecular Immunology, 2017, 9th Edition, Elsevier, ISBN 978-0323479783); Butterfield et al. (Cancer Immunotherapy Principles and Practice, 2017, 1st Edition, Demos Medical, ISBN 978-1620700976); Kenneth Murphy (Janeway's Immunobiology, 2016, 9th Edition, Garland Science, ISBN 978-0815345053); Stevens et al. (Clinical Immunology and Serology: A Laboratory Perspective, 2016, 4th Edition, Davis Company, ISBN 978-0803644663); E.A. Greenfield (Antibodies: A Laboratory Manual, 2014, Second edition, Cold Spring Harbor Laboratory Press, ISBN 978-1-936113-81-1); R.I. Freshney (Culture of Animal Cells: A Manual of Basic Technique and Specialized Applications, 2016, 7th Edition, Wiley-Blackwell, ISBN 978-1118873656); C.A. Pinkert (Transgenic Animal Technology, Third Edition: A Laboratory Handbook, 2014, Elsevier, ISBN 978-0124104907); H. Hedrich (The Laboratory Mouse, 2012, Second Edition, Academic Press, ISBN 978-0123820082); Behringer et al. (Manipulating the Mouse Embryo: A Laboratory Manual, 2013, Fourth Edition, Cold Spring Harbor Laboratory Press, ISBN 978-1936113019); McPherson et al. (PCR 2: A Practical Approach, 1995, IRL Press, ISBN 978-0199634248); J. M. Walker (Methods in Molecular Biology (Series), Humana Press, ISSN 1064-3745); Rio et al. (RNA: A Laboratory Manual, 2010, Cold Spring Harbor Laboratory Press, ISBN 978-0879698911); Methods in Enzymology (Series), Academic Press; Green et al. (Molecular Cloning: A Laboratory Manual, 2012, Fourth Edition, Cold Spring Harbor Laboratory Press, ISBN 978-1605500560); G. T. Hermanson (Bioconjugate Techniques, 2013, Third Edition, Academic Press, ISBN 978-0123822390).

Clustered regularly interspaced short palindromic repeats (CRISPR) and related CRISPR-associated proteins (Cas proteins) constitute CRISPR-Cas systems. The classification of CRISPR-Cas systems has had many iterations. Makarova et al. (Nat. Rev. Microbiol., 2020, 18:67-83) proposed a classification system that takes into consideration the signature cas genes specific for individual types and subtypes of CRISPR-Cas systems. The classification also considered sequence similarity between multiple shared Cas proteins, the phylogeny of the best-conserved Cas protein, gene organization, and the structure of the CRISPR array. This approach provided a classification scheme that divides CRISPR-Cas systems into two distinct classes: Class 1 and Class 2.

In Class 2, Type V, systems, the crRNA and target binding involves Cas12, as does the target nucleic acid cleavage. The RuvC-like nuclease domain of Cas12a, for instance, cleaves both strands of the target nucleic acid in a staggered configuration, producing 5′ overhangs, which is in contrast to the blunt ends generated by Cas9 cleavage. These 5′ overhangs may facilitate insertion of DNA through homologous recombination methods.

Other proteins associated with Type V crRNA and target binding and cleavage include Cas12b (formerly C2c1) and Cas12c (formerly C2c3). Cas12b and Cas12c proteins are similar in length to CRISPR Class 2 Type II Cas9 and CRISPR Class 2 Type V Cas12a proteins, ranging from approximately 1,100 amino acids to approximately 1,500 amino acids. C2c1 and C2c3 proteins also contain RuvC-like nuclease domains and have an architecture similar to Cas12a. C2c1 proteins are similar to Cas9 proteins in requiring a crRNA and a tracrRNA for target binding and cleavage, but have an optimal cleavage temperature of 50° C. C2c1 proteins target an AT-rich PAM, which similar to Cas12a, is 5′ of the target sequence. See, e.g., Shmakov et al. (Molecular Cell, 2015, 60 (3): 385-397).

The CRISPR Type V subtypes include the Cas12 proteins, and demonstrate a broad sequence and diversity in size; however, Cas12 subtypes share a common evolutionary origin from TnpB nucleases encoded by IS605-like transposons. Owing to the low sequence similarity, and likely evolution through multiple independent recombination events of Cas12 proteins, classification of Cas12 proteins into their respective subtypes has resulted in multiple naming conventions. Table 1 presents the classification and names for the Type V Cas12 proteins as well as their approximate size, guide requirements, preferred target polynucleotide, and a representative organism of origin.

TABLE 1
Classification of Type V Subtypes

| Type | Cas | Other | Effector | | Target | Representative |
|---|---|---|---|---|---|---|
| V | nomenclature | names | size | Guide | polynucleotide | organism |
| V-A | Cas12A | MAD7 | >1000aa | crRNA | dsDNA | Francisella cf. |
| | | | | | | novicida |
| V-B1 | Cas12b1 | c2c1 | >1000aa | crRNA, | dsDNA | Alicyclobacillus |
| | | | | tracrRNA | | acidoterrestris |
| V-B2 | Cas12b2 | — | | | | Planctomycetes |
| | | | | | | bacterium |
| | | | | | | RBG_13_46_10 |
| V-C | Cas12c | c2c3 | >1000aa | crRNA, | dsDNA | Oleiphilus spp. |
| | | | | tracrRNA | | |
| V-D | Cas12d | CasY | >1000aa | crRNA | dsDNA | Bacterium |
| | | | | | | CC09_39_24 |
| V-E | Cas12e | CasX | ~1000aa | crRNA, | dsDNA | Deltaproteobacteria |
| | | | | tracrRNA | | bacterium |
| V-F1 | Cas12f1 | Cas14a, | 400- | crRNA, | | Uncultured |
| | | c2c10, | 800aa | tracrRNA | | archaeon |
| | | V-U3 | | | | |
| V-F2 | Cas12f2 | Cas14b | | crRNA | | Bacillus |
| | | | | | | thuringiensis |
| | | | | | | HD-771 |
| V-F3 | Cas12f3 | Cas14c | | crRNA | | Candidatus |
| | | | | | | Micrarchaeota |
| | | | | | | archaeon |
| V-G | Cas12g | — | 700- | crRNA, | ssRNA | Hot springs |
| | | | 800aa | tracrRNA | | metagenome |
| V-H | Cas12h | — | ~1000aa | crRNA | ssDNA, | Hypersaline lake |
| | | | | | dsDNA | sediment |
| | | | | | | metagenome |
| V-I | Cas12i | — | ~1000aa | crRNA | ssDNA, | Freshwater |
| | | | | | dsDNA | metagenome |
| V-J | Cas12j | Casφ | 700- | crRNA | dsDNA | Biggiephage |
| | | (Cas- | 800aa | | | |
| | | phi) | | | | |
| V-K | Cas12K | c2c5 | >700aa | crRNA, | no nuclease | Cyanothece spp. |
| | | | | tracrRNA | activity | PCC 8801 |
| V-U | — | c2c4, | n.d. | n.d. | n.d. | Goronia otitidis |
| | | c2c8, | | | | |
| | | c2c9 | | | | |

Cas12 homologs can be identified using sequence similarity search methods known to those skilled in the art. Typically, a Cas12 protein is capable of interacting with a cognate Cas12 guide to form a Cas12 guide/nucleoprotein complex capable of binding to a target nucleic acid sequence. In some embodiments of the present disclosure, the Cas12 protein or homolog thereof is a Cas12a protein or homolog thereof.

Cas12a proteins include, but are not limited to, Cas12a from Parcubacteria bacterium GWC2011_GWC2_44_17 (PbCpf1), Lachnospiraceae bacterium MC2017 (Lb3 Cpf1), Butyrivibrio proteoclasticus (BpCpf1), Peregrinibacteria bacterium GW2011_GWA_33_10 (PeCpf1), Acidaminococcus spp. BV3L6 (AsCpf1), Porphyromonas macacae (PmCpf1), Lachnospiraceae bacterium ND2006 (LbCpf1), Porphyromonas crevioricanis (PcCpf1), Prevotella disiens (PdCpf1), Moraxella bovoculi 237 (MbCpf1), Smithella sp. SC_K08D17 (SsCpf1), Leptospira inadai (LiCpf1), Lachnospiraceae bacterium MA2020 (Lb2Cpf1), Franciscella novicida U112 (FnCpf1), Candidatus methanoplasma termitum (CMtCpf1), and Eubacterium eligens (EeCpf1).

In Type V systems, nucleic acid target sequence binding typically involves a Cas12 protein and a crRNA, as does the nucleic acid target sequence cleavage. In Type V systems, the RuvC-like nuclease domain of Cas12 protein cleaves both strands of the nucleic acid target sequence in a sequential fashion, see Swarts et al. (Mol. Cell, 2017, 66:221-233), producing 5′ overhangs, which contrasts with the blunt ends generated by Cas9 protein cleavage.

The Cas12 protein cleavage activity of Type V systems can be independent of a tracrRNA (e.g., Type V-A); and some Type V systems require only a single crRNA that has a stem-loop structure forming an internal duplex. Cas12 protein binds the crRNA in a sequence- and structure-specific manner by recognizing the stem loop and sequences adjacent to the stem loop, most notably the nucleotides 5′ of the spacer sequence, which hybridize to the nucleic acid target sequence. This stem-loop structure is typically in the range of 15 to 22 nucleotides in length. Substitutions that disrupt this stem-loop duplex abolish cleavage activity, whereas other substitutions that do not disrupt the stem-loop duplex do not abolish cleavage activity. Certain Type V systems require the hybridization between a crRNA and tracrRNA, such as the Type V-F1, V-G, V-C, V-E (CasX), V-K, and V-B. See, e.g., Yan et. al. (Science, 2019, 363 (6422): 88-91).

“Guide” and “guide polynucleotide” as used herein refer to one or more polynucleotides that form a nucleoprotein complex with a Cas protein, wherein the nucleoprotein complex preferentially binds a nucleic acid target sequence in a polynucleotide (relative to a polynucleotide that does not comprise the nucleic acid target sequence). Such guides can comprise ribonucleotide bases (e.g., RNA), deoxyribonucleotide bases (e.g., DNA), combinations of ribonucleotide bases and deoxyribonucleotide bases (e.g., RNA/DNA), nucleotide analogs, modified nucleotides, and the like, as well as synthetic, naturally occurring, and non-naturally occurring modified backbone residues or linkages. Many such guides are known, such as but not limited to, single-guide RNA (including miniature and truncated single-guide RNAs), crRNA, dual-guide RNAs, including but not limited to, crRNA/tracrRNA molecules, and the like, the use of which depends on the particular Cas protein. For example, a “Type V CRISPR-Cas12-associated guide” is a guide that specifically associates with a cognate Cas12 protein to form a nucleoprotein complex.

As used herein, a “CRISPR polynucleotide” is a polynucleotide sequence comprising a portion of a guide molecule. In some embodiments, the CRISPR polynucleotide includes a targeting region and/or an activating region.

With reference to a guide molecule, a “spacer,” “spacer sequence,” “spacer element,” or “targeting region,” as used herein refers to a polynucleotide sequence that can specifically hybridize to a target nucleic acid sequence. The targeting region interacts with the target nucleic acid sequence through hydrogen bonding between complementary base pairs (i.e., paired bases). A targeting region binds to a selected nucleic acid target sequence. In some embodiments, the target sequence is a sequence within a genome of a cell, either in vitro, ex vivo (such as in the generation of CAR-T cells), or in vivo (such as where compositions are administered directly to a subject). A guide molecule may comprise or consist of any sequence selected to target any target sequence. Exemplary target sequences include those that are unique in the target genome. Accordingly, the targeting region is the nucleic acid target-binding sequence. The targeting region determines the location of the site-specific binding and nucleolytic cleavage of a Cas12 protein. Variability of the functional length for a targeting region is known in the art.

As used herein, the term “activating region” refers to a portion of a polynucleotide capable of associating, or binding with, a Cas12 polypeptide, such as a Cas12a polypeptide.

As used herein, the terms “abasic,” “abasic site,” “abasic nucleotide,” “apurinic/apyrimidinic site,” and “AP site” are used interchangeably and refer to a site in a nucleotide sequence that lacks the purine or a pyrimidine base. In certain embodiments, abasic sites comprise a deoxyribose site. In other embodiments, abasic sites comprise a ribose site. In yet further embodiments, abasic sites comprise a modified backbone, such as phosphorothioate backbone or a morpholino backbone. An abasic site cannot form hydrogen base pair bonding with a complementary nitrogen base of a DNA or RNA nucleotide because it does not contain a nitrogen base.

As used herein, the terms “base analog,” “non-canonical base,” and “chemically-modified base” refer to a compound having structural similarity to a canonical purine or pyrimidine base occurring in DNA or RNA. The base analog may contain a modified sugar and/or a modified nucleobase, as compared to a purine or pyrimidine base occurring naturally in DNA or RNA. In some embodiments, the base analog is inosine or deoxyinosine, such as 2′-deoxyinosine. In other embodiments, the base analog is a 2′-deoxyribonucleoside, 2′-ribonucleoside, 2′-deoxyribonucleotide or a 2′-ribonucleotide, wherein the nucleobase includes a modified base (such as, for example, xanthine, uridine, oxanine (oxanosine), 7-methlguanosine, dihydrouridine, 5-methylcytidine, C3 spacer, 5-methyl dC, 5-hydroxybutynl-2′-deoxyuridine, 5-nitroindole, 5-methyl iso-deoxycytosine, iso deoxyguanosine, deoxyuridine, iso deoxycytidine, other 0-1 purine analogs, N-6-hydroxylaminopurine, nebularine, 7-deaza hypoxanthine, other 7-deazapurines, and 2-methyl purines). In some embodiments, the base analog may be selected from the group consisting of 7-deaza-2′-deoxyinosine, 2′-aza-2′-deoxyinosine, PNA-inosine, morpholino-inosine, LNA-inosine, phosphoramidite-inosine, 2′-O-methoxyethyl-inosine, and 2′-OMe-inosine. The term “base analog” also includes, for example, 2′-deoxyribonucleosides, 2′-ribonucleosides, 2′-deoxyribonucleotides or 2′-ribonucleotides, wherein the nucleobase is a substituted hypoxanthine. For instance, the substituted hypoxanthine may be substituted with a halogen, such as fluorine or chlorine. In some embodiments, the base analog may be a fluoroinosine or a chloroinosine, such as 2-chloroinosine, 6-chloroinosine, 8-chloroinosine, 2-fluoroinosine, 6-fluoroinosine, or 8-fluoroinosine. In other embodiments, the base analog is deoxyuridine. In other embodiments the base analog is a nucleic acid mimic (such as, for example, artificial nucleic acids and xeno nucleic acids (XNA)).

As used herein, the term “CRISPR hybrid RNA/DNA guide” (chRDNA) refers to a polynucleotide guide molecule comprising a targeting region, wherein the polynucleotide comprises RNA with DNA designed into the polynucleotide. In embodiments herein, the crRNA component of a Cas12a guide is a chRDNA.

As used herein, the term “Cas12 chRDNA guide/nucleoprotein complex” refers to a chRDNA guide molecule complexed with a Cas12 protein to form a nucleoprotein complex, wherein the nucleoprotein complex is capable of site-directed binding to a nucleic acid target sequence complementary to the nucleic acid target binding sequence present in the chRDNA guide molecule. As used herein, the term “Cas12a chRDNA guide/nucleoprotein complex” refers to a chRDNA guide molecule complexed with a Cas12a protein to form a nucleoprotein complex, wherein the nucleoprotein complex is capable of site-directed binding to a nucleic acid target sequence complementary to the nucleic acid target binding sequence present in the chRDNA guide molecule.

As used herein, a “stem element” or “stem structure” refers to two strands of nucleic acids that form a double-stranded region (the “stem element”). A “stem-loop element” or “stem-loop structure” refers to a stem structure wherein 3′-end sequences of one strand are covalently bonded to 5′-end sequences of the second strand by a nucleotide sequence of typically single-stranded nucleotides (“a stem-loop element nucleotide sequence”). In some embodiments, the loop element comprises a loop element nucleotide sequence of between about 3 and about 20 nucleotides in length, preferably between about 4 and about 10 nucleotides in length. In some embodiments, a loop element nucleotide sequence is a single-stranded nucleotide sequence of unpaired nucleic acid bases that do not interact through hydrogen bond formation to create a stem element within the loop element nucleotide sequence. The term “hairpin element” is also used herein to refer to stem-loop structures. Such structures are well known in the art. The base pairing may be exact; however, as is known in the art, a stem element does not require exact base pairing. Thus, the stem element may include one or more base mismatches or non-paired bases. A stem-loop element may further comprise a pseudoknot structure.

A “linker element nucleotide sequence,” “linker nucleotide sequence,” and “linker polynucleotide” are used interchangeably herein and refer to a sequence of one or more nucleotides covalently attached to a first nucleic acid sequence (5′-linker nucleotide sequence-first nucleic acid sequence-3′). In some embodiments, a linker nucleotide sequence connects two separate nucleic acid sequences to form a single polynucleotide (e.g., 5′-first nucleic acid sequence-linker nucleotide sequence-second nucleic acid sequence-3′). Other examples of linker sequences include, but are not limited to, 5′-first nucleic acid sequence-linker nucleotide sequence-3′, and 5′-linker nucleotide sequence-first first nucleic acid sequence-linker nucleotide sequence-3′. In some embodiments, the linker element nucleotide sequence can be a single-stranded nucleotide sequence of unpaired nucleic acid bases that do not interact with each other through hydrogen bond formation to create a secondary structure (e.g., a stem-loop structure) within the linker element nucleotide sequence. In some embodiments, two single-stranded linker element nucleotide sequences can interact with each other through hydrogen bonding between the two linker element nucleotide sequences. In some embodiments, a linker element nucleotide sequence can be between about 1 and about 50 nucleotides in length, preferably between about 1 and about 15 nucleotides in length.

As used herein, the term “cognate” typically refers to a Cas12 protein (e.g., Cas12a) and one or more Type V CRISPR-Cas12-associated guides (e.g., Cas12 chRDNA guides) that are capable of forming a nucleoprotein complex capable of site-directed binding to a nucleic acid target sequence complementary to the nucleic acid target binding sequence present in one of the one or more guides.

The terms “wild type,” “naturally occurring,” and “unmodified” are used herein to mean the typical (or most common) form, appearance, phenotype, or strain existing in nature; for example, the typical form of cells, organisms, polynucleotides, proteins, macromolecular complexes, genes, RNAs, DNAs, or genomes as they occur in, and can be isolated from, a source in nature. The wild-type form, appearance, phenotype, or strain serve as the original parent before an intentional modification. Thus, mutant, variant, engineered, recombinant, and modified forms are not wild-type forms.

By “isolated” is meant, when referring to a polypeptide, that the indicated molecule is separate and discrete from the whole organism with which the molecule is found in nature or is present in the substantial absence of other biological macromolecules of the same type. The term “isolated” with respect to a polynucleotide is a nucleic acid molecule devoid, in whole or part, of sequences normally associated with it in nature; or a sequence, as it exists in nature, but having heterologous sequences in association therewith; or a molecule disassociated from the chromosome.

The term “purified” as used herein preferably means at least 75% by weight, more preferably at least 85% by weight, more preferably still at least 95% by weight, and most preferably at least 98% by weight, of the same molecule is present.

The terms “engineered,” “genetically engineered,” “genetically modified,” “recombinant,” “modified,” “non-naturally occurring,” and “non-native” indicate intentional human manipulation of the genome of an organism or cell. The terms encompass methods of genomic modification that include genomic editing, as defined herein, as well as techniques that alter gene expression or inactivation, enzyme engineering, directed evolution, knowledge-based design, random mutagenesis methods, gene shuffling, codon optimization, and the like. Methods for genetic engineering are known in the art.

“Covalent bond,” “covalently attached,” “covalently bound,” “covalently linked,” “covalently connected,” and “molecular bond” are used interchangeably herein and refer to a chemical bond that involves the sharing of electron pairs between atoms. Examples of covalent bonds include, but are not limited to, phosphodiester bonds and phosphorothioate bonds.

“Non-covalent bond,” “non-covalently attached,” “non-covalently bound,” “non-covalently linked,” “non-covalent interaction,” and “non-covalently connected” are used interchangeably herein, and refer to any relatively weak chemical bond that does not involve sharing of a pair of electrons. Multiple non-covalent bonds often stabilize the conformation of macromolecules and mediate specific interactions between molecules. Examples of non-covalent bonds include, but are not limited to, hydrogen bonding, ionic interactions (e.g., Na⁺Cl⁻), van der Waals interactions, and hydrophobic bonds.

As used herein, “hydrogen bonding,” “hydrogen-base pairing,” and “hydrogen bonded” are used interchangeably and refer to canonical hydrogen bonding and non-canonical hydrogen bonding including, but not limited to, “Watson-Crick-hydrogen-bonded base pairs” (W-C-hydrogen-bonded base pairs or W-C hydrogen bonding); “Hoogsteen-hydrogen-bonded base pairs” (Hoogsteen hydrogen bonding); and “wobble-hydrogen-bonded base pairs” (wobble hydrogen bonding). W-C hydrogen bonding, including reverse W-C hydrogen bonding, refers to purine-pyrimidine base pairing, that is, adenine:thymine, guanine:cytosine, and uracil:adenine. Hoogsteen hydrogen bonding, including reverse Hoogsteen hydrogen bonding, refers to a variation of base pairing in nucleic acids wherein two nucleobases, one on each strand, are held together by hydrogen bonds in the major groove. This non-W-C hydrogen bonding can allow a third strand to wind around a duplex and form triple-stranded helices. Wobble hydrogen bonding, including reverse wobble hydrogen bonding, refers to a pairing between two nucleotides in RNA molecules that does not follow Watson-Crick base pair rules. There are four major wobble base pairs: guanine:uracil, inosine (hypoxanthine):uracil, inosine:adenine, and inosine:cytosine. Wobble base interactions are also known to occur between inosine:thymine and inosine:guanine. Inosine bases and deoxy inosine bases can be referred to as “universal pairing bases,” as they are capable of hydrogen bonding with the canonical DNA and RNA bases. See, e.g., Watkins et al. (Nucleic Acid Research, 2005, 33 (19): 6258-67). Rules for canonical hydrogen bonding and non-canonical hydrogen bonding are known to those of ordinary skill in the art. See, e.g., R. F. Gesteland (The RNA World, Third Edition (Cold Spring Harbor Monograph Series), 2005, Cold Spring Harbor Laboratory Press, ISBN 978-0879697396); R. F. Gesteland (The RNA World, Second Edition (Cold Spring Harbor Monograph Series), 1999, Cold Spring Harbor Laboratory Press, ISBN 978-0879695613); R. F. Gesteland (The RNA World, First Edition (Cold Spring Harbor Monograph Series), 1993, Cold Spring Harbor Laboratory Press, 978-0879694562) (see, e.g., Appendix 1: Structures of Base Pairs Involving at Least Two Hydrogen Bonds, I. Tinoco); W. Saenger (Principles of Nucleic Acid Structure, 1988, Springer International Publishing AG, ISBN 978-0-387-90761-1); S. Neidle (Principles of Nucleic Acid Structure, 2007, First Edition, Academic Press, ISBN 978-01236950791).

“Connect,” “connected,” and “connecting” are used interchangeably herein, and refer to a covalent bond or a non-covalent bond between two macromolecules (e.g., polynucleotides, proteins, and the like).

As used herein, the terms “nucleic acid sequence,” “nucleotide sequence,” and “oligonucleotide” are interchangeable and refer to a polymeric form of nucleotides. As used herein, the term “polynucleotide” refers to a polymeric form of nucleotides that has one 5′ end and one 3′ end and can comprise one or more nucleic acid sequences. The nucleotides may be deoxyribonucleotides (DNA), ribonucleotides (RNA), analogs thereof, or combinations thereof, and may be of any length. Polynucleotides may perform any function and may have various secondary and tertiary structures. The terms encompass known analogs of natural nucleotides and nucleotides that are modified in the base, sugar, and/or phosphate moieties. Analogs of a particular nucleotide have the same base-pairing specificity (e.g., an analog of A base pairs with T). A polynucleotide may comprise one modified nucleotide or multiple modified nucleotides. Examples of modified nucleotides include fluorinated nucleotides, methylated nucleotides, chemically modified sugars, and nucleotide analogs. Nucleotide structure may be modified before or after a polymer is assembled. Following polymerization, polynucleotides may be additionally modified via, for example, conjugation with a labeling component or target binding component. A nucleotide sequence may incorporate non-nucleotide components. The terms also encompass nucleic acids comprising modified backbone residues or linkages, that are synthetic, naturally occurring, and/or non-naturally occurring, and have similar binding properties as a reference polynucleotide (e.g., DNA or RNA). Examples of such analogs include, but are not limited to, phosphorothioates, phosphoramidites, methyl phosphonates, chiral-methyl phosphonates, 2-O-methyl ribonucleotides, peptide-nucleic acids (PNAs), Locked Nucleic Acid (LNA™) (Exiqon, Woburn, MA) nucleosides, glycol nucleic acid, bridged nucleic acids, and morpholino structures.

Peptide-nucleic acids (PNAs) are synthetic homologs of nucleic acids wherein the polynucleotide phosphate-sugar backbone is replaced by a flexible pseudo-peptide polymer. Nucleobases are linked to the polymer. PNAs have the capacity to hybridize with high affinity and specificity to complementary sequences of RNA and DNA.

In phosphorothioate nucleic acids, the phosphorothioate (PS) bond substitutes a sulfur atom for a non-bridging oxygen in the polynucleotide phosphate backbone. This modification makes the internucleotide linkage resistant to nuclease degradation. In some embodiments, phosphorothioate bonds are introduced between the last 3 to 5 nucleotides at the 5′-end or 3′-end sequences of a polynucleotide sequence to inhibit exonuclease degradation. Placement of phosphorothioate bonds throughout an entire oligonucleotide helps reduce degradation by endonucleases as well.

Threose nucleic acid (TNA) is an artificial genetic polymer. The backbone structure of TNA comprises repeating threose sugars linked by phosphodiester bonds. TNA polymers are resistant to nuclease degradation. TNA can self-assemble by base-pair hydrogen bonding into duplex structures.

Linkage inversions can be introduced into polynucleotides through use of “reversed phosphoramidites” (see, e.g., www.ucalgary.ca/dnalab/synthesis/-modifications/linkages). A 3′-3′ linkage at a terminus of a polynucleotide stabilizes the polynucleotide to exonuclease degradation by creating an oligonucleotide having two 5′-OH termini but lacking a 3′-OH terminus. Typically, such polynucleotides have phosphoramidite groups on 5′-OH position and a dimethoxytrityl (DMT) protecting group on 3′-OH position. Normally, the DMT protecting group is on 5′-OH and the phosphoramidite is on the 3′-OH.

Polynucleotide sequences are displayed herein in the conventional 5′ to 3′ orientation unless otherwise indicated.

As used herein, “sequence identity” generally refers to the percent identity of nucleotide bases or amino acids comparing a first polynucleotide or polypeptide to a second polynucleotide or polypeptide, respectively, using algorithms having various weighting parameters. Sequence identity between two polynucleotides or two polypeptides can be determined using sequence alignment by various methods and computer programs (e.g., BLAST, CS-BLAST, FASTA, HMMER, L-ALIGN, and the like) available through the worldwide web at sites including, but not limited to, GENBANK (www.ncbi.nlm.nih.gov/genbank/) and EMBL-EBI (www.ebi.ac.uk.). Sequence identity between two polynucleotides or two polypeptide sequences is generally calculated using the standard default parameters of the various methods or computer programs. A high degree of sequence identity between two polynucleotides or two polypeptides is typically between about 90% identity and 100% identity over the length of the reference polypeptide, for example, about 90% identity or higher, preferably about 95% identity or higher, more preferably about 98% identity or higher. A moderate degree of sequence identity between two polynucleotides or two polypeptides is typically between about 80% identity to about 85% identity, for example, about 80% identity or higher, preferably about 85% identity over the length of the reference polypeptide. A low degree of sequence identity between two polynucleotides or two polypeptides is typically between about 50% identity and 75% identity, for example, about 50% identity, preferably about 60% identity, more preferably about 75% identity over the length of the reference polypeptide. For example, a Cas12 protein (e.g., a Cas12 comprising amino acid substitutions) can have a low degree of sequence identity, a moderate degree of sequence identity, or a high degree of sequence identity, over its length to a reference Cas12 protein (e.g., a wild type Cas12) over its length. As another example, a guide molecule can have a low degree of sequence identity, a moderate degree of sequence identity, or a high degree of sequence identity, over its length compared to a reference wild type guide molecule over its length that complexes with the reference Cas12 protein (e.g., a polynucleotide that forms a complex with Cas12).

As used herein, “hybridization,” “hybridize,” or “hybridizing” is the process of combining two complementary single-stranded nucleic acid (e.g., DNA or RNA) molecules so as to form a single double-stranded molecule (e.g., DNA/DNA, DNA/RNA, RNA/RNA) through hydrogen base pairing. Hybridization stringency is typically determined by the hybridization temperature and the salt concentration of the hybridization buffer; e.g., high temperature and low salt provide high stringency hybridization conditions. Examples of salt concentration ranges and temperature ranges for different hybridization conditions are as follows: high stringency, approximately 0.01M to approximately 0.05M salt, hybridization temperature 5° C. to 10° C. below Tₘ; moderate stringency, approximately 0.16M to approximately 0.33M salt, hybridization temperature 20° C. to 29° C. below Tₘ; and low stringency, approximately 0.33M to approximately 0.82M salt, hybridization temperature 40° C. to 48° C. below Tₘ. Tₘ of duplex nucleic acid sequences is calculated by standard methods well-known in the art. See, e.g., Maniatis et al. (Molecular Cloning: A Laboratory Manual, 1982, Cold Spring Harbor Laboratory Press: New York); Casey et al. (Nucleic Acids Research, 1977, 4:1539-1552); Bodkin et al. (Journal of Virological Methods, 1985, 10 (1): 45-52); and Wallace et al. (Nucleic Acids Research, 1981, 9 (4): 879-894). Algorithm prediction tools to estimate Tₘ are also widely available. High stringency conditions for hybridization typically refer to conditions under which a polynucleotide complementary to a target sequence predominantly hybridizes with the target sequence, and substantially does not hybridize to non-target sequences. Typically, hybridization conditions are of moderate stringency, preferably high stringency.

As used herein, “complementarity” refers to the ability of a nucleic acid sequence to form hydrogen bonds with another nucleic acid sequence (e.g., through canonical Watson-Crick base pairing). A percent complementarity indicates the percentage of residues in a nucleic acid sequence that can form hydrogen bonds with a second nucleic acid sequence. If two nucleic acid sequences have 100% complementarity, the two sequences are perfectly complementary, i.e., all of the contiguous residues of a first polynucleotide hydrogen bond with the same number of contiguous residues in a second polynucleotide.

As used herein, the term “corresponding deoxyribonucleotide base,” with respect to a ribonucleotide base, refers to a deoxyribonucleotide base (including, e.g., a modified or variant version of a canonical deoxyribonucleotide base) that binds, through complementary (Watson-Crick) base pairing, to the same base as the ribonucleotide base does. For example, for the ribonucleotide bases A, C and G, the corresponding deoxyribonucleotide bases may be A, C and G, respectively. For the ribonucleotide base U, the corresponding deoxyribonucleotide base may be T, for example.

As used herein, “binding” refers to a non-covalent interaction between macromolecules (e.g., between a protein and a polynucleotide, between a polynucleotide and a polynucleotide, or between a protein and a protein, and the like). Such non-covalent interaction is also referred to as “associating” or “interacting” (e.g., if a first macromolecule interacts with a second macromolecule, the first macromolecule binds to second macromolecule in a non-covalent manner). Some portions of a binding interaction may be sequence-specific (the terms “sequence-specific binding,” “sequence-specifically bind,” “site-specific binding,” and “site specifically binds” are used interchangeably herein). Sequence-specific binding typically refers to one or more guide molecules capable of forming a complex with a protein (e.g., Cas12) to cause the protein to bind a nucleic acid sequence (e.g., a DNA sequence) comprising a nucleic acid target sequence (e.g., a target DNA sequence) preferentially relative to a second nucleic acid sequence (e.g., a second DNA sequence) without the nucleic acid target binding sequence (e.g., the DNA target binding sequence). All components of a binding interaction do not need to be sequence-specific, such as contacts of a protein with phosphate residues in a DNA backbone. Binding interactions can be characterized by a dissociation constant (Kd). “Binding affinity” refers to the strength of the binding interaction. An increased binding affinity is correlated with a lower Kd.

As used herein, a Cas12 protein is said to “target” a polynucleotide if a Cas12 guide/nucleoprotein complex binds or cleaves a polynucleotide at the nucleic acid target sequence within the polynucleotide.

A “protospacer adjacent motif” or “PAM” as used herein refers to double-stranded nucleic acid sequences comprising a Cas12 protein-binding recognition sequence, wherein amino acids of the Cas12 protein directly interact with the recognition sequence (e.g., Cas12a protein interacts with the PAM 5′-TTTN-3′ or the PAM 5′-TTTV-3′). PAM sequences are on the non-target strand and can be 5′ or 3′ of a target complement sequence (e.g., in CRISPR-Cas12a systems the PAM 5′-TTTN-3′ or the PAM 5′-TTTV-3′sequence is on the non-target strand and is 5′ of the target-complement sequence). PAMs are recognized by the Cas12 effector proteins (e.g., a Cas12a protein) prior to target sequence unwinding and hydrogen base-pair bonding between the target sequence and the nucleic acid target binding sequence.

“Target,” “target sequence,” “nucleic acid target sequence,” “target nucleic acid sequence,” and “on-target sequence” are used interchangeably herein to refer to a nucleic acid sequence that is wholly, or in part, complementary to a nucleic acid target binding sequence of a Cas12 polynucleotide (e.g., the targeting region). Typically, the nucleic acid target binding sequence is selected to be 100% complementary to a nucleic acid target sequence to which binding of a Cas12 nucleoprotein complex is being directed; however, to attenuate binding to a nucleic acid target sequence, lower percent complementarity can be used.

When the nucleic acid target binding sequence is 100% complementary to the target sequence, excluding abasic sites contained in the nucleic acid target binding sequence, the target sequence is referred to as an “on-target.” On-target sequence binding refers to binding of the Cas12 guide/nucleoprotein complex to a nucleic acid sequence having 100% complementarity to the non-abasic site portion of the nucleic acid target binding sequence (spacer). When the nucleic acid target binding sequence (spacer) has less than 100% complementary to the target sequence, excluding abasic sites contained in the nucleic acid target binding sequence, the target sequence can be referred to as an “off-target.” Off-target sequence binding refers to binding of the Cas12 guide/nucleoprotein complex to nucleic acid sequences having less than 100% complementarity to the non-abasic site portion of the nucleic acid target binding sequence (spacer). The nucleic acid target sequence can be a double-stranded or a single-stranded DNA molecule. The target sequence can be a double-stranded or single-stranded RNA molecule. The target sequence can be a RNA:DNA hybrid molecule. The target sequence can be present on the opposite strand of a PAM sequence.

As used herein, “double-strand break” (DSB) refers to both strands of a double-stranded segment of DNA being severed. In some instances, if such a break occurs, one strand can be said to have a “sticky end” wherein nucleotides are exposed and not hydrogen bonded to nucleotides on the other strand. In other instances, a “blunt end” can occur wherein both strands remain fully base paired with each other.

“Donor polynucleotide,” “donor oligonucleotide,” “donor template,” “non-viral donor,” and “non-viral template” are used interchangeably herein and can be a double-stranded polynucleotide (e.g., DNA), a single-stranded polynucleotide (e.g., DNA or RNA), or a combination thereof. Donor polynucleotides can comprise homology arms flanking the insertion sequence (e.g., DSBs in the DNA). The homology arms on each side can vary in length. Parameters for the design and construction of donor polynucleotides are well-known in the art. See, e.g., Ran et al. (Nature Protocols, 2013, 8 (11): 2281-2308); Smithies et al. (Nature, 1985, 317:230-234); Thomas et al. (Cell, 1986, 44:419-428); Wu et al. (Nature Protocols, 2008, 3:1056-1076); Singer et al. (Cell, 1982, 31:25-33); Shen et al. (Genetics, 1986, 112:441-457); Watt et al. (PNAS, 1985, 82:4768-4772); Sugawara et al. (Journal of Molecular Cell Biology, 1992, 12 (2): 563-575); Rubnitz et al. (Journal of Molecular Cell Biology, 1984, 4 (11): 2253-2258); Ayares et al. (PNAS, 1986, 83 (14): 5199-5203); and Liskay et al. (Genetics, 1987, 115 (1): 161-167). In some embodiments, a donor polynucleotide comprises a chimeric antigen receptor (CAR).

As used herein, “homology-directed repair” (HDR) refers to DNA repair that takes place in cells, for example, during repair of a DSB in DNA. HDR requires nucleotide sequence homology and uses a donor polynucleotide to repair the sequence wherein the DSB (e.g., within a target DNA sequence) occurred. The donor polynucleotide generally has the requisite sequence homology with the sequence flanking the DSB so that the donor polynucleotide can serve as a suitable template for repair. HDR results in the transfer of genetic information from, for example, the donor polynucleotide to the target DNA sequence. HDR may result in alteration of the target DNA sequence (e.g., insertion, deletion, or mutation) if the donor polynucleotide sequence differs from the target DNA sequence and part or all of the donor polynucleotide is incorporated into the target DNA sequence. In some embodiments, an entire donor polynucleotide, a portion of the donor polynucleotide, or a copy of the donor polynucleotide, is integrated at the site of the target DNA sequence. For example, a donor polynucleotide can be used for repair of the break in the target DNA sequence, wherein the repair results in the transfer of genetic information (e.g., polynucleotide sequences) from the donor polynucleotide at the site or in close proximity of the break in the DNA. Accordingly, new genetic information (e.g., polynucleotide sequences) may be inserted or copied at a target DNA sequence.

As used herein, “homology-independent target integration” (HITI) refers to DNA repair that takes place in a cell, for example, during repair of a DSB in DNA. HITI, unlike HDR, does not require nucleotide sequence homology and uses a donor polynucleotide to repair the sequence wherein the DSB occurred (e.g., within a target DNA sequence). HITI results in the transfer of genetic information from, for example, the donor polynucleotide to the target DNA sequence. HITI may result in alteration of the target DNA sequence (e.g., insertion, deletion, or mutation) if the donor polynucleotide sequence differs from the target DNA sequence and part or all of the donor polynucleotide is incorporated into the target DNA sequence. In some embodiments, an entire donor polynucleotide, a portion of a donor polynucleotide, or a copy of a donor polynucleotide, is integrated at the site of the target DNA sequence. For example, a donor polynucleotide can be used for repair of the break in the target DNA sequence, wherein the repair results in the transfer of genetic information (e.g., polynucleotide sequences) from the donor polynucleotide at the site or in close proximity to the break in the DNA. Accordingly, new genetic information (e.g., polynucleotide sequences) may be inserted or copied at a target DNA sequence.

A “genomic region” is a segment of a chromosome in the genome of a host cell that is present on either side of the nucleic acid target sequence site or, alternatively, also includes a portion of the nucleic acid target sequence site. The homology arms of the donor polynucleotide have sufficient homology to undergo homologous recombination with the corresponding genomic regions. In some embodiments, the homology arms of the donor polynucleotide share significant sequence homology to the genomic region immediately flanking the nucleic acid target sequence site; it is recognized that homology arms can be designed to have sufficient homology to genomic regions farther from the nucleic acid target sequence site.

As used herein, “non-homologous end joining” (NHEJ) refers to the repair of a DSB in DNA by direct ligation of one terminus of the break to the other terminus of the break without a requirement for a donor polynucleotide. NHEJ is a DNA repair pathway available to cells to repair DNA without the use of a repair template. NHEJ in the absence of a donor polynucleotide often results in nucleotides being randomly inserted or deleted at the site of the DSB.

“Microhomology-mediated end joining” (MMEJ) is pathway for repairing a DSB in DNA. MMEJ involves deletions flanking a DSB and alignment of microhomologous sequences internal to the break site before joining. MMEJ is genetically defined and requires the activity of, for example, CtIP, Poly(ADP-Ribose) Polymerase 1 (PARP1), DNA polymerase theta (Pol θ), DNA Ligase 1 (Lig 1), or DNA Ligase 3 (Lig 3). Additional genetic components are known in the art. See, e.g., Sfeir et al. (Trends in Biochemical Sciences, 2015, 40:701-714).

As used herein, “DNA repair” encompasses any process whereby cellular machinery repairs damage to a DNA molecule contained in the cell. The damage repaired can include single-strand breaks or double-strand breaks (DSBs). At least three mechanisms exist to repair DSBs: HDR, NHEJ, and MMEJ. “DNA repair” is also used herein to refer to DNA repair resulting from human manipulation, wherein a target locus is modified, e.g., by inserting, deleting, or substituting nucleotides, all of which represent forms of genome editing.

As used herein, “recombination” refers to a process of exchange of genetic information between two polynucleotides.

As used herein, the terms “regulatory sequences,” “regulatory elements,” and “control elements” are interchangeable and refer to polynucleotide sequences that are upstream (5′ non-coding sequences), within, or downstream (3′ non-translated sequences) of a polynucleotide target to be expressed. Regulatory sequences influence, for example, the timing of transcription, amount or level of transcription, RNA processing or stability, and/or translation of the related structural nucleotide sequence. Regulatory sequences may include activator binding sequences, enhancers, introns, polyadenylation recognition sequences, promoters, transcription start sites, repressor binding sequences, stem-loop structures, translational initiation sequences, internal ribosome entry sites (IRES), translation leader sequences, transcription termination sequences (e.g., polyadenylation signals and poly-U sequences), translation termination sequences, primer binding sites, and the like.

Regulatory elements include those that direct constitutive, inducible or repressible expression of a nucleotide sequence in many types of host cells and those that direct expression of the nucleotide sequence only in certain host cells (e.g., tissue-specific regulatory sequences). In some embodiments, a vector comprises one or more pol III promoters, one or more pol II promoters, one or more pol I promoters, or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer; see, e.g., Boshart et al. (Cell, 1985, 41:521-530)), the SV40 promoter, the dihydrofolate reductase promoter, the β-actin promoter, the phosphoglycerol kinase (PGK) promoter, and the EF1α promoter. It will be appreciated by those skilled in the art that the design of an expression vector may depend on such factors as the choice of the host cell to be transformed, the level of expression desired, and the like. A vector can be introduced into host cells to thereby produce transcripts, proteins, or peptides, including fusion proteins or peptides, encoded by nucleic acid sequences as described herein.

“Gene” as used herein refers to a polynucleotide sequence comprising exons and related regulatory sequences. A gene may further comprise introns and/or untranslated regions (UTRs).

As used herein, the term “operably linked” refers to polynucleotide sequences or amino acid sequences placed into a functional relationship with one another. For example, regulatory sequences (e.g., a promoter or enhancer) are “operably linked” to a polynucleotide encoding a gene product if the regulatory sequences regulate or contribute to the modulation of the transcription of the polynucleotide. Operably linked regulatory elements are typically contiguous with the coding sequence. However, enhancers can function if separated from a promoter by up to several kilobases or more. Accordingly, some regulatory elements may be operably linked to a polynucleotide sequence but not contiguous with the polynucleotide sequence. Similarly, translational regulatory elements contribute to the modulation of protein expression from a polynucleotide.

As used herein, “expression” refers to transcription of a polynucleotide from a DNA template, resulting in, for example, a messenger RNA (mRNA) or other RNA transcript (e.g., non-coding, such as structural or scaffolding RNAs). The term further refers to the process through which transcribed mRNA is translated into peptides, polypeptides, or proteins. Transcripts and encoded polypeptides may be referred to collectively as “gene products.” Expression may include splicing the mRNA in a eukaryotic cell, if the polynucleotide is derived from genomic DNA.

A “coding sequence” or a sequence that “encodes” a selected polypeptide, is a nucleic acid molecule that is transcribed (in the case of DNA) and translated (in the case of mRNA) into a polypeptide in vitro or in vivo when placed under the control of appropriate regulatory sequences. The boundaries of the coding sequence are determined by a start codon at the 5′ terminus and a translation stop codon at the 3′ terminus. A transcription termination sequence may be located 3′ to the coding sequence.

As used herein, the term “modulate” refers to a change in the quantity, degree or amount of a function. For example, a Cas12-guide/nucleoprotein complex, as disclosed herein, may modulate the activity of a promoter sequence by binding to a nucleic acid target sequence at or near the promoter. Depending on the action occurring after binding, the Cas12 guide/nucleoprotein complex can induce, enhance, suppress, or inhibit, transcription of a gene operatively linked to the promoter sequence. Thus, “modulation” of gene expression includes both gene activation and gene repression.

Modulation can be assayed by determining any characteristic directly or indirectly affected by the expression of the target gene. Such characteristics include, for example, changes in RNA or protein levels, protein activity, product levels, expression of the gene, or activity level of reporter genes. Accordingly, the terms “modulating expression,” “inhibiting expression,” and “activating expression,” of a gene can refer to the ability of a Cas12 guide/nucleoprotein complex to change, activate, or inhibit transcription of a gene.

“Vector” and “plasmid” as used herein refer to a polynucleotide vehicle to introduce genetic material into a cell. Vectors can be linear or circular. Vectors can contain a replication sequence capable of effecting replication of the vector in a suitable host cell (e.g., an origin of replication). Upon transformation of a suitable host, the vector can replicate and function independently of the host genome or integrate into the host genome. Vector design depends, among other things, on the intended use and host cell for the vector, and the design of a vector for a particular use and host cell is within the level of skill in the art. The four major types of vectors are plasmids, viral vectors, cosmids, and artificial chromosomes. Typically, vectors comprise an origin of replication, a multicloning site, and/or a selectable marker. An expression vector typically comprises an expression cassette. By “recombinant virus” is meant a virus that has been genetically altered, e.g., by the addition or insertion of a heterologous nucleic acid construct into a viral genome or portion thereof.

As used herein, “expression cassette” refers to a polynucleotide construct generated using recombinant methods or by synthetic means and comprising regulatory sequences operably linked to a selected polynucleotide to facilitate expression of the selected polynucleotide in a host cell. For example, the regulatory sequences can facilitate transcription of the selected polynucleotide in a host cell, or transcription and translation of the selected polynucleotide in a host cell. An expression cassette can, for example, be integrated in the genome of a host cell or be present in a vector to form an expression vector.

As used herein, a “targeting vector” is a recombinant DNA construct typically comprising tailored DNA arms, homologous to genomic DNA, that flank elements of a target gene or nucleic acid target sequence (e.g., a DSB). A targeting vector comprises a donor polynucleotide. Elements of the target gene can be modified in a number of ways including deletions and/or insertions. A defective target gene can be replaced by a functional target gene, or in the alternative a functional gene can be knocked out. Optionally, the donor polynucleotide of a targeting vector comprises a selection cassette comprising a selectable marker that is introduced into the target gene. Targeting regions adjacent or within a target gene can be used to affect regulation of gene expression.

By “gene editing” or “genome editing” as used herein is meant a type of genetic engineering that results in a genetic modification, such as an insertion, deletion, or replacement, of a nucleotide sequence, or even a single base, at a specific site in a cell genome. The terms include, without limitation, heterologous gene expression, gene or promoter insertion or deletion, nucleic acid mutation, and a disruptive genetic modification, as defined herein.

As used herein, the term “between” is inclusive of end values in a given range (e.g., between about 1 and about 50 nucleotides in length includes 1 nucleotide and 50 nucleotides).

As used herein, the term “amino acid” refers to natural and synthetic (unnatural) amino acids, including amino acid analogs, modified amino acids, peptidomimetics, glycine, and D or L optical isomers.

As used herein, the terms “peptide,” “polypeptide,” and “protein” are interchangeable and refer to polymers of amino acids. A polypeptide may be of any length. It may be branched or linear, it may be interrupted by non-amino acids, and it may comprise modified amino acids. The terms also refer to an amino acid polymer that has been modified through, for example, acetylation, disulfide bond formation, glycosylation, lipidation, phosphorylation, pegylation, biotinylation, cross-linking, and/or conjugation (e.g., with a labeling component or ligand). Polypeptide sequences are displayed herein in the conventional N-terminal to C-terminal orientation, unless otherwise indicated. Polypeptides and polynucleotides can be made using routine techniques in the field of molecular biology. Furthermore, essentially any polypeptide or polynucleotide is available from commercial sources.

The terms “fusion protein” and “chimeric protein” as used herein refer to a single protein created by joining two or more proteins, protein domains, or protein fragments, that do not naturally occur together in a single protein.

A fusion protein can also comprise epitope tags (e.g., histidine tags, FLAG® (Sigma Aldrich, St. Louis, MO) tags, Myc tags), reporter protein sequences (e.g., glutathione-S-transferase, beta-galactosidase, luciferase, green fluorescent protein, cyan fluorescent protein, yellow fluorescent protein), and/or nucleic acid sequence binding domains (e.g., a DNA binding domain or a RNA binding domain). A fusion protein can comprise at least one nuclear localization sequence (NLS), such as a simian virus 40 (SV40) NLS or a nucleoplasmin NLS. A fusion protein can also comprise activator domains (e.g., heat shock transcription factors, NFKB activators) or repressor domains (e.g., a KRAB domain). As described by Lupo et al. (Current Genomics, 2013, 14 (4): 268-278), the KRAB domain is a potent transcriptional repression module and is located in the amino-terminal sequence of most C2H2 zinc finger proteins. See, e.g., Margolin et al. (PNAS, 1994, 91:4509-4513); and Witzgall et al. (PNAS, 1994, 91:4514-4518 (1994). The KRAB domain typically binds to co-repressor proteins and/or transcription factors via protein-protein interactions, causing transcriptional repression of genes to which KRAB zinc finger proteins (KRAB-ZFPs) bind. See, e.g., Friedman et al. (Genes & Development, 1996, 10:2067-2678). In some embodiments, linker nucleic acid sequences are used to join the two or more proteins, protein domains, or protein fragments.

As used herein, the terms “nuclear localization sequence” (NLS) or “nuclear localization signal” refer to a polypeptide sequence of a protein that preferentially increases the subcellular localization of a protein to the nucleus of a cell. NLS sequences are typically positively changed stretches of amino acids located at the amino-terminus (“N-terminus”) of, the carboxyl-terminus (“C-terminus”) of, or internally within, a protein (or a combination thereof, i.e., one or more NLS at the N-terminus and one or more NLS at the C-terminus). NLS sequences can be covalently linked directly to the protein, or can be joined via a linker polypeptide. The length of the linker sequences can be optimized based on structural characteristics of the protein (e.g., solvent accessibility of the termini, the presence of other critical functional peptide sequences at the termini, etc.) to ensure the accessibility of the NLS sequence for cognate importin protein binding and trafficking. Additionally, optimal linker length can be screened for empirically (see, e.g., Example 11). NLS sequences can be fully synthetic or derived from endogenous or exogenous proteins sequences. Computation tools can be used to predict an NLS sequence in a protein (see, e.g., moseslab.csb.utoronto.ca/NLStradamus/, or nls-mapper.iab.keio.ac.jp/cgi-bin/NLS_Mapper_form.cgi). Examples of NLS sequences are presented in Table 2.

TABLE 2
Exemplary Nuclear Localization Sequences

| Source | Sequence | SEQ ID NO. |
|---|---|---|
| SV40 large T- | PKKKRKV | SEQ ID NO: 04 |
| antigen | | |
| Nucleoplasmin | KRPAATKKAGQAKKKK | SEQ ID NO: 05 |
| 53BP1 | GKRKLITSEEERSPAKRGRKS | SEQ ID NO: 493 |
| VACM-1/CUL5 | PKLKRQ | SEQ ID NO: 494 |
| CXCR4 | RPRK | SEQ ID NO: 495 |
| VP1 | RRARRPRG | SEQ ID NO: 496 |
| ING4 | KGKKGRTQKEKKAARARSKGKN | SEQ ID NO: 497 |
| IER5 | RKRCAAGVGGGPAGCPAPGSTPLKKPRR | SEQ ID NO: 498 |
| ERK5 | RKPVTAQERQREREEKRRRRQERAKERE | SEQ ID NO: 499 |
| | KRRQERER | |
| UL79 | TLLLRETMNNLGVSDHAVLSRKTPQPY | SEQ ID NO: 500 |
| EWS | PGKMDKGEHRQERRDRPY | SEQ ID NO: 501 |
| Hrp1 | RSGGNHRRNGRGGRGGYNRRNNGYHPY | SEQ ID NO: 502 |
| cMyc (1) | PAAKRCKLD | SEQ ID NO: 503 |
| cMyc (2) | RQRRNELKRSP | SEQ ID NO: 504 |
| Mouse c-able IV | SALIKKKKKNAP | SEQ ID NO: 505 |
| Mata2 | KIPIK | SEQ ID NO: 506 |
| MINIYO | KSGSRK | SEQ ID NO: 507 |

A “moiety” as used herein refers to a portion of a molecule. A moiety can be a functional group or describe a portion of a molecule with multiple functional groups (e.g., that share common structural aspects). The terms “moiety” and “functional group” are typically used interchangeably; however, a “functional group” can more specifically refer to a portion of a molecule that comprises some common chemical behavior. “Moiety” is often used as a structural description. In some embodiments, a 5′ terminus, a 3′ terminus, or a 5′ terminus and a 3′ terminus (e.g., a non-native 5′ terminus and/or a non-native 3′ terminus in a first stem element), can comprise one or more moieties.

The terms “modified protein,” “mutated protein,” “protein variant,” and “engineering protein” as used herein typically refers to a protein that has been modified such that it comprises a non-native sequence (i.e., the modified protein has a unique sequence compared to an unmodified protein).

“Transformation” as used herein refers to the insertion of an exogenous polynucleotide into a host cell, irrespective of the method used for insertion. For example, transformation can be by direct uptake, transfection, infection, and the like. The exogenous polynucleotide may be maintained as a nonintegrated vector, for example, an episome, or, alternatively, may be integrated into the host genome.

A “host cell” is a cell that has been transformed, or is capable of transformation, by an exogenous DNA sequence. A host cell can originate from any organism having one or more cells. Examples of host cells include, but are not limited to, a prokaryotic cell, a eukaryotic cell, a bacterial cell, an archacal cell, a cell of a single-cell eukaryotic organism, a protozoa cell, a cell from a plant, an algal cell, a fungal cell (e.g., a yeast cell, a cell from a mushroom), an animal cell, a cell from an invertebrate animal, a cell from a vertebrate animal, such as a cell from a mammal (e.g., a pig, a cow, a goat, a sheep, a rodent, a rat, a mouse, a non-human primate, a human, etc.). Furthermore, a host cell can be a stem cell or progenitor cell, or a cell of the immune system, such as any of the cells of the immune system described herein. The host cell can be a human cell. For example, host cells can be lymphocytes or stem cells, such as hematopoictic stem cells. Lymphocytes include T cells for cell-mediated, cytotoxic adaptive immunity, such as CD4+ and/or CD8+ cytotoxic T cells; natural killer (NK) cells that function in cell-mediated, cytotoxic innate immunity; and B cells for humoral, antibody-driven adaptive immunity. Also included are hematopoietic stem cells that give rise to lymphoid cells. Additionally, CAR-T cells, T-cell receptor (TCR) cells, including TCR-engineered CAR-T cells, tumor infiltrating lymphocytes (TILs), CAR TILs, CAR-NK cells, and the like, can be modified using the techniques herein. In some embodiments, the human cell is outside of the human body. In some embodiments, cells of a body of a living organism (e.g., a human body) are manipulated ex vivo (i.e., outside of the living body). Ex vivo often refers to a medical procedure in which an organ, cells, or tissue are taken from a living body (e.g., a human body) for a treatment or procedure, and then returned to the living body. In vivo often refers to a medical procedure in which an organ, cells, or tissue within a living body (e.g., a human body) are subject to a treatment or procedure.

The terms “subject,” “individual,” or “patient” are used interchangeably herein and refer to any member of the phylum Chordata, including, without limitation, humans and other primates, including non-human primates, such as rhesus macaques, chimpanzees, and other monkey and ape species; farm animals, such as cattle, sheep, pigs, goats, and horses; domestic mammals, such as dogs and cats; laboratory animals, including rabbits, mice, rats, and guinea pigs; birds, including domestic, wild, and game birds, such as chickens, turkeys, and other gallinaceous birds, ducks, and geese; and the like. The term does not denote a particular age or gender. Thus, the term includes adult, young, and newborn individuals, as well as males and females. In some embodiments, a host cell is derived from a subject (for example, lymphocytes, stem cells, progenitor cells, or tissue-specific cells). In some embodiments, the subject is a non-human subject.

The terms “effective amount” or “therapeutically effective amount” of a composition or agent, such as a genetically engineered adoptive cell as provided herein, refer to a sufficient amount of the composition or agent to provide the desired response. Preferably, the effective amount will prevent, avoid, or eliminate one or more harmful side-effects. Such responses will depend on the particular disease in question. For example, in a patient being treated for cancer using an adoptive cell therapy, a desired response may include, prevent, avoid, or eliminate, one or more of: treatment or prevention of the effects of graft versus host disease (GvHD), host versus graft rejection, cytokine release syndrome (CRS), cytokine storm, and the reduction of oncogenic transformations of administered genetically-modified cells. The exact treatment amount required will vary from subject to subject, depending on the species, age, and general condition of the subject, the severity of the condition being treated, and the particular modified lymphocyte used, mode of administration, and the like. An appropriate “effective” amount in any individual case may be determined by one of ordinary skill in the art using routine experimentation.

“Treatment” or “treating” a particular disease, such as a cancerous condition or GvHD, includes: preventing the disease, for example, preventing the development of the disease or causing the disease to occur with less intensity in a subject that may be predisposed to the disease, but does not yet experience or display symptoms of the disease; inhibiting the disease, for example, reducing the rate of development, arresting the development, or reversing the disease state; and/or relieving symptoms of the disease, for example, decreasing the number of symptoms experienced by the subject.

### Cas12 Guides

A Cas12 chRDNA guide molecule of the present disclosure is capable of forming a nucleoprotein complex with a cognate Cas12 protein, such as a Cas12a protein, wherein the complex is capable of targeting a target sequence complementary to the targeting region (spacer sequence).

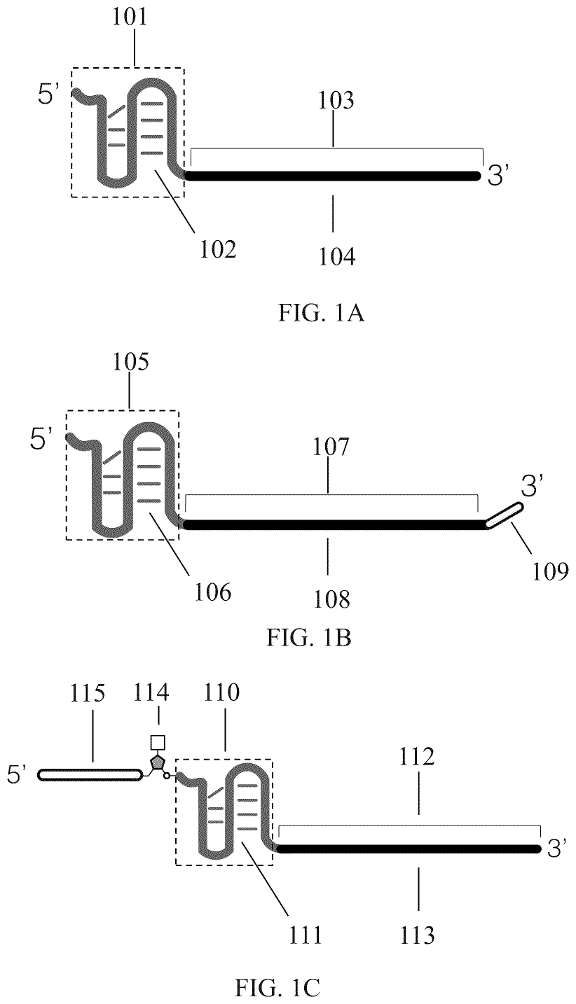

FIG. 1A illustrates an example of an Acidaminococcus spp. BV316 Cas12a guide molecule comprising the following: an activating region (FIG. 1A, 101), comprising a stem-loop duplex (FIG. 1A, 102); and a spacer sequence (FIG. 1A, 103), comprising a target binding sequence (FIG. 1A, 104). FIG. 1B illustrates an alternative Cas12a guide molecule comprising the following: an activating region (FIG. 1B, 105), comprising a stem-loop duplex (FIG. 1B, 106); and a spacer sequence (FIG. 1B, 107), comprising a target binding sequence (FIG. 1B, 108) and a 3′ extension (FIG. 1B, 109). The 3′ extension (FIG. 1B, 109) can be connected to the spacer sequence (FIG. 1B, 107) via a linker sequence. FIG. 1C illustrates an alternative Cas12a guide molecule comprising the following: an activating region (FIG. 1C, 110), comprising a stem-loop duplex (FIG. 1C, 111) and a linker nucleotide (FIG. 1C, 114) and a 5′ extension (FIG. 1C, 115); and a spacer sequence (FIG. 1C, 112), comprising a target binding sequence (FIG. 1C, 113).

In Cas12 chRDNA guide molecules of the present disclosure, the targeting region may comprise DNA, RNA, or a mixture of DNA and RNA. In some embodiments, the targeting region may comprise both DNA and RNA. In certain embodiments, the targeting region may also comprise other base analogs, modified nucleotides, abasic sites, and the like, as well as synthetic, naturally occurring, and non-naturally occurring modified backbone residues or linkages, or combinations thereof.

In Cas12 chRDNA guide molecules of the present disclosure, the activating region may comprise DNA, RNA, or a mixture of DNA and RNA. In some embodiments, the activating region may comprise both DNA and RNA. In certain embodiments, the activating region may also comprise other base analogs, modified nucleotides, abasic sites, and the like, as well as synthetic, naturally occurring, and non-naturally occurring modified backbone residues or linkages, or combinations thereof. In certain embodiments, an activating region is adjacent to a targeting region. In certain embodiments, the activating region is downstream from the targeting region. In certain embodiments, the activating region is upstream from the targeting region.

In some embodiments, Cas12 chRDNA guide molecules of the present disclosure comprise a nucleic acid sequence comprising ribonucleotide bases and about 2% or less, 3% or less, 4% or less, 5% or less, 6% or less, 7% or less, 8% or less, 9% or less, 10% or less, 11% or less, 12% or less, 13% or less, 14% or less, 15% or less, 16% or less, 17% or less, 18% or less, 19% or less, 20% or less, 21% or less, 22% or less, 23% or less, 24% or less, 25% or less, 26% or less, 27% or less, 28% or less, 29% or less, 30% or less, 31% or less, 32% or less, 33% or less, 34% or less, 35% or less, 36% or less, 37% or less, 38% or less, 39% or less, 40% or less, 41% or less, 42% or less, 43% or less, 44% or less, 45% or less, 46% or less, 47% or less, 48% or less, 49% or less, 50% or less, 55% or less, 60% or less, 65% or less, 70% or less, or 75% or less of deoxyribonucleotide bases, or variants or modified derivatives thereof.

The Cas12 chRDNA guides of the present disclosure may be, for example, between 30-75 bases in length, inclusive of abasic sites. In some embodiments, the Cas12 chRDNA guide is 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, or 75 bases in length, inclusive of abasic sites. In some embodiments, the Cas12 chRDNA guide is 40 bases in length, inclusive of abasic sites.

As used herein, the term “as a percentage of the total length” of a polynucleotide sequence, such as a Cas12 chRDNA guide, activating region, or targeting region, refers to the total length of the polynucleotide sequence including abasic sites, and modified and variant bases, for example.

In some embodiments, the activating region is between 10-25 bases in length, inclusive of abasic sites. In some embodiments, the activating region is 10, 11, 12, 13, 14, 15, 16, 17, 18, 19, 20, 21, 22, 23, 24, or 25 bases in length, inclusive of abasic sites. In some embodiments, the activating region is 20 bases in length, inclusive of abasic sites.

In some embodiments, the targeting region is between 10-30 bases in length, inclusive of abasic sites. In some embodiments, the targeting region is 10, 11, 12, 13, 14, 15, 16, 17, 18, 19, 20, 21, 22, 23, 24, 25, 26, 27, 28, 29, or 30 bases in length, inclusive of abasic sites. In some embodiments, the targeting region is 20 bases in length, inclusive of abasic sites.

In embodiments herein, the activating- and/or targeting region comprises ribonucleotide bases and one or more deoxyribonucleotide bases. The activating- and/or targeting region may also, in some embodiments, contain additional modifications including base analogs, modified nucleotides, abasic sites, or combinations thereof. In some embodiments, the activating- and/or targeting region may contain synthetic, naturally occurring, or non-naturally occurring modified backbone residues or linkages, or combinations thereof.

One or more deoxyribonucleotide bases may be present at any one or more positions in the targeting region. For instance, for a targeting region 30 bases in length, inclusive of abasic sites, the one or more deoxyribonucleotide bases may be present at one or more of positions 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30. For smaller targeting regions, the positions will be reduced accordingly. For example, for a targeting region 20 bases in length, inclusive of abasic sites, the one or more deoxyribonucleotide bases may be present at one or more of positions 1, 2, 3, 4, 5, 6, 7, 8, 9, 10, 11, 12, 13, 14, 15, 16, 17, 18, 19, or 20.

One or more additional modifications (such as base analogs, modified nucleotides, abasic sites, modified backbone residues or linkages, or combinations thereof) may be present at any one or more positions in the targeting region. For instance, for a targeting region 30 bases in length, inclusive of abasic sites, the one or more additional modifications may be present at one or more of positions 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30. For smaller targeting regions, the positions will be reduced accordingly. For example, for a targeting region 20 bases in length, inclusive of abasic sites, the one or more additional modifications may be present at one or more of positions 1, 2, 3, 4, 5, 6, 7, 8, 9, 10, 11, 12, 13, 14, 15, 16, 17, 18, 19, or 20.

One or more deoxyribonucleotide bases may be present at any one or more positions in the activating region. For instance, for an activating region 25 bases in length, inclusive of abasic sites, the one or more deoxyribonucleotide bases may be present at one or more of positions 1, 2, 3, 4, 5, 6, 7, 8, 9, 10, 11, 12, 13, 14, 15, 16, 17, 18, 19, 20, 21, 22, 23, 24, or 25. For smaller activating regions, the positions will be reduced accordingly. For example, for an activating region 20 bases in length, inclusive of abasic sites, the one or more deoxyribonucleotide bases may be present at one or more of positions 1, 2, 3, 4, 5, 6, 7, 8, 9, 10, 11, 12, 13, 14, 15, 16, 17, 18, 19, or 20.

One or more additional modifications (such as base analogs, modified nucleotides, abasic sites, modified backbone residues or linkages, or combinations thereof) may be present at any one or more positions in the activating region. For instance, for an activating region 25 bases in length, inclusive of abasic sites, the one or more additional modifications may be present at one or more of positions 1, 2, 3, 4, 5, 6, 7, 8, 9, 10, 11, 12, 13, 14, 15, 16, 17, 18, 19, 20, 21, 22, 23, 24, or 25. For smaller activating regions, the positions will be reduced accordingly. For example, for an activating region 20 bases in length, inclusive of abasic sites, the one or more additional modifications may be present at one or more of positions 1, 2, 3, 4, 5, 6, 7, 8, 9, 10, 11, 12, 13, 14, 15, 16, 17, 18, 19, or 20.

In some embodiments, the amount of deoxyribonucleotide bases, as a percentage of the total size of the Cas12 chRDNA guide inclusive of abasic sites, is preferably 75% or less. In some embodiments, the amount of deoxyribonucleotide bases, as a percentage of the total size of the Cas12 chRDNA guide inclusive of abasic sites, is 50% or less, 45% or less, 40% or less, 35% or less, 30% or less, 25% or less, 24% or less, 23% or less, 22% or less, 21% or less, 20% or less, 19% or less, 18% or less, 17% or less, 16% or less, 15% or less, 14% or less, 13% or less, 12% or less, 11% or less, 10% or less, 9% or less, 8% or less, 7% or less, 6% or less, or 5% or less.

In some embodiments, the amount of additional modifications (such as base analogs, modified nucleotides, abasic sites, modified backbone residues or linkages, or combinations thereof), as a percentage of the total size of the Cas12 chRDNA guide inclusive of abasic sites, is preferably 75% or less. In some embodiments, the amount of additional modifications, as a percentage of the total size of the Cas12 chRDNA guide inclusive of abasic sites, is 50% or less, 45% or less, 40% or less, 35% or less, 30% or less, 25% or less, 24% or less, 23% or less, 22% or less, 21% or less, 20% or less, 19% or less, 18% or less, 17% or less, 16% or less, 15% or less, 14% or less, 13% or less, 12% or less, 11% or less, 10% or less, 9% or less, 8% or less, 7% or less, 6% or less, or 5% or less.

In some embodiments, the amount of deoxyribonucleotide bases, as a percentage of the total size of the targeting region inclusive of abasic sites, is preferably 75% or less. In some embodiments, the amount of deoxyribonucleotide bases, as a percentage of the total size of the targeting region inclusive of abasic sites, is 50% or less, 45% or less, 40% or less, 35% or less, 30% or less, 25% or less, 24% or less, 23% or less, 22% or less, 21% or less, 20% or less, 19% or less, 18% or less, 17% or less, 16% or less, 15% or less, 14% or less, 13% or less, 12% or less, 11% or less, 10% or less, 9% or less, 8% or less, 7% or less, 6% or less, or 5% or less.

In some embodiments, the amount of additional modifications (such as base analogs, modified nucleotides, abasic sites, modified backbone residues or linkages, or combinations thereof), as a percentage of the total size of the targeting region inclusive of abasic sites, is preferably 75% or less. In some embodiments, the amount of additional modifications, as a percentage of the total size of the targeting region inclusive of abasic sites, is 50% or less, 45% or less, 40% or less, 35% or less, 30% or less, 25% or less, 24% or less, 23% or less, 22% or less, 21% or less, 20% or less, 19% or less, 18% or less, 17% or less, 16% or less, 15% or less, 14% or less, 13% or less, 12% or less, 11% or less, 10% or less, 9% or less, 8% or less, 7% or less, 6% or less, or 5% or less.

In some embodiments, the amount of deoxyribonucleotide bases, as a percentage of the total size of the activating region inclusive of abasic sites, is preferably 75% or less. In some embodiments, the amount of deoxyribonucleotide bases, as a percentage of the total size of the activating region inclusive of abasic sites, is 50% or less, 45% or less, 40% or less, 35% or less, 30% or less, 25% or less, 24% or less, 23% or less, 22% or less, 21% or less, 20% or less, 19% or less, 18% or less, 17% or less, 16% or less, 15% or less, 14% or less, 13% or less, 12% or less, 11% or less, 10% or less, 9% or less, 8% or less, 7% or less, 6% or less, or 5% or less.

In some embodiments, the amount of additional modifications (such as base analogs, modified nucleotides, abasic sites, modified backbone residues or linkages, or combinations thereof), as a percentage of the total size of the activating region inclusive of abasic sites, is preferably 75% or less. In some embodiments, the amount of additional modifications, as a percentage of the total size of the activating region inclusive of abasic sites, is 50% or less, 45% or less, 40% or less, 35% or less, 30% or less, 25% or less, 24% or less, 23% or less, 22% or less, 21% or less, 20% or less, 19% or less, 18% or less, 17% or less, 16% or less, 15% or less, 14% or less, 13% or less, 12% or less, 11% or less, 10% or less, 9% or less, 8% or less, 7% or less, 6% or less, or 5% or less.

In some embodiments, the amount of deoxyribonucleotide bases in the activating region and/or the targeting region is adjusted to provide a statistically significant difference as compared to, for example, the corresponding activating region and/or targeting region without deoxyribonucleotide bases. In some embodiments, the statistically significant difference is a difference in on-target or off-target editing.

In some embodiments, the activating region and the targeting region each contain one or more deoxyribonucleotide bases. In some embodiments, the activating region contains one or more deoxyribonucleotide bases, and the targeting region does not contain any deoxyribonucleotide bases (e.g., contains only RNA and/or modified ribonucleotides). In some embodiments, the targeting region contains one or more deoxyribonucleotide bases, and the activating region does not contain any deoxyribonucleotide bases (e.g., contains only RNA and/or modified ribonucleotides).

In some embodiments, the activating region and the targeting region each contain one or more additional modifications (such as base analogs, modified nucleotides, abasic sites, modified backbone residues or linkages, or combinations thereof). In some embodiments, the activating region contains one or more additional modifications (such as base analogs, modified nucleotides, abasic sites, modified backbone residues or linkages, or combinations thereof), and the targeting region does not contain any additional modifications (i.e., contains only RNA or DNA). In some embodiments, the targeting region contains one or more additional modifications (such as base analogs, modified nucleotides, abasic sites, modified backbone residues or linkages, or combinations thereof), and the activating region does not contain any additional modifications (i.e., contains only RNA or DNA).

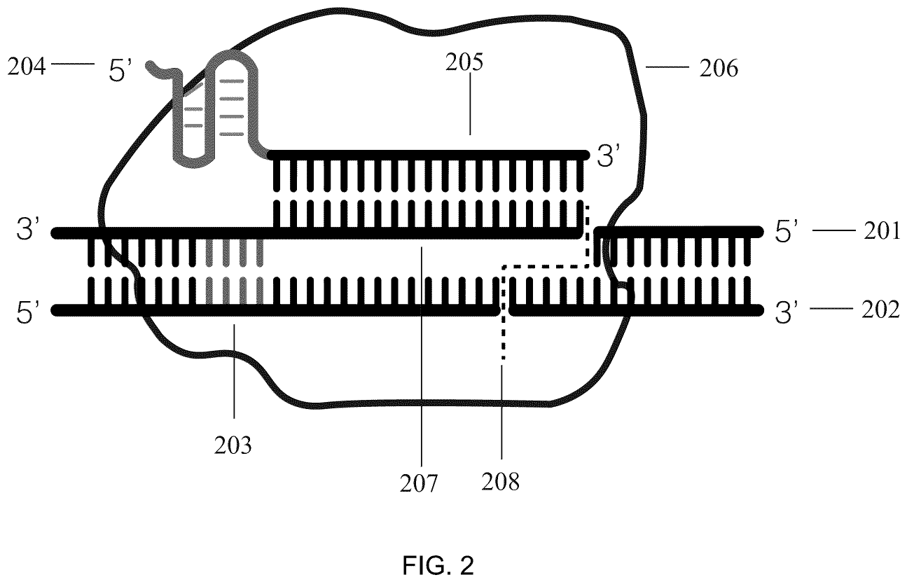

FIG. 2 illustrates a Cas12a protein (FIG. 2, 206) bound to a cognate Cas12a chRDNA guide molecule (FIG. 2, 204) comprising a target binding sequence (FIG. 2, 205). The Cas12a chRDNA guide/nucleoprotein complex unwinds a target polynucleotide comprising the target sequence, and the target binding sequence of the Cas12 chRDNA guide molecule (FIG. 2, 205) is connected via hydrogen bonds (FIG. 2, indicated by a vertical line between polynucleotides) to the target sequence (FIG. 2, 207). In FIG. 2, the target polynucleotide comprises a target strand (FIG. 2, 201) comprising the target sequence (FIG. 2, 207), and a non-target strand (FIG. 2, 202) comprising a PAM sequence (FIG. 2, 203). The PAM sequence (FIG. 2, 203) typically occurs upstream (i.e., in a 5′ direction) of the target sequence (FIG. 2, 207) on the non-target strand (FIG. 2, 202). Formation of hydrogen bonds between the target binding sequence of the Cas12a chRDNA guide molecule (FIG. 2, 205) and the target sequence (FIG. 2, 207) result in the staggered cleavage (FIG. 2, 208) of the target strand (FIG. 2, 201) and the non-target strand (FIG. 2, 202).

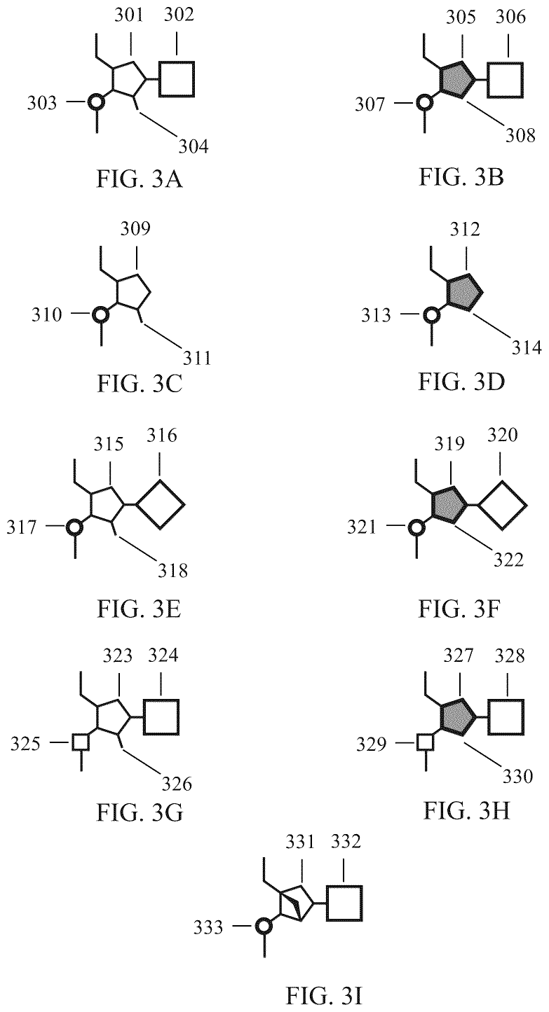

FIG. 3A-FIG. 3I illustrate various canonical and non-canonical nucleotides for use in Cas12 chRDNA guide molecules of the present disclosure. Table 3 presents a series of indicators used in FIG. 3A-FIG. 3I.

TABLE 3
Numerical Indicators of Canonical and Non-canonical
Nucleotides for Use in Cas12 crRNA and chRDNA Guides

| FIG. | Indicator | Description |
|---|---|---|
| FIG. | 301 | a ribose sugar |
| 3A | 302 | a nitrogen base |
| | 303 | a phosphate backbone |
| | 304 | a 2′hydroxyl group |
| FIG. | 305 | a deoxyribose sugar |
| 3B | 306 | a nitrogen base |
| | 307 | a phosphate backbone |
| | 308 | a lack of a 2′hydroxyl group |
| FIG. | 309 | a ribose sugar |
| 3C | 310 | a phosphate backbone |
| | 311 | a 2′hydroxyl group |
| FIG. | 312 | a deoxyribose sugar |
| 3D | 313 | a phosphate backbone |
| | 314 | a lack of a 2′hydroxyl group |
| FIG. 3E | 315 | a ribose sugar |
| | 316 | a non-canonical nitrogen base or base mimic |
| | 317 | a phosphate backbone |
| | 318 | a 2′hydroxyl group |
| FIG. 3F | 319 | a deoxyribose sugar |
| | 320 | a non-canonical base or base mimic |
| | 321 | a phosphate backbone |
| | 322 | a lack of a 2′hydroxyl group |
| FIG. | 323 | a ribose sugar |
| 3G | 324 | a nitrogen base |
| | 325 | a chemically modified backbone |
| | 326 | a 2′hydroxyl group |
| FIG. | 327 | a deoxyribose sugar |
| 3H | 328 | a nitrogen base |
| | 329 | a chemically modified backbone |
| | 330 | a lack of a 2′hydroxyl group |
| FIG. 3I | 331 | a non-canonical or chemically modified sugar |
| | 332 | a nitrogen base |
| | 333 | a phosphate backbone |

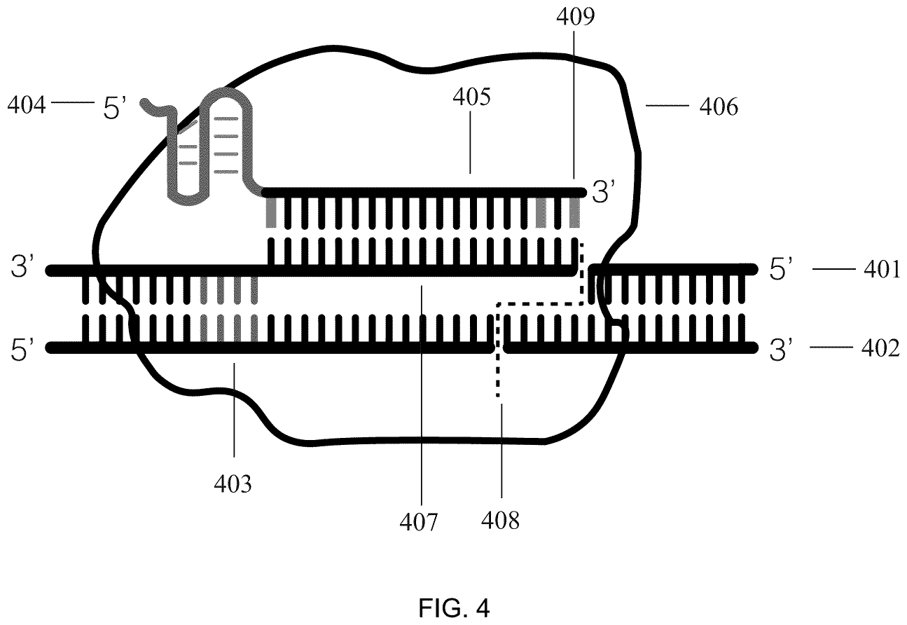

FIG. 4 illustrates a Cas12a protein (FIG. 4, 406) bound to a cognate Cas12a chRDNA guide molecule (FIG. 4, 404) comprising a target binding sequence (FIG. 4, 405), wherein the target binding sequence (FIG. 4, 405) comprises non-RNA nucleotides (FIG. 4, 409) such as a canonical and non-canonical nucleotide presented in FIG. 3B-FIG. 3I. The Cas12a chRDNA guide/nucleoprotein complex unwinds a target polynucleotide comprising the target sequence, and the target binding sequence of the Cas12 chRDNA guide molecule (FIG. 4, 405) is connected via hydrogen bonds (FIG. 4, indicated by a vertical line between polynucleotides) to the target sequence (FIG. 4, 407). In FIG. 4, the target polynucleotide comprises a target strand (FIG. 4, 401) comprising the target sequence (FIG. 4, 407), and a non-target strand (FIG. 4, 402) comprising a PAM sequence (FIG. 4, 403). The PAM sequence (FIG. 4, 403) typically occurs upstream (i.e., in a 5′ direction) of the target sequence (FIG. 4, 407) on the non-target strand (FIG. 4, 402). Formation of hydrogen bonds between the target binding sequence of the chRDNA guide molecule (FIG. 4, 405) and the target sequence (FIG. 4, 407) result in the staggered cleavage (FIG. 4, 408) of the target strand (FIG. 4, 401) and the non-target strand (FIG. 4, 402).

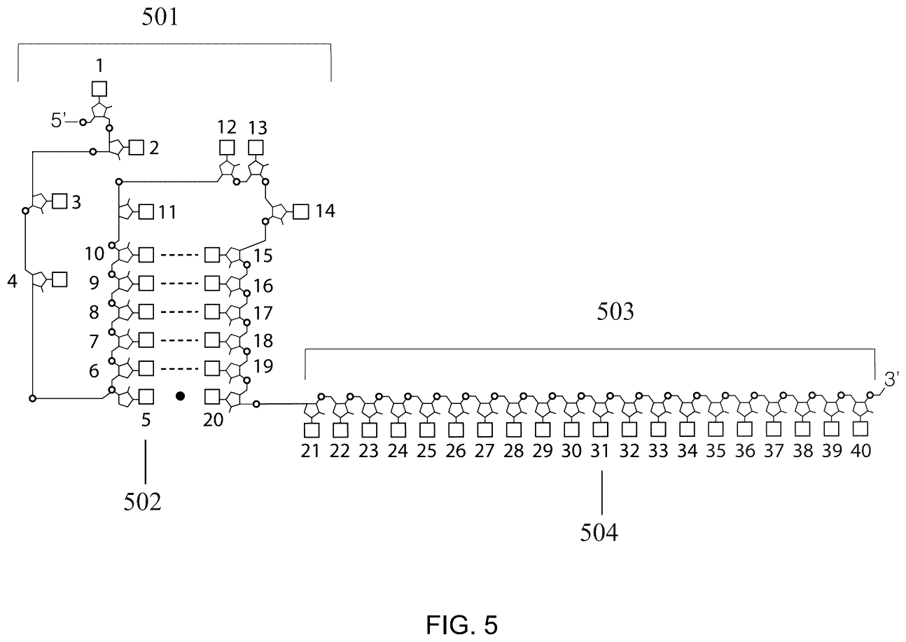

FIG. 5 illustrates an example of an Acidaminococcus spp. (strain BV3L6) Cas12a crRNA guide molecule comprising the following: an activating region (FIG. 5, 501), comprising a stem-loop duplex (FIG. 5, 502); and a spacer (FIG. 5, 503), comprising a target binding sequence (FIG. 5, 504). Each nucleotide position in the activating region (FIG. 5, 501) and in the spacer (FIG. 5, 503) is labeled starting at the 5′ end of the guide molecule, wherein the activating region and the target binding region each comprises RNA.

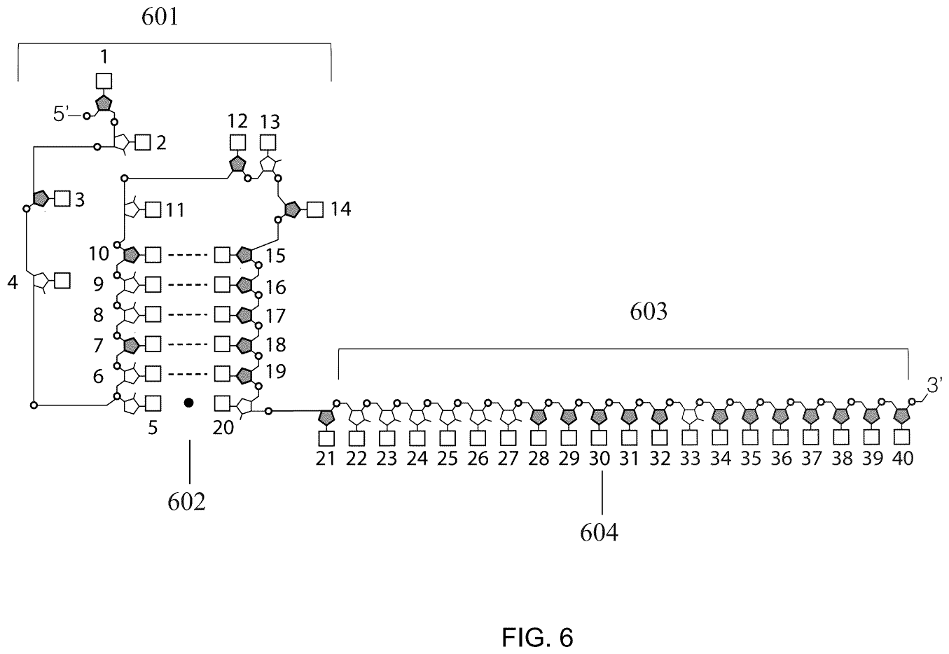

FIG. 6 illustrates an example of an Acidaminococcus spp. (strain BV3L6) Cas12a chRDNA guide molecule comprising the following: an activating region (FIG. 6, 601), comprising a stem-loop duplex (FIG. 6, 602); and a spacer (FIG. 6, 603), comprising a target binding sequence (FIG. 6, 604). Each nucleotide position in the activating region (FIG. 6, 601) in the spacer (FIG. 6, 603) is labeled starting at the 5′ end of the guide molecule, wherein the activating region comprises a mixture of RNA (white fill) and DNA (grey fill) and the target binding sequence comprises a mixture of RNA (white fill) and DNA (grey fill).

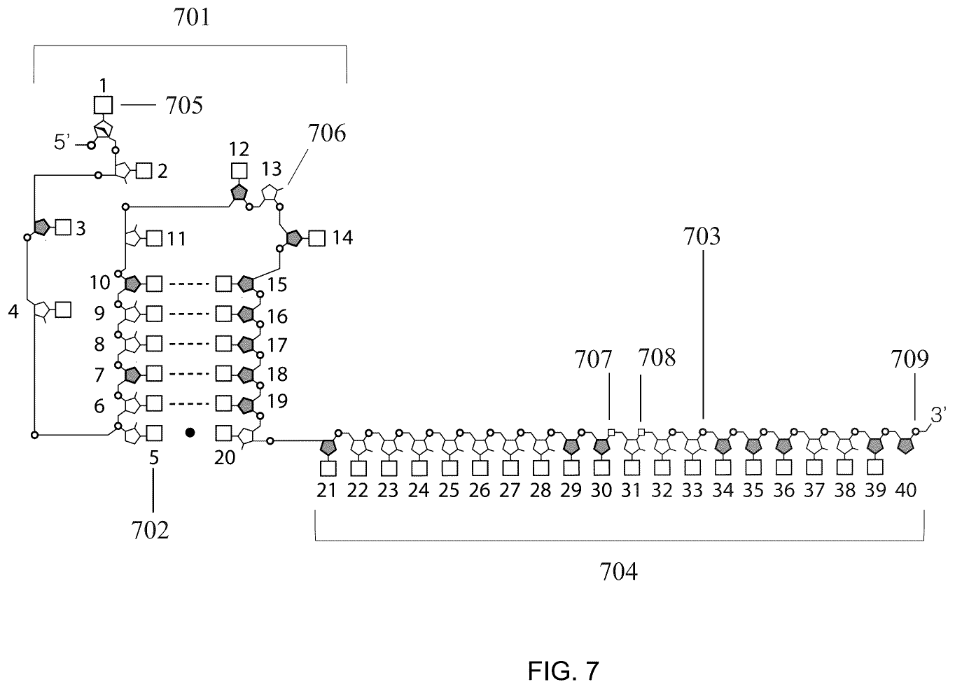

FIG. 7 illustrates an example of an Acidaminococcus spp. (strain BV3L6) Cas12a chRDNA guide molecule comprising the following: an activating region (FIG. 7, 701), comprising a stem-loop duplex (FIG. 7, 702), and a spacer (FIG. 7, 703), comprising a target binding sequence (FIG. 7, 704). Each nucleotide position in the activating region (FIG. 7, 701) and in the spacer (FIG. 7, 703) is labeled starting at the 5′ end of the guide molecule, wherein the activating region comprises a mixture of RNA (white fill) and DNA (grey fill). The Cas12a chRDNA guide molecule further comprises other non-canonical nucleotides, such as a chemically modified sugar nucleotide (FIG. 7, 705), an abasic ribonucleotide (FIG. 7, 706), a deoxy-ribonucleotide with a chemically modified backbone (FIG. 7, 707), a ribonucleotide with a chemically modified backbone (FIG. 7, 708), and an abasic deoxy-ribonucleotide (FIG. 7, 709).

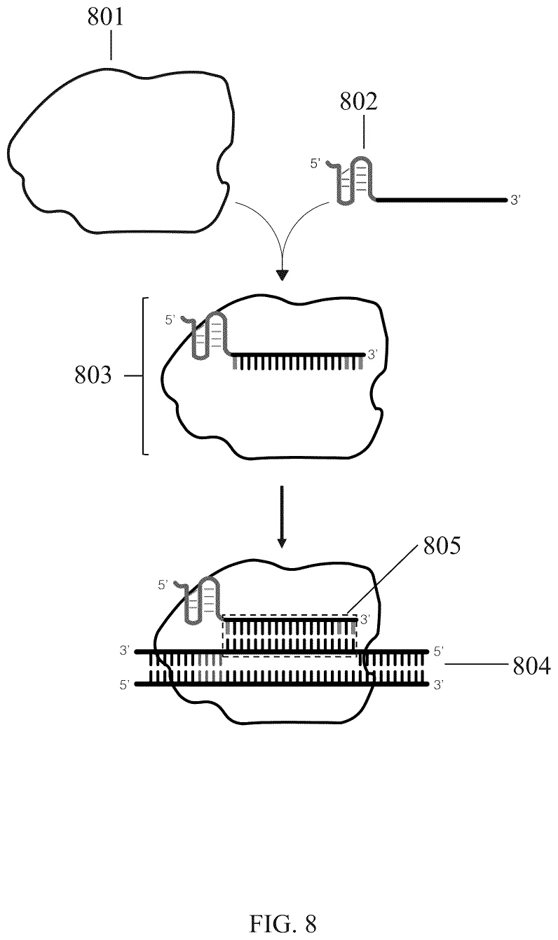

FIG. 8 illustrates the formation of a Cas12 chRDNA guide/nucleoprotein complex, wherein a Cas12 protein (FIG. 8, 801) binds a Cas12 chRDNA guide molecule (FIG. 8, 802) to form a Cas12 chRDNA guide/nucleoprotein complex (FIG. 8, 803). The Cas12 chRDNA guide/nucleoprotein complex (FIG. 8, 803) binds a target polynucleotide (FIG. 8, 804), wherein the target polynucleotide contains a target sequence complementary to the target binding sequence of the Cas12 chRDNA guide molecule, and hydrogen bonds form between the target binding sequence of the Cas12 chRDNA guide molecule and the target sequence (FIG. 8, 805).

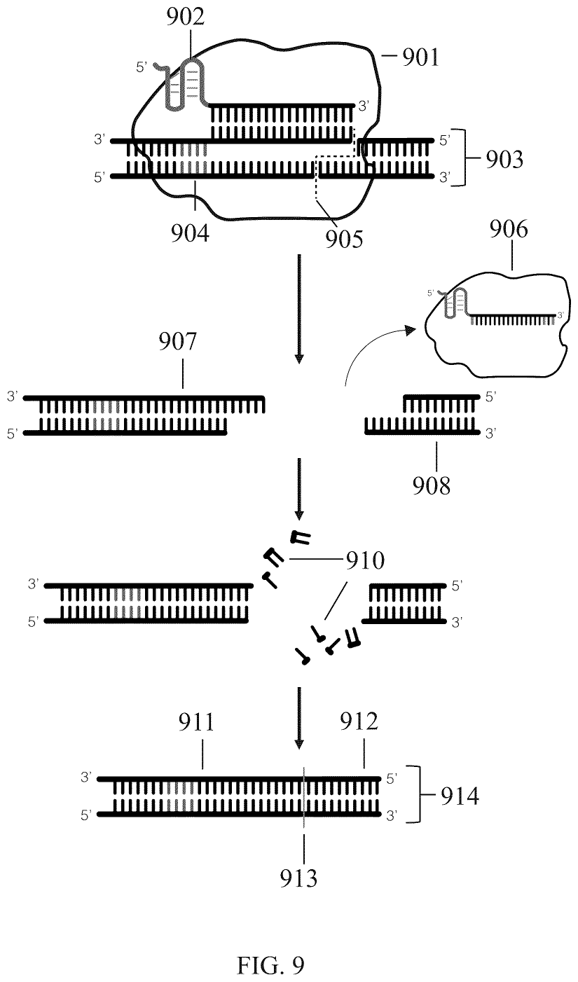

FIG. 9 illustrates the generation of insertion or deletion (indels) in a target polynucleotide by a Cas12 chRDNA guide/nucleoprotein complex, wherein a Cas12 protein (FIG. 9, 901) complexed with a Cas12 chRDNA guide molecule (FIG. 9, 902) binds a target polynucleotide (FIG. 9, 903) comprising a PAM (FIG. 9, 904), and the target polynucleotide is cleaved (FIG. 9, 905) by the Cas12 chRDNA guide/nucleoprotein complex. After targeting has occurred, the Cas12 chRDNA guide/nucleoprotein complex disassociates from the target polynucleotide (FIG. 9, 906), wherein the target polynucleotide comprises an upstream (i.e., in a 5′ direction) strand (FIG. 9, 907) and a downstream (i.e., in a 3′ direction) strand (FIG. 9, 908) relative to the PAM (FIG. 9, 904). The cellular DNA repair machinery repairs the target polynucleotide through insertion or deletion (FIG. 9, 910) of the sequence around the cleavage site in the target polynucleotide. The upstream strand (FIG. 9, 911) and a downstream strand (FIG. 9, 912) are rejoined and the edited target polynucleotide (FIG. 9, 914) comprises indels (FIG. 9, 913) at the cleavage site, wherein the edited target polynucleotide has a different sequence relative to an unedited target polynucleotide. In some embodiments, the generation of insertion or deletion (indels) in a target polynucleotide by a Cas12 chRDNA guide/nucleoprotein complex occurs inside a cell.

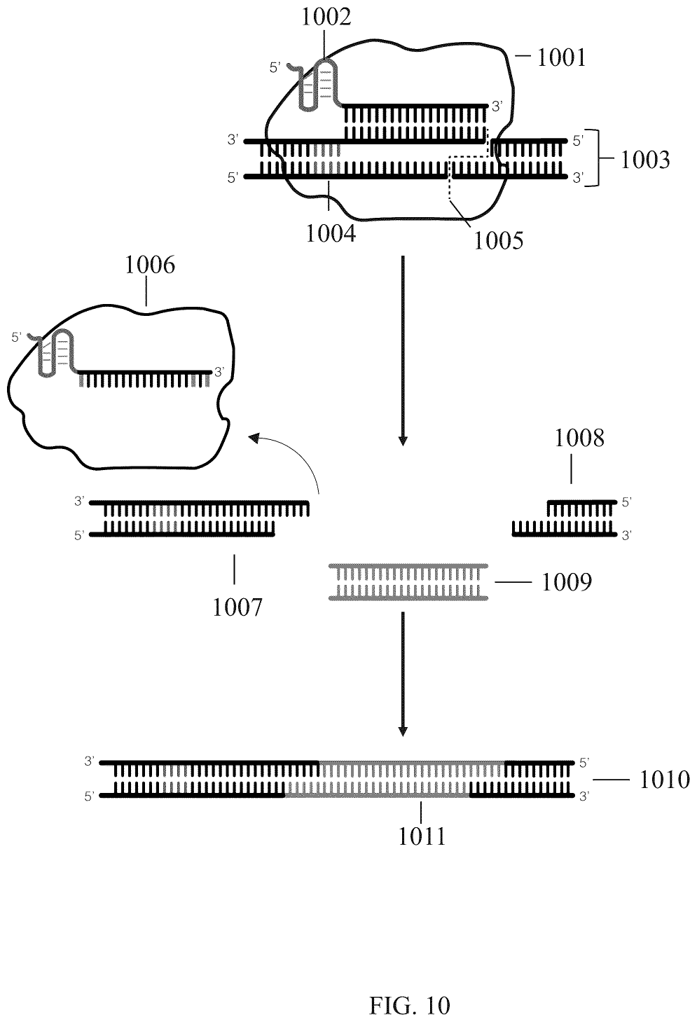

FIG. 10 illustrates incorporation of a donor polynucleotide sequence into a target polynucleotide, wherein a Cas12 protein (FIG. 10, 1001) complexed with a Cas12 chRDNA guide molecule (FIG. 10, 1002) binds a target polynucleotide (FIG. 10, 1003) comprising a PAM (FIG. 10, 1004), and the target polynucleotide is cleaved (FIG. 10, 1005) by the Cas12 chRDNA guide/nucleoprotein complex. After targeting has occurred, the Cas12 chRDNA guide/nucleoprotein complex disassociates from the target polynucleotide (FIG. 10, 1006), wherein the target polynucleotide comprises an upstream (i.e., in a 5′ direction) strand (FIG. 10, 1007) and a downstream (i.e., in a 3′ direction) strand (FIG. 10, 1008) relative to the PAM (FIG. 10, 1004), and wherein a donor polynucleotide is provided (FIG. 10, 1009). The cellular DNA repair machinery repairs the target polynucleotide (FIG. 10, 1010) using the donor polynucleotide (FIG. 10, 1011). The resulting edited target polynucleotide (FIG. 10, 1010) comprises the donor sequence (FIG. 10, 1011) at the target site. In some embodiments, the incorporation of a donor polynucleotide sequence into a target polynucleotide occurs inside a cell.

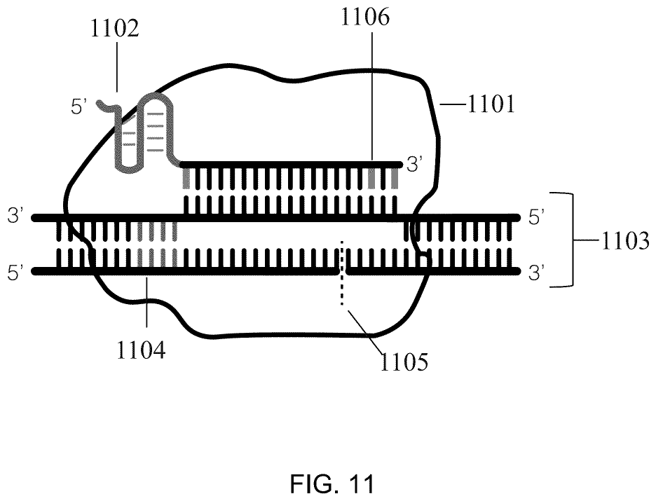

FIG. 11 illustrates nicking of a target polynucleotide, wherein a Cas12 protein (FIG. 11, 1101) complexed to a Cas12 chRDNA guide molecule (FIG. 11, 1102), comprising DNA bases in the target binding sequence (FIG. 11, 1106), binds a target polynucleotide (FIG. 11, 1103) comprising a PAM (FIG. 11, 1104), and the target polynucleotide is nicked (FIG. 11, 1105) in only one strand of the target polynucleotide by the Cas12 chRDNA guide/nucleoprotein complex.

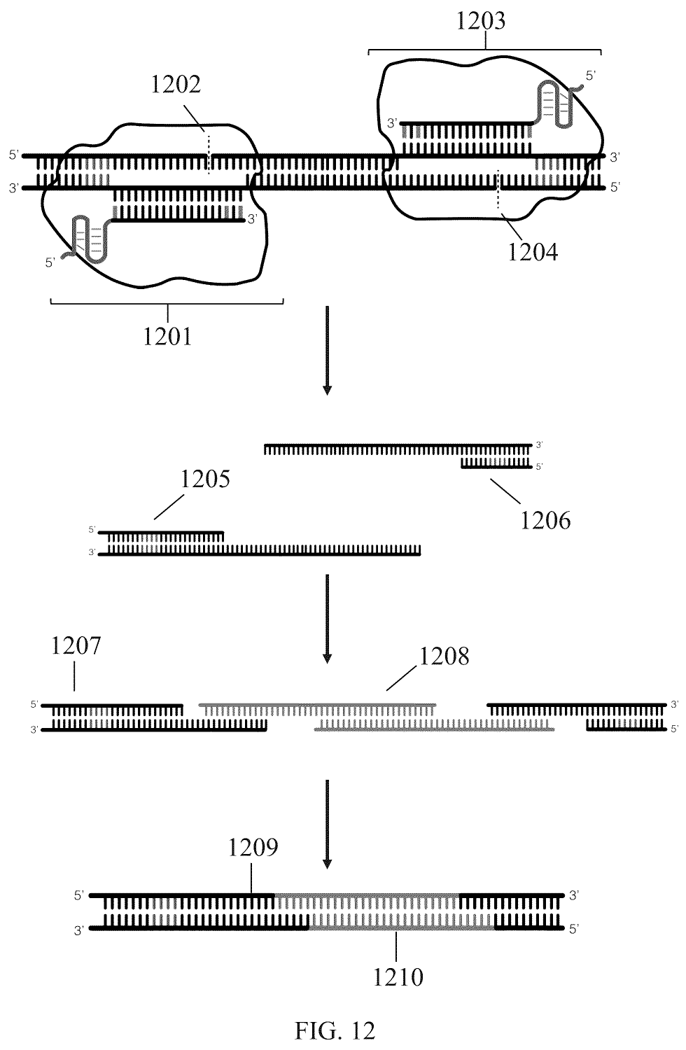

FIG. 12 illustrates the use of two nicking Cas12 chRDNA guide/nucleoprotein complexes to generate a staggered double-strand break in a target polynucleotide, wherein a first Cas12 chRDNA guide/nucleoprotein complex binds an upstream (i.e., in a 5′ direction) target sequence of a target polynucleotide (FIG. 12, 1201) creating a first nick in the target polynucleotide (FIG. 12, 1202) and a second Cas12 chRDNA guide/nucleoprotein complex binds a downstream (i.e., in a 3′ direction) target sequence of a target polynucleotide (FIG. 12, 1203) creating a second nick in the target polynucleotide (FIG. 12, 1204). After tandem nicking has occurred, the post-cleavage target polynucleotide comprises an upstream (i.e., in a 5′ direction) strand (FIG. 12, 1205) and a downstream (i.e., in a 3′ direction) strand (FIG. 12, 1206) with 5′ overhangs. A donor polynucleotide is provided, and the cellular DNA repair machinery repairs the target polynucleotide (FIG. 12, 1207) using the donor polynucleotide (FIG. 12, 1208). The resulting edited target polynucleotide (FIG. 12, 1209) comprises the donor sequence (FIG. 12, 1210) at the tandem nicked site. In some embodiments, the use of two nicking Cas12chRDNA guide/nucleoprotein complexes to generate a staggered DSB in the target polynucleotide occurs inside a cell.

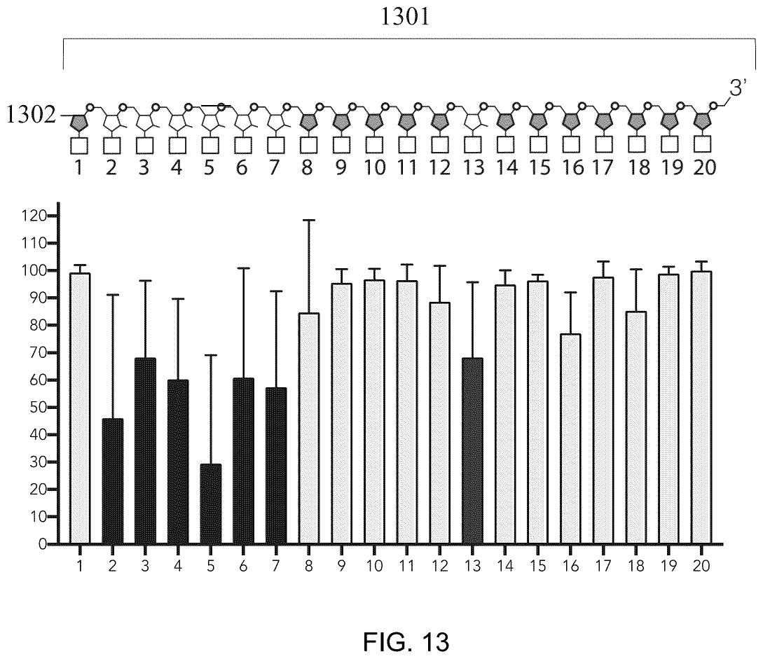

FIG. 13 illustrates the positions in the target binding sequence of a Acidaminococcus spp. (strain BV3L6) Cas12a chRDNA guide molecule amenable to DNA bases. The y-axis represents the normalized percent editing of multiple targets with DNA (see Example 5) at a single position in the target binding sequence (error bars show standard deviation). The x-axis indicates the positions (5′ to 3′) of each position in the target binding sequence. The target binding sequence is illustrated above the graph (FIG. 13, 1301) with preferred positions of DNA base utilization (i.e., greater than 70% average normalized editing) indicated with grey fill. The location of the Cas12a chRDNA activating regions is also indicated (FIG. 13, 1302).

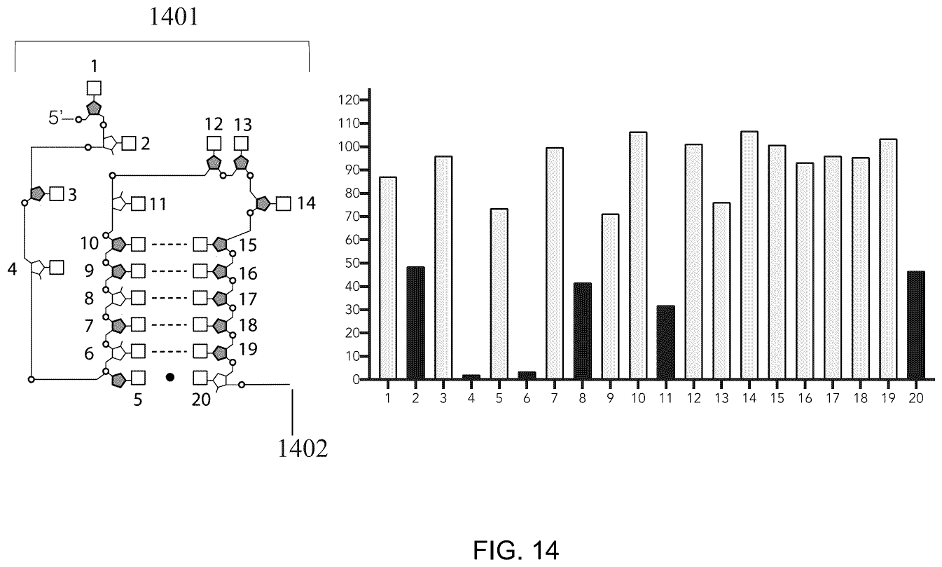

FIG. 14 illustrates the positions in the activating region of a Acidaminococcus spp. (strain BV3L6) Cas12a chRDNA guide molecule amenable to DNA bases (see Example 8). The y-axis represents the normalized percent editing of a guide molecule with DNA at a single position in the activating region. The x-axis indicates the positions (5′ to 3′) of each position in the activating region. The activating region is illustrated the left of the graph (FIG. 14, 1401) with preferred positions of DNA base utilization (i.e., greater than 70% average normalized editing) indicated with grey fill. The location of the Cas12a chRDNA guide target binding sequence is also indicated (FIG. 14, 1402).

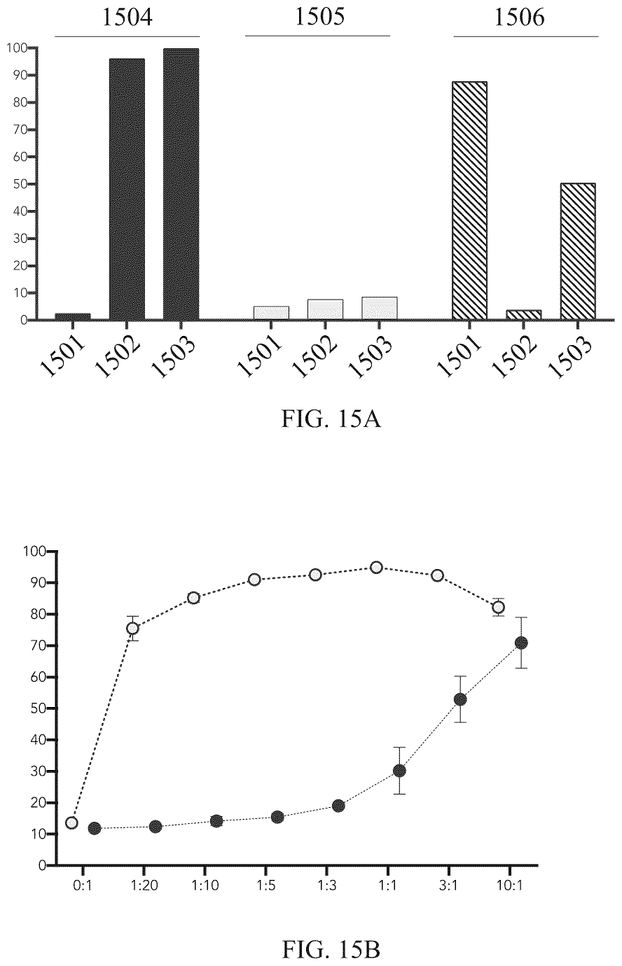

FIG. 15A and FIG. 15B illustrates flow cytometry analysis of CAR-T cells engineered using Cas12a/chRDNA nucleoprotein complexes. FIG. 15A shows the percent of cells expressing an anti-BCMA CAR (FIG. 15A, 1501), TRAC protein (FIG. 15A, 1502), and B2M protein (FIG. 15A, 1503). The x-axis indicates cells that were untreated (FIG. 15A, 1504), cells that were transfected with Cas12a chRDNA guide/nucleoprotein complexes targeting both the TRAC and B2M gene (FIG. 15A, 1505), and cells that were transfected with both Cas12a chRDNA guide/nucleoprotein complexes targeting both the TRAC and B2M gene and transduced with two viruses containing DNA donors encoding the anti-BCMA CAR and B2M-HLA-E fusion gene, respectively (FIG. 15A, 1506). The y-axis represents the percent-positive cells, as measured via flow cytometry, for the various cell surface markers. FIG. 15B illustrates the results from the in vitro cytotoxicity assay for anti-BCMA, B2M-HLA-E CAR-T cells (grey circles) and control TRAC KO T cells (black circles) against a BCMA-positive target cell line. The y-axis represents the percent of target cell killing, and the x-axis indicates the E:T ratio used. Each data point represents the average of 3 co-culture wells at each E:T ratio.

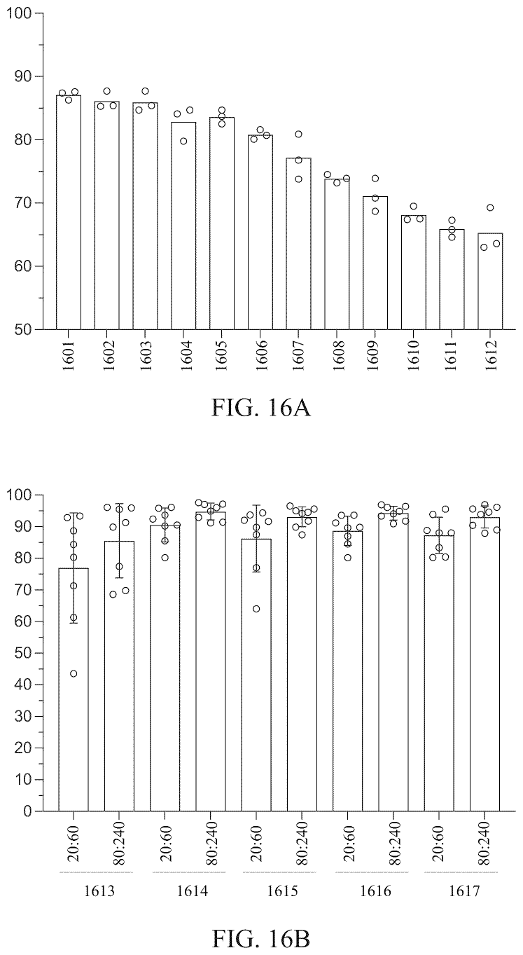

FIG. 16A and FIG. 16B illustrates the cellular editing activity of Cas12a/chRDNA nucleoprotein complex comprising multiple linker and nuclear localization sequence (NLS) configurations. The y-axis of the graphs in FIG. 16A and FIG. 16B indicated percent editing as measured by next generation sequencing. In FIG. 16A the x-axis indicates the each linker-NLS configuration, where each data point represents a replicate measurement. In FIG. 16B the x-axis indicates pmol concentration of Cas12a and chRDNA guide (20:60 or 80:240 pmol) of the top four linker-NLS designs shown in FIG. 16A as well as the ‘unoptimized’ linker-NLS configuration (FIG. 16B, 1613). Each data point represents a unique guide target sequence and is the average of three replicate measurements per data point.

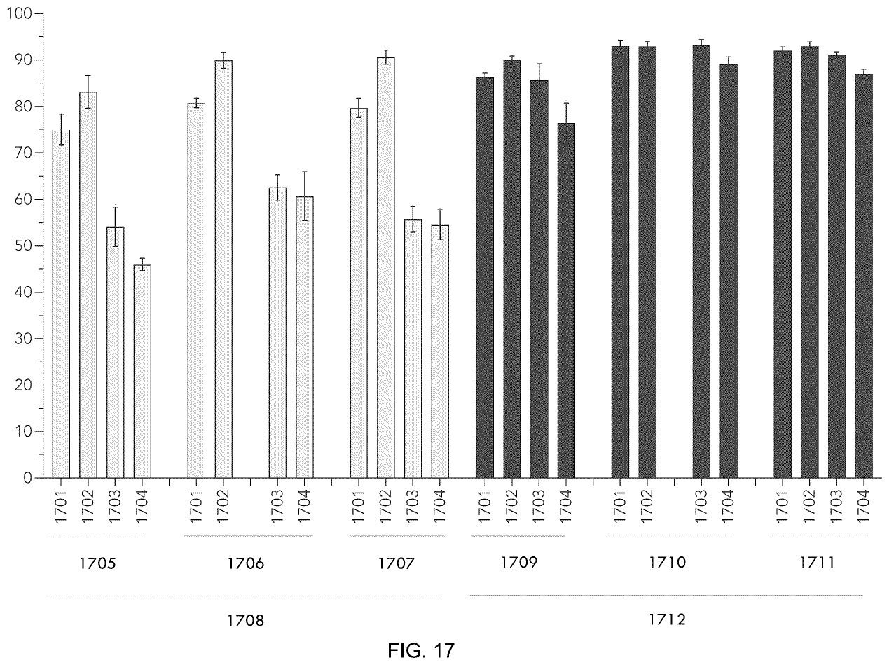

FIG. 17 illustrates the cellular editing activity of Cas12a chRDNA guide/nucleoprotein complexes with the GS-SV40 (FIG. 7, 1708; SEQ ID NO:479) and a (G4S) 2-NPL (FIG. 7, 1712; SEQ ID NO:489) when codelivering multiple Cas12a guides in a single transfection reaction. The y-axis of FIG. 17 indicated percent editing as measured by next generation sequencing. In FIG. 17, the x-axis indicates the target gene as the TRAC gene (FIG. 17, 1701; SEQ ID NO:36), the B2M gene (FIG. 17, 1702; SEQ ID NO:62), the CISH gene (FIG. 17, 1703; SEQ ID NO:158), or the CBLB gene (FIG. 17, 1704; SEQ ID NO: 171). Cas12a chRDNA guide/nucleoprotein complexes were used as single targeting complex per transfection (FIG. 17, 1705 and FIG. 17, 1709), two targeting complexes per transfection (FIG. 17, 1706 and FIG. 17, 1710), or four targeting complexes per transfection (FIG. 17, 1707 and FIG. 17, 1711). Each bar represents the average of two to three replicates.

Methods of designing particular Cas12 chRDNA guide molecules into which deoxyribonucleotide bases, and optionally additional modifications (such as base analogs, modified nucleotides, abasic sites, modified backbone residues or linkages, or combinations thereof) can be designed, are known. See, e.g., Briner et al. (Molecular Cell, 2014, 56:333-339). To do so, the genomic sequence for the gene to be targeted is first identified. The exact region of the selected gene to target will depend on the specific application. For example, in order to activate or repress a target gene using, for example, CRISPR activation or CRISPR inhibition, Cas12 chRDNA guide/nucleoprotein complexes can be targeted to the promoter driving expression of the gene of interest. For genetic knockouts, Cas12 chRDNA guide molecules may be designed to target 5′ constitutively expressed exons, to reduce the chance of removal of the targeted region from mRNA due to alternative splicing. Exons near the N-terminus can be targeted because frameshift mutations here will increase the likelihood of the production of a nonfunctional protein product. Alternatively, cognate Cas12 chRDNA guide molecules can be designed to target exons that code for known essential protein domains. In this regard, non-frameshift mutations such as insertions or deletions are more likely to alter protein function when they occur in protein domains that are essential for protein function. For gene editing using HDR, the target sequence should be close to the location of the desired edit. In this case, the location where the edit is desired is identified and a target sequence is selected nearby.

In some embodiments, a Cas12 chRDNA guide molecule can be designed such that the Cas12 chRDNA guide/nucleoprotein complex can bind outside of the cleavage site of the Cas12 protein. In this case, the target nucleic acid may not interact with the Cas12 chRDNA guide/nucleoprotein complex and the target nucleic acid can be excised (e.g., free from the Cas12 chRDNA guide/nucleoprotein complex). In some embodiments, a Cas12 chRDNA guide molecule can be designed such that the Cas12 chRDNA guide/nucleoprotein complex can bind inside of the cleavage site of the Cas12 protein. In this case, the target nucleic acid can interact with the Cas12 chRDNA guide/nucleoprotein complex and the target nucleic acid can be bound (e.g., bound to the Cas12 chRDNA guide/nucleoprotein complex).

Cas12 chRDNA guide molecules can be designed in such a way that the Cas12 chRDNA guide/nucleoprotein complex can hybridize to a plurality of locations within a nucleic acid sample. A plurality of Cas12 chRDNA guide/nucleoprotein complexes can be contacted to a nucleic acid sample. The plurality of Cas12 chRDNA guide/nucleoprotein complexes can comprise Cas12 chRDNA guide molecules designed to hybridize to the same sequence. The plurality of Cas12 chRDNA guide/nucleoprotein complexes can comprise Cas12 chRDNA guide molecules designed to hybridize to different target sequences.

The target sequences can be at different locations within a target nucleic acid. The locations can comprise the same, or similar, target nucleic acid sequences. The locations can comprise different target nucleic acid sequences. The locations can be a defined according to their distance from each other. The locations can be less than 10 kilobases (Kb) apart, less than 8 Kb apart, less than 6 Kb apart, less than 4 Kb apart, less than 2 Kb apart, less than 1 Kb apart, less than 900 nucleotides apart, less than 800 nucleotides apart, less than 700 nucleotides apart, less than 600 nucleotides apart, less than 500 nucleotides apart, less than 400 nucleotides apart, less than 300 nucleotides apart, less than 200 nucleotides apart, or less than 100 nucleotides apart.

The Cas12a chRDNA guide/nucleoprotein complexes can cleave the target nucleic acid, which can result in an excised target nucleic acid that can be less than 10 kilobases (Kb) long, less than 8 Kb long, less than 6 Kb long, less than 4 Kb long, less than 2 Kb long, less than 1 Kb long, less than 900 nucleotides long, less than 800 nucleotides long, less than 700 nucleotides long, less than 600 nucleotides long, less than 500 nucleotides long, less than 400 nucleotides long, less than 300 nucleotides long, less than 200 nucleotides long, or less than 100 nucleotides long.

The Cas12 chRDNA guide/nucleoprotein complexes can be bound to a fragmented target nucleic acid that can be less than 10 kilobases (Kb) long, less than 8 Kb long, less than 6 Kb long, less than 4 Kb long, less than 2 Kb long, less than 1 Kb long, less than 900 nucleotides long, less than 800 nucleotides long, less than 700 nucleotides long, less than 600 nucleotides long, less than 500 nucleotides long, less than 400 nucleotides long, less than 300 nucleotides long, less than 200 nucleotides long, or less than 100 nucleotides long.

The Cas12 chRDNA guide molecules of the present disclosure can be synthesized in vitro by known methods, such as chemically in solution or on a solid support, or can, in some instances, be recombinantly produced. A single production or synthesis technique, or a combination of production and synthesis techniques, may be employed in which deoxyribonucleotide bases and/or modifications may be introduced at one or more positions across the length of a sequence.

In some embodiments, a Cas12 chRDNA guide molecule, targeting region thereof, or activating region thereof, is designed to contain deoxyribonucleotide base(s) (and/or modified deoxyribonucleotide base(s)) at certain positions as compared to a reference Cas12 chRDNA guide molecule, a reference targeting region, or a reference activating region, each composed of ribonucleotide bases, respectively.

In some embodiments, a reference Cas12a chRDNA guide molecule contains the following RNA sequence: UAAUUUCUACUCUUGUAGAUGAGUCUCUCAGCUGGUACAC. Cas12a chRDNA guide molecules of the present disclosure, designed based on this reference RNA sequence, include Cas12 chRDNA guide molecules having one or more deoxyribonucleotide bases at one or more of positions 1, 3, 7, 10, 12, 14, 15, 16, 17, 18, 19, 21, 28, 29, 30, 31, 32, 34, 35, 36, 37, 38, 39, and 40. In some embodiments, 23 or less, 22 or less, 21 or less, 20 or less, 19 or less, 18 or less, 17 or less, 16 or less, 15 or less, 14 or less, 13 or less, 12 or less, 11 or less, 10 or less, 9 or less, 8 or less, 7 or less, 6 or less, 5 or less, 4 or less, 3 or less, 2 or less, or 1, of these listed positions are deoxyribonucleotide bases. In some embodiments, all of the one or more deoxyribonucleotide bases in the targeting region form canonical base pairs with the target sequence. In some embodiments, at least one of the one or more deoxyribonucleotide bases in the targeting region does not form a canonical base pair with the target sequence.

In some embodiments, a reference Cas12a chRDNA guide molecule contains the following RNA sequence: UAAUUUCUACUCUUGUAGAUAGUGGGGGUGAAUUCAGUGU. Cas12 chRDNA guide molecules of the present disclosure, designed based on this reference RNA sequence, include Cas12a chRDNA guide molecules having one or more deoxyribonucleotide bases at one or more of positions 1, 3, 7, 10, 12, 14, 15, 16, 17, 18, 19, 21, 28, 29, 30, 31, 32, 34, 35, 36, 37, 38, 39, and 40. In some embodiments, 23 or less, 22 or less, 21 or less, 20 or less, 19 or less, 18 or less, 17 or less, 16 or less, 15 or less, 14 or less, 13 or less, 12 or less, 11 or less, 10 or less, 9 or less, 8 or less, 7 or less, 6 or less, 5 or less, 4 or less, 3 or less, 2 or less, or 1, of these listed positions are deoxyribonucleotide bases. In some embodiments, all of the one or more deoxyribonucleotide bases in the targeting region form canonical base pairs with the target sequence. In some embodiments, at least one of the one or more deoxyribonucleotide bases in the targeting region does not form a canonical base pair with the target sequence.

In some embodiments, a reference activating region contains the following RNA sequence: UAAUUUCUACUCUUGUAGAU. Deoxyribonucleotide base-containing activating regions of the present disclosure, designed based on this reference RNA sequence, include activating regions having one or more deoxyribonucleotide bases at one or more of positions 1, 3, 7, 10, 12, 14, 15, 16, 17, 18, and 19. In some embodiments, 11 or less, 10 or less, 9 or less, 8 or less, 7 or less, 6 or less, 5 or less, 4 or less, 3 or less, 2 or less, or 1, of these listed positions are deoxyribonucleotide bases.

In some embodiments, a reference targeting region contains the following RNA sequence: GAGUCUCUCAGCUGGUACAC. Deoxyribonucleotide base-containing targeting regions of the present disclosure, designed based on this reference RNA sequence, include targeting regions having one or more deoxyribonucleotide bases at one or more of positions 1, 8, 9, 10, 11, 12, 14, 15, 16, 17, 18, 19, and 20. In some embodiments, 12 or less, 11 or less, 10 or less, 9 or less, 8 or less, 7 or less, 6 or less, 5 or less, 4 or less, 3 or less, 2 or less, or 1, of these listed positions are deoxyribonucleotide bases.

In some embodiments, a reference targeting region contains the following RNA sequence: AGUGGGGGUGAAUUCAGUGU. Deoxyribonucleotide base-containing targeting regions of the present disclosure, designed based on this reference RNA sequence, include targeting regions having one or more deoxyribonucleotide bases at one or more of positions 1, 8, 9, 10, 11, 12, 14, 15, 16, 17, 18, 19, and 20. In some embodiments, 12 or less, 11 or less, 10 or less, 9 or less, 8 or less, 7 or less, 6 or less, 5 or less, 4 or less, 3 or less, 2 or less, or 1, of these listed positions are deoxyribonucleotide bases.

In some embodiments, an activating region has the sequence TrAArUrUrUCrUrACrUCrUTGrUrArGArU (where an “r” precedes a ribonucleotide base; and the absence of an “r” preceding a base indicates a deoxyribonucleotide base).

In some embodiments, a targeting region has the sequence GrArGrUrCrUrCrUrCrAGrCrUrGrGrUrArCrAC (where an “r” precedes a ribonucleotide base; and the absence of an “r” preceding a base indicates a deoxyribonucleotide base).

In some embodiments, a targeting region has the sequence ArGrUrGrGrGrGrGrUrGArArUrUrCrArGrUrGT (where an “r” precedes a ribonucleotide base; and the absence of an “r” preceding a base indicates a deoxyribonucleotide base).

In some embodiments, a Cas12a chRDNA guide has the sequence TrAArUrUrUCrUrACrUCrUTGrUrArGArUGrArGrUrCrUrCrUrCrAGrCrUrGrGrUrArCrA C (where an “r” precedes a ribonucleotide base; and the absence of an “r” preceding a base indicates a deoxyribonucleotide base).

In some embodiments, a Cas12a chRDNA guide has the sequence TrAArUrUrUCrUrACrUCrUTGrUrArGArUArGrUrGrGrGrGrGrUrGArArUrUrCrArGrUrG T (where an “r” precedes a ribonucleotide base; and the absence of an “r” preceding a base indicates a deoxyribonucleotide base).

### Cas12 Proteins

Cas12 proteins of the present disclosure include, but are not limited to, Cas12 wild type proteins derived from Type V CRISPR-Cas systems, modified Cas12 proteins, variants of Cas12 proteins, Cas12 orthologs, and combinations thereof. In some embodiments, the Cas12 protein is a wild type Cas12a protein, a modified Cas12a protein, a variant of a Cas12a protein, a Cas12a ortholog, or a combination thereof.

A Cas12 protein can be modified. The modification can comprise modifications to an amino acid. The modifications can also alter the primary amino acid sequence and/or the secondary, tertiary, and/or quaternary amino acid structure. In some embodiments, one or more amino acid sequences of a Cas12 protein can be varied without a significant effect on the structure or function of the Cas12 protein. The type of mutation may be irrelevant if the alteration occurs in some regions (e.g., a non-critical region) of the protein. Depending upon the location of the replacement, the mutation may not have a major effect on the biological properties of the resulting variant. For example, properties and functions of certain Cas12 variants can be the same type as those of wild type Cas12.

In some cases, whether a mutation may critically impact the structure and/or function of a Cas12 protein may be determined using sequence and/or structural alignment. Sequence alignment can identify regions of a polypeptide that are similar and/or dissimilar (e.g., conserved, not conserved, hydrophobic, hydrophilic, etc.). In some instances, a region in the sequence of interest that is similar to other sequences is suitable for modification. In other instances, a region in the sequence of interest that is dissimilar from other sequences is suitable for modification. For example, sequence alignment can be performed by database search, pairwise alignment, multiple sequence alignment, genomic analysis, motif finding, benchmarking, and/or programs such as BLAST, CS-BLAST, HHPRED, psi-BLAST, LALIGN, PyMOL, and SEQALN. Structural alignment can be performed by programs such as Dali, PHYRE, Chimera, COOT, O, and PyMOL. Alignment can be performed by database search, pairwise alignment, multiple sequence alignment, genomic analysis, motif finding, or bench marking, or any combination thereof.

Cas12 proteins typically consist of six domains corresponding to the REC1, REC2, PAM interacting (PI), Nuclease (Nuc), Wedge (WED), and RuvC domains. See, e.g., Yamano et al. (Cell, 2016, 165 (4): 949-962). The WED domain and RuvC domain can have a tripartite sequence architecture, interrupted by sequences from other domains. For example, the Acidaminococcus spp. Cas12a WED domain sequence is interrupted by the REC1, REC2, and PI domain sequences. Additionally, certain subtypes of Cas12 proteins contain a bridge helix domain that occurs adjacent to, or between, the RuvC domain sequences.

Regions of the Cas12 protein can be modified to modulate the activity of the Cas12 protein. For example, regions of the Acidaminococcus spp. (strain BV3L6) Cas12a protein corresponding to residues of the PI domain (598-718) and WED domain (526-597 and 719-883) can be modified to alter PAM specificity. See, e.g., Tóth et al. (Nucleic Acid Research, 2020, 48 (7): 3722-3733). The region in the Acidaminococcus spp. (strain BV3L6) Cas12a protein corresponding to residues of the REC1 (24-319) and REC2 (320-526) domains can be modified to alter target engagement and cleavage kinetics. Regions of the REC1 (226-304) and REC2 (368-435) domains interact directly with the PAM distal end of the target binding sequence and target sequence, and can be engineered to modify efficiency of target sequence cleavage. Regions of the Nuc domain (1066-1261) and RuvC domain (940-956, 957-1065, and 1261-1307) can be modified to alter the cleavage efficiencies of the target strand, non-target strand, or target strand and non-target strand, of the target sequence. Engineering these regions can comprise introducing mutations, replacing with corresponding regions from other Cas12 orthologues, deletions, insertions, etc.

Modified Cas12 proteins can be used in combination with Cas12 chRDNA guide molecules to alter the activity or specificity of the Cas12 protein. In some instances, a Cas12 protein can be modified to provide enhanced activity or specificity when complexed with a Cas12 chRDNA guide molecule, wherein the Cas12 modifications occur in the REC1, REC2, RuvC, WED, and/or Nuc domain(s). In some instances, a Cas12 protein can be modified to provide enhanced activity or specificity when complexed with a Cas12 chRDNA guide molecule, wherein the Cas12a modifications occur in regions 226-304, 368-435, 940-956, 978-1158, 1159-1180, and 1181-1298 (numbering based on the Acidaminococcus spp. Cas12a sequence).

Such mutations can be produced by site-directed mutagenesis. Mutations can include substitutions, additions, deletions, or any combination thereof. In some instances, the mutation converts the mutated amino acid to alanine. In other instances, the mutation converts the mutated amino acid to another amino acid (e.g., glycine, serine, threonine, cysteine, valine, leucine, isoleucine, methionine, proline, phenylalanine, tyrosine, tryptophan, aspartic acid, glutamic acid, asparagine, glutamine, histidine, lysine, or arginine). The mutation can convert the mutated amino acid to a non-natural amino acid (e.g., selenomethionine). The mutation can convert the mutated amino acid to amino acid mimics (e.g., phosphomimics). The mutation can be a conservative mutation. For example, the mutation can convert the mutated amino acid to amino acids that resemble the size, shape, charge, polarity, conformation, and/or rotamers, of the mutated amino acids (e.g., cysteine/serine mutation, lysine/asparagine mutation, histidine/phenylalanine mutation).

In some embodiments, the Cas12 protein is an nCas12 protein. An nCas12 protein is a variant of a Cas12 protein that is nuclease-deficient, also termed a “nicking Cas12” or “Cas12-nickase.” Such molecules lack a portion of the endonuclease activity and therefore can only nick one strand of the target nucleic acid. See, e.g., Jinek et al. (Science, 2012, 337:816-821). This may be accomplished, for example, by introducing mutation(s) into the RuvC nuclease domain. Non-limiting examples of such modifications can include D917A, E1006A, and D1225A, to the RuvC nuclease domain of the F. novicida Cas12a protein. It is understood that the mutation of other catalytic residues to reduce activity of the RuvC nuclease domain can also be carried out by those skilled in the art. The resultant nCas12 protein is unable to cleave double-stranded DNA, but retains the ability to complex with a guide molecule, bind a target DNA sequence, and nick only one strand of the target DNA. Targeting specificity is determined by Cas12 protein binding to the PAM sequence, and by complementary base pairing of guide molecule to the genomic locus. In some embodiments of the present disclosure, the nCas12 protein is an nCas12a protein.

In some embodiments, the Cas12 protein is a dCas12 protein. A dCas12 protein is a variant of a Cas12 protein that is nuclease-deactivated, also termed a “catalytically inactive Cas12 protein,” an “enzymatically inactive Cas12,” a “catalytically dead Cas12,” or a “dead Cas12.” Such molecules lack endonuclease activity, and can therefore be used to regulate genes in an RNA-guided manner. See, e.g., Jinck et al. (Science, 2012, 337:816-821). Mutations of catalytic residues to eliminate activity of the RuvC domain can be carried out by those skilled in the art. The resultant dCas12 protein is unable to cleave double-stranded DNA, but retains the ability to complex with a guide molecule and bind a target DNA sequence. Targeting specificity is determined by Cas12 protein binding to the PAM sequence, and by complementary base pairing of guide molecule to the genomic locus. In some embodiments of the present disclosure, the dCas12 protein is a dCas12a protein.

Certain Cas12 protein subtypes lack nuclease activity, due to either inactivation of the RuvC-like nuclease domain, or absences in part or in whole of the RuvC-like nuclease domain. One such subtype, Type V-K and associated protein Cas12k, instead are associated with Tn7-like transposable elements tnsB, tnsC, tniQ. See, e.g., Strecker et al. (Science, 2019, 364 (6448): 48-53). Cas12k retains the ability to complex with a guide molecule, and to bind a target DNA sequence, and the associated Tn7-like proteins facilitate the RNA-guided transposition of DNA sequences. In some embodiments of the present disclosure, the Cas12 chRDNA guide/nucleoprotein complex is a Cas12k chRDNA guide/nucleoprotein complex.

Other amino acid alterations may include amino acids with glycosylated forms, aggregative conjugates with other molecules, and covalent conjugates with unrelated chemical moieties (e.g., pegylated molecules). Covalent variants can be prepared by linking functionalities to groups that are found in the amino acid chain or at the N- or C-terminal residue. In some cases, mutated site-directed polypeptides may also include allelic variants and species variants.

In certain embodiments, the Cas12 protein may be a fusion or chimeric protein containing a first domain from a Cas12 protein, and a second domain from a different protein, such as a Csy4 protein. The fusion modification to a Cas12 protein may confer additional activity on the modified Cas12 protein. Such activities can include nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity, glycosylase activity, acetyltransferase activity, deacetylase activity, kinase activity, phosphatase activity, ubiquitin ligase activity, reverse transcriptase activity, deubiquitinating activity, adenylation activity, deadenylation activity, SUMOylating activity, deSUMOylating activity, ribosylation activity, deribosylation activity, and/or myristoylation activity or demyristoylation activity that modifies a polypeptide associated with nucleic acid target sequence (e.g., a histone).

In certain embodiments, a Cas12 protein may contain one or more NLS sequences (e.g., appended to, and/or inserted within, the Cas12 protein sequence). An NLS sequence may be located, for example, at the N-terminus, the C-terminus, or internally within a Cas12 protein (such as a Cas12a protein), including combinations thereof (e.g., one or more NLS at the N-terminus and one or more NLS at the C-terminus)

In certain embodiments, a Cas12 protein, including a Cas12a protein, may contain a plurality of NLS sequences, such as, for example, at least 2, at least 3, at least 4, or at least 5 NLS sequences. The plurality of NLS sequences can be present at a single terminus of the Cas12a protein (e.g., NLS sequences are present only at the N-terminus or only at the C-terminus), or can be present at both termini (e.g., one or more NLS sequences at the N-terminus, and one or more NLS sequences at the C-terminus). NLS sequences can be fully synthetic, modified, or derived from endogenous or exogenous protein sequences. In some embodiments, a Cas12 protein, including a Cas12a protein, may contain an NLS sequence of, or modified or derived from, an NLS sequence selected from SV40 large T-antigen, nucleoplasmin, 53BP1, VACM-1/CUL5, CXCR4, VP1, ING4, IER5, ERK5, UL79, EWS, Hrp1, cMyc (1), cMyc (2), Mouse c-able IV, Matα2, and MINIYO.

In some embodiments, a Cas12 protein, including a Cas12a protein, may contain an NLS sequence of, or modified or derived from, an NLS sequence selected from any of SEQ ID Nos: 04, 05, and 493-507. A modified or derived NLS sequence may contain, for example: 5 or less, 4 or less, 3 or less, 2 or less, or 1, amino acid substitutions; 5 or less, 4 or less, 3 or less, 2 or less, or 1, amino acid deletions; and/or 5 or less, 4 or less, 3 or less, 2 or less, or 1 amino acid additions with respect to a reference NLS sequence (e.g., an NLS sequence selected from any of SEQ ID Nos: 04, 05, and 493-507).

In some embodiments, an NLS sequence may have, for example, at least 80%, at least 85%, at least 90%, at least 91%, at least 92%, at least 93%, at least 94%, at least 95%, at least 96%, at least 97%, at least 98%, or at least 99% sequence identity to an NLS sequence selected from any of SEQ ID Nos: 04, 05, and 493-507.

NLS sequences can be covalently attached (e.g., to a Cas12 protein, to another NLS sequence(s), or to a fusion peptide sequence attached to a Cas12 protein) either directly or via a linker polypeptide. The length of a linker sequence can be optimized depending on the structural characteristics of the particular Cas12 protein (e.g., solvent accessibility of the termini, the presence of other critical functional peptide sequences at the termini, etc.) to ensure the accessibility of the NLS sequence for cognate importin protein binding and trafficking. Additionally, as described in Example 11 herein, it is possible to screen empirically for desirable linker lengths.

In some embodiments, an NLS sequence is covalently attached (e.g., to a Cas12 protein, to another NLS sequence(s), or to a fusion peptide sequence attached to a Cas12 protein) via a linker sequence comprising one or more amino acids. In some embodiments, a linker sequence contains at least one glycine, serine, and/or threonine residue. In some embodiments, a linker sequence contains at least one glycine residue and at least one serine residue. In some embodiments, a linker sequence contains a plurality of glycine residues and at least one serine residue. In some embodiments, a linker sequence consists of or comprises a GS sequence. In some embodiments, a linker sequence consists of or comprises a GGGGS sequence. In some embodiments, a linker sequence consists of or comprises a GGGGSGGGGS sequence.

In some embodiments, a Cas12a protein comprises at least one linker sequence and at least one NLS sequence at the C-terminus. In some embodiments, the at least one NLS sequence is selected from SV40 large T-antigen and nucleoplasmin, or sequences modified or derived therefrom.

In certain embodiments, a Cas12a protein comprises a GGGGSGGGGS linker sequence and a nucleoplasmin NLS sequence at the C-terminus, wherein the nucleoplasmin NLS sequence is positioned on the C-terminal side of the GGGGSGGGGS linker sequence.

In certain embodiments, a Cas12a protein comprises at least one GS linker sequence, a SV40 large T-antigen NLS sequence, and a nucleoplasmin NLS sequence at the C-terminus, wherein the nucleoplasmin NLS sequence is positioned on the C-terminal side of the SV40 large T-antigen NLS sequence. In some embodiments thereof, a first GS linker sequence is present at the N-terminal side of the SV40 large T-antigen NLS sequence, and a second GS linker sequence is present between the SV40 large T-antigen NLS sequence and the nucleoplasmin NLS sequence.

In certain embodiments, a Cas12a protein comprises a GS linker sequence, a GGGGSGGGGS linker sequence, a SV40 large T-antigen NLS sequence, and a nucleoplasmin NLS sequence at the C-terminus, wherein the nucleoplasmin NLS sequence is positioned on the C-terminal side of the SV40 large T-antigen NLS sequence. In some embodiments thereof, a GGGGSGGGGS linker sequence is present at the N-terminal side of the SV40 large T-antigen NLS sequence, and a GS linker sequence is present between the SV40 large T-antigen NLS sequence and the nucleoplasmin NLS sequence.

In certain embodiments, a Cas12a protein comprises a GGGGSGGGGS linker sequence and a SV40 large T-antigen NLS sequence at the C-terminus, wherein the SV40 large T-antigen NLS sequence is positioned on the C-terminal side of the GGGGSGGGGS linker sequence.

In some embodiments, a Cas12a protein comprises a linker- and NLS-containing sequence at the C-terminus. In some embodiments, this linker- and NLS-containing sequence comprises or consists of an amino acid sequence selected from SEQ ID Nos: 479-490. In some embodiments, the linker- and NLS-containing sequence comprises or consists of an amino acid sequence having at least 80%, at least 85%, at least 90%, at least 91%, at least 92%, at least 93%, at least 94%, at least 95%, at least 96%, at least 97%, at least 98%, or at least 99% sequence identity to an amino acid sequence selected from SEQ ID Nos: 479-490.

Cognate Cas12 chRDNA guide/nucleoprotein complexes also can be produced using methods well known in the art. Cas12 protein components can be recombinantly produced and then the Cas12 chRDNA guide molecules and Cas12 proteins can be complexed together using methods known in the art. See, e.g., Example 2, which provides a non-limiting example of a method of assembling nucleoprotein complexes comprising a guide molecule/Cas12 protein.

Additionally, cell lines constitutively expressing Cas12 proteins can be developed and can be transfected with the Cas12 chRDNA guide components, and complexes can be purified from the cells using standard purification techniques, such as, but not limited to, affinity, ion exchange, and size exclusion chromatography. See, e.g., Jinek et al. (Science, 2012, 337:816-821).

According to known methods, Cas12 proteins can be produced using expression cassettes encoding a Cas12 protein. Expression cassettes typically comprise regulatory sequences functional in host cells into which they are introduced. Regulatory sequences are involved in one or more of the following: regulation of transcription, post-transcriptional regulation, and regulation of translation. Expression cassettes can be present in expression vectors and introduced into a wide variety of host cells, including bacterial cells, yeast cells, plant cells, and mammalian cells.

Cas12 proteins may be produced in vectors, including expression vectors, comprising polynucleotides encoding the Cas12 proteins. Vectors useful for producing Cas12 proteins include plasmids, viruses (including phage), and integratable nucleic acid fragments (i.e., fragments integratable into the host genome by homologous recombination). A vector replicates and functions independently of the host genome, or may, in some instances, integrate into the genome itself. Suitable replicating vectors will contain a replicon and control sequences derived from species compatible with the intended expression host cell. In some embodiments, a polynucleotide encoding a Cas12 protein is operably linked to an inducible promoter, a repressible promoter, or a constitutive promoter. Expression vectors can also include polynucleotides encoding protein tags (e.g., poly-His tags, hemagglutinin tags, fluorescent protein tags, bioluminescent tags, nuclear localization tags). The coding sequences for such protein tags can be fused to the coding sequence, or can be included in an expression cassette, for example, in a targeting vector.

General methods for construction of expression vectors are known in the art. Expression vectors for most host cells are commercially available. There are several commercial software products designed to facilitate selection of appropriate vectors and construction thereof, such as insect cell vectors for insect cell transformation and gene expression in insect cells, bacterial plasmids for bacterial transformation and gene expression in bacterial cells, yeast plasmids for cell transformation and gene expression in yeast and other fungi, mammalian vectors for mammalian cell transformation and gene expression in mammalian cells or mammals, viral vectors (including retroviral, lentiviral, and adenoviral vectors) for cell transformation, and gene expression and methods to easily enable cloning of such polynucleotides. SnapGene™ (GSL Biotech LLC, Chicago, Ill.; snapgene.com/resources/plasmid_files/your_time_is_valuable/), for example, provides an extensive list of vectors, individual vector sequences, and vector maps, as well as commercial sources for many of the vectors. A large number of mammalian vectors suitable for use are commercially available (e.g., from Life Technologies, Grand Island, NY; NeoBiolab, Cambridge, MA; Promega, Madison, WI; ATUM, Menlo Park, CA; Addgene, Cambridge, MA).

Vectors derived from mammalian viruses can also be used for expressing the Cas12 protein components of the present methods in mammalian cells. These include vectors derived from viruses such as adenovirus, adeno-associated virus, parvovirus, herpesvirus, polyomavirus, cytomegalovirus, lentivirus, retrovirus, vaccinia and Simian Virus 40 (SV40). See, e.g., Kaufman et al. (Mol. Biotech., 2000, 16:151-160); and Cooray et al. (Methods Enzymol., 2012, 507:29-57). Regulatory sequences operably linked to Cas12 protein-encoding sequences can include activator binding sequences, enhancers, introns, polyadenylation recognition sequences, promoters, repressor binding sequences, stem-loop structures, translational initiation sequences, translation leader sequences, transcription termination sequences, translation termination sequences, primer binding sites, and the like. Commonly used promoters are the constitutive mammalian promoters CMV, MND, EF1a, SV40, PGK1 (mouse or human), Ubc, CAG, CaMKIIa, and beta-Act, and others are known in the art. See, e.g., Khan et al. (Advanced Pharmaceutical Bulletin, 2013, 3:257-263). Furthermore, mammalian RNA polymerase III promoters, including H1 and U6, can be used.

Numerous mammalian cell lines have been utilized for expression of gene products including HEK 293 (human embryonic kidney) and CHO (Chinese hamster ovary). These cell lines can be transfected by standard methods (e.g., using calcium phosphate or polyethyleneimine (PEI), or electroporation). Other typical mammalian cell lines include, but are not limited to HeLa, U2OS, 549, HT1080, CAD, P19, NIH 3T3, L929, N2a, human embryonic kidney 293 cells, MCF-7, Y79, SO-Rb50, Hep G2, DUKX-X11, J558L, and baby hamster kidney (BHK) cells. Such cells are examples of cells that may be used to produce Cas12 proteins.

Vectors can be introduced into, and propagated in, a prokaryote. Prokaryotic vectors are well known in the art. Typically, a prokaryotic vector comprises an origin of replication suitable for the target host cell (e.g., oriC derived from E. coli, pUC derived from pBR322, pSC101 derived from Salmonella), 15A origin (derived from p15A) and bacterial artificial chromosomes). Vectors can include a selectable marker (e.g., genes encoding resistance for ampicillin, chloramphenicol, gentamicin, and kanamycin). Zeocin™ (Life Technologies, Grand Island, NY) can be used for selection in bacteria, fungi (including yeast), plants, and mammalian cell lines. Accordingly, vectors can be designed that carry only one drug resistance gene for Zeocin™ for selection work in a number of organisms. Useful promoters are known for expression of proteins in prokaryotes, for example, T5, T7, Rhamnose (inducible), Arabinose (inducible), and PhoA (inducible). Furthermore, T7 promoters are widely used in vectors that also encode the T7 RNA polymerase. Prokaryotic vectors can also include ribosome binding sites of varying strength, and secretion signals (e.g., mal, see, tat, ompC, and pelB). In addition, vectors can comprise RNA polymerase promoters for the expression of NATNAs. Prokaryotic RNA polymerase transcription termination sequences are also well known (e.g., transcription termination sequences from S. pyogenes).

Expression of proteins in prokaryotes is frequently carried out in E. coli with vectors containing constitutive or inducible promoters directing the expression of either fusion or non-fusion proteins. However, protein expression using other prokaryotic systems is within the scope of the present disclosure.

In some embodiments, a vector is a yeast expression vector. Examples of vectors for expression in Saccharomyces cerevisiae include, but are not limited to, the following: pYepSec1, pMFa, pJRY88, pYES2, and picZ. Methods for gene expression in yeast cells are known in the art. See, e.g., Christine Guthrie and Gerald R. Fink (“Guide to Yeast Genetics and Molecular and Cell Biology, Part A” in Methods in Enzymology, 2004, Volume 194, Elsevier Academic Press, San Diego, CA). Typically, expression of protein-encoding genes in yeast requires a promoter operably linked to a coding region of interest and a transcriptional terminator. Various yeast promoters can be used to construct expression cassettes for expression of genes in yeast.

Genomic Editing of Cells Using Cas12 chRDNA Guide/Nucleoprotein Complexes

Delivery of Cas12 chRDNA guide molecules, Cas12 proteins, and Cas12 chRDNA guide/nucleoprotein complexes of the present disclosure to cells, in vitro, ex vivo, or in vivo, may be achieved by a number of methods known to one of ordinary skill in the art. Non-limiting methods to introduce these components into a cell include viral vector delivery, sonoporation, cell squeezing, electroporation, nucleofection, lipofection, particle gun technology, microprojectile bombardment, or chemicals (e.g., cell penetrating peptides).

In some embodiments, electroporation can be used to deliver the Cas12 chRDNA guide molecules of the present disclosure to cells. Electroporation may also be used to deliver Cas12 chRDNA guide/nucleoprotein complexes of the present disclosure. In these methods, the chRDNA guide molecules, or the Cas12 chRDNA guide/nucleoprotein complexes, are mixed in an electroporation buffer with the target cells to form a suspension. This suspension is then subjected to an electrical pulse at an optimized voltage, which creates temporary pores in the phospholipid bilayer of the cell membrane, permitting charged molecules (like nucleic acids and proteins) to be driven through the pores and into the cell. Reagents and equipment to perform electroporation are sold commercially.

Example 3 illustrates nucleofection of activated T cells with Cas12 guide/nucleoprotein complexes. Example 5 illustrates nucleofection of activated T cells with Cas12 chRDNA guide/nucleoprotein complexes.

Cas12 chRDNA guide/nucleoprotein complexes can be used to cleave or bind to a target nucleic acid. A Cas12 chRDNA guide molecule can be introduced into cells with a Cas12 protein, thereby forming a Cas12 chRDNA guide/nucleoprotein complex. The Cas12 chRDNA guide/nucleoprotein complex can hybridize to a target nucleic acid, wherein the target nucleic acid comprises a PAM. In one embodiment, the present disclosure encompasses a method of binding a nucleic acid target sequence in a polynucleotide (e.g., in a double-stranded DNA (dsDNA)), comprising providing one or more Cas12 chRDNA guide/nucleoprotein complexes for introduction into a cell, and delivering the Cas12 nucleoprotein complex(es) into the cell, thereby facilitating contact of a Cas12 chRDNA guide/nucleoprotein complex(es) with the target polynucleotide sequence. In one embodiment, a first Cas12 chRDNA guide/nucleoprotein complex comprises a Cas12 chRDNA guide molecule having a first targeting region element complementary to a first nucleic acid target sequence in the polynucleotide; and a second Cas12 chRDNA guide/nucleoprotein complex comprises a Cas12 chRDNA guide molecule having a second targeting region complementary to a second nucleic acid target sequence in the polynucleotide. Contact of the Cas12 chRDNA guide/nucleoprotein complexes with the polynucleotide results in binding of the Cas12 chRDNA guide/nucleoprotein complexes to the nucleic acid target sequences in the polynucleotide. In one embodiment, a first Cas12a chRDNA guide/nucleoprotein complex binds to a first nucleic acid target sequence; and a second Cas12a chRDNA guide/nucleoprotein complex binds to a second nucleic acid target sequence, in the polynucleotide.

Such methods of binding a nucleic acid target sequence can be carried out in vitro (e.g., in a biochemical reaction or in cultured cells; in some embodiments, the cultured cells are human cultured cells that remain in culture and are not introduced into a human); in vivo (e.g., in cells of a living organism, with the proviso that, in some embodiments, the organism is a non-human organism); or ex vivo (e.g., cells removed from a subject, with the proviso that, in some embodiments, the subject is a non-human subject).

Delivery of Cas12 chRDNA guide molecules, Cas12 proteins, and Cas12 chRDNA guide/nucleoprotein complexes of the present disclosure to cells can be achieved by packaging the components into a biological compartment. A biological compartment comprising the components can be administered in vivo (e.g., in cells of a living organism, with the proviso that, in some embodiments, the organism is a non-human organism). Biological compartments can include, but are not limited to, viruses (lentivirus, adenovirus), nanospheres, liposomes, quantum dots, nanoparticles, microparticles, nanocapsules, vesicles, polyethylene glycol particles, hydrogels, and micelles.

For example, a biological compartment can comprise a liposome. A liposome can be a self-assembling structure comprising one or more lipid bilayers, each of which can comprise two monolayers containing oppositely oriented amphipathic lipid molecules. Amphipathic lipids can comprise a polar (hydrophilic) headgroup covalently linked to one or two or more non-polar (hydrophobic) acyl or alkyl chains. Energetically unfavorable contacts between the hydrophobic acyl chains and a surrounding aqueous medium induce amphipathic lipid molecules to arrange themselves such that polar headgroups can be oriented towards the bilayer's surface and acyl chains are oriented towards the interior of the bilayer, effectively shielding the acyl chains from contact with the aqueous environment.

Examples of preferred amphipathic compounds used in liposomes can include phosphoglycerides and sphingolipids, representative examples of which include phosphatidylcholine, phosphatidylethanolamine, phosphatidylserine, phosphatidylinositol, phosphatidic acid, phosphatidylglycerol, palmitoyloleoyl phosphatidylcholine, lysophosphatidylcholine, lysophosphatidylethanolamine, dimyristoylphosphatidylcholine (DMPC), dipalmitoylphosphatidylcholine (DPPC), diolcoylphosphatidylcholine, distearoylphosphatidylcholine (DSPC), dilinoleoylphosphatidylcholine, egg sphingomyelin, or any combination thereof.

A biological compartment can comprise a nanoparticle. A nanoparticle can comprise a diameter of from about 40 nanometers to about 1.5 micrometers, from about 50 nanometers to about 1.2 micrometers, from about 60 nanometers to about 1 micrometer, from about 70 nanometers to about 800 nanometers, from about 80 nanometers to about 600 nanometers, from about 90 nanometers to about 400 nanometers, or from about 100 nanometers to about 200 nanometers. In some instances, as the size of the nanoparticle increases, the release rate can be slowed or prolonged and as the size of the nanoparticle decreases, the release rate can be increased.

In certain embodiments, a Cas12 chRDNA guide/nucleoprotein complexes is packaged into a biological compartment. In some instances, a nucleic acid encoding a Cas12 and a chemically synthesized chRDNA guide are packaged into a biological compartment. In some instances, a mRNA encoding a Cas12 and a chemically synthesized chRDNA guide are packaged into a biological compartment.

A variety of methods are known in the art to evaluate and/or quantitate interactions between nucleic acid sequences and polypeptides including, but not limited to, the following: immunoprecipitation (ChIP) assays, DNA electrophoretic mobility shift assays (EMSA), DNA pull-down assays, and microplate capture and detection assays. Commercial kits, materials, and reagents are available to practice many of these methods and, for example, can be obtained from the following suppliers: Thermo Scientific (Wilmington, DE), Signosis (Santa Clara, CA), Bio-Rad (Hercules, CA), and Promega (Madison, WI). A common approach to detect interactions between a polypeptide and a nucleic acid sequence is EMSA (see, e.g., Hellman L. M., et al., Nature Protocols 2 (8): 1849-1861 (2007)).

In another embodiment, the present disclosure encompasses a method of cutting a nucleic acid target sequence in a polynucleotide (e.g., a single-strand cut in dsDNA, or a double-strand cut in dsDNA), comprising providing one or more Cas12 chRDNA guide/nucleoprotein complexes for introduction into a cell, and delivering the Cas12 chRDNA guide/nucleoprotein complex(es) into the cell, thereby facilitating contact of the Cas12 chRDNA guide/nucleoprotein complex(es) with the polynucleotide. In one embodiment, a first Cas12 chRDNA guide/nucleoprotein complex comprising a first Cas12 chRDNA guide molecule having a first targeting region complementary to a first nucleic acid target sequence in the polynucleotide; and a second Cas12 chRDNA guide/nucleoprotein complex comprising a second Cas12 chRDNA guide molecule having a second targeting region complementary to a second nucleic acid target sequence in the polynucleotide, are introduced into the cell. The contacting results in cutting of the nucleic acid target sequence in the polynucleotide (e.g., a dsDNA) by the Cas12 chRDNA guide/nucleoprotein complex(es). In one embodiment, a first Cas12a chRDNA guide/nucleoprotein complex binds to a first nucleic acid target sequence in dsDNA and cleaves the first strand of a dsDNA; and a second Cas12a chRDNA guide/nucleoprotein complex binds to a second nucleic acid target sequence in the dsDNA and cleaves the second strand of the dsDNA. In some embodiments, the nucleic acid target sequence is DNA or genomic DNA. Such methods of binding a nucleic acid target sequence are carried out in vitro, in cell (e.g., in cultured cells), ex vivo (e.g., stem cells removed from a subject), and in vivo.

The target nucleic acid sequence(s) can be appropriately selected, based upon, for example, a desired location in a polynucleotide sequence or genome; and/or a desired gene sequence in a polynucleotide sequence or genome to be deleted or disrupted.

In an additional embodiment of cutting a nucleic acid target sequence in a polynucleotide, a donor polynucleotide can also be introduced into a cell to facilitate incorporation of at least a portion of the donor polynucleotide into genomic DNA of the cell. Typically, a donor polynucleotide is brought into close proximity to a site-directed target nucleic acid break to enhance insertion (e.g., homologous recombination) of the donor polynucleotide into the site of the double-strand break. In some instances, a donor polynucleotide is brought into close proximity to the site of a double-strand break in a target nucleic acid, by binding it to the Cas12 protein that generates the double-strand break (e.g., Cas12a).

The donor polynucleotide sequence(s) can be appropriately selected, based upon, for example, the desired modification being pursued. For instance, a donor polynucleotide may encode all or part of a protein of interest. In some embodiments, the donor polynucleotide may encode a CAR.

The present disclosure further encompasses the delivery of a donor polynucleotide to a cell via a virus, wherein the donor polynucleotide encodes a CAR.

In some embodiments, the donor polynucleotide can be single-stranded. In some embodiments, the donor polynucleotide can be double-stranded. In some embodiments, the donor DNA can be a minicircle. In some embodiments, the donor polynucleotide can be a plasmid. In some embodiments, the plasmid can be supercoiled. In some embodiments, the donor polynucleotide can be methylated. In some embodiments, the donor polynucleotide can be unmethylated. The donor polynucleotide can comprise a modification. Modifications can include those described herein, including, but not limited to, biotinylation, chemical conjugate, and synthetic nucleotides.

### Therapeutic Compositions, Applications, and Methods

Cas12 chRDNA guide molecules, and Cas12 chRDNA guide/nucleoprotein complexes of the present disclosure can be used in the production of modified cells (such as CAR-expressing cells). Such modified cells can be used, for example, in the field of cell therapy (e.g., the treatment or prevention of a disease via the administration of cells), and especially for adoptive cell therapy. Such administered cells may be, for example, genetically-modified adoptive cells. Genetic modifications may be introduced into adoptive cells by way of the Cas12 chRDNA guide molecules and Cas12 chRDNA guide/nucleoprotein complexes disclosed herein, using one or more delivery techniques. The present disclosure encompasses, for example, the modification and administration of cells that are autologous or allogeneic with respect to the recipient to which they are to be administered. As used herein, the term “allogeneic” refers to a different, genetically non-identical, individual of the same species. For instance, an allogeneic cell refers to a cell derived from a different, genetically non-identical, individual of the same species (with respect to the recipient to be administered the cell). By contrast, the term “autologous” refers to the same individual. For instance, an autologous cell administered to an individual refers to a cell (modified or unmodified, or modified or unmodified progeny thereof) that is derived from that same individual.

An “adoptive cell” refers to a cell that can be genetically modified for use in a cell therapy treatment. Adoptive cells include, but are not limited to, stem cells, induced pluripotent stem cells, embryonic stem cells, cord blood stem cells, lymphocytes, natural killer cell, fibroblasts, endothelial cells, epithelial cells, pancreatic precursor cells, and the like.

A “stem cell” refers to a cell that has the capacity for self-renewal, i.e., the ability to go through numerous cycles of cell division while maintaining the undifferentiated state. Stem cells can be totipotent, pluripotent, multipotent, oligopotent, or unipotent. Stem cells are embryonic, fetal, amniotic, adult, or induced pluripotent stem cells.

An “induced pluripotent stem cell” (iPSCs) refers to a type of pluripotent stem cell that is artificially derived from a non-pluripotent cell, typically a somatic cell. In some embodiments, the somatic cell is a human somatic cell. Examples of somatic cells include, but are not limited to, dermal fibroblasts, bone marrow-derived mesenchymal cells, cardiac muscle cells, keratinocytes, liver cells, stomach cells, neural stem cells, lung cells, kidney cells, spleen cells, and pancreatic cells. Additional examples of somatic cells include cells of the immune system, including, but not limited to, B cells, dendritic cells, granulocytes, innate lymphoid cells, megakaryocytes, monocytes/macrophages, myeloid-derived suppressor cells, NK cells, T cells, thymocytes, and hematopoietic stem cells. Pluripotent stem cells can be differentiated into a plurality of cell types including somatic cells, NK cells, NK-like cells, T cells, T cell-like cells, NK-T cells, NK-T cell-like cells, dendritic cells, dendritic-like cells, macrophages, and macrophage-like cells. Pluripotent stem cells can be edited before or after differentiation, with a Cas12 chRDNA guide/nucleoprotein complex. An iPSC can be further modified, before or after differentiation, through the introduction of an exogenous gene or sequence into the genome, such as sequence encoding a CAR.

A “hematopoietic stem cell” refers to an undifferentiated cell that has the ability to differentiate into a hematopoietic cell, such as a lymphocyte.

A “lymphocyte” refers to a leukocyte (white blood cell) that is part of the vertebrate immune system. Also encompassed by the term “lymphocyte” is a hematopoietic stem cell that gives rise to lymphoid cells. Lymphocytes include T cells for cell-mediated, cytotoxic adaptive immunity, such as CD4+ and/or CD8+ cytotoxic T cells; alpha/beta T cells and gamma/delta T cells; regulatory T cells such as Treg cells; natural killer (NK) cells that function in cell-mediated, cytotoxic innate immunity; and B cells, for humoral, antibody-driven adaptive immunity; NK/T cells; cytokine induced killer cells (CIK cells); and antigen presenting cells (APCs), such as dendritic cells. The lymphocyte can be a mammalian cell, such as a human cell.

Also encompassed by the term “lymphocyte” as used herein are T cell receptor engineered T cells (TCRs), genetically engineered to express one or more specific, naturally occurring or engineered, T-cell receptor(s) that can recognize protein or (glyco) lipid antigens of target cells. Small pieces of these antigens, such as peptides or fatty acids, are shuttled to the target cell surface and presented to the T cell receptors as part of the major histocompatibility complex (MHC). T cell receptor binding to antigen-loaded MHCs activates the lymphocyte.

Tumor infiltrating lymphocytes (TILs) are also encompassed by the term “lymphocyte” as used herein. TILs are immune cells that have penetrated the environment in and around a tumor (the “tumor microenvironment”). TILs are typically isolated from tumor cells and the tumor microenvironment, and are selected in vitro for high reactivity against tumor antigens. TILs are grown in vitro under conditions that overcome the tolerizing influences that exist in vivo, and are then introduced into a subject for treatment.

The term “lymphocyte” also encompasses genetically-modified T cells and NK cells, such as those modified to produce chimeric antigen receptors (CARs) on the T or NK cell surface (CAR-T cells and CAR-NK cells).

Lymphocytes can be isolated from a subject, such as a human subject, for example from blood or from solid tumors, such as in the case of TILs, or from lymphoid organs such as the thymus, bone marrow, lymph nodes, and mucosal-associated lymphoid tissues. Techniques for isolating lymphocytes are well known in the art. For example, lymphocytes can be isolated from peripheral blood mononuclear cells (PBMCs), which are separated from whole blood using, e.g., Ficoll, a hydrophilic polysaccharide that separates layers of blood, and density gradient centrifugation. Generally, anticoagulant or defibrinated blood specimens are layered on top of a Ficoll solution and centrifuged to form different layers of cells. The bottom layer includes red blood cells (erythrocytes), which are collected or aggregated by the Ficoll medium and sink completely through to the bottom. The next layer contains primarily granulocytes, which also migrate down through the Ficoll-paque solution. The next layer includes lymphocytes, which are typically at the interface between the plasma and the Ficoll solution, along with monocytes and platelets. To isolate the lymphocytes, this layer is recovered, washed with a salt solution to remove platelets, Ficoll and plasma, then centrifuged again.

Other techniques for isolating lymphocytes include biopanning, which isolates cell populations from solution by binding cells of interest to antibody-coated plastic surfaces. Unwanted cells are then removed by treatment with specific antibody and complement. Additionally, fluorescence activated cell sorter (FACS) analysis can be used to detect and count lymphocytes. FACS analysis uses a flow cytometer that separates labelled cells based on differences in light scattering and fluorescence.

For TILs, lymphocytes are isolated from a tumor and grown, for example, in high-dose IL-2 and selected using cytokine release co-culture assays against either autologous tumor or HLA-matched tumor cell lines. Cultures with evidence of increased specific reactivity compared to allogeneic non-MHC matched controls can be selected for rapid expansion and then introduced into a subject in order to treat cancer. See, e.g., Rosenberg et al. (Clin. Cancer Res., 2011, 17:4550-4557); Dudly et al. (Science, 2002, 298:850-854); Dudly et al. (J. Clin. Oncol., 2008, 26:5233-5239); and Dudley et al. (J. Immunother., 2003, 26:332-342).

Upon isolation, lymphocytes can be characterized in terms of specificity, frequency, and function. Frequently used assays include an ELISPOT assay, which measures the frequency of T cell response.

After isolation, lymphocytes can be activated using techniques well known in the art in order to promote proliferation and differentiation into specialized effector lymphocytes. Surface markers for activated T cells include, for example, CD3, CD4, CD8, PD1, IL2R, and others. Activated cytotoxic lymphocytes can kill target cells after binding cognate receptors on the surface of target cells. Surface markers for NK cells include, for example, CD16, CD56, and others.

Following isolation and optionally activation, lymphocytes can be modified using Cas12 chRDNA guide/nucleoprotein complexes of the present disclosure, for use in adoptive T cell immunotherapies. Adoptive immunotherapy typically utilizes a patient's immune cells (autologous cells) to treat cancer. However, the present methods for the generation of adoptive immunotherapies also allow for the use of third-party donor cells (allogeneic cells), resulting in “off the shelf” therapies.

Thus, in some embodiments, lymphocytes for use in adoptive immunotherapies are isolated from a subject, modified ex vivo, and then reintroduced into the same subject. This technique is known as “autologous lymphocyte therapy.”

Alternatively, lymphocytes can be isolated, modified ex vivo, and introduced into a different subject. This technique is known as “allogeneic lymphocyte therapy.”

In certain embodiments, Cas12 chRDNA guide/nucleoprotein complexes are used for the production of therapeutic compositions comprising allogeneic cells. In a preferred embodiment, the allogeneic cells are T-cells. In a more preferred embodiment, the T-cells express a CAR. In an even more preferred embodiment, the CAR targets an antigen associated with a cancer.

In some embodiments, T cells can be modified to allow for safer and more efficient allogeneic therapies. For example, the T cell receptor α constant (TRAC) is a protein-coding gene that forms part of the αβ TCR. Selected mutations in TRAC, as well as knocking out expression of TRAC, can therefore help eliminate GvHD during allogeneic cell therapies. See, e.g., Poirot et al. (Cancer Res., 2015, 75:3853-3864). It has been shown that directing a CD19-specific CAR to the TRAC locus using a CRISPR-Cas9 system can result in tumor rejection. See, e.g., Eyquem et al. (Nature, 2017, 543:113). Similarly, T cell receptor β constant (TRBC) can also be targeted in order to prevent expression of the αβ TCR. See, e.g., Ren et al. (Clin. Cancer Res., 2017, 23:2255-2266).

Programmed cell death protein 1, also known as PD1, PDCD1, and CD279, is a cell surface receptor that plays an important role in down-regulating the immune system, and promoting self-tolerance by suppressing T cell inflammatory activity. PDCD1 binds to its cognate ligand, “programmed death-ligand 1,” also known as PD-L1, CD274, and B7 homolog 1 (B7-H1). PD1 guards against autoimmunity through a dual mechanism of promoting programmed cell death (apoptosis) in antigen-specific T cells in lymph nodes, while simultaneously reducing apoptosis in anti-inflammatory, suppressive T cells (regulatory T cells). Through these mechanisms, PD1 binding of PD-L1 inhibits the immune system, thus preventing autoimmune disorders, but also prevents the immune system from killing cancer cells. Accordingly, mutating or knocking out production of PD1 can be beneficial in T cell therapies.

PD1 is an example of an “immune checkpoint” molecule. Immune checkpoint molecules serve to down-modulate or inhibit an immune response. Immune checkpoint molecules include, but are not limited to, PD1, Cytotoxic T-Lymphocyte Antigen 4 (CTLA-4, also known as CD152), LAG3 (also known as CD223), Tim3 (also known as HAVCR2), BTLA (also known as CD272), BY55 (also known as CD160), TIGIT (also known as IVSTM3), LAIR1 (also known as CD305), SIGLEC10, 2B4 (also known as CD244), PPP2CA, PPP2CB, PTPN6, PTPN22, CD96, CRTAM, SIGLEC7, SIGLEC9, TNFRSF10B, TNFRSF10A, CASP8, CASP10, CASP3, CASP6, CASP7, FADD, FAS, TGFBRII, TGFRBRI, SMAD2, SMAD3, SMAD4, SMAD10, SKI, SKIL, TGIF1, IL10RA, IL10RB, HMOX2, IL6R, IL6ST, EIF2AK4, CSK, PAGI, SIT1, FOXP3, PRDM1, BATF, GUCY1A2, GUCY1A3, GUCY1B2, and GUCY1B3. In some embodiments, one or more immune checkpoint molecules are inactivated using Cas12 chRDNA guide/nucleoprotein complexes of the present disclosure. In some embodiments, the inactivation of one or more immune checkpoint molecules is combined with the inactivation of one or more TCR components, as described above.

Beta-2 microglobulin (B2M) is a component of MHC class I molecules present on nucleated cells. Beta-2 microglobulin is shed by cells, including tumor cells, into the blood and is essential for the assembly and expression of the HLA I complex. However, expression of HLA on the surface of allogeneic T cells causes rapid rejection by T cells of the host immune system. Thus, disrupting expression of beta-2 microglobulin is also desirable for increasing allogeneic T cell therapy efficiency. Additionally, lack of expression of MHC class I molecules on allogeneic T cells causes clearance by the host immune system. Thus, presentation of only a subset of the HLA molecules, preferably HLA-E, on the surface of cells is desirable.

Additional genes can be similarly targeted with the Cas12 chRDNA guides disclosed herein to enhance the efficacy of an adoptive immune cell therapy. Non-limiting examples of preferred genes and chromosomal locations (hg38 genome assembly) are provided in Table 4.

TABLE 4
Adaptive Cell Therapy Targets

| Encoded Protein/Alternative name | Locus coordinates |
|---|---|
| TRAC | chr14: 22, 509, 239-22, 552, 153 |
| TRBV proteins | TRBV locus chr7 |
| Beta-2 microglobulin (B2M) | chr15: 44, 711, 477-44, 718, 877 |
| PDCD1/PD1/CD279 | chr2: 241, 849, 881-241, 858, 908 |
| PD-L1 | chr9: 5, 450, 525-5, 470, 547 |
| CTLA-4/CD152 | chr2: 203, 867, 786-203, 873, 960 |
| LAG-3/CD223 | chr12: 6, 772, 520-6, 778, 453 |
| TIGIT/IVSTM3 | chr3: 114, 291, 059-114, 308, 290 |
| TIM3/HAVCR2 | chr5: 157, 085, 832-157, 109, 714 |
| HLA-E | HLA-E locus chr6 |
| HLA-A | HLA-A locus chr6 |
| ROR1 | chr1: 63, 774, 017-64, 181, 498 |
| CD47 | chr3: 108, 043, 091-108, 094, 200 |
| HLA-B | HLA-B locus chr6 |
| HLA-C | HLA-C locus chr6 |
| HLA-DRA | HLA-DRA locus chr6 |
| ADAM17 | chr2: 9, 488, 486-9, 555, 788 |
| BTLA/CD272 | chr3: 112, 463, 968-112, 499, 561 |
| P2X7 | chr12: 121, 132, 819-121, 188, 032 |
| CD160 | chr1: 145, 719, 520-145, 739, 153 |
| ADORA2A | chr22: 24, 417, 879-24, 442, 357 |
| SIGLEC10 | chr19: 51, 411, 054-51, 417, 698 |
| 2B4/CD244 | chr1: 160, 830, 160-160, 862, 855 |
| LAIR1/CD305 | chr19_KI270938v1_alt: 337, 398-347, 586 |
| CD52 | chr1: 26, 317, 957-26, 320, 523 |
| CD96 | chr3: 111, 542, 118-111, 652, 241 |
| VSIR or C10orf54/VISTA | chr10: 71, 747, 559-71, 773, 498 |
| KIR2DL1 | chr19_KI270933v1_alt: 71, 449-85, 961 |
| KIR2DL2 | chr19_KI270932v1_alt: 56, 346-70, 673 |
| KIR2DL3 | chr19: 54, 738, 515-54, 753, 052 |
| CEACAMI | chr19: 42, 507, 306-42, 528, 509 |
| CBLB | chr3: 105, 655, 461-105, 869, 043 |
| CISH | chr3: 50, 606, 522-50, 611, 831 |
| IL-1R8/TIR8/SIGIRR | chr11: 405, 716-417, 397 |
| AHR | chr7: 17, 298, 622-17, 346, 150 |
| Adenosine 2A receptor/ADORA2A | chr22: 24, 423, 597-24, 442, 356 |
| GMCSF/CSF2 | chr5: 132, 073, 789-132, 076, 170 |
| VISTA/VSIR | chr10: 71, 747, 556-71, 773, 520 |
| CII2A | chr16: 10, 877, 198-10, 936, 388 |
| NKG2A | chr12: 10, 446, 041-10, 454, 616 |

In some embodiments, a gene encoding TRAC is targeted within a cell using a Cas12 chRDNA guide as disclosed herein. In some embodiments, a gene encoding PD1 is targeted within a cell using a Cas12 chRDNA guide as disclosed herein. In some embodiments, a gene encoding B2M is targeted within a cell using a Cas12 chRDNA guide as disclosed herein. In some embodiments, a gene encoding TRAC and a gene encoding B2M are targeted within a cell, using Cas12 chRDNA guides as disclosed herein.

Cells modified using Cas12 chRDNA guide/nucleoprotein complexes of the present disclosure can be used, for example, in adoptive cell therapy for the treatment of cancer. In some embodiments thereof, the modified cell is a genetically-modified lymphocyte. Such genetically-modified lymphocytes, such as CAR-T cells, can be used to treat various types of cancers in a subject, including, but not limited to, prostate cancers; ovarian cancers; cervical cancers; colorectal cancers; intestinal cancers; testicular cancers; skin cancers; lung cancers; thyroid cancers; bone cancers; breast cancers; bladder cancers; uterine cancers; vaginal cancers; pancreatic cancers; liver cancers; kidney cancers; brain cancers; spinal cord cancers; oral cancers; parotid tumors; blood cancers; lymphomas, such as B cell lymphomas; and leukemias, etc. Preferably, an effective amount of modified cells is used for such treatment.

Table 5 lists representative B cell leukemias and lymphomas treatable using adoptive cells (such as CAR-T cells) produced using Cas12 chRDNA guide/nucleoprotein complexes of the present disclosure. It is to be understood that the lymphocytes modified by the Cas12 chRDNA guide/nucleoprotein complexes disclosed herein are not limited for treatment of the diseases listed in Table 5.

TABLE 5
B-Cell Leukemias/Lymphomas and Acronyms Thereof

| B-Cell leukemia/lymphoma | Acronym |
|---|---|
| Acute lymphoblastic leukemia | B-ALL |
| Acute myeloid leukemia | AML |
| Precursor B lymphoblastic leukemia | LBL |
| Hairy cell leukemia | HCL |
| B-cell prolymphocytic leukemia | B-PLL |
| Chronic lymphocytic leukemia/small | CLL/SLL |
| lymphocytic leukemia | |
| Diffuse large B-cell lymphoma | DLBCL |
| ALK-positive large B-cell lymphoma | ALK + LBCL |
| T-cell/histiocyte-rich large B-cell lymphoma | T/HRBCL |
| Follicular lymphoma | FL |
| Mantle cell lymphoma | MCL |
| Burkitt lymphoma (EBV) | BL |
| Multiple myeloma | MM |
| Lymphoplasmacytic lymphoma/ | MG |
| Waldenstrom macroglobulinemia | |
| Mucosa-associated lymphoid tissue lymphoma | MALT |
| Nodal marginal zone B-cell lymphoma | NMZL |
| Splenic marginal zone B-cell lymphoma | SMZL |
| Primary central nervous system lymphoma | PCNSL |
| Primary cutaneous follicle center lymphoma | PCFCL |
| Primary mediastinal large B-cell lymphoma | PMLBCL |
| Intravascular large B-cell lymphoma | ILBCL |
| Lymphomatoid granulomatosis (EBV) | LYG |
| Plasmablastic lymphoma (ARL) | PBL |
| Primary effusion lymphoma | PEL |

In other embodiments, other cell proliferative disorders can be treated using adoptive cells (such as CAR-T cells) produced using Cas12 chRDNA guide/nucleoprotein complexes of the present disclosure, including precancerous conditions; hematologic disorders; and immune disorders, such as autoimmune disorders including, without limitation, Addison's disease, celiac disease, diabetes mellitus type 1, Grave's disease, Hashimoto's disease, inflammatory bowel disease, multiple sclerosis, psoriasis, rheumatoid arthritis, scleroderma, and systemic lupus erythematosus.

The adoptive cell therapy treatments described herein can be combined, at the same or different times, with one or more additional therapies selected from the group consisting of antibody therapy, chemotherapy, cytokine therapy, dendritic cell therapy, gene therapy, hormone therapy, laser light therapy, and radiation therapy.

The administration of modified cells of the present disclosure to subjects may be carried out in any convenient manner, including by aerosol inhalation, injection, ingestion, transfusion, implantation or transplantation. The compositions described herein may be administered to a patient subcutaneously, intradermally, intratumorally, intranodally, intramedullary, intramuscularly, by intravenous or intralymphatic injection, or intraperitoneally.

In one embodiment, the modified cell compositions of the present disclosure are preferably administered by intravenous injection. The administration may comprise the administration of 10⁴-10⁹ cells per kg body weight, preferably 10⁵ to 10⁶ cells/kg body weight. The cells can be administrated in one or more doses. In some embodiments, an effective amount of modified cells is administrated as a single dose. In other embodiments, an effective amount of cells is administrated as more than one dose over a period time. The determination of optimal ranges of effective amounts of a given cell type for a particular disease or condition is within the skill of those in the art.

### Chimeric Antigen Receptor (CAR) Cells

In some embodiments, an adoptive cell is a CAR-expressing cell. A CAR is a receptor engineered to recognize and bind to a specific antigen or epitope. The receptor is chimeric because it combines both antigen-binding and T-cell activating functions into a single receptor. A CAR is typically a fusion protein comprising an extracellular ligand-binding domain capable of binding to an antigen, a transmembrane domain, and at least one intracellular signaling domain. An extracellular ligand-binding domain may comprise a single-chain variable fragment (scFv) comprising a fusion of two or more variable regions connected by one or more linkers. A CAR may further comprise a hinge region. A CAR is sometimes called a “chimeric receptor,” a “T-body,” or a “chimeric immune receptor (CIR).”

In some embodiments, the CAR can be a TRUCK, Universal CAR, Self-driving CAR, TanCAR, Armored CAR, Self-destruct CAR, Conditional CAR, Marked CAR, TenCAR, Dual CAR, or sCAR.

TRUCKs (T cells redirected for universal cytokine killing) co-express a chimeric antigen receptor (CAR) and an antitumor cytokine. Cytokine expression may be constitutive or induced by T cell activation. Targeted by CAR specificity, localized production of pro-inflammatory cytokines recruits endogenous immune cells to tumor sites and may potentiate an antitumor response.

Universal, allogeneic CAR-T cells are engineered to no longer express endogenous T cell receptor (TCR) and/or major histocompatibility complex (MHC) molecules, thereby preventing graft-versus-host disease (GVHD) or rejection, respectively.

Self-driving CARs co-express a CAR and a chemokine receptor, which binds to a tumor ligand, thereby enhancing tumor homing.

CAR-T cells engineered to be resistant to immunosuppression (Armored CARs) may be genetically modified to no longer express various immune checkpoint molecules (for example, cytotoxic T lymphocyte-associated antigen 4 (CTLA4) or programmed cell death protein 1 (PD1)), with an immune checkpoint switch receptor, or may be administered with a monoclonal antibody that blocks immune checkpoint signaling.

A self-destruct CAR may be designed using RNA delivered by electroporation to encode the CAR. Alternatively, inducible apoptosis of the T cell may be achieved based on ganciclovir binding to thymidine kinase in gene-modified lymphocytes or the more recently described system of activation of human caspase 9 by a small-molecule dimerizer.

A conditional CAR-T cell is by default unresponsive, or switched ‘off’, until the addition of a small molecule to complete the circuit, enabling full transduction of both signal 1 and signal 2, thereby activating the CAR-T cell. Alternatively, T cells may be engineered to express an adaptor-specific receptor with affinity for subsequently administered secondary antibodies directed at target antigen.

Marked CAR-T cells express a CAR and a tumor epitope to which an existing monoclonal antibody agent binds. In the setting of intolerable adverse effects, administration of the monoclonal antibody clears the CAR-T cells and alleviates symptoms with no additional off-tumor effects.

A tandem CAR (TanCAR) T cell expresses a single CAR comprising two linked scFvs that have different affinities and are fused to one or more intracellular co-stimulatory domain(s) and a CD32 signaling domain. TanCAR-T cell activation requires only one antigen to be present on target cells; however, the presence of both antigens facilitates a synergistic activation. In certain embodiments, an scFv of the TanCAR comprises a heavy chain variable region (VH) and light chain variable region (VL), a pair of two heavy chain variable regions (VH), or a pair of two light chain variable regions (VL). In another embodiment, the two scFvs of the TanCAR can occur in a stacked configuration. In yet another embodiment, the two scFvs of the TanCAR can occur in series, or in a looped configuration. In specific embodiments, at least one of the scFvs of the tandem CAR is an anti-CD20 scFv, and the second scFv is selected to target a specific antigen on cancer cells, such as an anti-BCMA scFv, an anti-CD19 scFv, an anti-CD30 scFv, an anti-CD22 scFv, an anti-CD70 scFv, an anti-ROR1 scFv, or an anti-kappa light chain scFv.

A dual CAR-T cell expresses two separate CARs with different ligand binding targets; one CAR includes only the CD3ζ. domain and the other CAR includes only the co-stimulatory domain(s). Dual CAR-T cell activation requires co-expression of both targets on the tumor.

A safety CAR (sCAR) consists of an extracellular scFv fused to an intracellular inhibitory domain, sCAR-T cells co-expressing a standard CAR become activated only when encountering target cells that possess the standard CAR target but lack the sCAR target.

The extracellular (antigen recognition) domain of a CAR is preferably a single chain antibody, and more preferably an scFv. In one embodiment, the antigen-binding domain comprises an scFv. However, any suitable moiety that binds a given target with high affinity can be used as an antigen recognition region. The extracellular domain of a CAR capable of binding to an antigen may be, for example, any oligopeptide or polypeptide that can bind to a certain antigen.

Depending on the desired antigen to be targeted, a CAR of the present disclosure can be engineered to include the appropriate antigen-binding moiety that is specific to the desired antigen target. For example, if BCMA is the desired antigen that is to be targeted, an antibody or antibody fragment (for example an scFv) targeting BCMA can be used as the antigen-binding moiety for incorporation into the CAR of the present disclosure.

Preferable cellular targets and the CAR scFvs/binding proteins that target them are set forth in Table 6.

TABLE 6
List of Exemplary Cellular Targets and CAR scFvs

| Cellular Target | CAR scFv/binding portion |
|---|---|
| B7-H3 | anti-B7-H3 |
| B7-H6 | anti-B7-H6 |
| B-cell maturation antigen | anti-BCMA |
| (BCMA) | |
| CD123 | anti-CD123 |
| CD138 | anti-CD138 |
| CD171/L1CAM | anti-CD171 |
| CD19 | anti-CD19 |
| CD20 | anti-CD20 |
| CD22 | anti-CD22 |
| CD30 | anti-CD30 |
| CD33 | anti-CD33 |
| CD37 | anti-CD37 |
| CD38 | anti-CD38 |
| CD4 | anti-CD4 |
| CD47 | anti-CD47 |
| CD70 | anti-CD70 |
| CD73 | anti-CD73 |
| CD79b | anti-CD79b |
| CD371 | anti-CD371 |
| CEA | anti-CEA |
| Claudin 18.1 | anti-CLDN 18.1 |
| Claudin 18.2 | anti-CLDN 18.2 |
| CS-1 | anti-CS1 |
| CSPG4 | anti-CSPG4 |
| EFGRvIII | anti-EFGRvIII |
| ENPP3 | anti-ENPP3 |
| EpCAM | anti-EpCAM |
| EphA2 | anti-EphA2 |
| Epidermal growth factor | anti-epidermal growth factor |
| receptor | receptor |
| ErbB | anti-ErbB |
| ErbB2 (HER2) | anti-ErbB2 (HER2) |
| FAP | anti-FAP |
| FRα | anti-FRα |
| GD2 | anti-GD2 |
| GD3 | anti-GD3 |
| Glypican 3 | anti-glypican 3 |
| Her2 | anti-Her2 |
| IL-11Rα | anti-IL-11Rα |
| IL-13Rα2 | anti-IL-13Rα2 |
| IL13 receptor alpha | IL13 |
| kappa light chain | anti-kappa light chain |
| Lewis Y/Le Y | Anti-LeY |
| Mesothelin | anti-mesothelin |
| MUC1 | anti-MUC1 |
| MUC16 | anti-MUC16 |
| NGFR | anti-NGFR |
| NKG2D ligands | anti-NKG2D ligands |
| PD1 | anti-PD1 |
| PD-L1 | anti-PD-L1 |
| PSCA | anti-PSCA |
| PSMA | anti-PSMA |
| ROR-1 | anti-ROR-1 |
| SLAMF7 | anti-SLAMF7 |
| TACI | anti-TACI |
| TAG72 | anti-TAG72 |
| ULBP and MICA/B proteins | NKG2D |
| VEGF2 | anti-VEGF2 |
| VEGFR2 | anti-VEGFR2 |
| WT1 | anti-WT1 |

In certain embodiments, the cellular target to which the CAR binds is more preferably selected from BCMA, CD19, CD20, CD22, CD47, CD79b, CD371, ROR-1, EphA2, MUC16, Glypican 3, PSCA, and Claudin 18.2.

In an even more preferable embodiment, the cellular target to which the CAR binds is BCMA.

In an even more preferable embodiment, the cellular target to which the CAR binds is CD371.

The intracellular domain of a CAR may be an oligopeptide or polypeptide known to function as a domain that transmits a signal to cause activation or inhibition of a biological process in a cell. The intracellular domain may comprise an activation domain comprising all or a portion of the intracellular signaling domain of a T-cell receptor (TCR) and/or a co-receptor, as long as it transduces the effector function signal. Cytoplasmic signaling sequences that regulate primary activation of the TCR complex that act in a stimulatory manner may contain signaling motifs which are known as immunoreceptor tyrosine-based activation motifs (ITAMs). Examples of ITAM containing cytoplasmic signaling sequences include those derived from CD8, CD3ζ, CD3δ, CD3γ, CD3ε, CD32 (FcγRIIa), DAP10, DAP12, CD79a, CD79b, FcγRly, FcγRIIIγ, FcεRIβ (FCERIB), and FcεRIγ (FCERIG).

In preferred embodiments, the activation domain of the intracellular signaling domain is derived from CD35.

The intracellular signaling domain of a CAR of the present disclosure can be designed to comprise an activation domain, such as a CD35 signaling domain, either by itself or combined with any other desired cytoplasmic domain(s) useful in the context of a CAR of the present disclosure. For example, the intracellular signaling domain of the CAR may comprise an activation domain, such as a CD35 chain portion, in addition to a costimulatory domain. The costimulatory domain refers to a portion of the CAR comprising the intracellular domain of a costimulatory molecule.

A costimulatory molecule is a molecule other than an antigen receptor or its ligands that is required for an efficient response of lymphocytes to an antigen. Examples of such co-stimulatory molecules, in which all or a part thereof can be used in a costimulatory domain of a CAR of the present disclosure, include CD27, CD28, 4-1BB (CD137), OX40, CD30, CD40, ICOS-1, GITR, lymphocyte function-associated antigen-1 (LFA-1), CD2, CD7, LIGHT, NKG2C and B7-H3.

In preferred embodiments, the CAR contains a costimulatory domain derived from at least 4-1BB.

The transmembrane domain may be derived from either a natural or synthetic source. Where the source is natural, the domain may be derived from any membrane-bound or transmembrane protein. For example, the transmembrane region may be derived from (i.e., comprise at least a part of) the transmembrane region(s) of the alpha, beta or zeta chain of the T-cell receptor, CD28, CD35, CD38, CD45, CD4, CD5, CD8 (e.g., CD8a, CD8B), CD9, CD16, CD22, CD33, CD37, CD64, CD80, CD86, CD134, CD137, or CD154, KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), CD160, CD19, IL2RB, IL2RY, IL7Rα, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, and PAG/Cbp.

Alternatively the transmembrane domain may be synthetic, in which case it will comprise predominantly hydrophobic residues such as leucine and valine. In some cases, a triplet of phenylalanine, tryptophan and valine will be found at each end of a synthetic transmembrane domain. A short oligo- or polypeptide linker, such as between 2 and 10 amino acids in length, may form the linkage between the transmembrane domain and the endoplasmic domain of the CAR.

In a preferred embodiment, the transmembrane domain is derived from CD8.

In some embodiments, the CAR has more than one transmembrane domain, which can be a repeat of the same transmembrane domain, or can be different transmembrane domains.

The hinge region may comprise a polypeptide hinge of variable length, such as one or more amino acids, a CD8 portion, or a IgG4 region, and combinations thereof.

In a preferred embodiment, the hinge region is derived from CD8.

CARs can also be incorporated into TILs, NK cells, macrophages, dendritic cells, induced pluripotent stem cells (iPSCs), or TCRs resulting in CAR-TILs, CAR-NK cells, CAR-M, CAR-DC, and TCR engineered CAR-T cells, respectively. For descriptions of CAR-T cells, methods of making CAR-T cells, and uses thereof, see, e.g., Brudno et al. (Nature Rev. Clin. Oncol., 2018, 15:31-46); Maude et al. (N. Engl. J. Med., 2014, 371:1507-1517); and Sadelain et al. (Cancer Disc., 2013, 3:388-398).

In some embodiments, the CAR expression cassette is transduced into an adoptive cell, and the cassette is integrated into a Cas12 protein-mediated break site. Example 9 herein illustrates the transduction of primary cells with an adeno-associated virus (AAV) vector comprising a CAR cassette.

In some embodiments, the CAR expression cassette comprises a promoter to drive CAR expression. Commonly used promoters include the constitutive mammalian promoters CMV, MND, EF1a, SV40, PGK1 (mouse or human), Ubc, CAG, CaMKIIa, and beta-Act, and others are known in the art. See, e.g., Khan et al. (Advanced Pharmaceutical Bulletin, 2013, 3:257-263). Alternatively, a CAR expression cassette can comprise a ribosomal skipping sequence (also called a self-cleaving peptide) and can be introduced in-frame of an endogenously expressed gene. Commonly used ribosomal skipping sequences include T2A, P2A, E2A, and F2A. For a description of ribosomal skipping sequences and uses thereof, see, e.g., Chng et al. (MAbs, 2015, 7 (2): 403-412). Similarly, non-CAR expression cassettes can comprise similar promoters or ribosomal skipping sequences.

In certain embodiments, Cas12 chRDNA guide/nucleoprotein complexes are used to treat genetic disorders that are caused by pathogenic, autosomal “dominant negative” mutations that are present on a single allele in a patient. In some instances, the underlying genetic mutation may be a single nucleotide polymorphism (SNP) on one of the alleles. chRDNA guide/nucleoprotein complexes may be engineered to target the SNP allele, but not target the wild type allele, thereby disrupting only the SNP allele. See, e.g., Example 7, which provides a non-limiting example of a method of designing Cas12a chRDNA guide/nucleoprotein complexes to target a wild type sequence that are capable of reduced editing at off-target sequences comprising a SNP.

In some embodiments, Cas12 chRDNA guide/nucleoprotein complexes can be used to selectively edit (e.g., knock-out, or revert back to wild type with homology directed repair) the SNP-containing allele, while not modifying the wild type allele. In some embodiments, such editing may lead to gene disruption. In other embodiments, such editing may restore the allele back to a “wild type” state, such as through homology directed repair. For instance, a number of genetic diseases that lead to progressive vision loss are due to pathogenic, autosomal “dominant-negative” mutations. Examples of SNP correction strategies of dominant negative disease include, but are not limited to, targeting of SNP mutations in Rhodopsin gene causing retinitis pigmentosa, see, e.g., Li et al. (CRISPR J., 2018, 1 (1): 55-64); targeting of SNP mutations in the transforming growth factor, beta-induced (TGFBI) gene causing corneal dystrophies, see, e.g., Christie et al. (Scientific Reports, 2017, 7 (1): 16174).

Cas12 chRDNA guide/nucleoprotein complexes of the present disclosure can be delivered, for example, to ocular tissues that are affected by autosomal, pathogenic “dominant-negative” genetic mutations. In some embodiments thereof, the chRDNA guide/nucleoprotein complex is designed to selectively disrupt the disease allele, while not targeting the wild type allele, to treat the underlying pathology. Such diseases may include, but are not limited to, macular dystrophies, rod-cone dystrophies, cone-rod dystrophies, or chorioretinopathies. It is understood that the Cas12 chRDNA guide/nucleoprotein complexes disclosed herein are not limited for the treatment of genetic diseases that cause progressive vision loss.

### Experimental

Non-limiting embodiments of the present disclosure are illustrated in the following Examples. Efforts have been made to ensure accuracy with respect to numbers used (e.g., amounts, concentrations, percent changes, and the like), but some experimental errors and deviations should be accounted for. Unless indicated otherwise, temperature is in degrees Centigrade and pressure is at or near atmospheric. It should be understood that these Examples are given by way of illustration only and are not intended to limit the scope of what the inventor regards as various embodiments of the present disclosure. Not all of the following steps set forth in each Example are required nor must the order of the steps in each Example be as presented. As used herein, an “r” preceding a nucleotide indicates RNA; and all other nucleotides are DNA (see, e.g., Tables 15 and 17), unless otherwise noted. Phosphorothioate bonds are represented with an “*” between the adjacent bases.

### Example 1

Preparation of Cytotoxic T Cells (CD4+ and CD8+) from PBMCs and Culture of Primary Cells

This Example illustrates the preparation of CD4+ and CD8+ T cells from donor peripheral blood mononuclear cells (PBMCs).

CD4+ and CD8+ T cells were prepared from donor PBMCs essentially as follows. T cells were isolated from peripheral blood mononuclear cells (PBMCs) using RoboSep-S(STEMCELL Technologies, Cambridge, MA) and EasySep™ Human T cell Isolation Kit (STEMCELL Technologies, Cambridge, MA) and activated for 3 days in the presence of anti-CD3/CD28 beads (Dynabeads™; Gibco 11132D) in ImmunoCult-XF complete medium (ImmunoCult-XF T Cell Expansion Medium (STEMCELL Technologies, Cambridge, MA), CTS Immune Cell SR (Gibco A2596102), Antibiotics-Antimycotics (100×, Corning 30-004-C1)) supplemented with recombinant human (rh) IL-2 (100 units/mL). After 3 days, beads were removed via magnetic separation and cells were expanded for 1 day in ImmunoCult-XF complete medium supplemented with IL-2 (100 units/mL).

### Example 2

### Cloning, Expression, Production, and Assembly of Cas12a Guide/Nucleoprotein Complexes

This Example describes a method for cloning, expressing, and purifying Cas12a guide/nucleoprotein complexes, as well as methods of producing Cas12a guide components.

### A. Cloning of a Cas12 Protein

The Acidaminococcus spp. (strain BV3L6) catalytically active Cas12a protein sequence (SEQ ID NO: 1) was codon optimized for expression in E. coli cells. At the C-terminus, a glycine-serine linker and one nuclear localization sequence (NLS) (SEQ ID NO: 4) was added. Oligonucleotide sequences coding for the Cas12a-NLS protein (referred to as the AsCas12a and Cas12a protein in the following Examples) were provided to commercial manufacturers for synthesis. DNA sequences were then cloned into suitable bacterial expression vectors using standard cloning methods.

### B. Expression and Purification of a Cas12a Protein

The AsCas12a protein was expressed in E. coli using an expression vector and purified using affinity chromatography, ion exchange, and size exclusion chromatography, essentially as described in, for example, Swarts et al. (Molecular Cell, 2017, 66:221-233).

### C. Production of Cas12a Guide Components

Cas12a guides were produced by linking a targeting region to a particular Cas12a guide activating region. A targeting region, or spacer, preferably comprised a 20-nucleotide target binding sequence. The target binding sequence was complementary to a target sequence that occurred downstream (in a 3′ direction) of a 5′-TTTV or 5′-TTTN PAM. Exemplary Cas12a guide activating region sequences are SEQ ID NO: 6, SEQ ID NO: 8, and SEQ ID NO: 10, for the Acidaminococcus spp., L. bacterium, and F. novicida Cas12a species, respectively.

Cas12a guide sequences (such as crRNAs and chRDNA) were provided to a commercial manufacturer for synthesis.

Guide RNA components (such as crRNAs) can be produced by in vitro transcription (e.g., T7 Quick High Yield RNA Synthesis Kit; New England Biolabs, Ipswich, MA) from double-stranded (ds) DNA templates by incorporating a T7 promoter at the 5′ end of the dsDNA template sequences.

### D. Assembly of a Cas12a Guide/Nucleoprotein Complex

Acidaminococcus spp. Cas12a (AsCas12a) tagged with a C-terminal nuclear localization sequence (NLS) was recombinantly expressed in E. coli and purified using chromatographic methods. Nucleoprotein complexes were formed at a concentration of 80 pmol Cas12a protein: 240 pmol guide, unless otherwise stated. Prior to assembly with Cas12a protein, each of the guide components (e.g., crRNA or chRDNA) was adjusted to the desired total concentration (240 μmol) in a final volume of 1 μl, incubated for 2 minutes at 95° C., removed from a thermocycler, and allowed to equilibrate to room temperature. The Cas12a protein was diluted to an appropriate concentration in binding buffer (60 mM TRIS-acetate, 150 mM potassium acetate, 30 mM magnesium acetate, at pH 7.9) to a final volume of 1.5 μl and mixed with the 1 μl of the guide components, followed by incubation at 37° C. for 10 minutes.

### Example 3

Nucleofection of T Cells (CD4+ and CD8+) from PBMCs with Cas12a Guide/Nucleoprotein Complexes

This Example describes the nucleofection of activated T cells with a Cas12a guide/nucleoprotein complex.

The Cas12a guide/nucleoprotein complexes of Example 2 were transfected into primary activated T cells (CD4+ and CD8+) (prepared as described in Example 1) using the Nucleofector™ 96-well Shuttle System (Lonza, Allendale, NJ). The Cas12a guide/nucleoprotein complex were dispensed in a 2.5 μl final volume into individual wells of a 96-well plate. The suspended T cells were pelleted by centrifugation for 10 minutes at 200×g, washed with calcium and magnesium-free phosphate buffered saline (PBS), and the cell pellet was resuspended in 10 ml of calcium and magnesium-free PBS. The cells were counted using the Countess® II Automated Cell Counter (Life Technologies; Grand Island, NY).

2.2e7 cells were transferred to a 15 ml conical tube and pelleted. The PBS was aspirated, and the cells resuspended in Nucleofector™ P4 or P3 (Lonza, Allendale, NJ) solution to a density of 2e5-1e6 cells/ml per sample. 20 μl of the cell suspension was then added to each well containing 2.5 μl of the Cas12a guide/nucleoprotein complexes, and the entire volume from each well was transferred to a well of a 96-well Nucleocuvette™ Plate (Lonza, Allendale, NJ). The plate was loaded onto the Nucleofector™ 96-well Shuttle (Lonza, Allendale, NJ) and cells nucleofected using the CA137 Nucleofector™ program (Lonza, Allendale, NJ). Post-nucleofection, 77.5 μl of ImmunoCult-XF complete medium supplemented with IL-2 (100 units/mL) was added to each well, and the entire volume of transfected cell suspension was transferred to a 96-well cell culture plate containing 100 μl pre-warmed ImmunoCult-XF complete medium supplemented with IL-2 (100 units/mL). The plate was transferred to a tissue culture incubator and maintained at 37° C. in 5% CO₂ for 48 hours before downstream analysis.

### Example 4

Tiling of Human Genes with Cas12a Guide/Nucleoprotein Complexes

This Example describes the design and use of Cas12a guide/nucleoprotein complexes to target the genes encoding human T cell alpha constant region (TRAC), beta-2-microglubulin (B2M), programmed cell death 1 (PDCD1), cytokine-inducible SH2-containing protein (CISH), and Cb1 Proto-Oncogene B (CBL-B), in human T cells.

A. Designing the AsCas12a crRNA Guides

All 20-nucleotide sequences downstream (in a 3′ direction) of a 5′-TTTV PAM motif in the coding regions of the genes encoding human TRAC, B2M, PDCD1, CISH, and CBL-B, were selected for targeting (SEQ ID Nos: 12-189). Target selection criteria included, but were not limited to, homology to other regions in the genome; percent G-C content; melting temperature; and presence of homopolymer within the spacer.

The identified 20-nucleotide sequences were appended downstream (in a 3′ direction) to the AsCas12a activating region sequence (SEQ ID NO: 6)

Sequences were provided to commercial manufacturers for synthesis. Then, individual Cas12a guide/nucleoprotein complexes were prepared as described in Example 2 and transfected into primary T cells as described in Example 3.

### B. Determining Genome Editing Efficiency

(1) Target dsDNA Sequence Generation for Deep Sequencing

gDNA was isolated from the nucleofected primary T cells 48 hours after transfection using the Cas12a guide/nucleoprotein complexes and 50 μL QuickExtract™ DNA extraction solution (Epicentre, Madison, WI) per well, followed by incubation at 37° C. for 10 minutes, 65° C. for 30 minutes, and 95° C. for 3 minutes to stop the reaction. The isolated gDNA was diluted with 50 μL sterile water and samples were stored at −80° C.

Using the isolated gDNA, a first PCR was performed using Q5 Hot Start High-Fidelity 2× Master Mix (New England Biolabs, Ipswich, MA) at 1× concentration, primers designed to amplify the region around the Cas12a target were used at 0.5 μM each, and 3.75 μL of gDNA was used in a final volume of 10 μL. Amplification was conducted by an initial cycle at 98° C. for 1 minute, 35 cycles of 10 s at 98° C., and 20 seconds at 60° C., 30 seconds at 72° C.; and a final extension at 72° C. for 2 minutes. The PCR reactions were diluted 1:100 in water.

A unique set of index primers for a barcoding PCR were used to facilitate multiplex sequencing for each sample. Barcoding PCRs were performed using a reaction mix comprising Q5 Hot Start High-Fidelity 2× Master Mix (New England Biolabs, Ipswich, MA) at 1× concentration, primers at 0.5 μM each, and 1 μL of 1:100 diluted first PCR in a final volume of 10 μL. The reaction mixtures were amplified as follows: 98° C. for 1 minute; followed by 12 cycles of 10 s at 98° C., 20 seconds at 60° C., and 30 seconds at 72° C.; with a final extension reaction at 72° C. for 2 minutes.

### (2) SPRIselect Clean-Up

The PCR reactions were pooled and transferred into a single microfuge tube for SPRIselect (Beckman Coulter, Pasadena, CA) bead-based cleanup of amplicons for sequencing.

To the amplicon, 0.9× volumes of SPRIselect beads were added, mixed, and incubated at room temperature for 10 minutes. The microfuge tube was placed on a magnetic tube stand until the solution cleared. Supernatant was removed and discarded, the residual beads were washed with 1 volume of 85% ethanol, and the beads were incubated at room temperature for 30 seconds. After incubation, ethanol was aspirated, and the beads were air-dried at room temperature for 10 minutes. The microfuge tube was removed from the magnetic stand and 0.25× volumes of Qiagen EB buffer (Qiagen, Venlo, Netherlands) was added to the beads, mixed vigorously, and incubated for 2 minutes at room temperature. The microfuge tube was returned to the magnet, incubated until the solution had cleared, and supernatant containing the purified amplicons was dispensed into a clean microfuge tube. The purified amplicons were quantified using the Nanodrop™ 2000 System (Thermo Scientific, Wilmington, DE) and library quality analyzed using the Fragment Analyzer™ System (Advanced Analytical Technologies, Ames, IA) and the DNF-910 dsDNA Reagent Kit (Advanced Analytical Technologies, Ames, IA).

### (3) Deep Sequencing Set-Up

The pooled amplicons were normalized to a 4 nM concentration as calculated from the Nanodrop™ 2000 System values and the average size of the amplicons. The library was analyzed on a MiSeq Sequencer (Illumina, San Diego, CA) with MiSeq Reagent Kit v2 (Illumina, San Diego, CA) for 300 cycles with two 151-cycle paired-end runs and two 8-cycle index reads.

### (4) Deep Sequencing Data Analysis

The identities of products in the sequencing data were determined based on the index barcode sequences adapted onto the amplicons in the barcoding PCR. A computational script was used to process the MiSeq data that executes, for example, the following tasks:
a. Reads were aligned to the human genome (build GRCh38/38) using Bowtie (bowtie-bio.sourceforge.net/index.shtml) software;
b. Aligned reads were compared to the expected wild type genomic locus sequence, and reads not aligning to any part of the wild type locus discarded;
c. Reads matching wild type sequence were tallied;
d. Reads with indels (insertion or deletion of bases) were categorized by indel type and tallied; and
e. Total indel reads were divided by the sum of wild type reads and indel reads to give percent-mutated reads.

Through the identification of indel sequences at regions targeted by the Cas12a guide/nucleoprotein complexes, the resulting genome editing efficiency of the Cas12a guide/nucleoprotein complexes was determined. The results of the in-cell editing experiment are shown in Table 7.

TABLE 7
Percent Indels Detected with Cas12a
crRNA/nucleoprotein Complexes

| SEQ | Target | % | |
|---|---|---|---|
| ID NO: | ID | editing | StDev |
| SEQ ID | TRAC- | 4.1% | 0.7% |
| NO: 12 | tgt1 | | |
| SEQ ID | TRAC- | 9.7% | 1.9% |
| NO: 13 | tgt2 | | |
| SEQ ID | TRAC- | 89.6% | 1.0% |
| NO: 14 | tgt3 | | |
| SEQ ID | TRAC- | 42.5% | 4.4% |
| NO: 15 | tgt4 | | |
| SEQ ID | TRAC- | 4.6% | 0.8% |
| NO: 16 | tgt5 | | |
| SEQ ID | TRAC- | 87.3% | 0.9% |
| NO: 17 | tgt6 | | |
| SEQ ID | TRAC- | 43.8% | 2.9% |
| NO: 18 | tgt7 | | |
| SEQ ID | TRAC- | 1.0% | 0.9% |
| NO: 19 | tgt8 | | |
| SEQ ID | TRAC- | 32.5% | 4.1% |
| NO: 20 | tgt9 | | |
| SEQ ID | TRAC- | 32.3% | 2.2% |
| NO: 21 | tgt10 | | |
| SEQ ID | TRAC- | 26.3% | 2.6% |
| NO: 22 | tgt11 | | |
| SEQ ID | TRAC- | 94.3% | 0.6% |
| NO: 23 | tgt12 | | |
| SEQ ID | TRAC- | 4.6% | 0.5% |
| NO: 24 | tgt13 | | |
| SEQ ID | TRAC- | 5.4% | 1.1% |
| NO: 25 | tgt14 | | |
| SEQ ID | TRAC- | 42.2% | 1.7% |
| NO: 26 | tgt15 | | |
| SEQ ID | TRAC- | 0.5% | 0.1% |
| NO: 27 | tgt16 | | |
| SEQ ID | TRAC- | 90.6% | 0.9% |
| NO: 28 | tgt17 | | |
| SEQ ID | TRAC- | 7.2% | 0.6% |
| NO: 29 | tgt18 | | |
| SEQ ID | TRAC- | 27.1% | 3.9% |
| NO: 30 | tgt19 | | |
| SEQ ID | TRAC- | 53.1% | 3.0% |
| NO: 31 | tgt20 | | |
| SEQ ID | TRAC- | 57.3% | 3.4% |
| NO: 32 | tgt21 | | |
| SEQ ID | TRAC- | 51.8% | 2.4% |
| NO: 33 | tgt22 | | |
| SEQ ID | TRAC- | 64.2% | 4.5% |
| NO: 34 | tgt23 | | |
| SEQ ID | TRAC- | 5.7% | 1.0% |
| NO: 35 | tgt24 | | |
| SEQ ID | TRAC- | 96.9% | 0.7% |
| NO: 36 | tgt25 | | |
| SEQ ID | TRAC- | 42.3% | 2.6% |
| NO: 37 | tgt26 | | |
| SEQ ID | TRAC- | 19.4% | 1.4% |
| NO: 38 | tgt27 | | |
| SEQ ID | TRAC- | 95.2% | 0.3% |
| NO: 39 | tgt28 | | |
| SEQ ID | TRAC- | 72.8% | 0.9% |
| NO: 40 | tgt29 | | |
| SEQ ID | TRAC- | 94.9% | 0.6% |
| NO: 41 | tgt30 | | |
| SEQ ID | TRAC- | 16.4% | 2.6% |
| NO: 42 | tgt31 | | |
| SEQ ID | TRAC- | 63.5% | 7.4% |
| NO: 43 | tgt32 | | |
| SEQ ID | TRAC- | 81.4% | 2.2% |
| NO: 44 | tgt33 | | |
| SEQ ID | TRAC- | 0.1% | 0.0% |
| NO: 45 | tgt34 | | |
| SEQ ID | TRAC- | 0.4% | 0.0% |
| NO: 46 | tgt35 | | |
| SEQ ID | TRAC- | 21.7% | 1.7% |
| NO: 47 | tgt36 | | |
| SEQ ID | TRAC- | 33.2% | 2.6% |
| NO: 48 | tgt37 | | |
| SEQ ID | TRAC- | 95.6% | 0.6% |
| NO: 49 | tgt38 | | |
| SEQ ID | TRAC- | 72.8% | 2.5% |
| NO: 50 | tgt39 | | |
| SEQ ID | B2M- | 98.0% | 0.8% |
| NO: 51 | tgt1 | | |
| SEQ ID | B2M- | 39.0% | 2.5% |
| NO: 52 | tgt2 | | |
| SEQ ID | B2M- | 68.3% | 2.4% |
| NO: 53 | tgt3 | | |
| SEQ ID | B2M- | 18.4% | 1.2% |
| NO: 54 | tgt4 | | |
| SEQ ID | B2M- | 34.0% | 3.9% |
| NO: 55 | tgt5 | | |
| SEQ ID | B2M- | 33.2% | 3.6% |
| NO: 56 | tgt6 | | |
| SEQ ID | B2M- | 42.0% | 1.8% |
| NO: 57 | tgt7 | | |
| SEQ ID | B2M- | 32.4% | 1.0% |
| NO: 58 | tgt8 | | |
| SEQ ID | B2M- | 22.6% | 1.6% |
| NO: 59 | tgt9 | | |
| SEQ ID | B2M- | 40.6% | 4.4% |
| NO: 60 | tgt10 | | |
| SEQ ID | B2M- | 21.5% | 2.2% |
| NO: 61 | tgt11 | | |
| SEQ ID | B2M- | 95.8% | 0.7% |
| NO: 62 | tgt12 | | |
| SEQ ID | B2M- | 17.3% | 1.3% |
| NO: 63 | tgt13 | | |
| SEQ ID | B2M- | 3.9% | 0.5% |
| NO: 64 | tgt14 | | |
| SEQ ID | B2M- | 69.4% | 3.7% |
| NO: 65 | tgt15 | | |
| SEQ ID | B2M- | 12.2% | 1.7% |
| NO: 66 | tgt16 | | |
| SEQ ID | B2M- | 1.5% | 0.5% |
| NO: 67 | tgt17 | | |
| SEQ ID | B2M- | 25.4% | 1.3% |
| NO: 68 | tgt18 | | |
| SEQ ID | B2M- | 53.8% | 4.1% |
| NO: 69 | tgt19 | | |
| SEQ ID | B2M- | 8.0% | 0.9% |
| NO: 70 | tgt20 | | |
| SEQ ID | B2M- | 6.1% | 0.7% |
| NO: 71 | tgt21 | | |
| SEQ ID | B2M- | 10.3% | 0.3% |
| NO: 72 | tgt22 | | |
| SEQ ID | B2M- | 29.6% | 2.7% |
| NO: 73 | tgt23 | | |
| SEQ ID | B2M- | 82.8% | 2.6% |
| NO: 74 | tgt24 | | |
| SEQ ID | B2M- | 24.4% | 3.6% |
| NO: 75 | tgt25 | | |
| SEQ ID | B2M- | 0.2% | 0.0% |
| NO: 76 | tgt26 | | |
| SEQ ID | B2M- | 31.3% | 2.6% |
| NO: 77 | tgt27 | | |
| SEQ ID | B2M- | 27.3% | 4.0% |
| NO: 78 | tgt28 | | |
| SEQ ID | B2M- | 2.8% | 0.5% |
| NO: 79 | tgt29 | | |
| SEQ ID | B2M- | 31.7% | 2.2% |
| NO: 80 | tgt30 | | |
| SEQ ID | B2M- | 14.8% | 1.9% |
| NO: 81 | tgt31 | | |
| SEQ ID | B2M- | 54.4% | 4.4% |
| NO: 82 | tgt32 | | |
| SEQ ID | B2M- | 0.4% | 0.2% |
| NO: 83 | tgt33 | | |
| SEQ ID | B2M- | 77.1% | 1.8% |
| NO: 84 | tgt34 | | |
| SEQ ID | B2M- | 15.3% | 1.5% |
| NO: 85 | tgt35 | | |
| SEQ ID | B2M- | 60.4% | 5.9% |
| NO: 86 | tgt36 | | |
| SEQ ID | B2M- | 3.4% | 0.7% |
| NO: 87 | tgt37 | | |
| SEQ ID | B2M- | 3.7% | 0.4% |
| NO: 88 | tgt38 | | |
| SEQ ID | B2M- | 48.2% | 4.0% |
| NO: 89 | tgt39 | | |
| SEQ ID | B2M- | 7.0% | 0.8% |
| NO: 90 | tgt40 | | |
| SEQ ID | B2M- | 1.1% | 0.4% |
| NO: 91 | tgt41 | | |
| SEQ ID | B2M- | 1.0% | 0.1% |
| NO: 92 | tgt42 | | |
| SEQ ID | B2M- | 3.0% | 0.4% |
| NO: 93 | tgt43 | | |
| SEQ ID | B2M- | 35.6% | 3.0% |
| NO: 94 | tgt44 | | |
| SEQ ID | B2M- | 4.9% | 0.8% |
| NO: 95 | tgt45 | | |
| SEQ ID | B2M- | 3.5% | 0.4% |
| NO: 96 | tgt46 | | |
| SEQ ID | B2M- | 6.3% | 0.9% |
| NO: 97 | tgt47 | | |
| SEQ ID | B2M- | 7.9% | 0.6% |
| NO: 98 | tgt48 | | |
| SEQ ID | B2M- | 38.7% | 2.6% |
| NO: 99 | tgt49 | | |
| SEQ ID | B2M- | 6.4% | 0.8% |
| NO: 100 | tgt50 | | |
| SEQ ID | B2M- | 87.6% | 1.3% |
| NO: 101 | tgt51 | | |
| SEQ ID | B2M- | 88.7% | 0.9% |
| NO: 102 | tgt52 | | |
| SEQ ID | B2M- | 50.1% | 4.2% |
| NO: 103 | tgt53 | | |
| SEQ ID | B2M- | 94.7% | 0.2% |
| NO: 104 | tgt54 | | |
| SEQ ID | B2M- | 88.0% | 0.8% |
| NO: 105 | tgt55 | | |
| SEQ ID | B2M- | 1.9% | 0.2% |
| NO: 106 | tgt56 | | |
| SEQ ID | B2M- | 1.8% | 0.4% |
| NO: 107 | tgt57 | | |
| SEQ ID | B2M- | 75.6% | 1.5% |
| NO: 108 | tgt58 | | |
| SEQ ID | B2M- | 42.8% | 0.9% |
| NO: 109 | tgt59 | | |
| SEQ ID | B2M- | 21.5% | 0.3% |
| NO: 110 | tgt60 | | |
| SEQ ID | B2M- | 7.1% | 0.5% |
| NO: 111 | tgt61 | | |
| SEQ ID | B2M- | 38.6% | 0.8% |
| NO: 112 | tgt62 | | |
| SEQ ID | B2M- | 85.6% | 3.7% |
| NO: 113 | tgt63 | | |
| SEQ ID | B2M- | 91.5% | 2.4% |
| NO: 114 | tgt64 | | |
| SEQ ID | B2M- | 79.7% | 2.3% |
| NO: 115 | tgt65 | | |
| SEQ ID | B2M- | 35.9% | 1.5% |
| NO: 116 | tgt66 | | |
| SEQ ID | B2M- | 75.5% | 1.4% |
| NO: 117 | tgt67 | | |
| SEQ ID | B2M- | 55.9% | 1.5% |
| NO: 118 | tgt68 | | |
| SEQ ID | B2M- | 0.6% | 0.1% |
| NO: 119 | tgt69 | | |
| SEQ ID | B2M- | 9.8% | 0.4% |
| NO: 120 | tgt70 | | |
| SEQ ID | B2M- | 50.8% | 1.2% |
| NO: 121 | tgt71 | | |
| SEQ ID | B2M- | 5.2% | 0.5% |
| NO: 122 | tgt72 | | |
| SEQ ID | B2M- | 29.7% | 1.4% |
| NO: 123 | tgt73 | | |
| SEQ ID | B2M- | 71.0% | 1.1% |
| NO: 124 | tgt74 | | |
| SEQ ID | B2M- | 4.0% | 0.5% |
| NO: 125 | tgt75 | | |
| SEQ ID | B2M- | 14.1% | 1.1% |
| NO: 126 | tgt76 | | |
| SEQ ID | B2M- | 16.6% | 0.5% |
| NO: 127 | tgt77 | | |
| SEQ ID | B2M- | 8.3% | 1.1% |
| NO: 128 | tgt78 | | |
| SEQ ID | B2M- | 3.4% | 0.7% |
| NO: 129 | tgt79 | | |
| SEQ ID | B2M- | 13.7% | 2.0% |
| NO: 130 | tgt80 | | |
| SEQ ID | B2M- | 7.2% | 1.0% |
| NO: 131 | tgt81 | | |
| SEQ ID | B2M- | 1.6% | 0.2% |
| NO: 132 | tgt82 | | |
| SEQ ID | B2M- | 83.6% | 1.0% |
| NO: 133 | tgt83 | | |
| SEQ ID | PDCD1- | 75.3% | 4.0% |
| NO: 134 | tgt1 | | |
| SEQ ID | PDCD1- | 19.2% | 1.1% |
| NO: 135 | tgt2 | | |
| SEQ ID | PDCD1- | 0.0% | 0.0% |
| NO: 136 | tgt3 | | |
| SEQ ID | PDCD1- | 35.6% | 1.7% |
| NO: 137 | tgt4 | | |
| SEQ ID | PDCD1- | 25.6% | 2.6% |
| NO: 138 | tgt5 | | |
| SEQ ID | PDCD1- | 22.5% | 0.7% |
| NO: 139 | tgt6 | | |
| SEQ ID | PDCD1- | 6.4% | 0.3% |
| NO: 140 | tgt7 | | |
| SEQ ID | PDCD1- | 14.7% | 1.0% |
| NO: 141 | tgt8 | | |
| SEQ ID | PDCD1- | 90.0% | 1.5% |
| NO: 142 | tgt9 | | |
| SEQ ID | PDCD1- | 52.6% | 4.3% |
| NO: 143 | tgt10 | | |
| SEQ ID | PDCD1- | 32.6% | 2.6% |
| NO: 144 | tgt11 | | |
| SEQ ID | PDCD1- | 8.3% | 0.6% |
| NO: 145 | tgt12 | | |
| SEQ ID | PDCD1- | 10.9% | 2.1% |
| NO: 146 | tgt13 | | |
| SEQ ID | PDCD1- | 7.4% | 0.4% |
| NO: 147 | tgt14 | | |
| SEQ ID | PDCD1- | 83.0% | 1.0% |
| NO: 148 | tgt15 | | |
| SEQ ID | PDCD1- | 0.2% | 0.0% |
| NO: 149 | tgt16 | | |
| SEQ ID | PDCD1- | 0.1% | 0.1% |
| NO: 150 | tgt17 | | |
| SEQ ID | PDCD1- | 0.3% | 0.1% |
| NO: 151 | tgt18 | | |
| SEQ ID | PDCD1- | 87.7% | 1.0% |
| NO: 152 | tgt19 | | |
| SEQ ID | PDCD1- | 92.8% | 0.8% |
| NO: 153 | tgt20 | | |
| SEQ ID | PDCD1- | 0.9% | 0.0% |
| NO: 154 | tgt21 | | |
| SEQ ID | PDCD1- | 0.1% | 0.1% |
| NO: 155 | tgt22 | | |
| SEQ ID | CISH- | 56.0% | 1.4% |
| NO: 156 | tgt1 | | |
| SEQ ID | CISH- | 20.0% | 0.9% |
| NO: 157 | tgt2 | | |
| SEQ ID | CISH- | 78.3% | 1.6% |
| NO: 158 | tgt3 | | |
| SEQ ID | CISH- | 40.8% | 1.3% |
| NO: 159 | tgt4 | | |
| SEQ ID | CISH- | 82.2% | 1.1% |
| NO: 160 | tgt5 | | |
| SEQ ID | CISH- | 21.6% | 2.7% |
| NO: 161 | tgt6 | | |
| SEQ ID | CISH- | 79.6% | 1.7% |
| NO: 162 | tgt7 | | |
| SEQ ID | CISH- | 61.9% | 1.6% |
| NO: 163 | tgt8 | | |
| SEQ ID | CISH- | 79.7% | 1.2% |
| NO: 164 | tgt9 | | |
| SEQ ID | CISH- | 70.9% | 2.5% |
| NO: 165 | tgt10 | | |
| SEQ ID | CBLB- | 12.2% | 6.4% |
| NO: 166 | tgt1 | | |
| SEQ ID | CBLB- | 14.6% | 1.7% |
| NO: 167 | tgt2 | | |
| SEQ ID | CBLB- | 48.5% | 1.6% |
| NO: 168 | tgt3 | | |
| SEQ ID | CBLB- | 71.9% | 1.5% |
| NO: 169 | tgt4 | | |
| SEQ ID | CBLB- | 27.1% | 2.8% |
| NO: 170 | tgt5 | | |
| SEQ ID | CBLB- | 85.8% | 1.8% |
| NO: 171 | tgt6 | | |
| SEQ ID | CBLB- | 16.8% | 1.8% |
| NO: 172 | tgt7 | | |
| SEQ ID | CBLB- | 4.1% | 0.9% |
| NO: 173 | tgt8 | | |
| SEQ ID | CBLB- | 1.5% | 0.0% |
| NO: 174 | tgt9 | | |
| SEQ ID | CBLB- | 6.4% | 0.9% |
| NO: 175 | tgt10 | | |
| SEQ ID | CBLB- | 17.2% | 2.1% |
| NO: 176 | tgt11 | | |
| SEQ ID | CBLB- | 31.2% | 3.0% |
| NO: 177 | tgt12 | | |
| SEQ ID | CBLB- | 0.4% | 0.0% |
| NO: 178 | tgt13 | | |
| SEQ ID | CBLB- | 0.2% | 0.1% |
| NO: 179 | tgt14 | | |
| SEQ ID | CBLB- | 7.0% | 0.5% |
| NO: 180 | tgt15 | | |
| SEQ ID | CBLB- | 5.2% | 0.4% |
| NO: 181 | tgt16 | | |
| SEQ ID | CBLB- | 22.2% | 2.1% |
| NO: 182 | tgt17 | | |
| SEQ ID | CBLB- | 0.0% | 0.0% |
| NO: 183 | tgt17 | | |
| SEQ ID | CBLB- | 0.0% | 0.0% |
| NO: 184 | tgt17 | | |
| SEQ ID | CBLB- | 2.4% | 0.3% |
| NO: 185 | tgt20 | | |
| SEQ ID | CBLB- | 6.2% | 0.9% |
| NO: 186 | tgt21 | | |
| SEQ ID | CBLB- | 17.6% | 5.2% |
| NO: 187 | tgt22 | | |
| SEQ ID | CBLB- | 19.8% | 2.8% |
| NO: 188 | tgt23 | | |
| SEQ ID | CBLB- | 0.1% | 0.0% |
| NO: 189 | tgt24 | | |

StDev = standard deviation; n = 3

The data presented in Table 7 demonstrate that Cas12a crRNA/nucleoprotein complexes are capable of on-editing multiple genes in human primary T cells. Other genes, such as those described elsewhere herein, can be targeted in a similar manner, using AsCas12a or other Cas12a proteins (such as L. bacterium or F. novicida).

### Example 5

Engineering Cas12a chRDNA Guide Molecules with DNA in the Target Binding Sequence

The following Example describes the engineering of AsCas12a chRDNA guide molecules to comprise DNA bases in the target binding sequence.

A. In Silico Cas12a chRDNA Guide Design

The 20-nucleotide target binding sequence of the AsCas12a guide was selected for engineering, and an individual DNA base was utilized at each position in the target binding sequence. The location of DNA bases in the target binding sequence of the AsCas12a chRDNA guide molecules are shown in Table 8 (DNA bases are shown with a “d” and RNA bases are shown with a “R”; a control crRNA is also shown).

TABLE 8
AsCas12a chRDNA Guide Target Binding Sequences

| | DNA | |
|---|---|---|
| SEQ ID NO: | position | Sequence |
| SEQ ID NO: 190 | crRNA | RRRRRRRRRRRRRRRRRRRR |
| SEQ ID NO: 191 | 1 | dRRRRRRRRRRRRRRRRRRR |
| SEQ ID NO: 192 | 2 | RdRRRRRRRRRRRRRRRRRR |
| SEQ ID NO: 193 | 3 | RRdRRRRRRRRRRRRRRRRR |
| SEQ ID NO: 194 | 4 | RRRdRRRRRRRRRRRRRRRR |
| SEQ ID NO: 195 | 5 | RRRRdRRRRRRRRRRRRRRR |
| SEQ ID NO: 196 | 6 | RRRRRdRRRRRRRRRRRRRR |
| SEQ ID NO: 197 | 7 | RRRRRRdRRRRRRRRRRRRR |
| SEQ ID NO: 198 | 8 | RRRRRRRdRRRRRRRRRRRR |
| SEQ ID NO: 199 | 9 | RRRRRRRRdRRRRRRRRRRR |
| SEQ ID NO: 200 | 10 | RRRRRRRRRdRRRRRRRRRR |
| SEQ ID NO: 201 | 11 | RRRRRRRRRRdRRRRRRRRR |
| SEQ ID NO: 202 | 12 | RRRRRRRRRRRdRRRRRRRR |
| SEQ ID NO: 203 | 13 | RRRRRRRRRRRRdRRRRRRR |
| SEQ ID NO: 204 | 14 | RRRRRRRRRRRRRdRRRRRR |
| SEQ ID NO: 205 | 15 | RRRRRRRRRRRRRRdRRRRR |
| SEQ ID NO: 206 | 16 | RRRRRRRRRRRRRRRdRRRR |
| SEQ ID NO: 207 | 17 | RRRRRRRRRRRRRRRRdRRR |
| SEQ ID NO: 208 | 18 | RRRRRRRRRRRRRRRRRdRR |
| SEQ ID NO: 209 | 19 | RRRRRRRRRRRRRRRRRRdR |
| SQ ID NO: 210 | 20 | RRRRRRRRRRRRRRRRRRRd |

Three target sequences in the gene encoding human B2M (B2M-tgt12, B2M-tgt1, B2M-intron-tgt12), a target sequence in the gene encoding human TRAC (TRAC-tgt12), and a target sequence in the gene encoding human DNA methyltransferase 1 (DNMT1) (DNMT1-tgt1), were selected for editing. A Cas12a chRDNA guide for each target comprising a target binding sequence containing a single DNA base at each position (see Table 8), as well as a Cas12a crRNA control sequence, were provided to a commercial manufacturer for synthesis.

### B. Cell Transfection and Analysis

Individual Cas12a guide/nucleoprotein complexes for screening were prepared essentially as described in Example 2. The nucleoprotein complexes were transfected into primary T cells as described in Example 3, and the resulting genome editing efficiency of the Cas12a guide/nucleoprotein complexes was determined as described in Example 4. The results of the in-cell editing experiment are shown in Table 9.

TABLE 9
Editing Rates of Cas12a Guide/nucleoprotein Complexes

| | SEQ | DNA | % | |
|---|---|---|---|---|
| Target | ID NO: | position | Editing | StDev |
| B2M- | SEQ ID NO: 211 | crRNA | 87.00% | 1.10% |
| tgt12 | SEQ ID NO: 212 | 1 | 86.40% | 1.40% |
| | SEQ ID NO: 213 | 2 | 84.60% | 2.70% |
| | SEQ ID NO: 214 | 3 | 84.80% | 2.20% |
| | SEQ ID NO: 215 | 4 | 87.00% | 1.20% |
| | SEQ ID NO: 216 | 5 | 86.00% | 1.10% |
| | SEQ ID NO: 217 | 6 | 86.60% | 1.00% |
| | SEQ ID NO: 218 | 7 | 86.30% | 0.40% |
| | SEQ ID NO: 219 | 8 | 85.00% | 1.60% |
| | SEQ ID NO: 220 | 9 | 85.30% | 1.50% |
| | SEQ ID NO: 221 | 10 | 87.00% | 2.20% |
| | SEQ ID NO: 222 | 11 | 85.50% | 2.00% |
| | SEQ ID NO: 223 | 12 | 86.30% | 0.90% |
| | SEQ ID NO: 224 | 13 | 85.70% | 1.30% |
| | SEQ ID NO: 225 | 14 | 80.80% | 1.40% |
| | SEQ ID NO: 226 | 15 | 85.10% | 1.40% |
| | SEQ ID NO: 227 | 16 | 55.30% | 2.10% |
| | SEQ ID NO: 228 | 17 | 86.20% | 1.90% |
| | SEQ ID NO: 229 | 18 | 86.00% | 2.20% |
| | SEQ ID NO: 230 | 19 | 87.10% | 2.30% |
| | SEQ ID NO: 231 | 20 | 86.20% | 1.40% |
| TRAC- | SEQ ID NO: 232 | crRNA | 90.57% | 1.16% |
| tgt12 | SEQ ID NO: 233 | 1 | 85.87% | 0.52% |
| | SEQ ID NO: 234 | 2 | 83.90% | 0.93% |
| | SEQ ID NO: 235 | 3 | 28.67% | 1.42% |
| | SEQ ID NO: 236 | 4 | 61.27% | 1.97% |
| | SEQ ID NO: 237 | 5 | 16.43% | 1.14% |
| | SEQ ID NO: 238 | 6 | 2.75% | 0.02% |
| | SEQ ID NO: 239 | 7 | 70.90% | 0.71% |
| | SEQ ID NO: 240 | 8 | 22.27% | 0.99% |
| | SEQ ID NO: 241 | 9 | 78.90% | 1.13% |
| | SEQ ID NO: 242 | 10 | 83.50% | 0.67% |
| | SEQ ID NO: 243 | 11 | 86.97% | 1.21% |
| | SEQ ID NO: 244 | 12 | 76.30% | 0.24% |
| | SEQ ID NO: 245 | 13 | 58.53% | 1.67% |
| | SEQ ID NO: 246 | 14 | 79.27% | 1.21% |
| | SEQ ID NO: 247 | 15 | 87.40% | 1.42% |
| | SEQ ID NO: 248 | 16 | 73.87% | 0.87% |
| | SEQ ID NO: 249 | 17 | 90.97% | 1.47% |
| | SEQ ID NO: 250 | 18 | 67.97% | 0.78% |
| | SEQ ID NO: 251 | 19 | 90.27% | 0.90% |
| | SEQ ID NO: 252 | 20 | 95.10% | 0.70% |
| DNMT1- | SEQ ID NO: 253 | crRNA | 67.10% | 2.50% |
| tgt1 | SEQ ID NO: 254 | 1 | 68.30% | 1.30% |
| | SEQ ID NO: 255 | 2 | 2.10% | 0.20% |
| | SEQ ID NO: 256 | 3 | 36.40% | 0.80% |
| | SEQ ID NO: 257 | 4 | 12.30% | 0.30% |
| | SEQ ID NO: 258 | 5 | 0.30% | 0.10% |
| | SEQ ID NO: 259 | 6 | 28.00% | 0.60% |
| | SEQ ID NO: 260 | 7 | 8.20% | 0.30% |
| | SEQ ID NO: 261 | 8 | 67.30% | 1.20% |
| | SEQ ID NO: 262 | 9 | 63.80% | 1.90% |
| | SEQ ID NO: 263 | 10 | 62.60% | 1.30% |
| | SEQ ID NO: 264 | 11 | 58.50% | 4.10% |
| | SEQ ID NO: 265 | 12 | 45.60% | 0.40% |
| | SEQ ID NO: 266 | 13 | 18.90% | 1.00% |
| | SEQ ID NO: 267 | 14 | 64.10% | 1.10% |
| | SEQ ID NO: 268 | 15 | 62.30% | 1.00% |
| | SEQ ID NO: 269 | 16 | 42.90% | 1.10% |
| | SEQ ID NO: 270 | 17 | 59.20% | 1.50% |
| | SEQ ID NO: 271 | 18 | 43.40% | 1.00% |
| | SEQ ID NO: 272 | 19 | 63.40% | 1.60% |
| | SEQ ID NO: 273 | 20 | 64.60% | 2.20% |
| B2M- | SEQ ID NO: 274 | crRNA | 95.20% | 0.60% |
| tgt1 | SEQ ID NO: 275 | 1 | 94.80% | 1.00% |
| | SEQ ID NO: 276 | 2 | 20.70% | 1.40% |
| | SEQ ID NO: 277 | 3 | 90.90% | 1.20% |
| | SEQ ID NO: 278 | 4 | 49.50% | 2.30% |
| | SEQ ID NO: 279 | 5 | 20.00% | 0.70% |
| | SEQ ID NO: 280 | 6 | 90.40% | 0.60% |
| | SEQ ID NO: 281 | 7 | 61.90% | 2.10% |
| | SEQ ID NO: 282 | 8 | 95.50% | 1.00% |
| | SEQ ID NO: 283 | 9 | 94.50% | 1.40% |
| | SEQ ID NO: 284 | 10 | 93.50% | 1.70% |
| | SEQ ID NO: 285 | 11 | 94.90% | 1.00% |
| | SEQ ID NO: 286 | 12 | 94.40% | 0.60% |
| | SEQ ID NO: 287 | 13 | 84.70% | 3.30% |
| | SEQ ID NO: 288 | 14 | 95.70% | 0.90% |
| | SEQ ID NO: 289 | 15 | 92.90% | 5.00% |
| | SEQ ID NO: 290 | 16 | 94.90% | 1.10% |
| | SEQ ID NO: 291 | 17 | 95.40% | 0.90% |
| | SEQ ID NO: 292 | 18 | 94.20% | 1.10% |
| | SEQ ID NO: 293 | 19 | 95.60% | 0.80% |
| | SEQ ID NO: 294 | 20 | 93.80% | 1.00% |
| B2M- | SEQ ID NO: 295 | crRNA | 86.40% | 6.60% |
| intron- | SEQ ID NO: 296 | 1 | 87.50% | 2.00% |
| tgt1 | SEQ ID NO: 297 | 2 | 13.60% | 1.00% |
| | SEQ ID NO: 298 | 3 | 53.80% | 1.00% |
| | SEQ ID NO: 299 | 4 | 54.70% | 3.90% |
| | SEQ ID NO: 300 | 5 | 7.70% | 0.40% |
| | SEQ ID NO: 301 | 6 | 56.40% | 6.80% |
| | SEQ ID NO: 302 | 7 | 28.20% | 3.00% |
| | SEQ ID NO: 303 | 8 | 87.30% | 0.40% |
| | SEQ ID NO: 304 | 9 | 85.00% | 1.70% |
| | SEQ ID NO: 305 | 10 | 86.80% | 1.70% |
| | SEQ ID NO: 306 | 11 | 87.70% | 0.60% |
| | SEQ ID NO: 307 | 12 | 80.10% | 1.20% |
| | SEQ ID NO: 308 | 13 | 53.10% | 2.60% |
| | SEQ ID NO: 309 | 14 | 85.20% | 2.70% |
| | SEQ ID NO: 310 | 15 | 84.10% | 3.00% |
| | SEQ ID NO: 311 | 16 | 66.50% | 2.30% |
| | SEQ ID NO: 312 | 17 | 87.80% | 1.10% |
| | SEQ ID NO: 313 | 18 | 77.10% | 2.00% |
| | SEQ ID NO: 314 | 19 | 86.20% | 1.10% |
| | SEQ ID NO: 315 | 20 | 87.40% | 1.60% |

StDev = standard deviation; n = 3

The editing results in Table 9 demonstrate that Cas12a chRDNA guide molecules comprising DNA in the spacer are capable of editing at a rate comparable to the crRNA across multiple targets (compare SEQ ID NO: 211 to SEQ ID NO: 224; SEQ ID NO: 232 to SEQ ID NO: 234, or SEQ ID NO: 274 to SEQ ID NO: 293). The editing rates of the chRDNA guide designs in Table 9 were normalized to the editing rates of the crRNA for each target. Average normalized editing rates are presented in FIG. 13, where position within the target binding sequence is plotted as a function of average normalized editing. The preferred positions of DNA base utilization (i.e., greater than 70% average normalized editing) are indicated with grey fill, and include positions 1, 8, 9, 10, 11, 12, 14, 15, 16, 17, 18, 19, and 20. The data presented in the present example, as well as the data in Table 9 and FIG. 13, can be used to determine which positions within the target binding sequence of a Cas12a guide can be engineering as DNA.

### Example 6

Cas12a chRDNA Guide Molecules with Multiple DNA Bases in the Target Binding Sequence

This Example describes the designing and testing of Cas12a chRDNA guide molecules with multiple DNA bases in the target binding sequence.

A. In Silico Design of Cas12a chRDNA Guides

The 20-nucleotide sequence of three targets in the gene encoding human B2M (B2M-tgt12, B2M-tgt1, B2M-intron-tgt12), a target in the gene encoding human TRAC (TRAC-tgt12), and a target in the gene encoding human DNA methyltransferase 1 (DNMT1-tgt1), were selected for editing. For each target, between 1 and 7 nucleotides of DNA were designed into the target binding sequence of each AsCas12a guide. Design criteria for the position of DNA bases included, but were not limited to, previously single position screen data (see Example 5), prior consensus of positions tolerant to DNA (see FIG. 13), distance between individual DNA bases in target binding sequence, and known location of mismatches in an off-target sequence. Cas12a chRDNA guide designs, as well as a crRNA control sequence, were provided to a commercial manufacturer for synthesis.

### B. Cell Transfection and Analysis

Individual Cas12a guide/nucleoprotein complexes for screening were prepared essentially as described in Example 2. The Cas12a guide/nucleoprotein complexes were transfected into primary T cells as described in Example 3, and the resulting genome editing efficiency of the Cas12a guide/nucleoprotein complexes was determined as described in Example 4. The results of the in-cell editing experiment, and the location of DNA bases in the target binding sequence of each Cas12a chRDNA guide, are shown in Table 10.

TABLE 10
Editing Rates of Cas12a Guide/nucleoprotein Complexes

| | | | % | |
|---|---|---|---|---|
| Target | SEQ ID NO: | DNA Position | Editing | StDev |
| TRAC- | SEQ ID NO: 316 | — | 89.00% | 1.45% |
| tgt12 | SEQ ID NO: 317 | 20, 19 | 90.00% | 0.64% |
| | SEQ ID NO: 318 | 20, 17 | 90.80% | 1.84% |
| | SEQ ID NO: 319 | 19, 15 | 80.17% | 2.23% |
| | SEQ ID NO: 320 | 11, 10 | 87.10% | 1.06% |
| | SEQ ID NO: 321 | 20, 11, 1 | 90.27% | 0.78% |
| | SEQ ID NO: 322 | 17, 11 | 87.03% | 2.36% |
| | SEQ ID NO: 323 | 2, 1 | 23.90% | 0.30% |
| | SEQ ID NO: 324 | 15, 10, 11 | 60.93% | 0.93% |
| | SEQ ID NO: 325 | 20, 17, 10, 1 | 59.93% | 6.09% |
| | SEQ ID NO: 326 | 18, 15, 2 | 10.87% | 0.23% |
| | SEQ ID NO: 327 | 18, 15, 11, 10, 3 | 5.20% | 0.20% |
| | SEQ ID NO: 328 | 10, 17, 15, 10, 1 | 74.17% | 0.93% |
| | SEQ ID NO: 329 | 20, 19, 17, 15, 11, 10, 2 | 1.29% | 0.11% |
| B2M- | SEQ ID NO: 330 | — | 95.20% | 0.60% |
| tgt12 | SEQ ID NO: 331 | 20, 17 | 90.83% | 0.17% |
| | SEQ ID NO: 332 | 15, 11 | 90.77% | 1.27% |
| | SEQ ID NO: 333 | 15, 14 | 92.17% | 1.53% |
| | SEQ ID NO: 334 | 11, 10 | 90.83% | 1.05% |
| | SEQ ID NO: 335 | 20, 11, 1 | 93.60% | 0.92% |
| | SEQ ID NO: 336 | 17, 11 | 92.57% | 1.52% |
| | SEQ ID NO: 337 | 2, 1 | 90.70% | 0.64% |
| | SEQ ID NO: 338 | 20, 17, 15, 2 | 65.30% | 8.02% |
| | SEQ ID NO: 339 | 15, 14, 11, 10 | 72.63% | 1.62% |
| | SEQ ID NO: 340 | 20, 17, 11, 10, 2, 1, | 50.37% | 8.52% |
| | SEQ ID NO: 341 | 17, 15, 14, 11, 10, 9, 3, 1 | 21.53% | 1.15% |
| | SEQ ID NO: 342 | 19, 16, 13, 11, 10, 1 | 0.51% | 0.09% |
| | SEQ ID NO: 343 | 16, 9, 3, 2, 1 | 4.41% | 0.25% |
| | SEQ ID NO: 344 | 20-11 | 0.00% | 0.05% |
| B2M- | SEQ ID NO: 345 | — | 97.30% | 0.20% |
| tgt1 | SEQ ID NO: 346 | 20, 11, 1 | 85.00% | 0.30% |
| | SEQ ID NO: 347 | 19, 14, 8 | 94.90% | 0.60% |
| | SEQ ID NO: 348 | 19, 17, 14, 8 | 52.70% | 4.80% |
| | SEQ ID NO: 349 | 12, 11, 8 | 65.70% | 1.70% |
| | SEQ ID NO: 350 | 18, 16, 10 | 59.20% | 5.30% |
| | SEQ ID NO: 351 | 19, 17, 15, 1 | 74.10% | 2.40% |
| | SEQ ID NO: 352 | 19-16 | 4.50% | 0.60% |
| B2M- | SEQ ID NO: 353 | — | 79.80% | 3.00% |
| intron- | SEQ ID NO: 354 | 20, 11, 1 | 79.10% | 3.90% |
| tgt1 | SEQ ID NO: 355 | 20, 17, 11 | 78.30% | 2.00% |
| | SEQ ID NO: 356 | 8-Nov | 65.40% | 1.70% |
| | SEQ ID NO: 357 | 17, 10, 8, 1 | 74.20% | 2.10% |
| | SEQ ID NO: 358 | 20, 19, 17 | 61.40% | 4.00% |
| | SEQ ID NO: 359 | 20, 19, 9, 8 | 66.60% | 1.90% |
| | SEQ ID NO: 360 | 20, 19, 17, 11, 10, 1 | 42.40% | 6.10% |
| DNMT1- | SEQ ID NO: 361 | — | 69.00% | 1.06% |
| tgt1 | SEQ ID NO: 362 | 1, 2 | 63.43% | 3.69% |
| | SEQ ID NO: 363 | 14, 2 | 53.00% | 0.26% |
| | SEQ ID NO: 364 | 8, 19 | 65.80% | 2.35% |
| | SEQ ID NO: 365 | 8, 14 | 59.93% | 1.42% |
| | SEQ ID NO: 366 | 1, 8, 20 | 67.43% | 0.85% |
| | SEQ ID NO: 367 | 10-Aug | 51.73% | 1.01% |
| | SEQ ID NO: 368 | 8, 9, 15 | 54.90% | 2.96% |
| | SEQ ID NO: 369 | 1, 8, 19 | 54.10% | 4.11% |

StDev = standard deviation;
n = 3

The editing results in Table 10 demonstrate that Cas12a chRDNA guide molecules comprising multiple DNA bases in the target binding sequence are capable of editing at a rate comparable to the crRNA across multiple targets (compare SEQ ID NO: 316 to SEQ ID NO: 321; SEQ ID NO: 330 to SEQ ID NO: 335, or SEQ ID NO: 345 to SEQ ID NO: 347).

### Example 7

Reduced Off-Target Editing with Cas12a chRDNA Guides

This Example describes the identification of Cas12a off-targets using the SITE-Seq® assay (Cameron, P., et al., (2017). Mapping the genomic landscape of CRISPR-Cas9 cleavage. Nature Methods, 14 (6). 600-606. and reduction in off-target editing rates of a Cas12a chRDNA guide compared to a Cas12a crRNA guide.

### A. SITE-Seq® Assay

Human primary T cells were grown as described in Example 1. After expansion of cells in 50 ml conical tubes, high molecular weight genomic DNA (gDNA) was extracted from the human primary T cells using the Blood and Cell Culture DNA Maxi Kit (Qiagen, Hilden, Germany), according to the manufacturer's protocol.

A 20-nucleotide target in the gene encoding human Ribosomal Protein L32 (RPL32) (RPL32-tgt1) and in the gene encoding DNMT1 (DNMT1-tgt1) were selected for evaluation using the SITE-Seq® assay (Cameron, P., et al., (2017). Nature Methods, 14 (6). 600-606). Each Cas12a guide component was serially diluted to the corresponding nucleoprotein concentration, incubated at 95° C. for 2 minutes, and then allowed to slowly come to room temperature over 5 minutes. Cas12a nucleoprotein complexes for RPL32 (SEQ ID NO: 375) and DNMT1 (SEQ ID NO: 404) targets were formed by combining incubated Cas12a guides with Cas12a protein at a 3:1 ratio in cleavage reaction buffer (60 mM TRIS-acetate, 150 mM potassium acetate, 30 mM magnesium acetate, at pH 7.9), and incubated at 37° C. for 10 minutes. Individual cleavage of 10 μg of genomic DNA (gDNA) occurred at 6 Cas12a guide/nucleoprotein complex concentrations (8 nM, 16 nM, 32 nM, 48 nM, 64 nM, 96 nM, and 128 nM) in a total volume of 50 μL. Negative control reactions (0 nM) were assembled in parallel, and did not include any nucleoprotein complex. All cleavage reactions, including the negative controls, were all assembled in triplicate in a 96-well format plate. The cleavage reactions were allowed to incubate at 37° C. for 4 hours.

Library preparation and sequencing were done essentially as described by Cameron et al. (Nature Meth., 2017, 14:600-606), with the exception of the dA-tailing step post-nucleoprotein complex cleavage. An additional enzymatic end-repair step was needed due to Cas12a nucleoprotein complex cleavage resulting in a staggered (5′ overhang) end. The volumes added remained the same, but the original dA-tailing kit components were swapped out for the End Prep Enzyme Mix (3 μL) and 10× End Repair Reaction Buffer (5 μL) components of the NEBNext® Ultra™ End Repair/dA-Tailing Module from New England BioLabs (NEB #E7442L). Samples were incubated at 20° C. for 30 minutes and then 65° C. for 30 minutes, and the standard procedure was resumed. NGS sequencing was performed using the Illumina NextSeq platform (Illumina, San Diego, CA), and ˜3 million reads were obtained for each sample. Any sites recovered by the SITE-Seq® assay (Cameron, P., et al., (2017). Nature Methods, 14 (6), 600-606) without off-target motifs located within 1 nucleotide of a cut-site were considered false-positives and discarded. The number of recovered targeted sites from the SITE-Seq® off-target assay experiment is shown in Table 11.

TABLE 11
Number of SITE-Seq ® Assay Recovered Sites

| SEQ ID | Nucleoprotein complex concentration | | | | | | |
|---|---|---|---|---|---|---|---|
| NO: | 8 nM | 16 nM | 32 nM | 48 nM | 64 nM | 96 nM | 128 nM |
| SEQ ID | 0 | 6 | 51 | 158 | 221 | 262 | 280 |
| NO: 370 | | | | | | | |
| SEQ ID | 2 | 4 | 10 | 22 | 57 | 88 | 109 |
| NO: 404 | | | | | | | |

The data presented in Table 11 demonstrate that the SITE-Seq® assay (Cameron, P., et al., (2017). Mapping the genomic landscape of CRISPR-Cas9 cleavage. Nature Methods, 14 (6), 600-606) recovers biochemical off-targets of Cas12 guide for further in-cell evaluation.

### B. In-Cell Validation of the SITE-Seq® Off-Target Assay Recovered Sites

To measure indel frequencies at SITE-Seq® off-target sites shown in Table 11, targeted deep sequencing analyses were performed on a subset of the sites recovered in the RPL32 and DNMT1 samples for the lowest (e.g., 8 nM and 16 nM) Cas12a nucleoprotein complex concentration. Two off-target sites from the RPL32 sample (SEQ ID NO: 371 and SEQ ID NO: 372) and a single off-target in DNMT1 (SEQ ID NO: 374) were selected for evaluation of in-cell off-target editing rates with crRNA. Forward and reverse amplicon primers were designed for each off-target site and ordered from a commercial manufacturer.

Human primary T cells were cultured as described in Example 1. RPL32 (SEQ ID NO: 375) and DNMT1 (SEQ ID NO: 404) Cas12a nucleoprotein complexes were prepared essentially as described in Example 2. The nucleoprotein complexes were transfected into primary T cells as described in Example 3, and the resulting genome editing efficiency of the Cas12a guide/nucleoprotein complexes was determined as described in Example 4. An un-transfected pool of cells was used as a wild type reference. Mutant reads (% indels) were defined as any non-reference variant calls within 20 base pairs (bp) of the cut site. Sites were discarded that had low sequencing coverage (<1,000 reads in the combined, Cas12a nucleoprotein complex-treated samples or <200 reads in the reference samples) or >2% variant calls in the reference samples. Sites were tallied as cellular off-targets if they accumulated >0.1% mutant reads in the combined, Cas12a nucleoprotein complex-treated samples. The results of the targeted deep sequencing of recovered SITE-Seq® (Cameron, P., et al., (2017). Nature Methods, 14 (6), 600-606) off-target sites are presented in Table 12, wherein mismatched nucleotides are underlined.

TABLE 12
In cell Validation of SITE-Seq ® Assay Off-target Sites

| SEQ ID | | hg38 | | | |
|---|---|---|---|---|---|
| NO. | Name | coordinates | Sequence | Average | StDev |
| SEQ ID | RPL32_on | chr3:129389653- | TTTGGGGTGATC | 47.1% | 1.7% |
| NO: 370 | | 129389676 | AGACCCAACAGC | | |
| SEQ ID | RPL_32-off.1 | chr2:88944276- | TTTGGGGTGATC | 26.4% | 0.3% |
| NO: 371 | | 88944299 | AGACCCAACACC | | |
| SEQ ID | RPL_32-off.2 | chr6:33355564- | TTTGGGGTGATC | 23.6% | 0.6% |
| NO: 372 | | 33355587 | AGACCCAACACC | | |
| SEQ ID | DNMT1_on | chr19:10133770- | TTTCCTGATGGTC | 41.1% | 0.6% |
| NO: 373 | | 10133793 | CATGTCTGTTA | | |
| SEQ ID | DNMT1_off-1 | chr1:213204014- | TTTCCTGATGGTC | 0.2% | 0.0% |
| NO: 374 | | 213204037 | CATGTCTGAAT | | |

StDev = standard deviation; n = 3

The data presented in Table 12 demonstrate that the SITE-Seq® assay recovers off-target that are edited in T-cells by Cas12a crRNAs guide.

C. In Silico Design of chRDNA Guides

A 20-nucleotide target in the gene encoding human RPL32 (RPL32-tgt1) and in the gene encoding DNMT1 (DNMT1-tgt1) were selected for editing. For each target, between 1 and 4 nucleotides of DNA were designed into the target binding sequence of each AsCas12a chRDNA guide. Design criteria for the position of DNA bases included, but were not limited to, previously single position screen data (see Example 5), prior consensus of positions tolerant to DNA (see FIG. 13), distance between individual DNA bases in target binding sequence, and known location of mismatches in an off-target sequence. Cas12a chRDNA guides, as well as Cas12a crRNA control sequences, were provided to a commercial manufacturer for synthesis.

### D. Cell Transfection and Analysis

Individual Cas12a guide/nucleoprotein complexes were prepared essentially as described in Example 2. The nucleoprotein complexes were transfected into primary T cells as described in Example 3, and the resulting genome editing efficiency of the Cas12a guide/nucleoprotein complexes was determined as described in Example 4. The results of the on- and off-target (see Table 12) cellular editing experiment and location of DNA bases in the target binding sequence of each Cas12a chRDNA guide are shown for the RPL32 (Table 13) and DNMT1 (Table 14) targets. Additionally, Cas12a chRDNA guide molecules with an abasic deoxyribose site were also tested, and the location of the abasic site is indicated by dN.

TABLE 13
RPL32 crRNA and chRDNA Guide On- and Off-target Editing

| SEQ ID | DNA | On | | Off-target 1 | | Off-target 2 | |
|---|---|---|---|---|---|---|---|
| NO. | Position | Average | StDev | Average | StDev | Average | StDev |
| SEQ ID | — | 81.0% | 0.8% | 35.8% | 0.8% | 41.1% | 0.1% |
| NO: 375 | | | | | | | |
| SEQ ID | 1 | 58.8% | 2.4% | 23.9% | 1.7% | 21.4% | 1.5% |
| NO: 376 | | | | | | | |
| SEQ ID | 8 | 37.5% | 3.1% | 10.5% | 1.3% | 8.8% | 1.5% |
| NO: 377 | | | | | | | |
| SEQ ID | 9 | 52.7% | 1.7% | 13.0% | 0.7% | 11.9% | 0.6% |
| NO: 378 | | | | | | | |
| SEQ ID | 10 | 55.1% | 1.9% | 19.9% | 1.8% | 17.9% | 1.8% |
| NO: 379 | | | | | | | |
| SEQ ID | 11 | 66.6% | 3.8% | 23.7% | 3.1% | 24.4% | 3.4% |
| NO: 380 | | | | | | | |
| SEQ ID | 12 | 64.8% | 3.3% | 13.5% | 1.6% | 14.9% | 1.9% |
| NO: 381 | | | | | | | |
| SEQ ID | 14 | 67.2% | 2.6% | 17.0% | 1.7% | 16.7% | 1.0% |
| NO: 382 | | | | | | | |
| SEQ ID | 15 | 70.0% | 1.6% | 16.0% | 1.6% | 18.3% | 1.5% |
| NO: 383 | | | | | | | |
| SEQ ID | 16 | 45.1% | 1.9% | 0.8% | 0.2% | 1.1% | 0.2% |
| NO: 384 | | | | | | | |
| SEQ ID | 17 | 56.1% | 2.0% | 14.2% | 0.5% | 14.3% | 1.2% |
| NO: 385 | | | | | | | |
| SEQ ID | 18 | 38.8% | 3.2% | 5.8% | 0.6% | 6.6% | 0.7% |
| NO: 386 | | | | | | | |
| SEQ ID | 19 | 54.7% | 1.7% | 18.0% | 0.7% | 17.7% | 1.4% |
| NO: 387 | | | | | | | |
| SEQ ID | 20 | 56.1% | 2.0% | 22.3% | 1.9% | 20.1% | 0.4% |
| NO: 388 | | | | | | | |
| SEQ ID | 13 | 64.5% | 2.4% | 0.3% | 0.0% | 0.4% | 0.0% |
| NO: 389 | | | | | | | |
| SEQ ID | 11, 12 | 80.3% | 1.0% | 5.4% | 0.2% | 6.0% | 0.4% |
| NO: 390 | | | | | | | |
| SEQ ID | 12, 14 | 79.1% | 0.8% | 1.6% | 0.1% | 1.8% | 0.3% |
| NO: 391 | | | | | | | |
| SEQ ID | 14, 15 | 72.8% | 2.6% | 1.4% | 0.2% | 1.6% | 0.3% |
| NO: 392 | | | | | | | |
| SEQ ID | 11, 15 | 81.8% | 0.9% | 6.0% | 0.1% | 6.8% | 0.3% |
| NO: 393 | | | | | | | |
| SEQ ID | 11, 12, 14 | 77.7% | 0.3% | 0.9% | 0.2% | 1.1% | 0.2% |
| NO: 394 | | | | | | | |
| SEQ ID | 12, 14, 15 | 64.5% | 2.5% | 0.2% | 0.1% | 0.2% | 0.1% |
| NO: 395 | | | | | | | |
| SEQ ID | 11, 14, 15 | 74.4% | 2.0% | 0.9% | 0.1% | 1.0% | 0.1% |
| NO: 396 | | | | | | | |
| SEQ ID | 11, 12, 14, 15 | 8.3% | 0.5% | 0.0% | 0.0% | 0.0% | 0.0% |
| NO: 397 | | | | | | | |
| SEQ ID | dN(20) | 59.2% | 2.9% | 4.5% | 0.9% | 4.9% | 0.6% |
| NO: 398 | | | | | | | |
| SEQ ID | 14, dN(20) | 40.3% | 1.1% | 0.6% | 0.1% | 0.5% | 0.1% |
| NO: 399 | | | | | | | |
| SEQ ID | 15, dN(20) | 20.2% | 2.2% | 0.1% | 0.0% | 0.2% | 0.1% |
| NO: 400 | | | | | | | |
| SEQ ID | 12, dN(20) | 50.2% | 3.3% | 0.5% | 0.1% | 0.6% | 0.1% |
| NO: 401 | | | | | | | |
| SEQ ID | 11, dN(20) | 25.6% | 1.0% | 0.7% | 0.2% | 0.7% | 0.1% |
| NO: 402 | | | | | | | |
| SEQ ID | 14, dN(20) | 19.5% | 1.6% | 0.1% | 0.0% | 0.1% | 0.0% |
| NO: 403 | | | | | | | |

StDev = standard deviation; n = 3

TABLE 14
DNMT1 crRNA and chRDNA Guide On- and Off-target Editing

| SEQ ID | DNA | On | | Off-target 1 | |
|---|---|---|---|---|---|
| NO. | Position | Average | StDev | Average | StDev |
| SEQ ID | — | 68.4% | 1.3% | 0.9% | 0.2% |
| NO: 404 | | | | | |
| SEQ ID | 1, 20 | 68.2% | 0.4% | 0.6% | 0.0% |
| NO: 405 | | | | | |
| SEQ ID | 8, 19 | 63.6% | 4.6% | 0.5% | 0.1% |
| NO: 406 | | | | | |
| SEQ ID | 9 | 63.8% | 1.9% | 0.3% | 0.0% |
| NO: 407 | | | | | |
| SEQ ID | 8, 14 | 61.1% | 1.5% | 0.2% | 0.1% |
| NO: 408 | | | | | |
| SEQ ID | 10, 17 | 56.4% | 1.8% | 0.1% | 0.0% |
| NO: 409 | | | | | |
| SEQ ID | 1, 8, 20 | 57.5% | 4.4% | 0.1% | 0.1% |
| NO: 410 | | | | | |
| SEQ ID | 1, 8, 19 | 52.3% | 1.1% | <0.1% | 0.1% |
| NO: 411 | | | | | |
| SEQ ID | 8, 9, 15 | 52.5% | 0.6% | <0.1% | 0.0% |
| NO: 412 | | | | | |

StDev = standard deviation;
n = 3

The editing results in Table 13 and Table 14 demonstrate that Cas12a chRDNA guide molecules are capable of reduced editing at off-target sites (compare results in Table 13 of off-target editing of SEQ ID NO: 375 to SEQ ID NO: 391; SEQ ID NO: 375 to SEQ ID NO: 394; compare results in Table 14 of off-target editing of SEQ ID NO: 404 to SEQ ID NO: 408; or SEQ ID NO: 404 to SEQ ID NO: 412), even at sites with a single nucleotide mismatch (see, e.g., Table 12, SEQ ID NO: 371 and SEQ ID NO: 372).

### Example 8

Cas12a chRDNA Guide Molecules with DNA Bases in the Activating Region

This Example describes the designing and testing of AsCas12a chRDNA guide molecules with DNA bases in the activating region.

A. In Silico Design of chRDNA Guides

The 20-nucleotide activating region of the AsCas12a guide was selected for engineering, where an individual DNA base was utilized at each position in the activating region. The location of DNA bases in the activating region of the AsCas12a guide are shown in Table 15.

TABLE 15
AsCas12a chRDNA Guide Molecules with DNA in the Activating Region

| | DNA | |
|---|---|---|
| SEQ ID NO: | Position | Sequence |
| SEQ ID NO: 417 | crRNA | rUrArArUrUrUrCrUrArCrUrCrUrUrGrUrArGrArU |
| SEQ ID NO: 418 | 1 | TrArArUrUrUrCrUrArCrUrCrUrUrGrUrArGrArU |
| SEQ ID NO: 419 | 2 | rUArArUrUrUrCrUrArCrUrCrUrUrGrUrArGrArU |
| SEQ ID NO: 420 | 3 | rUrAArUrUrUrCrUrArCrUrCrUrUrGrUrArGrArU |
| SEQ ID NO: 421 | 4 | rUrArATrUrUrCrUrArCrUrCrUrUrGrUrArGrArU |
| SEQ ID NO: 422 | 5 | rUrArArUTrUrCrUrArCrUrCrUrUrGrUrArGrArU |
| SEQ ID NO: 423 | 6 | rUrArArUrUTrCrUrArCrUrCrUrUrGrUrArGrArU |
| SEQ ID NO: 424 | 7 | rUrArArUrUrUCrUrArCrUrCrUrUrGrUrArGrArU |
| SEQ ID NO: 425 | 8 | rUrArArUrUrUrCTrArCrUrCrUrUrGrUrArGrArU |
| SEQ ID NO: 426 | 9 | rUrArArUrUrUrCrUArCrUrCrUrUrGrUrArGrArU |
| SEQ ID NO: 427 | 10 | rUrArArUrUrUrCrUrACrUrCrUrUrGrUrArGrArU |
| SEQ ID NO: 428 | 11 | rUrArArUrUrUrCrUrArCTrCrUrUrGrUrArGrArU |
| SEQ ID NO: 429 | 12 | rUrArArUrUrUrCrUrArCrUCrUrUrGrUrArGrArU |
| SEQ ID NO: 430 | 13 | rUrArArUrUrUrCrUrArCrUrCTrUrGrUrArGrArU |
| SEQ ID NO: 431 | 14 | rUrArArUrUrUrCrUrArCrUrCrUTrGrUrArGrArU |
| SEQ ID NO: 432 | 15 | rUrArArUrUrUrCrUrArCrUrCrUrUGrUrArGrArU |
| SEQ ID NO: 433 | 16 | rUrArArUrUrUrCrUrArCrUrCrUrUrGTrArGrArU |
| SEQ ID NO: 434 | 17 | rUrArArUrUrUrCrUrArCrUrCrUrUrGrUArGrArU |
| SEQ ID NO: 435 | 18 | rUrArArUrUrUrCrUrArCrUrCrUrUrGrUrAGrArU |
| SEQ ID NO: 436 | 19 | rUrArArUrUrUrCrUrArCrUrCrUrUrGrUrArGArU |
| SEQ ID NO: 437 | 20 | rUrArArUrUrUrCrUrArCrUrCrUrUrGrUrArGrAT |

A target in the gene encoding human DNMT1 (SEQ ID NO: 361) was selected for editing. The DNMT target binding sequence was appended downstream (i.e., in a 3′ direction) to the activating region sequences containing a single DNA base at each position (see Table 15). Sequences were provided to a commercial manufacturer for synthesis.

### B. Cell Transfection and Analysis

Individual Cas12a guide/nucleoprotein complexes for screening were prepared essentially as described in Example 2. The nucleoprotein complexes were transfected into primary T cells as described in Example 3, and the resulting genome editing efficiency of the Cas12a guide/nucleoprotein complexes was determined as described in Example 4. The results of the in-cell editing experiment are shown in Table 16.

TABLE 16
Editing of DNMT1 Using Cas12 chRDNA Guides with
DNA in the Activating Region

| | DNA | | |
|---|---|---|---|
| SEQ ID NO: | Position | Average | StDev |
| SEQ ID NO: 438 | crRNA | 60.8% | 1.0% |
| SEQ ID NO: 439 | 1 | 53.1% | 0.5% |
| SEQ ID NO: 440 | 2 | 29.6% | 0.3% |
| SEQ ID NO: 441 | 3 | 58.5% | 0.1% |
| SEQ ID NO: 442 | 4 | 1.4% | 0.1% |
| SEQ ID NO: 443 | 5 | 44.8% | 0.6% |
| SEQ ID NO: 444 | 6 | 2.2% | 0.1% |
| SEQ ID NO: 445 | 7 | 60.8% | 1.0% |
| SEQ ID NO: 446 | 8 | 25.4% | 4.9% |
| SEQ ID NO: 447 | 9 | 43.4% | 2.2% |
| SEQ ID NO: 448 | 10 | 64.8% | 3.3% |
| SEQ ID NO: 449 | 11 | 19.5% | 0.6% |
| SEQ ID NO: 450 | 12 | 61.6% | 1.4% |
| SEQ ID NO: 451 | 13 | 46.4% | 1.6% |
| SEQ ID NO: 452 | 14 | 65.0% | 1.4% |
| SEQ ID NO: 453 | 15 | 61.4% | 1.1% |
| SEQ ID NO: 454 | 16 | 56.8% | 0.6% |
| SEQ ID NO: 455 | 17 | 58.5% | 2.0% |
| SEQ ID NO: 456 | 18 | 58.1% | 1.0% |
| SEQ ID NO: 457 | 19 | 63.0% | 0.6% |
| SEQ ID NO: 458 | 20 | 28.4% | 0.4% |

StDev = standard deviation;
n = 3

The editing results in Table 16 demonstrate that Cas12a chRDNAs with DNA in the activating region are capable of editing at a rate comparable to the crRNA across multiple targets (compare SEQ ID NO: 438 to SEQ ID NO: 445; SEQ ID NO: 438 to SEQ ID NO: 450, or SEQ ID NO: 438 to SEQ ID NO: 457). The editing rates of the chRDNA designs in Table 16 were normalized to the editing rates of the crRNA. Average normalized editing rates are presented in FIG. 14, where position within the activating region are plotted as a function of average normalized editing. The preferred positions of DNA base utilization (i.e., greater than 70% average normalized editing) are indicated with grey fill, and include positions 1, 3, 5, 7, 9, 10, 12, 13, 14, 15, 16, 17, 18, and 19.

D. chRDNAs with Multiple DNA Bases in the Activating Region

Based on the results presented in Table 10, individual DNA positions were combined into activating region designs containing multiple DNA bases. The design and location of DNA bases in the activating region of the AsCas12a guide are shown in Table 17.

TABLE 17
Cas12a chRDNA Guide Molecules with Multiple DNA Bases in the Activating Region

| SEQ ID NO: | DNA Positions | Sequence |
|---|---|---|
| SEQ ID NO: 459 | 1, 3, 7, 10, 12, 14, 15, 19 | TrAArUrUrUCrUrACrUCrUTGrUrArGArU |
| SEQ ID NO: 460 | 1, 15-19 | TrArArUrUrUrCrUrArCrUrCrUrUGTAGArU |
| SEQ ID NO: 461 | 1, 13, 16, 17 | TrArArUrUrUrCrUrArCrUrCTrUrGTArGrArU |
| SEQ ID NO: 462 | 7, 10, 12, 14, 15, 17, 19 | rUrArArUrUrUCrUrACrUCrUTGrUArGArU |
| SEQ ID NO: 463 | 1, 3, 12, 14, 16, 17, 19 | TrAArUrUrUrCrUrArCrUCrUTrGTArGArU |
| SEQ ID NO: 464 | 7, 10, 12, 14, 17, 19 | rUrArArUrUrUCrUrACrUCrUTrGrUArGArU |
| SEQ ID NO: 465 | 1, 3, 7, 10, 12, 14, 15, 19 | TrAArUrUrUCrUrACrUCrUTrGrUArGArU |

Activating region designs presented in Table 17 were combined with a 20-nucleotide target sequence in the gene encoding human TRAC designed with DNA nucleotides in the target binding sequence (SEQ ID NO: 321). The TRAC target binding sequence was appended downstream (i.e., in a 3′ direction) to the activating region sequences presented in Table 17, and the chRDNA designs, as well as a crRNA control sequence, were provided to a commercial manufacturer for synthesis.

### E. Cell Transfection and Analysis

Individual Cas12a guide/nucleoprotein complexes for screening were prepared essentially as described in Example 2. The Cas12a guide/nucleoprotein complexes were transfected into primary T cells as described in Example 3, and the resulting genome editing efficiency of the Cas12a guide/nucleoprotein complexes was determined as described in Example 4. The results of the in-cell editing experiment and the location of DNA bases in the activating region and target binding sequence of each chRDNA are shown in Table 18.

TABLE 18
Editing of TRAC Using Cas12a chRDNA Guide Molecules with
DNA in the Activating Region and in the Target Binding Sequence

| | | DNA | | |
|---|---|---|---|---|
| | | positions | | |
| | | in target | | |
| | DNA positions in | binding | | |
| SEQ ID NO: | activating region | sequence | Average | StDev |
| SEQ ID NO: 466 | 0 | 1, 10, 20 | 78.7% | 1.2% |
| SEQ ID NO: 467 | 1, 3, 7, 10, 12, 14, 15, 19 | 1, 10, 20 | 82.3% | 1.1% |
| SEQ ID NO: 468 | 1, 15-19 | 1, 10, 20 | 75.9% | 1.3% |
| SEQ ID NO: 469 | 1, 13, 16, 17 | 1, 10, 20 | 76.9% | 2.4% |
| SEQ ID NO: 470 | 7, 10, 12, 14, 15, 17, 19 | 1, 10, 20 | 71.3% | 1.0% |
| SEQ ID NO: 471 | 1, 3, 12, 14, 16, 17, 19 | 1, 10, 20 | 73.3% | 1.9% |
| SEQ ID NO: 472 | 7, 10, 12, 14, 17, 19 | 1, 10, 20 | 67.0% | 2.2% |
| SEQ ID NO: 473 | 1, 3, 7, 10, 12, 14, 15, 19 | 1, 10, 20 | 59.3% | 4.1% |

StDev = standard deviation;
n = 3

The editing results in Table 18 demonstrate that Cas12a chRDNA guide molecules with DNA in both the activating region and the target binding sequence are capable of editing at a rate comparable to the crRNA (compare SEQ ID NO: 466 to SEQ ID NO: 467; SEQ ID NO: 466 to SEQ ID NO: 468, or SEQ ID NO: 466 to SEQ ID NO: 469).

### Example 9

### Cloning of AAV Donor Cassette, AAV Production, and AAV Transduction of Primary Cells

This Example describes the design and cloning of a DNA donor cassette into an AAV vector, production of AAV, delivery of Cas12a chRDNA guide/nucleoprotein complexes into primary cells, and transduction of primary cells with AAV for site-specific integration of a CAR expression cassette into primary cells.

AAV can be engineered to deliver DNA donor polynucleotides to mammalian cells. If AAV delivery is combined with a genomic cleavage event, and the DNA donor polynucleotide in the AAV is flanked by homology arms, the DNA donor polynucleotide can be seamlessly inserted into the genomic cut site by HDR, as described in, for example, Eyquem et al. (Nature, 2017, 543:113-117).

A. In Silico Design of AAV Donor Cassettes and rAAV Production

The design of CAR receptors has been described. See, e.g., Kochenderfer et al. (J. Immunotherapy, 2009, 32:689-702). The CAR construct was designed to contain an N-terminal secretion signal (CD8a signal peptide), an scFv portion specific for BCMA, followed by a CD8 hinge region and transmembrane, a 4-1BB effector region, a CD35 effector region, and a C-terminal BGH polyadenylation signal sequence. A mammalian promoter sequence was inserted upstream of the CAR polynucleotide. In order to site-specifically insert DNA donor polynucleotides into the host cell genome after site-specific cleavage, a target site was chosen in the endogenous TRAC locus (SEQ ID NO: 23). Then, 500 bp long homology arms 5′ and 3′ of the cut site were identified. The 5′ and 3′ homology arms were appended to the end of the DNA donor polynucleotides, wherein the DNA donor polynucleotides were orientated in a reverse orientation (i.e., 3′ to 5′) relative to the homology arms. The resulting DNA donor polynucleotide is presented in SEQ ID NO: 413.

The design for B2M and HLA class I histocompatibility antigen, alpha chain E (HLA-E), has been described. See, e.g., Gornalusse et al. (Nature Biotechnology, 2017, 35 (8): 765-772). The fusion construct was designed with an N-terminal B2M secretion signal, followed by an HLA-G derived peptide sequence, a first linker sequence, the B2M sequence, a second linker sequence, an HLA-E sequence, and a C-terminal BGH polyadenylation signal sequence. An EF1α mammalian promoter sequence was inserted upstream of the B2M-HLA-E polynucleotide. In order to site-specifically insert DNA donor polynucleotide into the host cell genome after site specific cleavage, a target site in the endogenous B2M locus was chosen (SEQ ID NO: 62). Then, 500 bp long homology arms 5′ and 3′ of the cut site were identified. The 5′ and 3′ homology arms were appended to the end of the DNA donor polynucleotides, wherein the DNA donor polynucleotides were orientated in a reverse orientation (i.e., 3′ to 5′) relative to the homology arms. The resulting DNA donor polynucleotide is presented in SEQ ID NO: 414.

Oligonucleotide sequences coding for DNA donor polynucleotides were provided to a commercial manufacturer for synthesis into a suitable recombinant AAV (rAAV) plasmid. A rAAV plasmid containing SEQ ID NO: 413 and a separate rAAV plasmid containing SEQ ID NO: 414 were provided to a commercial manufacturer for packaging into two separate AAV6 viruses.

B. Primary T Cell Transduction with rAAV

Primary activated T cells were obtained from PBMCs as described in Example 1. Cas12a chRDNA guide/nucleoprotein complexes targeting the genes encoding TRAC (SEQ ID NO: 415) and B2M (SEQ ID NO: 416) were prepared as described in Example 2. T cells were transfected with TRAC (SEQ ID NO: 415)-targeting Cas12a chRDNA guide/nucleoprotein complexes, and between 1 minute and 4 hours after nucleofection, cells were infected with the AAV6 virus packaged with CAR donor sequence (SEQ ID NO: 413) at an MOI of 1×10⁶. Additionally, T cells were transfected with B2M (SEQ ID NO: 416)-targeting Cas12a chRDNA guide/nucleoprotein complexes, and between 1 minute and 4 hours after nucleofection, cells were infected with the AAV6 virus packaged with B2M-HLA-E donor sequence (SEQ ID NO: 414) at an MOI of 1×10⁶. T cells were cultured in ImmunoCult-XF complete medium (STEMCELL Technologies, Cambridge, MA) supplemented with IL-2 (100 units/mL) for 24 hours after the transductions. The next day, the transduced T cells were transferred to 50 mL conical tubes and centrifuged at 300×g for approximately 7-10 minutes to pellet cells. The supernatant was discarded, and the pellet was gently resuspended, and the T cells pooled in an appropriate volume of ImmunoCult-XF complete medium (STEMCELL Technologies, Cambridge, MA) supplemented with IL-2 (100 units/mL).

The enumerated T cells were resuspended at 1×10⁶ cells/mL in ImmunoCult-XF complete medium STEMCELL Technologies, Cambridge, MA) supplemented with IL-2 (100 units/mL), and plated into as many T-175 suspension flasks as required (max volume per flask is 250 mL).

### C. Expression of Anti-BCMA, B2M-HLA-E CAR-T Cells

In vitro characterization of anti-BCMA, B2M-HLA-E CAR-T cells, as well as control (TRAC knock out (KO) and B2M KO) and wild type T cells, was performed 7 days after transduction.

CAR-T cells were evaluated for expression via flow cytometry for either anti-BCMA CAR expression using recombinant BCMA protein conjugated to phycoerythrin (PE); expression of TRAC using an anti-TCR a/b specific antibody conjugate to Alexa Fluor® 647 (ThermoFisher Scientific, Waltham, MA), or B2M expression using anti-B2M specific antibody conjugated to PE. The results from the flow cytometry analysis is presented in FIG. 15A, where CAR positive (FIG. 15A, 1501), TRAC positive (FIG. 15A, 1502), and B2M positive (FIG. 15A, 1503) rates are shown for cells that were untreated (wild type T cells; FIG. 15A, 1504), cells that were transfected with both Cas12a chRDNA guide/nucleoprotein complexes only (TRAC KO/B2M KO; FIG. 15A, 1505), and cells that were transfected with both Cas12a chRDNA guide/nucleoprotein complexes and transduced with both viruses (anti-BCMA, B2M-HLA-E CAR-T; FIG. 15A, 1506). The y-axis represents the percent-positive cells, as measured via FACS, for the various cell surface markers. Results are also provided in Table 19.

TABLE 19
Flow Cytometry Analysis of Cas12a/AAV Treated Cells

| | wild type | TRAC KO/B2M | anti-BCMA, B2M- |
|---|---|---|---|
| | T cells | KO | HLA-E CAR-T cells |
| CAR positive | 2.6% | 5.2% | 87.9% |
| TRAC positive | 96.3% | 7.8% | 3.9% |
| B2M positive | 100.0% | 8.7% | 50.5% |

The results presented in FIG. 15A and Table 19 demonstrate the Cas12a chRDNA guide-mediated KO of endogenous TRAC and B2M expression, and AAV6 mediated introduction and expression of anti-BCMA CAR and exogenous B2M-HLA-E proteins.

### D. In Vitro Cytotoxicity of Anti-BCMA, B2M-HLA-E CAR-T Cells

The cytotoxicity of anti-BCMA, B2M-HLA-E CAR-T cells was evaluated in vitro against a multiple myeloma NCI-H929 cells line, which present the BCMA antigen. TRAC KO T cells were used as a control for CAR-mediated killing. Briefly, target cells (NCI-H929 (T)) were labeled with CellTrace™ Violet (CTV; Thermo Fisher C34557) to distinguish them from effector anti-BCMA, B2M-HLA-E CAR-T (E), and cells were co-cultured at E:T ratios of 0:1, 1:20, 1:10, 1:5, 1:3, 1:1, 3:1, and 10:1 (3 co-culture wells/E:T ratio). Cytotoxicity was measured by gating on CTV cell population (target cells) and live cells as measured by propidium iodide (PI) after 48 hours in co-culture. Data was analyzed by flow cytometry (Intellicyt iQue Screener Plus). Specific lysis was calculated using the following equation for each well: Specific lysis=1−(number of live target cells in test sample/number of live target cells in control sample).

The results from the in vitro cytotoxicity assay are presented in FIG. 15B for the anti-BCMA, B2M-HLA-E CAR-T cells (grey circles) and control TRAC KO T cells (black circles). The y-axis represents the percent of target cell killing, and the x-axis indicates the ET ratio used. The data presented in FIG. 15B is also presented in Table 20.

TABLE 20
Anti-BCMA CAR-Mediated Cytotoxicity

| | | | anti-BCMA, B2M- | |
|---|---|---|---|---|
| | TRAC KO T cells | | HLA-E CAR-T cells | |
| E:T | Average | StDev | Average | StDev |
| 0:1 | 12% | 1% | 14% | 1% |
| 1:20 | 12% | 1% | 76% | 4% |
| 1:10 | 14% | 1% | 85% | 1% |
| 1:5 | 15% | 1% | 91% | 1% |
| 1:3 | 19% | 1% | 93% | 1% |
| 1:1 | 30% | 7% | 95% | 1% |
| 3:1 | 53% | 7% | 92% | 0% |
| 10:1 | 71% | 8% | 82% | 3% |

StDev = standard deviation;
n = 3

The results presented in FIG. 15B and Table 20 demonstrate the CAR-T cells manufactured using Cas12a chRDNA guide molecules are capable of antigen-specific killing of target cells.

The methods presented herein can be used to manufacture other cells using Cas12a chRDNA guide molecules for the site-specific introduction of donor polynucleotides comprising a chimeric antigen receptor (CAR). Additional donor polynucleotides expressing non-CAR polypeptides (i.e., B2M-HLA-E fusions construct) can be similarly introduced using the guidance herein.

### Example 10

Generation of an Anti-BCMA CAR-T with Endogenous B2M Promoter Driven Expression of a B2M-HLA-E Fusion

This Example describes the design for transduction of primary cells with AAV for site-specific integration of a CAR polynucleotide and a B2M-HLA-E polynucleotide expression cassette at Cas12a chRDNA mediated break sites into the genome of primary cells.

A. In Silico Design of AAV Donor Cassettes and rAAV Production

The anti-BCMA CAR was designed as described in Example 9.

The donor cassette polynucleotide for the P2A-B2M-HLA-E fusion construct was designed with polynucleotide encoding an N-terminal B2M secretion signal, followed by a polynucleotide encoding an HLA-G derived peptide sequence, a polynucleotide encoding a first linker sequence, a polynucleotide encoding the B2M sequence, a polynucleotide encoding a second linker sequence, a polynucleotide encoding an HLA-E sequence, and a polynucleotide encoding a C-terminal BGH polyadenylation signal sequence. A polynucleotide encoding a P2A ribosomal skipping sequence was inserted upstream of the B2M-HLA-E, such that expression of the fusion construct was under the control of the endogenous B2M promoter. In order to site-specifically insert DNA donor polynucleotides into the host cell genome after site-specific cleavage, a target site in the endogenous B2M locus was chosen (SEQ ID NO: 62). Then, 500 base pair long homology arms 5′ and 3′ of the cut site were identified. The 5′ and 3′ homology arms were appended to the 5′ and 3′ ends of the DNA donor polynucleotides, wherein the DNA donor polynucleotides were orientated in a forward orientation (i.e., 5′ to 3′) relative to the homology arms. The resulting DNA donor polynucleotide is presented in SEQ ID NO: 479.

Oligonucleotide sequences coding for DNA donor polynucleotides were provided to a commercial manufacturer for synthesis into a suitable recombinant AAV (rAAV) plasmid. A rAAV plasmid containing SEQ ID NO: 413 and a separate rAAV plasmid containing SEQ ID NO: 479 were provided to a commercial manufacturer for packaging into two separate AAV6 viruses.

B. Primary T Cell Transduction with rAAV

Primary activated T cells were obtained from PBMCs as described in Example 1. Cas12a chRDNA guide/nucleoprotein complexes targeting the genes encoding TRAC (SEQ ID NO: 415) and B2M (SEQ ID NO: 416) were prepared as described in Example 2. T cells were transfected with TRAC (SEQ ID NO: 415)-targeting Cas12a chRDNA guide/nucleoprotein complexes, and, between 1 minute and 4 hours after nucleofection, cells were infected with the AAV6 virus packaged with CAR donor sequence (SEQ ID NO: 413) at an MOI of 1×10⁶. Additionally, T cells were transfected with B2M (SEQ ID NO: 416)-targeting Cas12a chRDNA guide/nucleoprotein complexes, and, between 1 minute and 4 hours after nucleofection, cells were infected with the AAV6 virus packaged with P2A-B2M-HLA-E donor sequence (SEQ ID NO: 479) at an MOI of 1×10⁶. T cells were cultured in ImmunoCult-XF complete medium (STEMCELL Technologies, Cambridge, MA) supplemented with IL-2 (100 units/mL) for 24 hours after the transductions. The next day, the transduced T cells were transferred to 50 mL conical tubes and centrifuged at 300×g for approximately 7-10 minutes to pellet cells. The supernatant was discarded, and the pellet was gently resuspended, and the T cells pooled in an appropriate volume of ImmunoCult-XF complete medium (STEMCELL Technologies, Cambridge, MA) supplemented with IL-2 (100 units/mL).

The enumerated T cells were resuspended at 1×10⁶ cells/mL in ImmunoCult-XF complete medium (STEMCELL Technologies, Cambridge, MA) supplemented with IL-2 (100 units/mL), and plated into as many T-175 suspension flasks as required (max volume per flask is 250 mL).

### C. Expression of Anti-BCMA CAR and B2M-HLA-E on CAR-T Cells

In vitro characterization of anti-BCMA, P2A-B2M-HLA-E CAR-T cells, as well as control (TRAC knock out (KO) and B2M KO) and wild type T cells, was performed 7 days after transduction.

CAR-T cells were evaluated via flow cytometry for either expression of anti-BCMA CAR using recombinant BCMA protein conjugated to phycoerythrin (PE); expression of TRAC using an anti-TCR a/b specific antibody conjugate to Alexa Fluor® 647 (ThermoFisher Scientific, Waltham, MA), or expression of B2M using anti-B2M specific antibody conjugated to PE. The results from the flow cytometry analysis are presented in Table 21, where CAR positive, TRAC positive, and B2M positive rates are shown for cells that were untreated (wild type T cells), cells that were transfected with both Cas12a chRDNA guide/nucleoprotein complexes only (TRAC KO/B2M KO), and cells that were transfected with both Cas12a chRDNA guide/nucleoprotein complexes and transduced with both viruses (anti-BCMA, B2M-HLA-E CAR-T).

TABLE 21
Flow Cytometry Analysis of Cas12a/AAV Treated Cells

| | Wild Type | TRAC KO/B2M | anti-BCMA, B2M- |
|---|---|---|---|
| | T cells | KO | HLA-E CAR-T cells |
| CAR positive | 2.5% | 4.5% | 91.6% |
| TRAC positive | 94.4% | 5.1% | 3.5% |
| B2M positive | 99.0% | 4.5% | 21.0% |

The results presented in Table 21 demonstrate the Cas12a chRDNA guide-mediated KO of endogenous TRAC and B2M expression, and AAV6-mediated introduction and exogenous expression of anti-BCMA CAR donor cassette and endogenous B2M promoter driven expression of an exogenous B2M-HLA-E donor cassette.

The methods presented herein can be used to identify Cas12 chRDNA guide designs for other targets. The activating region and target binding sequences of other Cas12 chRDNA guides can be screened in a similar manner to the methods described herein.

### Example 11

Cas12a Guide/Nucleoprotein Complexes with Alternative Linker-NLS Configuration

This Example describes the design and comparison of Cas12a guide/nucleoprotein complexes with different linker and nuclear localization signal (NLS) configurations compared to an ‘unoptimized’ design with a glycine-serine linker and the Simian Vacuolating Virus 40 large T antigen NLS (SV40; SEQ ID NO:04) used in previous examples in this application.

### A. In Silico Design of Cas12a Linker-NLS Sequences

The Acidaminococcus sp. (strain BV3L6) Cas12a protein (SEQ ID NO:01) was selected for engineering, and two NLS sequences, SV40 (SEQ ID NO:04) and nucleoplasmin sequence (NPL; SEQ ID NO:05), were selected for covalent addition to the Cas12a protein using either a glycine-serine (GS) or a pair of glycine-glycine-glycine-glycine-serine (G4S) amino acid linkers. Designs comprising two NLSs with variable linkers were also generated for testing. Linker-NLS sequence designs are presented in Table 22:

TABLE 22
Linker-NLS Sequences Designs

| SEQ ID | | |
|---|---|---|
| NO: | Name | Amino Acid Sequence |
| SEQ ID | GS-SV40* | GSPKKKRKV |
| NO: 479 | | |
| SEQ ID | GS-SV40-GS-SV40 | GSPKKKRKVGSPKKKRKV |
| NO: 480 | | |
| SEQ ID | GS-NPL | GSKRPAATKKAGQAKKKK |
| NO: 481 | | |
| SEQ ID | GS-NPL-GS-NPL | GSKRPAATKKAGQAKKKKGSKRP |
| NO: 482 | | AATKKAGQAKKKK |
| SEQ ID | GS-SV40-GS-NPL | GSPKKKRKVGSKRPAATKKAGQA |
| NO: 483 | | KKKK |
| SEQ ID | GS-NPL-GS-SV40 | GSKRPAATKKAGQAKKKKGSPKK |
| NO: 484 | | KRKV |
| SEQ ID | (G4S)2-SV40-GS- | GGGGSGGGGSPKKKRKVGSKRPA |
| NO: 485 | NPL | ATKKAGQAKKKK |
| SEQ ID | (G4S)2-NPL-GS- | GGGGSGGGGSKRPAATKKAGQAK |
| NO: 486 | SV40 | KKKGSPKKKRKV |
| SEQ ID | (G4S)2-SV40 | GGGGSGGGGSPKKKRKV |
| NO: 487 | | |
| SEQ ID | (G4S)2-SV40-GS- | GGGGSGGGGSPKKKRKVGSPKKK |
| NO: 488 | SV40 | RKV |
| SEQ ID | (G4S)2-NPL | GGGGSGGGGSKRPAATKKAGQAK |
| NO: 489 | | KKK |
| SEQ ID | (G4S)2-NPL-GS- | GGGGSGGGGSKRPAATKKAGQAK |
| NO: 490 | NPL | KKKGSKRPAATKKAGQAKKKK |

* = NLS design used in previous examples

NLS sequences presented in Table 22 were cloned in at the C-terminal end of the Acidaminococcus sp. (strain BV3L6) Cas12a protein (SEQ ID NO:01), and recombinant protein was expressed as described in Example 2.

### B. Cellular Activity of Cas12a Linker-NLS Designs

The purified recombinant Cas12a protein comprising the linker-NLS sequences presented in Table 22 were complexed with a chRDNA guide targeting the TRAC gene (SEQ ID NO:467) as described in Example 2, and transfected into primary T cells as described in Example 3. 48 hours after transfection, the resulting genome editing efficiency of each Cas12a guide/nucleoprotein complex was determined as described in Example 4. The results of the cellular editing experiment are shown in FIG. 16A and presented in Table 23.

TABLE 23
Cas12a Linker-NLS Cellular Editing Screen

| | | FIG. 16A | | |
|---|---|---|---|---|
| SEQ ID NO: | Name | labels | Average | StDev |
| SEQ ID NO: 489 | (G4S)2-NPL | 1601 | 87.1% | 0.7% |
| SEQ ID NO: 485 | (G4S)2-SV40-GS-NPL | 1602 | 86.1% | 1.4% |
| SEQ ID NO: 487 | (G4S)2-SV40 | 1603 | 85.9% | 1.6% |
| SEQ ID NO: 483 | GS-SV40-GS-NPL | 1604 | 82.9% | 2.7% |
| SEQ ID NO: 490 | (G4S)2-NPL-GS-NPL | 1605 | 83.6% | 1.1% |
| SEQ ID NO: 488 | (G4S)2-SV40-GS-SV40 | 1606 | 80.8% | 0.8% |
| SEQ ID NO: 482 | GS-NPL-GS-NPL | 1607 | 77.2% | 3.6% |
| SEQ ID NO: 486 | (G4S)2-NPL-GS-SV40 | 1608 | 73.9% | 0.7% |
| SEQ ID NO: 480 | GS-SV40-GS-SV40 | 1609 | 71.1% | 2.6% |
| SEQ ID NO: 484 | GS-NPL-GS-SV40 | 1610 | 68.1% | 1.2% |
| SEQ ID NO: 479 | GS-SV40* | 1611 | 65.9% | 1.4% |
| SEQ ID NO: 481 | GS-NPL | 1612 | 65.3% | 3.5 |

StDev = standard deviation;
n = 3
* = NLS design used in previous examples

The data presented in FIG. 16A as well as Table 23 of the present example show that alternative linkers and NLS sequences can result in increased editing compared to a design with a single GS-SV40 NLS configuration.

### C. Cellular Activity of Alternative Cas12a Linker-NLS Sequences Across Targets

The top four Cas12a linker-NLS configurations presented in Table 23 (SEQ ID NO: 489, SEQ ID NO:485, SEQ ID NO:487, and SEQ ID NO:483) and the ‘unoptimized’ design (SEQ ID NO:479) were selected for comparison using a mixed panel of crRNA and chRDNAs. The targeting region, including position of DNA bases in chRDNA designs, are presented in Table 24.

TABLE 24

| | | | chRDNA |
|---|---|---|---|
| SEQ ID NO. | Target | Guide Type | Design |
| SEQ ID NO: 396 | RPL32-tgt1 | chRDNA | 11, 14, 15 |
| SEQ ID NO: 404 | DNMT1-tgt3 | crRNA | — |
| SEQ ID NO: 321 | TRAC-tgt12 | chRDNA | 1, 11, 20 |
| SEQ ID NO: 491 | TRAC-tgt25 | crRNA | — |
| SEQ ID NO: 492 | TRAC-tgt28 | crRNA | — |
| SEQ ID NO: 335 | B2M-tgt12 | chRDNA | 1, 11, 20 |
| SEQ ID NO: 347 | B2M-tgt1 | chRDNA | 8, 14, 19 |
| SEQ ID NO: 355 | B2M-intron-tgt12 | chRDNA | 11, 17, 20 |

Targeting regions presented in Table 24 were appended to the 3′ end of an activating region (SEQ ID NO:459), and provided to a commercial manufacturer for synthesis.

Individual Cas12a linker-NLS configurations complexed to each guide were prepared essentially as described in Example 2, with the deviation that complexes were assembled at two concentrations of 20:60 and 80:240 pmols of Cas12a to guide for each Cas12a linker-NLS and guide combination. The Cas12a guide/nucleoprotein complexes were transfected into primary T cells as described in Example 3, and the resulting genome editing efficiency of the Cas12a guide/nucleoprotein complexes was determined as described in Example 4.

The results of the editing of the Cas12a linker-NLS configuration complexed to the guide shown in Table 24 are presented in FIG. 16B, and Tables 25 and 26.

TABLE 25
Cellular Editing Activity of Cas12a Linker-NLS Constructs Across Targets

| | | | | SEQ ID NO | | | |
|---|---|---|---|---|---|---|---|
| | | | | SEQ ID | SEQ ID | SEQ ID | SEQ ID |
| | | | | NO: 396 | NO: 404 | NO: 321 | NO: 491 |
| | FIG. | Cas12a-: | | Target Name | | | |
| Cas12a- | 16B | guide | | RPL32- | DNMT1- | TRAC- | TRAC- |
| NLS | identifier | [pmol] | | tgt1 | tgt3 | tgt12 | tgt25 |
| GS- | FIG. | 20:60 | Average | 21.8% | 61.3% | 71.3% | 93.4% |
| SV40* | 16B- | | StDev | 0.0% | 3.8% | 5.5% | 2.2% |
| (SEQ ID | 1613 | 80:240 | Average | 69.8% | 68.6% | 77.4% | 96.1% |
| NO: | | | StDev | 0.4% | 1.6% | 3.4% | 0.0% |
| 479) | | | | | | | |
| GS- | FIG. | 20:60 | Average | 85.5% | 80.2% | 90.5% | 96.1% |
| SV40- | 16B- | | StDev | 3.7% | 16.5% | 4.1% | 1.0% |
| GS-NPL | 1614 | 80:240 | Average | 91.2% | 91.4% | 92.9% | 97.6% |
| (SEQ ID | | | StDev | 0.1% | 0.1% | 0.7% | 0.4% |
| NO: | | | | | | | |
| 483) | | | | | | | |
| (G4S)2- | FIG. | 20:60 | Average | 32.0% | 77.1% | 87.5% | 94.4% |
| SV40 | 16B- | | StDev | 45.3% | 11.2% | 2.9% | 1.5% |
| (SEQ ID | 1615 | 80:240 | Average | 89.9% | 87.4% | 91.8% | 95.6% |
| NO: | | | StDev | 0.9% | 0.3% | 0.8% | 0.8% |
| 487) | | | | | | | |
| (G4S)2- | FIG. | 20:60 | Average | 40.1% | 84.6% | 87.0% | 93.6% |
| NPL | 16B- | | StDev | 56.7% | 0.4% | 1.9% | 0.4% |
| (SEQ ID | 1616 | 80:240 | Average | 91.8% | 90.9% | 93.4% | 96.4% |
| NO: | | | StDev | 1.1% | 0.4% | 0.1% | 0.0% |
| 489) | | | | | | | |
| (G4S)2- | FIG. | 20:60 | Average | 88.8% | 80.4% | 83.4% | 95.5% |
| SV40- | 16B- | | StDev | 1.7% | 3.7% | 1.5% | 0.3% |
| GS-NPL | 1617 | 80:240 | Average | 87.9% | 89.0% | 90.4% | 97.0% |
| (SEQ ID | | | StDev | 0.3% | 5.6% | 5.7% | 0.5% |
| NO: | | | | | | | |
| 485) | | | | | | | |

TABLE 26
Cellular Editing Activity of Cas12a Linker-NLS Constructs Across Targets

| | | | | SEQ ID NO: | | | |
|---|---|---|---|---|---|---|---|
| | | | | SEQ ID | SEQ ID | SEQ ID | SEQ ID |
| | | | | NO: 492 | NO: 335 | NO: 347 | NO: 355 |
| | | | | Target Name | | | |
| | FIG. | Cas12a-: | | | | | B2M- |
| Cas12a- | 16B | guide | | TRAC- | B2M- | B2M- | intron- |
| NLS | identifier | [pmol] | | tgt28 | tgt12 | tgt1 | tgt12 |
| GS- | FIG. | 20:60 | Average | 84.4% | 92.9% | 88.7% | 80.3% |
| SV40* | 16B- | | StDev | 5.2% | 1.6% | 1.4% | 0.6% |
| (SEQ ID | 1613 | 80:240 | Average | 89.9% | 95.5% | 95.9% | 91.3% |
| NO: | | | StDev | 0.1% | 0.2% | 0.0% | 0.4% |
| 479) | | | | | | | |
| GS- | FIG. | 20:60 | Average | 93.7% | 95.8% | 90.2% | 92.4% |
| SV40- | 16B- | | StDev | 0.8% | 0.6% | 0.8% | 2.3% |
| GS-NPL | 1614 | 80:240 | Average | 94.9% | 97.1% | 97.2% | 96.0% |
| (SEQ ID | | | StDev | 1.7% | 0.1% | 0.2% | 0.1% |
| NO: | | | | | | | |
| 483) | | | | | | | |
| (G4S)2- | FIG. | 20:60 | Average | 92.1% | 93.7% | 91.7% | 89.8% |
| SV40 | 16B- | | StDev | 0.5% | 1.7% | 0.6% | 1.8% |
| (SEQ ID | 1615 | 80:240 | Average | 94.6% | 95.0% | 96.5% | 94.2% |
| NO: | | | StDev | 1.2% | 0.0% | 1.3% | 0.9% |
| 487) | | | | | | | |
| (G4S)2- | FIG. | 20:60 | Average | 91.2% | 93.7% | 89.7% | 89.8% |
| NPL | 16B- | | StDev | 0.6% | 1.3% | 0.8% | 0.8% |
| (SEQ ID | 1616 | 80:240 | Average | 94.1% | 96.1% | 96.6% | 94.9% |
| NO: | | | StDev | 0.5% | 0.0% | 0.7% | 0.9% |
| 489) | | | | | | | |
| (G4S)2- | FIG. | 20:60 | Average | 88.1% | 93.9% | 80.3% | 88.1% |
| SV40- | 16B- | | StDev | 0.4% | 0.4% | 2.9% | 2.6% |
| GS-NPL | 1617 | 80:240 | Average | 94.6% | 96.2% | 95.6% | 93.9% |
| (SEQ ID | | | StDev | 0.3% | 0.5% | 0.7% | 0.5% |
| NO: | | | | | | | |
| 485) | | | | | | | |

StDev = standard deviation; n = 2;
*= NLS design used in previous examples

The data presented in FIG. 16B and Tables 25 and 26 demonstrates the improved activity of various NLS configurations across multiple targets in human primary T cells (see, i.e., FIG. 16B average editing of FIG. 16B 1613 compared to average editing of FIG. 16B 1616 or FIG. 16B 1617). Alternative NLS sequences, linkers, and Cas nucleases can be screened in a similar manner to the methods described herein.

### Example 12

Multiplexing with Cas12a chRDNA Guide/Nucleoprotein Complexes

This Example describes the codelivery of multiple Cas12a chRDNAs in a single transfection reaction (multiplexing) to a cell and comparison of multiplexed editing rates of Cas12a chRDNA guide/nucleoprotein complexes with the GS-SV40 (SEQ ID NO: 479; “unoptimized NLS”) and a (G4S) 2-NPL (SEQ ID NO:489; “optimized NLS”).

A. In Silico Design of Cas12a chRDNAs Linker-NLS Sequences

The Acidaminococcus sp. (strain BV3L6) Cas12a protein (SEQ ID NO:01) was engineered with either a first C-terminal linker-NLS sequence (SEQ ID NO:479) or a second C-terminal linker-NLS sequence (SEQ ID NO:489) and the Cas12a recombinant protein was expressed as described in Example 2.

### B. Cellular Multiplexing Activity of Cas12a Linker-NLS

The purified recombinant Cas12a proteins comprising either the linker-NLS sequence SEQ ID NO:479 or the linker-NLS sequence SEQ ID NO:489 were complexed with either a TRAC targeting chRDNA (SEQ ID NO:508), a B2M targeting chRDNA gene (SEQ ID NO: 416), a CISH targeting chRDNA (SEQ ID NO:509), or a CBLB targeting chRDNA (SEQ ID NO:510) as described in Example 2, with the deviation that complexes were assembled at a ratio of 40:80 pmols of Cas12a to guide. Each Cas12a chRDNA guide/nucleoprotein complex was used as a single targeting complex; as both TRAC and B2M targeting Cas12a chRDNA guide/nucleoprotein complexes combined into one mix; as both CISH and CBLB targeting Cas12a chRDNA guide/nucleoprotein complexes combined into one mix; or as all TRAC, B2M, CISH, and CBLB targeting Cas12a chRDNA guide/nucleoprotein complexes combined into one mix. Each Cas12a chRDNA/nucleoprotein composition was transfected into primary T cells as described in Example 3. Forty-eight hours after transfection, the resulting genome editing efficiency of each Cas12a chRDNA/nucleoprotein complex was determined as described in Example 4. The results of the cellular editing experiment are shown in FIG. 17 and presented in Table 27.

TABLE 27
Cellular Editing Activity of Multiplex Cas12a Linker-NLS Constructs

| Number of | Target | SEQ ID | Unoptimized NLS | | Optimized NLS | |
|---|---|---|---|---|---|---|
| multiplexed guides | Name | NO. | rep-1 | rep-2 | rep-1 | rep-2 |
| One guide | TRAC | SEQ ID | 75.07% | 3.35% | 86.33% | 0.91% |
| | | NO: 36 | | | | |
| | B2M | SEQ ID | 83.60% | 86.50% | 89.60% | 91.00% |
| | | NO: 62 | | | | |
| | CISH | SEQ ID | 51.40% | 59.00% | 83.10% | 89.60% |
| | | NO: 158 | | | | |
| | CBLB | SEQ ID | 47.50% | 44.90% | 74.00% | 81.40% |
| | | NO: 171 | | | | |
| Two guides | TRAC | SEQ ID | 81.70% | 80.80% | 93.90% | 92.20% |
| | | NO: 36 | | | | |
| | B2M | SEQ ID | 88.00% | 91.30% | 93.70% | 92.20% |
| | | NO: 62 | | | | |
| Two guides | CISH | SEQ ID | 94.80% | 63.30% | 94.50% | 93.10% |
| | | NO: 158 | | | | |
| | CBLB | SEQ ID | 66.20% | 60.20% | 90.90% | 88.30% |
| | | NO: 171 | | | | |
| Four guides | TRAC | SEQ ID | 78.40% | 82.00% | 91.20% | 93.10% |
| | | NO: 36 | | | | |
| | B2M | SEQ ID | 90.40% | 92.20% | 92.40% | 94.20% |
| | | NO: 62 | | | | |
| | CISH | SEQ ID | 53.20% | 58.70% | 90.80% | 91.80% |
| | | NO: 158 | | | | |
| | CBLB | SEQ ID | 51.60% | 58.00% | 86.40% | 88.20% |
| | | NO: 171 | | | | |

The data presented in FIG. 17 and Table 27 demonstrates the improved activity of a linker-NLS construct when used for multiplexing in human primary T cells. See, e.g., average editing of series FIG. 17, 1707 with the unoptimized GS-SV40 linker-NLS (SEQ ID NO: 479, FIG. 17 1708) and average editing of series FIG. 17, 1711 with the optimized (G4S) 2-NPL linker NLS (SEQ ID NO:489, FIG. 17, 1712). Alternative NLS sequences, linkers, Cas12 nucleases, and targets for multiplexing can be screened in a similar manner to the methods described herein.

### Example 13

Editing with Cas12a chRDNA Guide/Nucleoprotein Complexes Comprising Chemical Modifications

This Example describes the cellular editing activities of Cas12a chRDNAs containing phosphorothioate chemical modifications in the activating and targeting region of the Cas12a guide RNA.

A. In Silico Design of Cas12a chRDNA with Chemical Modifications

The TRAC target-12 sequence (SEQ ID NO:316) was selected for engineering. Two phosphorothioate bonds were designed into 5′ end of the Cas12a guide (i.e., 5′-terminal nucleotides of the activating region) and two phosphorothioate bonds were designed into the 3′ end of the Cas12a guide (i.e., 3′-terminal nucleotides of the targeting region). A series of Cas12a guides were then designed with DNA bases in the activating region in addition to the end-protecting phosphorothioate modifications. Sequences of the Cas12a Cas12a chRDNA with chemical modifications are shown in Table 28. The sequences were provided to a commercial manufacturer for synthesis (the phosphorothioate bond and DNA base position listed correspond to the numbers shown in FIG. 5).

TABLE 28
Cas12a chRDNAs with Chemical Modification

| | Phosphorothioate | | DNA | |
|---|---|---|---|---|
| SEQ ID | Activating | | Activating | |
| NO. | Region | Spacer | Region | Sequence |
| SEQ ID | — | — | — | rUrArArUrUrUrCrUrArCrUrCrUrUr |
| NO: 511 | | | | GrUrArGrArUrGrArGrUrCrUrCrUrC |
| | | | | rArGrCrUrGrGrUrArCrArC |
| SEQ ID | 1, 2 | 38, 39 | | rU*rA*rArUrUrUrCrUrArCrUrCrUr |
| NO: 512 | | | | UrGrUrArGrArUrGrArGrUrCrUrCrU |
| | | | | rCrArGrCrUrGrGrUrArC*rA*rC |
| SEQ ID | 1, 2 | 38, 39 | 1 | T*rA*rArUrUrUrCrUrArCrUrCrUrU |
| NO: 513 | | | | rGrUrArGrArUrGrArGrUrCrUrCrUr |
| | | | | CrArGrCrUrGrGrUrArC*rA*rC |
| SEQ ID | 1, 2, 7 | 38, 39 | 7 | rU*rA*rArUrUrUC*rUrArCrUrCrUr |
| NO: 514 | | | | UrGrUrArGrArUrGrArGrUrCrUrCrU |
| | | | | rCrArGrCrUrGrGrUrArC*rA*rC |
| SEQ ID | 1, 2, 10 | 38, 39 | 10 | rU*rA*rArUrUrUrCrUrAC*rUrCrUr |
| NO: 515 | | | | UrGrUrArGrArUrGrArGrUrCrUrCrU |
| | | | | rCrArGrCrUrGrGrUrArC*rA*rC |
| SEQ ID | 1, 2, 12 | 38, 39 | 12 | rU*rA*rArUrUrUrCrUrArCrUC*rUr |
| NO: 516 | | | | UrGrUrArGrArUrGrArGrUrCrUrCrU |
| | | | | rCrArGrCrUrGrGrUrArC*rA*rC |
| SEQ ID | 1, 2, 14 | 38, 39 | 14 | rU*rA*rArUrUrUrCrUrArCrUrCrUT |
| NO: 517 | | | | *rGrUrArGrArUrGrArGrUrCrUrCrU |
| | | | | rCrArGrCrUrGrGrUrArC*rA*rC |

Phosphorothioate bonds are represented with an “*”

### B. Cell Transfection and Analysis

Individual Cas12a guide/nucleoprotein complexes were prepared essentially as described in Example 2. The nucleoprotein complexes were transfected into primary T cells as described in Example 3, and the resulting genome editing efficiency of the Cas12a guide/nucleoprotein complexes was determined as described in Example 4. The results of the in-cell editing experiment are shown in Table 29.

TABLE 29
Cellular Editing of Cas12a chRDNA with Chemical Modification

| | Phosphorothioate Bond | | DNA Base | | |
|---|---|---|---|---|---|
| | Position | | Position | | |
| | Activating | Targeting | Activating | | |
| SEQ ID NO. | Region | Region | Region | Average | StDev |
| SEQ ID NO: 511 | — | — | — | 74.8% | 1.6% |
| SEQ ID NO: 512 | 1, 2 | 38, 39 | | 78.6% | 4.7% |
| SEQ ID NO: 513 | 1, 2, 1 | 38, 39 | 1 | 71.4% | 1.9% |
| SEQ ID NO: 514 | 1, 2, 7 | 38, 39 | 7 | 75.8% | 3.7% |
| SEQ ID NO: 515 | 1, 2, 10 | 38, 39 | 10 | 79.8% | 2.4% |
| SEQ ID NO: 516 | 1, 2, 12 | 38, 39 | 12 | 70.2% | 0.7% |
| SEQ ID NO: 517 | 1, 2, 14 | 38, 39 | 14 | 72.4% | 5.0% |

StDev = standard deviation; n = 2

The data presented in Table 29 demonstrates the editing activity of Cas12a guides comprising either phosphorothioate bonds alone or phosphorothioate bonds and DNA bases. These guides are capable of robust editing in human primary T cells compared to an all-RNA guide (see, i.e., Table 29 average editing of SEQ ID NO:511 compared to average editing of SEQ ID NO:512 or SEQ ID NO:515). Alternative combination and positions of chemical modification can be screened in a similar manner to the methods described herein.

### Example 14

Transfection of Human Induced Pluripotent Stem Cells with Cas12a chRDNA/Nucleoprotein Complexes

This Example describes the cellular editing of human induced pluripotent stem cells (iPSCs) with Cas12a chRDNA/nucleoprotein complexes.

A. In Silico Design of chRDNA Guides

The AsCas12a guide sequence targeting the CISH gene (SEQ ID NO:509) comprising DNA bases in the activating region of the guide was selected for further engineering and the introduction of additional DNA bases into the targeting region (SEQ ID NO: 518-SEQ ID NO: 529). Sequences were provided to a commercial manufacturer for synthesis.

### B. Cell Transfection and Analysis

Individual Cas12a guide/nucleoprotein complexes were prepared essentially as described in Example 2. The nucleoprotein complexes were transfected into primary T cells as described in Example 3.

iPSCs were handled and transfected in a manner similar to the methods for handling and transfecting primary T cells described in Example 3, with the following modifications. iPSCs were cultured in mTeSR-plus medium (STEMCELL Technologies, Cambridge, MA), supplemented with Rho-associated, coiled-coil-containing protein kinase inhibitor (“ROCKi,” MilliporeSigma, Burlington, MA) at a final concentration of 10 uM for 3 hours at 37° C. prior to transfection. The mTeSR-plus/ROCKi media was removed and the iPSCs were washed with 10 mL of PBS, followed by the addition of 3 mL of accutase (STEMCELL Technologies, Cambridge, MA) and the cells were incubated for 5-10 minutes at 37° C. 7 mL of mTeSR-pulse and ROCKi was then added to the cells, and the cells were mixed and counted. Cells were then centrifuged, and the medium was removed, and the cells were washed with 10 mL of PBS, centrifuged again and PBS removed. Cells were resuspended in a Nucleofector™ P3 (Lonza, Allendale, NJ) solution to a density of 2e5 cells/mL, mixed with Cas12a chRDNA guide/nucleoprotein complexes, and transfected using pulse code CA158. The resulting genome editing efficiencies of the Cas12a guide/nucleoprotein complexes were determined as described in Example 4 and are presented in Table 29.

TABLE 29
Cellular Editing of iPSCs with Cas12a chRDNAs

| | | DNA | | |
|---|---|---|---|---|
| | | Positions in | | |
| | | Target | | |
| | DNA Positions in | Recognition | | |
| SEQ ID NO. | Activating Region | Sequence | Average | StDev |
| SEQ ID NO: 509 | 1, 3, 7, 10, 12, 14, 15, 19 | — | 91.5% | 0.4% |
| SEQ ID NO: 518 | 1, 3, 7, 10, 12, 14, 15, 19 | 1 | 75.5% | 3.9% |
| SEQ ID NO: 519 | 1, 3, 7, 10, 12, 14, 15, 19 | 8 | 92.0% | 0.0% |
| SEQ ID NO: 520 | 1, 3, 7, 10, 12, 14, 15, 19 | 9 | 92.0% | 2.1% |
| SEQ ID NO: 521 | 1, 3, 7, 10, 12, 14, 15, 19 | 10 | 96.0% | 0.7% |
| SEQ ID NO: 522 | 1, 3, 7, 10, 12, 14, 15, 19 | 11 | 95.0% | 0.0% |
| SEQ ID NO: 523 | 1, 3, 7, 10, 12, 14, 15, 19 | 12 | 94.5% | 0.4% |
| SEQ ID NO: 524 | 1, 3, 7, 10, 12, 14, 15, 19 | 14 | 92.5% | 1.1% |
| SEQ ID NO: 525 | 1, 3, 7, 10, 12, 14, 15, 19 | 15 | 89.5% | 4.6% |
| SEQ ID NO: 526 | 1, 3, 7, 10, 12, 14, 15, 19 | 17 | 91.0% | 2.8% |
| SEQ ID NO: 527 | 1, 3, 7, 10, 12, 14, 15, 19 | 18 | 88.0% | — |
| SEQ ID NO: 528 | 1, 3, 7, 10, 12, 14, 15, 19 | 19 | 85.5% | 3.2% |
| SEQ ID NO: 529 | 1, 3, 7, 10, 12, 14, 15, 19 | 20 | 81.5% | 0.4% |

StDev = standard deviation;
n = 1-2

The data presented in Table 29 demonstrate that Cas12a chRDNA guide/nucleoprotein complexes can be used for engineering of human iPSCs. Other cell types can be edited in a similar manner to the methods described herein.

As is apparent to one of skill in the art, modifications and variations of the above embodiments can be made without departing from the spirit and scope of this disclosure. Such modifications and variations are within the scope of this disclosure.

What is claimed is:

1. A CRISPR guide molecule, comprising:
a targeting region capable of binding a target nucleic acid sequence and
an activating region comprising the RNA sequence UAAUUUCUACUCUUGUAGAU including at least one deoxyribonucleotide in place of a ribonucleotide, wherein the activating region is capable of forming a nucleoprotein complex with a Cas12 protein.

2. The CRISPR guide molecule of claim 1, wherein one or more of positions 1, 3, 7, 10, 12, 14, 15, 16, 17, 18, and 19 in the activating region comprise a deoxyribonucleotide base.

3. The CRISPR guide molecule of claim 1, wherein ten or less of positions 1, 3, 7, 10, 12, 14, 15, 16, 17, 18, and 19 in the activating region comprise deoxyribonucleotide bases.

4. The CRISPR guide molecule of claim 1, comprising one or more chemical modifications selected from the group consisting of base modifications including inosine, deoxy-inosine, deoxy-uracil, xanthosine, C3 spacer, 5-methyl dC, 5-hydroxybutynl-2′-deoxyuridine, 5-nitroindole, 5-methyl iso-deoxycytosine, iso deoxyguanosine, deoxyuridine, iso-deoxycytidine, and an abasic site, and backbone modification, including a phosphorothioate modification.

5. The CRISPR guide molecule of claim 1, wherein the targeting region targets the B2M gene and comprises the RNA sequence AGUGGGGGUGAAUUCAGUGU, wherein optionally, at least one of the bases in the sequence is replaced with a base analog or an abasic site.

6. The CRISPR guide molecule of claim 5, wherein one or more of positions 1, 8, 9, 10, 11, 12, 14, 15, 16, 17, 18, 19, and 20 in the targeting region comprise a deoxyribonucleotide base.

7. The CRISPR guide molecule of claim 5, wherein five or less of positions 1, 8, 9, 10, 11, 12, 14, 15, 16, 17, 18, 19, and 20 in the targeting region comprise deoxyribonucleotide bases.

8. The CRISPR guide molecule of claim 5, wherein the targeting region is capable of hybridizing to a sequence selected from SEQ ID NOs: 51-133.

9. The CRISPR guide molecule of claim 5 comprising a sequence selected from SEQ ID NOs: 212-231, 275-315, and 331-350.

10. The CRISPR guide molecule of claim 5 comprising the sequence of SEQ ID NO: 416.

11. The CRISPR guide molecule of claim 1, wherein the targeting region targets the TRAC gene and comprises the RNA sequence GAGUCUCUCAGCUGGUACAC, wherein optionally, at least one of the bases in the sequence is replaced with a base analog or an abasic site.

12. The CRISPR guide molecule of claim 11, wherein one or more of positions 1, 8, 9, 10, 11, 12, 14, 15, 16, 17, 18, 19, and 20 in the targeting region comprise a deoxyribonucleotide base.

13. The CRISPR guide molecule of claim 11, wherein five or less of positions 1, 8, 9, 10, 11, 12, 14, 15, 16, 17, 18, 19, and 20 in the targeting region comprise deoxyribonucleotide bases.

14. The CRISPR guide molecule of claim 11, wherein the targeting region is capable of hybridizing to a sequence selected from SEQ ID NOs: 15-20.

15. The CRISPR guide molecule of claim 11 comprising a sequence selected from SEQ ID NOs: 233-252, 317-329, 491-492, and 508.

16. The CRISPR guide molecule of claim 11 further comprising a chemical modification, wherein the CRISPR guide molecule comprises a sequence selected from SEQ ID Nos: 512-517.

17. The CRISPR guide molecule of claim 11 comprising the sequence of SEQ ID NO: 415.

18. The CRISPR guide molecule of claim 1, wherein the targeting region targets the CISH gene and is capable of hybridizing to a sequence selected from SEQ ID NOs: 157-165.

19. The CRISPR guide molecule of claim 18, comprising the sequence selected from SEQ ID NO: 509, and 519-529.

20. The CRISPR guide molecule of claim 1, wherein the targeting region targets the PDCD1 gene and is capable of hybridizing to a sequence selected from SEQ ID NOs: 135-155.

21. The CRISPR guide molecule of claim 1, wherein the targeting region targets the CBLB gene and is capable of hybridizing to a sequence selected from SEQ ID NOs: 167-189.

22. The CRISPR guide molecule of claim 21, comprising the sequence of SEQ ID NO: 510.

23. A CRISPR nucleic acid/protein composition, comprising the CRISPR guide molecule of claim 1 and a Cas12 protein.

24. The CRISPR nucleic acid/protein composition of claim 23, wherein the Cas12 protein is a Cas12a protein comprising at the C-terminus, a linker- and a nuclear localization signal (NLS)-containing sequence having at least 80% sequence identity to an amino acid sequence selected from the group consisting of SEQ ID NOs: 479-490.

25. A cell, comprising the CRISPR nucleic acid/protein composition of claim 1, wherein the cell is a lymphocyte, a chimeric antigen receptor (CAR) T cell, a T cell receptor (TCR) cell, a TCR-engineered CAR-T cell, a tumor infiltrating lymphocyte (TIL), a CAR TIL, a dendritic cell (DC), a CAR-DC, a macrophage, a CAR-macrophage (CAR-M), a natural killer (NK) cell, an induced pluripotent stem cell (iPSC), a cell differentiated from an iPSC cell, or a CAR-NK cell.

26. A method for producing a chimeric antigen receptor (CAR)-expressing cell, said method comprising:
a) contacting a first target nucleic acid comprising a TRAC sequence in a cell with a nucleoprotein complex comprising a catalytically active Cas12 protein and a first CRISPR guide molecule having a targeting region capable of binding the first target nucleic acid sequence; and an activating region capable of forming a nucleoprotein complex with the Cas12 protein, wherein said CRISPR guide molecule comprises ribonucleotide bases and at least one deoxyribonucleotide base in the activating region, the targeting region, or both, and the nucleoprotein complex is capable of cleaving the first target nucleic acid sequence;
b) contacting a second target nucleic acid sequence comprising a B2M sequence in the same cell with a nucleoprotein complex comprising a catalytically active Cas12 protein and a second CRISPR guide molecule having a targeting region capable of binding the second target nucleic acid sequence; and an activating region capable of forming a nucleoprotein complex with the Cas12 protein, wherein said CRISPR guide molecule comprises ribonucleotide bases and at least one deoxyribonucleotide base in the activating region, the targeting region, or both, and the nucleoprotein complex is capable of cleaving the second target nucleic acid sequence;
c) providing a first donor polynucleotide encoding a CAR comprising an scFv, a transmembrane domain, a co-stimulatory domain, and an activating domain, wherein the CAR is capable of being inserted into the cleavage site in the first target nucleic acid sequence;
d) providing a second donor polynucleotide encoding a B2M-HLA-E fusion construct comprising a B2M secretion signal, a HLA-G peptide signal sequence, a first linker sequence, a B2M sequence, a second linker sequence, and a HLA-E sequence, wherein the B2M-HLA-E fusion construct is capable of being inserted into the cleavage site in the second target nucleic acid sequence;
e) cleaving the first target nucleic acid sequence and inserting at least a portion of the first donor polynucleotide into the cleavage site; and
f) cleaving the second target nucleic acid sequence and inserting at least a portion of the second donor polynucleotide into the cleavage site.

27. The method of claim 26, wherein the second donor polynucleotide further comprises a P2A sequence at 5′-end of the B2M-HLA-E fusion construct.

28. The method of claim 26, wherein the first donor polynucleotide comprises the SEQ ID NO: 413.

29. The method of claim 26, wherein the second donor polynucleotide comprises the SEQ ID NO: 414.

30. The method of claim 26, wherein the scFv in the CAR is capable of binding to a cellular target selected from the group consisting of a CD37, a CD38, a CD47, a CD73, a CD4, a CS1, a PD-L1, a NGFR, a ENPP3, a PSCA, a CD79B, a TACI, a VEGFR2, a B7-H3, a B7-H6, a B-cell maturation antigen (BCMA), a CD123, a CD138, a CD171/L1CAM, a CD19, a CD20, a CD22, a CD30, a CD33, a CD70, a CD371, a CEA, a Claudin 18.1, a Claudin 18.2, a CSPG4, a EFGRvIII, a EpCAM, a EphA2, a Epidermal growth factor receptor, a ErbB, a ErbB2 (HER2), a FAP, a FRα, a GD2, a GD3, a Glypican 3, a IL-11Rα, a IL-13Rα2, a IL13 receptor alpha, a LewisY/LeY, a Mesothelin, a MUC1, a MUC16, a NKG2D ligands, a PD1, a PSMA, a ROR-1, a SLAMF7, a TAG72, a ULBP and a MICA/B proteins, a VEGF2, and a WT1.

31. The method of claim 26, wherein the scFv is capable of binding BCMA and comprises a first variable region comprising the amino acid sequence of SEQ ID NO: 474, a second variable region comprising the amino acid sequence of SEQ ID NO: 475, and a linker between the first and second variable regions comprising the amino acid sequence of SEQ ID NO: 476.

32. The method of claim 26, wherein the scFv comprises the amino acid sequence of SEQ ID NO: 477.

33. The method of claim 26, wherein the transmembrane domain of the CAR is derived from a T cell receptor α chain, a T cell receptor β chain, a CD3ζ chain, a CD28, a CD3ε, a CD45, a CD4, a CD5, a CD8, a CD9, a CD16, a CD22, a CD33, a CD37, a CD64, a CD80, a CD86, a CD134, a CD137, an ICOS, a CD154, or a GITR.

34. The method of claim 26, wherein the co-stimulatory domain of the CAR is derived from a CD28, a 4-1BB, a GITR, an ICOS-1, a CD27, an OX-40, or a DAP10.

35. The method of claim 26, wherein the CAR comprises a transmembrane domain derived from a CD8, a 4-1BB co-stimulatory domain, and a CD35 activating domain.

36. The method of claim 26, wherein the vector containing the CAR sequence comprises a leader sequence having the nucleic acid sequence of SEQ ID NO: 478.

37. The method of claim 26, wherein the catalytically active Cas12 protein comprises, at the C-terminus, a linker, and a nuclear localization signal (NLS)-containing sequence having at least 80% sequence identity to an amino acid sequence selected from the group consisting of SEQ ID NOs: 479-490.

38. The method of claim 26, further comprising:
a. contacting a third target nucleic acid sequence in the same cell with a nucleoprotein complex comprising a catalytically active Cas12 protein and a third CRISPR guide molecule having a targeting region capable of binding the third target nucleic acid sequence; and an activating region capable of forming a nucleoprotein complex with the Cas12 protein, wherein said CRISPR guide molecule comprises ribonucleotide bases and at least one deoxyribonucleotide base in the activating region, the targeting region, or both, and the nucleoprotein complex is capable of cleaving the third target nucleic acid sequence; and
b. cleaving the third target nucleic acid sequence and deleting one or more nucleotides from the third target nucleic acid sequence at the cleavage site, wherein the third target nucleic acid sequence is selected from a PDCD gene, a CISH gene, and a CBLB gene.

39. The method of claim 26, wherein the CAR-expressing cell is an allogeneic or autologous CAR-T cell produced from a T-lymphocyte.

40. A CAR-expressing cell produced by the method of claim 26, wherein the cell is selected from a lymphocyte, a CAR-T cell, a TCR cell, a TCR-engineered CAR-T cell, a TIL, a CAR TIL, a dendritic cell, a CAR-DC, a macrophage, a CAR-M, an iPSC cell, a cell differentiated from an iPSC cell, an NK cell, or a CAR-NK cell.

41. A method of adaptive cell therapy, comprising administering to a subject in need thereof the CAR-expressing cell of claim 40.

42. The method of claim 41, wherein the adaptive cell therapy comprises killing BCMA-positive cancer cells.

43. The method of claim 42, wherein the BCMA-positive cancer cells comprise multiple myeloma cancer cells.


SEQUENCE LISTING (533 sequences)

SEQ ID NO: 1 - protein, 1307 aa, Acidaminococcus sp.
  source 1..1307: note = Acidaminococcus sp. (strain BV3L6); note = Cas12a
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 2 - protein, 1228 aa, unidentified
  source 1..1228: note = Description of Unknown: Lachnospiraceae bacterium ND2006; note = Cas12a
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 3 - protein, 1300 aa, Francisella tularensis
  source 1..1300: note = Francisella tularensis subsp. novicida (strain U112); note = Cas12a
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNM

SEQ ID NO: 4 - protein, 7 aa, synthetic construct
  source 1..7: note = Description of Artificial Sequence: Synthetic peptide; note = Nuclear localization sequence (NLS)
PKKKRKV

SEQ ID NO: 5 - protein, 16 aa, synthetic construct
  source 1..16: note = Description of Artificial Sequence: Synthetic peptide; note = Nuclear localization sequence (NLS)
KRPAATKKAGQAKKKK

SEQ ID NO: 6 - RNA, 20 nt, Acidaminococcus sp.
  source 1..20: mol_type = unassigned RNA; note = Acidaminococcus sp. (strain BV3L6); note = AsCas12 CRISPR repeat
taatttctactcttgtagat

SEQ ID NO: 7 - RNA, 40 nt, synthetic construct
  source 1..40: mol_type = other RNA; note = Description of Artificial Sequence: Synthetic oligonucleotide; note = AsCas12 crRNA
  misc_difference 21: note = modified_base - a, c, g, or u
  misc_difference 22: note = modified_base - a, c, g, or u
  misc_difference 23: note = modified_base - a, c, g, or u
  misc_difference 24: note = modified_base - a, c, g, or u
  misc_difference 25: note = modified_base - a, c, g, or u
  misc_difference 26: note = modified_base - a, c, g, or u
  misc_difference 27: note = modified_base - a, c, g, or u
  misc_difference 28: note = modified_base - a, c, g, or u
  misc_difference 29: note = modified_base - a, c, g, or u
  misc_difference 30: note = modified_base - a, c, g, or u
  misc_difference 31: note = modified_base - a, c, g, or u
  misc_difference 32: note = modified_base - a, c, g, or u
  misc_difference 33: note = modified_base - a, c, g, or u
  misc_difference 34: note = modified_base - a, c, g, or u
  misc_difference 35: note = modified_base - a, c, g, or u
  misc_difference 36: note = modified_base - a, c, g, or u
  misc_difference 37: note = modified_base - a, c, g, or u
  misc_difference 38: note = modified_base - a, c, g, or u
  misc_difference 39: note = modified_base - a, c, g, or u
  misc_difference 40: note = modified_base - a, c, g, or u
taatttctactcttgtagatnnnnnnnnnnnnnnnnnnnn

SEQ ID NO: 8 - RNA, 21 nt, unidentified
  source 1..21: mol_type = unassigned RNA; note = Description of Unknown: Lachnospiraceae bacterium ND2006; note = LbaCas12 CRISPR repeat
taatttctactaagtgtagat

SEQ ID NO: 9 - RNA, 41 nt, synthetic construct
  source 1..41: mol_type = other RNA; note = Description of Artificial Sequence: Synthetic oligonucleotide; note = LbaCas12 crRNA
  misc_difference 22: note = modified_base - a, c, g, or u
  misc_difference 23: note = modified_base - a, c, g, or u
  misc_difference 24: note = modified_base - a, c, g, or u
  misc_difference 25: note = modified_base - a, c, g, or u
  misc_difference 26: note = modified_base - a, c, g, or u
  misc_difference 27: note = modified_base - a, c, g, or u
  misc_difference 28: note = modified_base - a, c, g, or u
  misc_difference 29: note = modified_base - a, c, g, or u
  misc_difference 30: note = modified_base - a, c, g, or u
  misc_difference 31: note = modified_base - a, c, g, or u
  misc_difference 32: note = modified_base - a, c, g, or u
  misc_difference 33: note = modified_base - a, c, g, or u
  misc_difference 34: note = modified_base - a, c, g, or u
  misc_difference 35: note = modified_base - a, c, g, or u
  misc_difference 36: note = modified_base - a, c, g, or u
  misc_difference 37: note = modified_base - a, c, g, or u
  misc_difference 38: note = modified_base - a, c, g, or u
  misc_difference 39: note = modified_base - a, c, g, or u
  misc_difference 40: note = modified_base - a, c, g, or u
  misc_difference 41: note = modified_base - a, c, g, or u
taatttctactaagtgtagatnnnnnnnnnnnnnnnnnnnn

SEQ ID NO: 10 - RNA, 20 nt, Francisella tularensis
  source 1..20: mol_type = unassigned RNA; note = Francisella tularensis subsp. novicida (strain U112); note = FnoCas12 CRISPR repeat
taatttctactgttgtagat

SEQ ID NO: 11 - RNA, 40 nt, synthetic construct
  source 1..40: mol_type = other RNA; note = Description of Artificial Sequence: Synthetic oligonucleotide; note = FnoCas12 crRNA
  misc_difference 21: note = modified_base - a, c, g, or u
  misc_difference 22: note = modified_base - a, c, g, or u
  misc_difference 23: note = modified_base - a, c, g, or u
  misc_difference 24: note = modified_base - a, c, g, or u
  misc_difference 25: note = modified_base - a, c, g, or u
  misc_difference 26: note = modified_base - a, c, g, or u
  misc_difference 27: note = modified_base - a, c, g, or u
  misc_difference 28: note = modified_base - a, c, g, or u
  misc_difference 29: note = modified_base - a, c, g, or u
  misc_difference 30: note = modified_base - a, c, g, or u
  misc_difference 31: note = modified_base - a, c, g, or u
  misc_difference 32: note = modified_base - a, c, g, or u
  misc_difference 33: note = modified_base - a, c, g, or u
  misc_difference 34: note = modified_base - a, c, g, or u
  misc_difference 35: note = modified_base - a, c, g, or u
  misc_difference 36: note = modified_base - a, c, g, or u
  misc_difference 37: note = modified_base - a, c, g, or u
  misc_difference 38: note = modified_base - a, c, g, or u
  misc_difference 39: note = modified_base - a, c, g, or u
  misc_difference 40: note = modified_base - a, c, g, or u
taatttctactgttgtagatnnnnnnnnnnnnnnnnnnnn

SEQ ID NO: 12 - DNA, 24 bp, Homo sapiens
  source 1..24: mol_type = unassigned DNA; note = TRAC-tgt1
tttctctcctctttcccacagaat

SEQ ID NO: 13 - DNA, 24 bp, Homo sapiens
  source 1..24: mol_type = unassigned DNA; note = TRAC-tgt2
tttcccacagaatttagcatggct

SEQ ID NO: 14 - DNA, 24 bp, Homo sapiens
  source 1..24: mol_type = unassigned DNA; note = TRAC-tgt3
tttagcatggctcagacagtcact

SEQ ID NO: 15 - DNA, 24 bp, Homo sapiens
  source 1..24: mol_type = unassigned DNA; note = TRAC-tgt4
tttattctggtacaagcagcctcc

SEQ ID NO: 16 - DNA, 24 bp, Homo sapiens
  source 1..24: mol_type = unassigned DNA; note = TRAC-tgt5
tttctggaagttcacagagaaacg

SEQ ID NO: 17 - DNA, 24 bp, Homo sapiens
  source 1..24: mol_type = unassigned DNA; note = TRAC-tgt6
tttctctgtgaacttccagaaagc

SEQ ID NO: 18 - DNA, 24 bp, Homo sapiens
  source 1..24: mol_type = unassigned DNA; note = TRAC-tgt7
tttggctgctttctggaagttcac

SEQ ID NO: 19 - DNA, 24 bp, Homo sapiens
  source 1..24: mol_type = unassigned DNA; note = TRAC-tgt8
tttatcaacctcaccaactcacac

SEQ ID NO: 20 - DNA, 24 bp, Homo sapiens
  source 1..24: mol_type = unassigned DNA; note = TRAC-tgt9
tttgctgtcagtccaagctccatg

SEQ ID NO: 21 - DNA, 24 bp, Homo sapiens
  source 1..24: mol_type = unassigned DNA; note = TRAC-tgt10
tttccaaagacaagaggtgtgttt

SEQ ID NO: 22 - DNA, 24 bp, Homo sapiens
  source 1..24: mol_type = unassigned DNA; note = TRAC-tgt11
tttggaaagggcacaagactttct

SEQ ID NO: 23 - DNA, 24 bp, Homo sapiens
  source 1..24: mol_type = unassigned DNA; note = TRAC-tgt12
tttagagtctctcagctggtacac

SEQ ID NO: 24 - DNA, 24 bp, Homo sapiens
  source 1..24: mol_type = unassigned DNA; note = TRAC-tgt13
tttgagaatcaaaatcggtgaata

SEQ ID NO: 25 - DNA, 24 bp, Homo sapiens
  source 1..24: mol_type = unassigned DNA; note = TRAC-tgt14
tttgtttgagaatcaaaatcggtg

SEQ ID NO: 26 - DNA, 24 bp, Homo sapiens
  source 1..24: mol_type = unassigned DNA; note = TRAC-tgt15
tttgattctcaaacaaatgtgtca

SEQ ID NO: 27 - DNA, 24 bp, Homo sapiens
  source 1..24: mol_type = unassigned DNA; note = TRAC-tgt16
tttgtgacacatttgtttgagaat

SEQ ID NO: 28 - DNA, 24 bp, Homo sapiens
  source 1..24: mol_type = unassigned DNA; note = TRAC-tgt17
tttgtctgtgatatacacatcaga

SEQ ID NO: 29 - DNA, 24 bp, Homo sapiens
  source 1..24: mol_type = unassigned DNA; note = TRAC-tgt18
tttgttgctccaggccacagcact

SEQ ID NO: 30 - DNA, 24 bp, Homo sapiens
  source 1..24: mol_type = unassigned DNA; note = TRAC-tgt19
tttgcacatgcaaagtcagatttg

SEQ ID NO: 31 - DNA, 24 bp, Homo sapiens
  source 1..24: mol_type = unassigned DNA; note = TRAC-tgt20
tttgcatgtgcaaacgccttcaac

SEQ ID NO: 32 - DNA, 24 bp, Homo sapiens
  source 1..24: mol_type = unassigned DNA; note = TRAC-tgt21
tttccttttagaaagttcctgtga

SEQ ID NO: 33 - DNA, 24 bp, Homo sapiens
  source 1..24: mol_type = unassigned DNA; note = TRAC-tgt22
tttagaaagttcctgtgatgtcaa

SEQ ID NO: 34 - DNA, 24 bp, Homo sapiens
  source 1..24: mol_type = unassigned DNA; note = TRAC-tgt23
tttctcgaccagcttgacatcaca

SEQ ID NO: 35 - DNA, 24 bp, Homo sapiens
  source 1..24: mol_type = unassigned DNA; note = TRAC-tgt24
tttcaaagcttttctcgaccagct

SEQ ID NO: 36 - DNA, 24 bp, Homo sapiens
  source 1..24: mol_type = unassigned DNA; note = TRAC-tgt25
tttacagatacgaacctaaacttt

SEQ ID NO: 37 - DNA, 24 bp, Homo sapiens
  source 1..24: mol_type = unassigned DNA; note = TRAC-tgt26
tttgaaagtttaggttcgtatctg

SEQ ID NO: 38 - DNA, 24 bp, Homo sapiens
  source 1..24: mol_type = unassigned DNA; note = TRAC-tgt27
tttcaaaacctgtcagtgattggg

SEQ ID NO: 39 - DNA, 24 bp, Homo sapiens
  source 1..24: mol_type = unassigned DNA; note = TRAC-tgt28
tttcaggaggaggattcggaaccc

SEQ ID NO: 40 - DNA, 24 bp, Homo sapiens
  source 1..24: mol_type = unassigned DNA; note = TRAC-tgt29
tttaatctgctcatgacgctgcgg

SEQ ID NO: 41 - DNA, 24 bp, Homo sapiens
  source 1..24: mol_type = unassigned DNA; note = TRAC-tgt30
tttggagagggagaagaggggcaa

SEQ ID NO: 42 - DNA, 24 bp, Homo sapiens
  source 1..24: mol_type = unassigned DNA; note = TRAC-tgt31
tttcacctccttgggggtaggaga

SEQ ID NO: 43 - DNA, 24 bp, Homo sapiens
  source 1..24: mol_type = unassigned DNA; note = TRAC-tgt32
tttatgattaagattgctgaagag

SEQ ID NO: 44 - DNA, 24 bp, Homo sapiens
  source 1..24: mol_type = unassigned DNA; note = TRAC-tgt33
tttggcagctcttcagcaatctta

SEQ ID NO: 45 - DNA, 24 bp, Homo sapiens
  source 1..24: mol_type = unassigned DNA; note = TRAC-tgt34
tttatttttttttaatagtgttca

SEQ ID NO: 46 - DNA, 24 bp, Homo sapiens
  source 1..24: mol_type = unassigned DNA; note = TRAC-tgt35
tttatgaacactattaaaaaaaaa

SEQ ID NO: 47 - DNA, 24 bp, Homo sapiens
  source 1..24: mol_type = unassigned DNA; note = TRAC-tgt36
tttctttatgaacactattaaaaa

SEQ ID NO: 48 - DNA, 24 bp, Homo sapiens
  source 1..24: mol_type = unassigned DNA; note = TRAC-tgt37
tttaatagtgttcataaagaaata

SEQ ID NO: 49 - DNA, 24 bp, Homo sapiens
  source 1..24: mol_type = unassigned DNA; note = TRAC-tgt38
tttccccccacgtcttgagaagaa

SEQ ID NO: 50 - DNA, 24 bp, Homo sapiens
  source 1..24: mol_type = unassigned DNA; note = TRAC-tgt39
tttaatgaaggcatcggcagcagg

SEQ ID NO: 51 - DNA, 24 bp, Homo sapiens
  source 1..24: mol_type = unassigned DNA; note = B2M-tgt1
tttaatataagtggaggcgtcgcg

SEQ ID NO: 52 - DNA, 24 bp, Homo sapiens
  source 1..24: mol_type = unassigned DNA; note = B2M-tgt2
tttctggcctggaggctatccagc

SEQ ID NO: 53 - DNA, 24 bp, Homo sapiens
  source 1..24: mol_type = unassigned DNA; note = B2M-tgt3
tttcccgatattcctcaggtactc

SEQ ID NO: 54 - DNA, 24 bp, Homo sapiens
  source 1..24: mol_type = unassigned DNA; note = B2M-tgt4
tttactcacgtcatccagcagaga

SEQ ID NO: 55 - DNA, 24 bp, Homo sapiens
  source 1..24: mol_type = unassigned DNA; note = B2M-tgt5
tttccattctctgctggatgacgt

SEQ ID NO: 56 - DNA, 24 bp, Homo sapiens
  source 1..24: mol_type = unassigned DNA; note = B2M-tgt6
tttgactttccattctctgctgga

SEQ ID NO: 57 - DNA, 24 bp, Homo sapiens
  source 1..24: mol_type = unassigned DNA; note = B2M-tgt7
tttcctgaattgctatgtgtctgg

SEQ ID NO: 58 - DNA, 24 bp, Homo sapiens
  source 1..24: mol_type = unassigned DNA; note = B2M-tgt8
tttcatccatccgacattgaagtt

SEQ ID NO: 59 - DNA, 24 bp, Homo sapiens
  source 1..24: mol_type = unassigned DNA; note = B2M-tgt9
tttcaattctctctccattcttca

SEQ ID NO: 60 - DNA, 24 bp, Homo sapiens
  source 1..24: mol_type = unassigned DNA; note = B2M-tgt10
tttcagcaaggactggtctttcta

SEQ ID NO: 61 - DNA, 24 bp, Homo sapiens
  source 1..24: mol_type = unassigned DNA; note = B2M-tgt11
tttctatctcttgtactacactga

SEQ ID NO: 62 - DNA, 24 bp, Homo sapiens
  source 1..24: mol_type = unassigned DNA; note = B2M-tgt12
tttcagtgggggtgaattcagtgt

SEQ ID NO: 63 - DNA, 24 bp, Homo sapiens
  source 1..24: mol_type = unassigned DNA; note = B2M-tgt13
tttgtcacagcccaagatagttaa

SEQ ID NO: 64 - DNA, 24 bp, Homo sapiens
  source 1..24: mol_type = unassigned DNA; note = B2M-tgt14
tttctccactgtctttttcataga

SEQ ID NO: 65 - DNA, 24 bp, Homo sapiens
  source 1..24: mol_type = unassigned DNA; note = B2M-tgt15
tttcatagatcgagacatgtaagc

SEQ ID NO: 66 - DNA, 24 bp, Homo sapiens
  source 1..24: mol_type = unassigned DNA; note = B2M-tgt16
tttctcaaggtcaaaaacttacct

SEQ ID NO: 67 - DNA, 24 bp, Homo sapiens
  source 1..24: mol_type = unassigned DNA; note = B2M-tgt17
tttcttttcttttcaggtttgaag

SEQ ID NO: 68 - DNA, 24 bp, Homo sapiens
  source 1..24: mol_type = unassigned DNA; note = B2M-tgt18
tttcttttcaggtttgaagatgcc

SEQ ID NO: 69 - DNA, 24 bp, Homo sapiens
  source 1..24: mol_type = unassigned DNA; note = B2M-tgt19
tttcaggtttgaagatgccgcatt

SEQ ID NO: 70 - DNA, 24 bp, Homo sapiens
  source 1..24: mol_type = unassigned DNA; note = B2M-tgt20
tttgaagatgccgcatttggattg

SEQ ID NO: 71 - DNA, 24 bp, Homo sapiens
  source 1..24: mol_type = unassigned DNA; note = B2M-tgt21
tttggaattcatccaatccaaatg

SEQ ID NO: 72 - DNA, 24 bp, Homo sapiens
  source 1..24: mol_type = unassigned DNA; note = B2M-tgt22
tttggattggatgaattccaaatt

SEQ ID NO: 73 - DNA, 24 bp, Homo sapiens
  source 1..24: mol_type = unassigned DNA; note = B2M-tgt23
tttaatattgatatgcttatacac

SEQ ID NO: 74 - DNA, 24 bp, Homo sapiens
  source 1..24: mol_type = unassigned DNA; note = B2M-tgt24
tttgtgcataaagtgtaagtgtat

SEQ ID NO: 75 - DNA, 24 bp, Homo sapiens
  source 1..24: mol_type = unassigned DNA; note = B2M-tgt25
tttatgcacaaaatgtagggttat

SEQ ID NO: 76 - DNA, 24 bp, Homo sapiens
  source 1..24: mol_type = unassigned DNA; note = B2M-tgt26
tttataattctactttgagtgctg

SEQ ID NO: 77 - DNA, 24 bp, Homo sapiens
  source 1..24: mol_type = unassigned DNA; note = B2M-tgt27
tttgagtgctgtctccatgtttga

SEQ ID NO: 78 - DNA, 24 bp, Homo sapiens
  source 1..24: mol_type = unassigned DNA; note = B2M-tgt28
tttgatgtatctgagcaggttgct

SEQ ID NO: 79 - DNA, 24 bp, Homo sapiens
  source 1..24: mol_type = unassigned DNA; note = B2M-tgt29
tttgaactcttcaatctcttgcac

SEQ ID NO: 80 - DNA, 24 bp, Homo sapiens
  source 1..24: mol_type = unassigned DNA; note = B2M-tgt30
tttgagtgcaagagattgaagagt

SEQ ID NO: 81 - DNA, 24 bp, Homo sapiens
  source 1..24: mol_type = unassigned DNA; note = B2M-tgt31
tttacatactctgcttagaatttg

SEQ ID NO: 82 - DNA, 24 bp, Homo sapiens
  source 1..24: mol_type = unassigned DNA; note = B2M-tgt32
tttcccccaaattctaagcagagt

SEQ ID NO: 83 - DNA, 24 bp, Homo sapiens
  source 1..24: mol_type = unassigned DNA; note = B2M-tgt33
tttctaaattttcccccaaattct

SEQ ID NO: 84 - DNA, 24 bp, Homo sapiens
  source 1..24: mol_type = unassigned DNA; note = B2M-tgt34
tttgggggaaaatttagaaatata

SEQ ID NO: 85 - DNA, 24 bp, Homo sapiens
  source 1..24: mol_type = unassigned DNA; note = B2M-tgt35
tttagaaatataattgacaggatt

SEQ ID NO: 86 - DNA, 24 bp, Homo sapiens
  source 1..24: mol_type = unassigned DNA; note = B2M-tgt36
tttccaataatcctgtcaattata

SEQ ID NO: 87 - DNA, 24 bp, Homo sapiens
  source 1..24: mol_type = unassigned DNA; note = B2M-tgt37
tttcattcattataacaaatttcc

SEQ ID NO: 88 - DNA, 24 bp, Homo sapiens
  source 1..24: mol_type = unassigned DNA; note = B2M-tgt38
tttgttataatgaatgaaacattt

SEQ ID NO: 89 - DNA, 24 bp, Homo sapiens
  source 1..24: mol_type = unassigned DNA; note = B2M-tgt39
tttgtcatataagattcatattta

SEQ ID NO: 90 - DNA, 24 bp, Homo sapiens
  source 1..24: mol_type = unassigned DNA; note = B2M-tgt40
tttacttcttatacatttgataaa

SEQ ID NO: 91 - DNA, 24 bp, Homo sapiens
  source 1..24: mol_type = unassigned DNA; note = B2M-tgt41
tttatcaaatgtataagaagtaaa

SEQ ID NO: 92 - DNA, 24 bp, Homo sapiens
  source 1..24: mol_type = unassigned DNA; note = B2M-tgt42
tttgataaagtaaggcatggttgt

SEQ ID NO: 93 - DNA, 24 bp, Homo sapiens
  source 1..24: mol_type = unassigned DNA; note = B2M-tgt43
tttatttttgttccacaagttaaa

SEQ ID NO: 94 - DNA, 24 bp, Homo sapiens
  source 1..24: mol_type = unassigned DNA; note = B2M-tgt44
tttaacttgtggaacaaaaataaa

SEQ ID NO: 95 - DNA, 24 bp, Homo sapiens
  source 1..24: mol_type = unassigned DNA; note = B2M-tgt45
tttatttaacttgtggaacaaaaa

SEQ ID NO: 96 - DNA, 24 bp, Homo sapiens
  source 1..24: mol_type = unassigned DNA; note = B2M-tgt46
tttgttccacaagttaaataaatc

SEQ ID NO: 97 - DNA, 24 bp, Homo sapiens
  source 1..24: mol_type = unassigned DNA; note = B2M-tgt47
tttatgatttatttaacttgtgga

SEQ ID NO: 98 - DNA, 24 bp, Homo sapiens
  source 1..24: mol_type = unassigned DNA; note = B2M-tgt48
tttgaaaataaaggggtaatagtg

SEQ ID NO: 99 - DNA, 24 bp, Homo sapiens
  source 1..24: mol_type = unassigned DNA; note = B2M-tgt49
tttccctgtttgaaaataaagggg

SEQ ID NO: 100 - DNA, 24 bp, Homo sapiens
  source 1..24: mol_type = unassigned DNA; note = B2M-tgt50
tttattttcaaacagggaaacagt

SEQ ID NO: 101 - DNA, 24 bp, Homo sapiens
  source 1..24: mol_type = unassigned DNA; note = B2M-tgt51
tttcaaacagggaaacagtcttca

SEQ ID NO: 102 - DNA, 24 bp, Homo sapiens
  source 1..24: mol_type = unassigned DNA; note = B2M-tgt52
tttaccaagtggaacttgaagact

SEQ ID NO: 103 - DNA, 24 bp, Homo sapiens
  source 1..24: mol_type = unassigned DNA; note = B2M-tgt53
tttacactgtgagccaaactctat

SEQ ID NO: 104 - DNA, 24 bp, Homo sapiens
  source 1..24: mol_type = unassigned DNA; note = B2M-tgt54
tttggctcacagtgtaaagggcct

SEQ ID NO: 105 - DNA, 24 bp, Homo sapiens
  source 1..24: mol_type = unassigned DNA; note = B2M-tgt55
tttccagattaggaatctgatgct

SEQ ID NO: 106 - DNA, 24 bp, Homo sapiens
  source 1..24: mol_type = unassigned DNA; note = B2M-tgt56
tttgagcatcagattcctaatctg

SEQ ID NO: 107 - DNA, 24 bp, Homo sapiens
  source 1..24: mol_type = unassigned DNA; note = B2M-tgt57
tttaacttctttgagcatcagatt

SEQ ID NO: 108 - DNA, 24 bp, Homo sapiens
  source 1..24: mol_type = unassigned DNA; note = B2M-tgt58
tttagccagcgttctttcctgcgg

SEQ ID NO: 109 - DNA, 24 bp, Homo sapiens
  source 1..24: mol_type = unassigned DNA; note = B2M-tgt59
tttcctgcgggccaggtcatgagg

SEQ ID NO: 110 - DNA, 24 bp, Homo sapiens
  source 1..24: mol_type = unassigned DNA; note = B2M-tgt60
tttgctccctcttagagtctgcat

SEQ ID NO: 111 - DNA, 24 bp, Homo sapiens
  source 1..24: mol_type = unassigned DNA; note = B2M-tgt61
tttaatattttagcaaggaataga

SEQ ID NO: 112 - DNA, 24 bp, Homo sapiens
  source 1..24: mol_type = unassigned DNA; note = B2M-tgt62
tttagcaaggaatagatatacaat

SEQ ID NO: 113 - DNA, 24 bp, Homo sapiens
  source 1..24: mol_type = unassigned DNA; note = B2M-tgt63
tttcaggaccccttcttggacacc

SEQ ID NO: 114 - DNA, 24 bp, Homo sapiens
  source 1..24: mol_type = unassigned DNA; note = B2M-tgt64
tttggtgtccaagaaggggtcctg

SEQ ID NO: 115 - DNA, 24 bp, Homo sapiens
  source 1..24: mol_type = unassigned DNA; note = B2M-tgt65
tttatagaagggacttaaatatcc

SEQ ID NO: 116 - DNA, 24 bp, Homo sapiens
  source 1..24: mol_type = unassigned DNA; note = B2M-tgt66
tttaagtcccttctataaaatggt

SEQ ID NO: 117 - DNA, 24 bp, Homo sapiens
  source 1..24: mol_type = unassigned DNA; note = B2M-tgt67
tttgcatataacctatccacatcc

SEQ ID NO: 118 - DNA, 24 bp, Homo sapiens
  source 1..24: mol_type = unassigned DNA; note = B2M-tgt68
tttaaagtatacaggaggatgtgg

SEQ ID NO: 119 - DNA, 24 bp, Homo sapiens
  source 1..24: mol_type = unassigned DNA; note = B2M-tgt69
tttaaatcatttctagattacttg

SEQ ID NO: 120 - DNA, 24 bp, Homo sapiens
  source 1..24: mol_type = unassigned DNA; note = B2M-tgt70
tttctagattacttgtaataccta

SEQ ID NO: 121 - DNA, 24 bp, Homo sapiens
  source 1..24: mol_type = unassigned DNA; note = B2M-tgt71
tttacattgtattaggtattacaa

SEQ ID NO: 122 - DNA, 24 bp, Homo sapiens
  source 1..24: mol_type = unassigned DNA; note = B2M-tgt72
tttgcatagcatttacattgtatt

SEQ ID NO: 123 - DNA, 24 bp, Homo sapiens
  source 1..24: mol_type = unassigned DNA; note = B2M-tgt73
tttcttgtcattattccttaaaca

SEQ ID NO: 124 - DNA, 24 bp, Homo sapiens
  source 1..24: mol_type = unassigned DNA; note = B2M-tgt74
tttaaggaataatgacaagaaaaa

SEQ ID NO: 125 - DNA, 24 bp, Homo sapiens
  source 1..24: mol_type = unassigned DNA; note = B2M-tgt75
tttactgagcatgtacagactttt

SEQ ID NO: 126 - DNA, 24 bp, Homo sapiens
  source 1..24: mol_type = unassigned DNA; note = B2M-tgt76
tttccccagtgtttttgatccatg

SEQ ID NO: 127 - DNA, 24 bp, Homo sapiens
  source 1..24: mol_type = unassigned DNA; note = B2M-tgt77
tttgatccatggtttgctgaatcc

SEQ ID NO: 128 - DNA, 24 bp, Homo sapiens
  source 1..24: mol_type = unassigned DNA; note = B2M-tgt78
tttgctgaatccacagatgtggag

SEQ ID NO: 129 - DNA, 24 bp, Homo sapiens
  source 1..24: mol_type = unassigned DNA; note = B2M-tgt79
tttgtcattcaaagtacagcgggc

SEQ ID NO: 130 - DNA, 24 bp, Homo sapiens
  source 1..24: mol_type = unassigned DNA; note = B2M-tgt80
tttgaatgacaaataacagattta

SEQ ID NO: 131 - DNA, 24 bp, Homo sapiens
  source 1..24: mol_type = unassigned DNA; note = B2M-tgt81
tttaaatctgttatttgtcattca

SEQ ID NO: 132 - DNA, 24 bp, Homo sapiens
  source 1..24: mol_type = unassigned DNA; note = B2M-tgt82
tttaaaattttcaaggcatagttt

SEQ ID NO: 133 - DNA, 24 bp, Homo sapiens
  source 1..24: mol_type = unassigned DNA; note = B2M-tgt83
tttcaaggcatagttttatacctg

SEQ ID NO: 134 - DNA, 24 bp, Homo sapiens
  source 1..24: mol_type = unassigned DNA; note = PDCD1-tgt1
tttaattataattataatataata

SEQ ID NO: 135 - DNA, 24 bp, Homo sapiens
  source 1..24: mol_type = unassigned DNA; note = PDCD1-tgt2
tttaaaggggttggccgggctccc

SEQ ID NO: 136 - DNA, 24 bp, Homo sapiens
  source 1..24: mol_type = unassigned DNA; note = PDCD1-tgt3
tttaaataatttcaggaatgggtt

SEQ ID NO: 137 - DNA, 24 bp, Homo sapiens
  source 1..24: mol_type = unassigned DNA; note = PDCD1-tgt4
tttcaggaatgggttccaaggaga

SEQ ID NO: 138 - DNA, 24 bp, Homo sapiens
  source 1..24: mol_type = unassigned DNA; note = PDCD1-tgt5
tttcaggcccagccagcactctgg

SEQ ID NO: 139 - DNA, 24 bp, Homo sapiens
  source 1..24: mol_type = unassigned DNA; note = PDCD1-tgt6
tttgtggggcagggaagctgaggc

SEQ ID NO: 140 - DNA, 24 bp, Homo sapiens
  source 1..24: mol_type = unassigned DNA; note = PDCD1-tgt7
tttacacatgcccaggcagcacct

SEQ ID NO: 141 - DNA, 24 bp, Homo sapiens
  source 1..24: mol_type = unassigned DNA; note = PDCD1-tgt8
tttcctgccctgcccaccacagcc

SEQ ID NO: 142 - DNA, 24 bp, Homo sapiens
  source 1..24: mol_type = unassigned DNA; note = PDCD1-tgt9
tttcagggaaggtcagaagagctc

SEQ ID NO: 143 - DNA, 24 bp, Homo sapiens
  source 1..24: mol_type = unassigned DNA; note = PDCD1-tgt10
tttcgggatgccactgccaggggc

SEQ ID NO: 144 - DNA, 24 bp, Homo sapiens
  source 1..24: mol_type = unassigned DNA; note = PDCD1-tgt11
tttcctcaggagaagcaggcaggg

SEQ ID NO: 145 - DNA, 24 bp, Homo sapiens
  source 1..24: mol_type = unassigned DNA; note = PDCD1-tgt12
tttcctagcggaatgggcacctca

SEQ ID NO: 146 - DNA, 24 bp, Homo sapiens
  source 1..24: mol_type = unassigned DNA; note = PDCD1-tgt13
tttccagtggcgagagaagacccc

SEQ ID NO: 147 - DNA, 24 bp, Homo sapiens
  source 1..24: mol_type = unassigned DNA; note = PDCD1-tgt14
tttctcctcaaagaaggaggaccc

SEQ ID NO: 148 - DNA, 24 bp, Homo sapiens
  source 1..24: mol_type = unassigned DNA; note = PDCD1-tgt15
tttctctgcagggacaataggagc

SEQ ID NO: 149 - DNA, 24 bp, Homo sapiens
  source 1..24: mol_type = unassigned DNA; note = PDCD1-tgt16
tttggaactggccggctggcctgg

SEQ ID NO: 150 - DNA, 24 bp, Homo sapiens
  source 1..24: mol_type = unassigned DNA; note = PDCD1-tgt17
tttgtgcccttccagagagaaggg

SEQ ID NO: 151 - DNA, 24 bp, Homo sapiens
  source 1..24: mol_type = unassigned DNA; note = PDCD1-tgt18
tttgatctgcgccttgggggccag

SEQ ID NO: 152 - DNA, 24 bp, Homo sapiens
  source 1..24: mol_type = unassigned DNA; note = PDCD1-tgt19
tttagcacgaagctctccgatgtg

SEQ ID NO: 153 - DNA, 24 bp, Homo sapiens
  source 1..24: mol_type = unassigned DNA; note = PDCD1-tgt20
tttcccttccgctcacctccgcct

SEQ ID NO: 154 - DNA, 24 bp, Homo sapiens
  source 1..24: mol_type = unassigned DNA; note = PDCD1-tgt21
cttactgcctcagcttccctgccc

SEQ ID NO: 155 - DNA, 24 bp, Homo sapiens
  source 1..24: mol_type = unassigned DNA; note = PDCD1-tgt22
cttagcatgctctcatatttaatt

SEQ ID NO: 156 - DNA, 24 bp, Homo sapiens
  source 1..24: mol_type = unassigned DNA; note = CISH-tgt1
tttaccctaggtgagcaccccctt

SEQ ID NO: 157 - DNA, 24 bp, Homo sapiens
  source 1..24: mol_type = unassigned DNA; note = CISH-tgt2
tttgttccctgacacccaacagta

SEQ ID NO: 158 - DNA, 24 bp, Homo sapiens
  source 1..24: mol_type = unassigned DNA; note = CISH-tgt3
tttagactgctgcgctccaaagcg

SEQ ID NO: 159 - DNA, 24 bp, Homo sapiens
  source 1..24: mol_type = unassigned DNA; note = CISH-tgt4
tttggagcgcagcagtctaaaact

SEQ ID NO: 160 - DNA, 24 bp, Homo sapiens
  source 1..24: mol_type = unassigned DNA; note = CISH-tgt5
tttgtacgcagaagcagtgcccgc

SEQ ID NO: 161 - DNA, 24 bp, Homo sapiens
  source 1..24: mol_type = unassigned DNA; note = CISH-tgt6
tttaggtgtacagcagtggctggt

SEQ ID NO: 162 - DNA, 24 bp, Homo sapiens
  source 1..24: mol_type = unassigned DNA; note = CISH-tgt7
tttccggatgtggtcagccttgtg

SEQ ID NO: 163 - DNA, 24 bp, Homo sapiens
  source 1..24: mol_type = unassigned DNA; note = CISH-tgt8
tttcactgacagcgtgaacaggta

SEQ ID NO: 164 - DNA, 24 bp, Homo sapiens
  source 1..24: mol_type = unassigned DNA; note = CISH-tgt9
tttggctcactctctgtctgggct

SEQ ID NO: 165 - DNA, 24 bp, Homo sapiens
  source 1..24: mol_type = unassigned DNA; note = CISH-tgt10
tttgctggctgtggagcggactgg

SEQ ID NO: 166 - DNA, 24 bp, Homo sapiens
  source 1..24: mol_type = unassigned DNA; note = CBLB-tgt1
tttctgccattcattgagtttgcc

SEQ ID NO: 167 - DNA, 24 bp, Homo sapiens
  source 1..24: mol_type = unassigned DNA; note = CBLB-tgt2
tttcctcctcgaccaccagggttt

SEQ ID NO: 168 - DNA, 24 bp, Homo sapiens
  source 1..24: mol_type = unassigned DNA; note = CBLB-tgt3
tttcggggatttcctcctcgacca

SEQ ID NO: 169 - DNA, 24 bp, Homo sapiens
  source 1..24: mol_type = unassigned DNA; note = CBLB-tgt4
tttgggtattattgatgctattca

SEQ ID NO: 170 - DNA, 24 bp, Homo sapiens
  source 1..24: mol_type = unassigned DNA; note = CBLB-tgt5
tttgggattttggcacagtcttac

SEQ ID NO: 171 - DNA, 24 bp, Homo sapiens
  source 1..24: mol_type = unassigned DNA; note = CBLB-tgt6
tttgcctgatacatatcagcattt

SEQ ID NO: 172 - DNA, 24 bp, Homo sapiens
  source 1..24: mol_type = unassigned DNA; note = CBLB-tgt7
tttaaagtactcattctcactgag

SEQ ID NO: 173 - DNA, 24 bp, Homo sapiens
  source 1..24: mol_type = unassigned DNA; note = CBLB-tgt8
tttgactttttcataaggctatca

SEQ ID NO: 174 - DNA, 24 bp, Homo sapiens
  source 1..24: mol_type = unassigned DNA; note = CBLB-tgt9
tttaaagagtcttattgcccgttt

SEQ ID NO: 175 - DNA, 24 bp, Homo sapiens
  source 1..24: mol_type = unassigned DNA; note = CBLB-tgt10
tttcttagctgtgacacatccagg

SEQ ID NO: 176 - DNA, 24 bp, Homo sapiens
  source 1..24: mol_type = unassigned DNA; note = CBLB-tgt11
tttctgtagtcgtgctttaacttc

SEQ ID NO: 177 - DNA, 24 bp, Homo sapiens
  source 1..24: mol_type = unassigned DNA; note = CBLB-tgt12
tttggtgctatatttctgtagtcg

SEQ ID NO: 178 - DNA, 24 bp, Homo sapiens
  source 1..24: mol_type = unassigned DNA; note = CBLB-tgt13
tttcacaatataattcatattgtt

SEQ ID NO: 179 - DNA, 24 bp, Homo sapiens
  source 1..24: mol_type = unassigned DNA; note = CBLB-tgt14
tttgtcattctctgcacaaatctt

SEQ ID NO: 180 - DNA, 24 bp, Homo sapiens
  source 1..24: mol_type = unassigned DNA; note = CBLB-tgt15
tttcagctctgtaagatttgtgca

SEQ ID NO: 181 - DNA, 24 bp, Homo sapiens
  source 1..24: mol_type = unassigned DNA; note = CBLB-tgt16
tttgtgcagagaatgacaaagatg

SEQ ID NO: 182 - DNA, 24 bp, Homo sapiens
  source 1..24: mol_type = unassigned DNA; note = CBLB-tgt17
tttgatgtgcacctcttgccttac

SEQ ID NO: 183 - DNA, 24 bp, Homo sapiens
  source 1..24: mol_type = unassigned DNA; note = CBLB-tgt17
tttgatgtgcacctcttgccttac

SEQ ID NO: 184 - DNA, 24 bp, Homo sapiens
  source 1..24: mol_type = unassigned DNA; note = CBLB-tgt17
tttgatgtgcacctcttgccttac

SEQ ID NO: 185 - DNA, 24 bp, Homo sapiens
  source 1..24: mol_type = unassigned DNA; note = CBLB-tgt20
tttctgtcgttgtgaaataaaagg

SEQ ID NO: 186 - DNA, 24 bp, Homo sapiens
  source 1..24: mol_type = unassigned DNA; note = CBLB-tgt21
tttcaggtggcggtggaggaggat

SEQ ID NO: 187 - DNA, 24 bp, Homo sapiens
  source 1..24: mol_type = unassigned DNA; note = CBLB-tgt22
tttccacatgatggatgtgtctac

SEQ ID NO: 188 - DNA, 24 bp, Homo sapiens
  source 1..24: mol_type = unassigned DNA; note = CBLB-tgt23
tttgggactaatcagcttgtggga

SEQ ID NO: 189 - DNA, 24 bp, Homo sapiens
  source 1..24: mol_type = unassigned DNA; note = CBLB-tgt24
tttgaactcgctgtgattccaggt

SEQ ID NO: 190-210 - skipped (21 entries)

SEQ ID NO: 211 - RNA, 20 nt, synthetic construct
  source 1..20: mol_type = other RNA; note = Description of Artificial Sequence: Synthetic oligonucleotide; note = BM.t12_cr
agtgggggtgaattcagtgt

SEQ ID NO: 212 - DNA, 20 bp, synthetic construct
  source 1..20: mol_type = other DNA; note = Description of Artificial Sequence: Synthetic oligonucleotide; note = BM.t12_d1
  misc_feature 1: note = DNA
  misc_feature 2..20: note = RNA
agtgggggtgaattcagtgt

SEQ ID NO: 213 - DNA, 20 bp, synthetic construct
  source 1..20: mol_type = other DNA; note = Description of Artificial Sequence: Synthetic oligonucleotide; note = BM.t12_d2
  misc_feature 1: note = RNA
  misc_feature 2: note = DNA
  misc_feature 3..20: note = RNA
agtgggggtgaattcagtgt

SEQ ID NO: 214 - DNA, 20 bp, synthetic construct
  source 1..20: mol_type = other DNA; note = Description of Artificial Sequence: Synthetic oligonucleotide; note = BM.t12_d3
  misc_feature 1..2: note = RNA
  misc_feature 3: note = DNA
  misc_feature 4..20: note = RNA
agtgggggtgaattcagtgt

SEQ ID NO: 215 - DNA, 20 bp, synthetic construct
  source 1..20: mol_type = other DNA; note = Description of Artificial Sequence: Synthetic oligonucleotide; note = BM.t12_d4
  misc_feature 1..3: note = RNA
  misc_feature 4: note = DNA
  misc_feature 5..20: note = RNA
agtgggggtgaattcagtgt

SEQ ID NO: 216 - DNA, 20 bp, synthetic construct
  source 1..20: mol_type = other DNA; note = Description of Artificial Sequence: Synthetic oligonucleotide; note = BM.t12_d5
  misc_feature 1..4: note = RNA
  misc_feature 5: note = DNA
  misc_feature 6..20: note = RNA
agtgggggtgaattcagtgt

SEQ ID NO: 217 - DNA, 20 bp, synthetic construct
  source 1..20: mol_type = other DNA; note = Description of Artificial Sequence: Synthetic oligonucleotide; note = BM.t12_d6
  misc_feature 1..5: note = RNA
  misc_feature 6: note = DNA
  misc_feature 7..20: note = RNA
agtgggggtgaattcagtgt

SEQ ID NO: 218 - DNA, 20 bp, synthetic construct
  source 1..20: mol_type = other DNA; note = Description of Artificial Sequence: Synthetic oligonucleotide; note = BM.t12_d7
  misc_feature 1..6: note = RNA
  misc_feature 7: note = DNA
  misc_feature 8..20: note = RNA
agtgggggtgaattcagtgt

SEQ ID NO: 219 - DNA, 20 bp, synthetic construct
  source 1..20: mol_type = other DNA; note = Description of Artificial Sequence: Synthetic oligonucleotide; note = BM.t12_d8
  misc_feature 1..7: note = RNA
  misc_feature 8: note = DNA
  misc_feature 9..20: note = RNA
agtgggggtgaattcagtgt

SEQ ID NO: 220 - DNA, 20 bp, synthetic construct
  source 1..20: mol_type = other DNA; note = Description of Artificial Sequence: Synthetic oligonucleotide; note = BM.t12_d9
  misc_feature 1..8: note = RNA
  misc_feature 9: note = DNA
  misc_feature 10..20: note = RNA
agtgggggtgaattcagtgt

SEQ ID NO: 221 - DNA, 20 bp, synthetic construct
  source 1..20: mol_type = other DNA; note = Description of Artificial Sequence: Synthetic oligonucleotide; note = BM.t12_d10
  misc_feature 1..9: note = RNA
  misc_feature 10: note = DNA
  misc_feature 11..20: note = RNA
agtgggggtgaattcagtgt

SEQ ID NO: 222 - DNA, 20 bp, synthetic construct
  source 1..20: mol_type = other DNA; note = Description of Artificial Sequence: Synthetic oligonucleotide; note = BM.t12_d11
  misc_feature 1..10: note = RNA
  misc_feature 11: note = DNA
  misc_feature 12..20: note = RNA
agtgggggtgaattcagtgt

SEQ ID NO: 223 - DNA, 20 bp, synthetic construct
  source 1..20: mol_type = other DNA; note = Description of Artificial Sequence: Synthetic oligonucleotide; note = BM.t12_d12
  misc_feature 1..11: note = RNA
  misc_feature 12: note = DNA
  misc_feature 13..20: note = RNA
agtgggggtgaattcagtgt

SEQ ID NO: 224 - DNA, 20 bp, synthetic construct
  source 1..20: mol_type = other DNA; note = Description of Artificial Sequence: Synthetic oligonucleotide; note = BM.t12_d13
  misc_feature 1..12: note = RNA
  misc_feature 13: note = DNA
  misc_feature 14..20: note = RNA
agtgggggtgaattcagtgt

SEQ ID NO: 225 - DNA, 20 bp, synthetic construct
  source 1..20: mol_type = other DNA; note = Description of Artificial Sequence: Synthetic oligonucleotide; note = BM.t12_d14
  misc_feature 1..13: note = RNA
  misc_feature 14: note = DNA
  misc_feature 15..20: note = RNA
agtgggggtgaattcagtgt

SEQ ID NO: 226 - DNA, 20 bp, synthetic construct
  source 1..20: mol_type = other DNA; note = Description of Artificial Sequence: Synthetic oligonucleotide; note = BM.t12_d15
  misc_feature 1..14: note = RNA
  misc_feature 15: note = DNA
  misc_feature 16..20: note = RNA
agtgggggtgaattcagtgt

SEQ ID NO: 227 - DNA, 20 bp, synthetic construct
  source 1..20: mol_type = other DNA; note = Description of Artificial Sequence: Synthetic oligonucleotide; note = BM.t12_d16
  misc_feature 1..15: note = RNA
  misc_feature 16: note = DNA
  misc_feature 17..20: note = RNA
agtgggggtgaattcagtgt

SEQ ID NO: 228 - DNA, 20 bp, synthetic construct
  source 1..20: mol_type = other DNA; note = Description of Artificial Sequence: Synthetic oligonucleotide; note = BM.t12_d17
  misc_feature 1..16: note = RNA
  misc_feature 17: note = DNA
  misc_feature 18..20: note = RNA
agtgggggtgaattcagtgt

SEQ ID NO: 229 - DNA, 20 bp, synthetic construct
  source 1..20: mol_type = other DNA; note = Description of Artificial Sequence: Synthetic oligonucleotide; note = BM.t12_d18
  misc_feature 1..17: note = RNA
  misc_feature 18: note = DNA
  misc_feature 19..20: note = RNA
agtgggggtgaattcagtgt

SEQ ID NO: 230 - DNA, 20 bp, synthetic construct
  source 1..20: mol_type = other DNA; note = Description of Artificial Sequence: Synthetic oligonucleotide; note = BM.t12_d19
  misc_feature 1..18: note = RNA
  misc_feature 19: note = DNA
  misc_feature 20: note = RNA
agtgggggtgaattcagtgt

SEQ ID NO: 231 - DNA, 20 bp, synthetic construct
  source 1..20: mol_type = other DNA; note = Description of Artificial Sequence: Synthetic oligonucleotide; note = BM.t12_d20
  misc_feature 1..19: note = RNA
  misc_feature 20: note = DNA
agtgggggtgaattcagtgt

SEQ ID NO: 232 - RNA, 20 nt, synthetic construct
  source 1..20: mol_type = other RNA; note = Description of Artificial Sequence: Synthetic oligonucleotide; note = TR.t12_cr
gagtctctcagctggtacac

SEQ ID NO: 233 - DNA, 20 bp, synthetic construct
  source 1..20: mol_type = other DNA; note = Description of Artificial Sequence: Synthetic oligonucleotide; note = TR.t12_d1
  misc_feature 1: note = DNA
  misc_feature 2..20: note = RNA
gagtctctcagctggtacac

SEQ ID NO: 234 - DNA, 20 bp, synthetic construct
  source 1..20: mol_type = other DNA; note = Description of Artificial Sequence: Synthetic oligonucleotide; note = TR.t12_d2
  misc_feature 1: note = RNA
  misc_feature 2: note = DNA
  misc_feature 3..20: note = RNA
gagtctctcagctggtacac

SEQ ID NO: 235 - DNA, 20 bp, synthetic construct
  source 1..20: mol_type = other DNA; note = Description of Artificial Sequence: Synthetic oligonucleotide; note = TR.t12_d3
  misc_feature 1..2: note = RNA
  misc_feature 3: note = DNA
  misc_feature 4..20: note = RNA
gagtctctcagctggtacac

SEQ ID NO: 236 - DNA, 20 bp, synthetic construct
  source 1..20: mol_type = other DNA; note = Description of Artificial Sequence: Synthetic oligonucleotide; note = TR.t12_d4
  misc_feature 1..3: note = RNA
  misc_feature 4: note = DNA
  misc_feature 5..20: note = RNA
gagtctctcagctggtacac

SEQ ID NO: 237 - DNA, 20 bp, synthetic construct
  source 1..20: mol_type = other DNA; note = Description of Artificial Sequence: Synthetic oligonucleotide; note = TR.t12_d5
  misc_feature 1..4: note = RNA
  misc_feature 5: note = DNA
  misc_feature 6..20: note = RNA
gagtctctcagctggtacac

SEQ ID NO: 238 - DNA, 20 bp, synthetic construct
  source 1..20: mol_type = other DNA; note = Description of Artificial Sequence: Synthetic oligonucleotide; note = TR.t12_d6
  misc_feature 1..5: note = RNA
  misc_feature 6: note = DNA
  misc_feature 7..20: note = RNA
gagtctctcagctggtacac

SEQ ID NO: 239 - DNA, 20 bp, synthetic construct
  source 1..20: mol_type = other DNA; note = Description of Artificial Sequence: Synthetic oligonucleotide; note = TR.t12_d7
  misc_feature 1..6: note = RNA
  misc_feature 7: note = DNA
  misc_feature 8..20: note = RNA
gagtctctcagctggtacac

SEQ ID NO: 240 - DNA, 20 bp, synthetic construct
  source 1..20: mol_type = other DNA; note = Description of Artificial Sequence: Synthetic oligonucleotide; note = TR.t12_d8
  misc_feature 1..7: note = RNA
  misc_feature 8: note = DNA
  misc_feature 9..20: note = RNA
gagtctctcagctggtacac

SEQ ID NO: 241 - DNA, 20 bp, synthetic construct
  source 1..20: mol_type = other DNA; note = Description of Artificial Sequence: Synthetic oligonucleotide; note = TR.t12_d9
  misc_feature 1..8: note = RNA
  misc_feature 9: note = DNA
  misc_feature 10..20: note = RNA
gagtctctcagctggtacac

SEQ ID NO: 242 - DNA, 20 bp, synthetic construct
  source 1..20: mol_type = other DNA; note = Description of Artificial Sequence: Synthetic oligonucleotide; note = TR.t12_d10
  misc_feature 1..9: note = RNA
  misc_feature 10: note = DNA
  misc_feature 11..20: note = RNA
gagtctctcagctggtacac

SEQ ID NO: 243 - DNA, 20 bp, synthetic construct
  source 1..20: mol_type = other DNA; note = Description of Artificial Sequence: Synthetic oligonucleotide; note = TR.t12_d11
  misc_feature 1..10: note = RNA
  misc_feature 11: note = DNA
  misc_feature 12..20: note = RNA
gagtctctcagctggtacac

SEQ ID NO: 244 - DNA, 20 bp, synthetic construct
  source 1..20: mol_type = other DNA; note = Description of Artificial Sequence: Synthetic oligonucleotide; note = TR.t12_d12
  misc_feature 1..11: note = RNA
  misc_feature 12: note = DNA
  misc_feature 13..20: note = RNA
gagtctctcagctggtacac

SEQ ID NO: 245 - DNA, 20 bp, synthetic construct
  source 1..20: mol_type = other DNA; note = Description of Artificial Sequence: Synthetic oligonucleotide; note = TR.t12_d13
  misc_feature 1..12: note = RNA
  misc_feature 13: note = DNA
  misc_feature 14..20: note = RNA
gagtctctcagctggtacac

SEQ ID NO: 246 - DNA, 20 bp, synthetic construct
  source 1..20: mol_type = other DNA; note = Description of Artificial Sequence: Synthetic oligonucleotide; note = TR.t12_d14
  misc_feature 1..13: note = RNA
  misc_feature 14: note = DNA
  misc_feature 15..20: note = RNA
gagtctctcagctggtacac

SEQ ID NO: 247 - DNA, 20 bp, synthetic construct
  source 1..20: mol_type = other DNA; note = Description of Artificial Sequence: Synthetic oligonucleotide; note = TR.t12_d15
  misc_feature 1..14: note = RNA
  misc_feature 15: note = DNA
  misc_feature 16..20: note = RNA
gagtctctcagctggtacac

SEQ ID NO: 248 - DNA, 20 bp, synthetic construct
  source 1..20: mol_type = other DNA; note = Description of Artificial Sequence: Synthetic oligonucleotide; note = TR.t12_d16
  misc_feature 1..15: note = RNA
  misc_feature 16: note = DNA
  misc_feature 17..20: note = RNA
gagtctctcagctggtacac

SEQ ID NO: 249 - DNA, 20 bp, synthetic construct
  source 1..20: mol_type = other DNA; note = Description of Artificial Sequence: Synthetic oligonucleotide; note = TR.t12_d17
  misc_feature 1..16: note = RNA
  misc_feature 17: note = DNA
  misc_feature 18..20: note = RNA
gagtctctcagctggtacac

SEQ ID NO: 250 - DNA, 20 bp, synthetic construct
  source 1..20: mol_type = other DNA; note = Description of Artificial Sequence: Synthetic oligonucleotide; note = TR.t12_d18
  misc_feature 1..17: note = RNA
  misc_feature 18: note = DNA
  misc_feature 19..20: note = RNA
gagtctctcagctggtacac

SEQ ID NO: 251 - DNA, 20 bp, synthetic construct
  source 1..20: mol_type = other DNA; note = Description of Artificial Sequence: Synthetic oligonucleotide; note = TR.t12_d19
  misc_feature 1..18: note = RNA
  misc_feature 19: note = DNA
  misc_feature 20: note = RNA
gagtctctcagctggtacac

SEQ ID NO: 252 - DNA, 20 bp, synthetic construct
  source 1..20: mol_type = other DNA; note = Description of Artificial Sequence: Synthetic oligonucleotide; note = TR.t12_d20
  misc_feature 1..19: note = RNA
  misc_feature 20: note = DNA
gagtctctcagctggtacac

SEQ ID NO: 253 - RNA, 20 nt, synthetic construct
  source 1..20: mol_type = other RNA; note = Description of Artificial Sequence: Synthetic oligonucleotide; note = DMT.t3_cr
ctgatggtccatgtctgtta

SEQ ID NO: 254 - DNA, 20 bp, synthetic construct
  source 1..20: mol_type = other DNA; note = Description of Artificial Sequence: Synthetic oligonucleotide; note = DMT.t3_d1
  misc_feature 1: note = DNA
  misc_feature 2..20: note = RNA
ctgatggtccatgtctgtta

SEQ ID NO: 255 - DNA, 20 bp, synthetic construct
  source 1..20: mol_type = other DNA; note = Description of Artificial Sequence: Synthetic oligonucleotide; note = DMT.t3_d2
  misc_feature 1: note = RNA
  misc_feature 2: note = DNA
  misc_feature 3..20: note = RNA
ctgatggtccatgtctgtta

SEQ ID NO: 256 - DNA, 20 bp, synthetic construct
  source 1..20: mol_type = other DNA; note = Description of Artificial Sequence: Synthetic oligonucleotide; note = DMT.t3_d3
  misc_feature 1..2: note = RNA
  misc_feature 3: note = DNA
  misc_feature 4..20: note = RNA
ctgatggtccatgtctgtta

SEQ ID NO: 257 - DNA, 20 bp, synthetic construct
  source 1..20: mol_type = other DNA; note = Description of Artificial Sequence: Synthetic oligonucleotide; note = DMT.t3_d4
  misc_feature 1..3: note = RNA
  misc_feature 4: note = DNA
  misc_feature 5..20: note = RNA
ctgatggtccatgtctgtta

SEQ ID NO: 258 - DNA, 20 bp, synthetic construct
  source 1..20: mol_type = other DNA; note = Description of Artificial Sequence: Synthetic oligonucleotide; note = DMT.t3_d5
  misc_feature 1..4: note = RNA
  misc_feature 5: note = DNA
  misc_feature 6..20: note = RNA
ctgatggtccatgtctgtta

SEQ ID NO: 259 - DNA, 20 bp, synthetic construct
  source 1..20: mol_type = other DNA; note = Description of Artificial Sequence: Synthetic oligonucleotide; note = DMT.t3_d6
  misc_feature 1..5: note = RNA
  misc_feature 6: note = DNA
  misc_feature 7..20: note = RNA
ctgatggtccatgtctgtta

SEQ ID NO: 260 - DNA, 20 bp, synthetic construct
  source 1..20: mol_type = other DNA; note = Description of Artificial Sequence: Synthetic oligonucleotide; note = DMT.t3_d7
  misc_feature 1..6: note = RNA
  misc_feature 7: note = DNA
  misc_feature 8..20: note = RNA
ctgatggtccatgtctgtta

SEQ ID NO: 261 - DNA, 20 bp, synthetic construct
  source 1..20: mol_type = other DNA; note = Description of Artificial Sequence: Synthetic oligonucleotide; note = DMT.t3_d8
  misc_feature 1..7: note = RNA
  misc_feature 8: note = DNA
  misc_feature 9..20: note = RNA
ctgatggtccatgtctgtta

SEQ ID NO: 262 - DNA, 20 bp, synthetic construct
  source 1..20: mol_type = other DNA; note = Description of Artificial Sequence: Synthetic oligonucleotide; note = DMT.t3_d9
  misc_feature 1..8: note = RNA
  misc_feature 9: note = DNA
  misc_feature 10..20: note = RNA
ctgatggtccatgtctgtta

SEQ ID NO: 263 - DNA, 20 bp, synthetic construct
  source 1..20: mol_type = other DNA; note = Description of Artificial Sequence: Synthetic oligonucleotide; note = DMT.t3_d10
  misc_feature 1..9: note = RNA
  misc_feature 10: note = DNA
  misc_feature 11..20: note = RNA
ctgatggtccatgtctgtta

SEQ ID NO: 264 - DNA, 20 bp, synthetic construct
  source 1..20: mol_type = other DNA; note = Description of Artificial Sequence: Synthetic oligonucleotide; note = DMT.t3_d11
  misc_feature 1..10: note = RNA
  misc_feature 11: note = DNA
  misc_feature 12..20: note = RNA
ctgatggtccatgtctgtta

SEQ ID NO: 265 - DNA, 20 bp, synthetic construct
  source 1..20: mol_type = other DNA; note = Description of Artificial Sequence: Synthetic oligonucleotide; note = DMT.t3_d12
  misc_feature 1..11: note = RNA
  misc_feature 12: note = DNA
  misc_feature 13..20: note = RNA
ctgatggtccatgtctgtta

SEQ ID NO: 266 - DNA, 20 bp, synthetic construct
  source 1..20: mol_type = other DNA; note = Description of Artificial Sequence: Synthetic oligonucleotide; note = DMT.t3_d13
  misc_feature 1..12: note = RNA
  misc_feature 13: note = DNA
  misc_feature 14..20: note = RNA
ctgatggtccatgtctgtta

SEQ ID NO: 267 - DNA, 20 bp, synthetic construct
  source 1..20: mol_type = other DNA; note = Description of Artificial Sequence: Synthetic oligonucleotide; note = DMT.t3_d14
  misc_feature 1..13: note = RNA
  misc_feature 14: note = DNA
  misc_feature 15..20: note = RNA
ctgatggtccatgtctgtta

SEQ ID NO: 268 - DNA, 20 bp, synthetic construct
  source 1..20: mol_type = other DNA; note = Description of Artificial Sequence: Synthetic oligonucleotide; note = DMT.t3_d15
  misc_feature 1..14: note = RNA
  misc_feature 15: note = DNA
  misc_feature 16..20: note = RNA
ctgatggtccatgtctgtta

SEQ ID NO: 269 - DNA, 20 bp, synthetic construct
  source 1..20: mol_type = other DNA; note = Description of Artificial Sequence: Synthetic oligonucleotide; note = DMT.t3_d16
  misc_feature 1..15: note = RNA
  misc_feature 16: note = DNA
  misc_feature 17..20: note = RNA
ctgatggtccatgtctgtta

SEQ ID NO: 270 - DNA, 20 bp, synthetic construct
  source 1..20: mol_type = other DNA; note = Description of Artificial Sequence: Synthetic oligonucleotide; note = DMT.t3_d17
  misc_feature 1..16: note = RNA
  misc_feature 17: note = DNA
  misc_feature 18..20: note = RNA
ctgatggtccatgtctgtta

SEQ ID NO: 271 - DNA, 20 bp, synthetic construct
  source 1..20: mol_type = other DNA; note = Description of Artificial Sequence: Synthetic oligonucleotide; note = DMT.t3_d18
  misc_feature 1..17: note = RNA
  misc_feature 18: note = DNA
  misc_feature 19..20: note = RNA
ctgatggtccatgtctgtta

SEQ ID NO: 272 - DNA, 20 bp, synthetic construct
  source 1..20: mol_type = other DNA; note = Description of Artificial Sequence: Synthetic oligonucleotide; note = DMT.t3_d19
  misc_feature 1..18: note = RNA
  misc_feature 19: note = DNA
  misc_feature 20: note = RNA
ctgatggtccatgtctgtta

SEQ ID NO: 273 - DNA, 20 bp, synthetic construct
  source 1..20: mol_type = other DNA; note = Description of Artificial Sequence: Synthetic oligonucleotide; note = DMT.t3_d20
  misc_feature 1..19: note = RNA
  misc_feature 20: note = DNA
ctgatggtccatgtctgtta

SEQ ID NO: 274 - RNA, 20 nt, synthetic construct
  source 1..20: mol_type = other RNA; note = Description of Artificial Sequence: Synthetic oligonucleotide; note = BM_tgt1_act.3'_crRNA
atataagtggaggcgtcgcg

SEQ ID NO: 275 - DNA, 20 bp, synthetic construct
  source 1..20: mol_type = other DNA; note = Description of Artificial Sequence: Synthetic oligonucleotide; note = BM_tgt1_act.3'_d.1
  misc_feature 1: note = DNA
  misc_feature 2..20: note = RNA
atataagtggaggcgtcgcg

SEQ ID NO: 276 - DNA, 20 bp, synthetic construct
  source 1..20: mol_type = other DNA; note = Description of Artificial Sequence: Synthetic oligonucleotide; note = BM_tgt1_act.3'_d.2
  misc_feature 1: note = RNA
  misc_feature 2: note = DNA
  misc_feature 3..20: note = RNA
atataagtggaggcgtcgcg

SEQ ID NO: 277 - DNA, 20 bp, synthetic construct
  source 1..20: mol_type = other DNA; note = Description of Artificial Sequence: Synthetic oligonucleotide; note = BM_tgt1_act.3'_d.3
  misc_feature 1..2: note = RNA
  misc_feature 3: note = DNA
  misc_feature 4..20: note = RNA
atataagtggaggcgtcgcg

SEQ ID NO: 278 - DNA, 20 bp, synthetic construct
  source 1..20: mol_type = other DNA; note = Description of Artificial Sequence: Synthetic oligonucleotide; note = BM_tgt1_act.3'_d.4
  misc_feature 1..3: note = RNA
  misc_feature 4: note = DNA
  misc_feature 5..20: note = RNA
atataagtggaggcgtcgcg

SEQ ID NO: 279 - DNA, 20 bp, synthetic construct
  source 1..20: mol_type = other DNA; note = Description of Artificial Sequence: Synthetic oligonucleotide; note = BM_tgt1_act.3'_d.5
  misc_feature 1..4: note = RNA
  misc_feature 5: note = DNA
  misc_feature 6..20: note = RNA
atataagtggaggcgtcgcg

SEQ ID NO: 280 - DNA, 20 bp, synthetic construct
  source 1..20: mol_type = other DNA; note = Description of Artificial Sequence: Synthetic oligonucleotide; note = BM_tgt1_act.3'_d.6
  misc_feature 1..5: note = RNA
  misc_feature 6: note = DNA
  misc_feature 7..20: note = RNA
atataagtggaggcgtcgcg

SEQ ID NO: 281 - DNA, 20 bp, synthetic construct
  source 1..20: mol_type = other DNA; note = Description of Artificial Sequence: Synthetic oligonucleotide; note = BM_tgt1_act.3'_d.7
  misc_feature 1..6: note = RNA
  misc_feature 7: note = DNA
  misc_feature 8..20: note = RNA
atataagtggaggcgtcgcg

SEQ ID NO: 282 - DNA, 20 bp, synthetic construct
  source 1..20: mol_type = other DNA; note = Description of Artificial Sequence: Synthetic oligonucleotide; note = BM_tgt1_act.3'_d.8
  misc_feature 1..7: note = RNA
  misc_feature 8: note = DNA
  misc_feature 9..20: note = RNA
atataagtggaggcgtcgcg

SEQ ID NO: 283 - DNA, 20 bp, synthetic construct
  source 1..20: mol_type = other DNA; note = Description of Artificial Sequence: Synthetic oligonucleotide; note = BM_tgt1_act.3'_d.9
  misc_feature 1..8: note = RNA
  misc_feature 9: note = DNA
  misc_feature 10..20: note = RNA
atataagtggaggcgtcgcg

SEQ ID NO: 284 - DNA, 20 bp, synthetic construct
  source 1..20: mol_type = other DNA; note = Description of Artificial Sequence: Synthetic oligonucleotide; note = BM_tgt1_act.3'_d.10
  misc_feature 1..9: note = RNA
  misc_feature 10: note = DNA
  misc_feature 11..20: note = RNA
atataagtggaggcgtcgcg

SEQ ID NO: 285 - DNA, 20 bp, synthetic construct
  source 1..20: mol_type = other DNA; note = Description of Artificial Sequence: Synthetic oligonucleotide; note = BM_tgt1_act.3'_d.11
  misc_feature 1..10: note = RNA
  misc_feature 11: note = DNA
  misc_feature 12..20: note = RNA
atataagtggaggcgtcgcg

SEQ ID NO: 286 - DNA, 20 bp, synthetic construct
  source 1..20: mol_type = other DNA; note = Description of Artificial Sequence: Synthetic oligonucleotide; note = BM_tgt1_act.3'_d.12
  misc_feature 1..11: note = RNA
  misc_feature 12: note = DNA
  misc_feature 13..20: note = RNA
atataagtggaggcgtcgcg

SEQ ID NO: 287 - DNA, 20 bp, synthetic construct
  source 1..20: mol_type = other DNA; note = Description of Artificial Sequence: Synthetic oligonucleotide; note = BM_tgt1_act.3'_d.13
  misc_feature 1..12: note = RNA
  misc_feature 13: note = DNA
  misc_feature 14..20: note = RNA
atataagtggaggcgtcgcg

SEQ ID NO: 288 - DNA, 20 bp, synthetic construct
  source 1..20: mol_type = other DNA; note = Description of Artificial Sequence: Synthetic oligonucleotide; note = BM_tgt1_act.3'_d.14
  misc_feature 1..13: note = RNA
  misc_feature 14: note = DNA
  misc_feature 15..20: note = RNA
atataagtggaggcgtcgcg

SEQ ID NO: 289 - DNA, 20 bp, synthetic construct
  source 1..20: mol_type = other DNA; note = Description of Artificial Sequence: Synthetic oligonucleotide; note = BM_tgt1_act.3'_d.15
  misc_feature 1..14: note = RNA
  misc_feature 15: note = DNA
  misc_feature 16..20: note = RNA
atataagtggaggcgtcgcg

SEQ ID NO: 290 - DNA, 20 bp, synthetic construct
  source 1..20: mol_type = other DNA; note = Description of Artificial Sequence: Synthetic oligonucleotide; note = BM_tgt1_act.3'_d.16
  misc_feature 1..15: note = RNA
  misc_feature 16: note = DNA
  misc_feature 17..20: note = RNA
atataagtggaggcgtcgcg

SEQ ID NO: 291 - DNA, 20 bp, synthetic construct
  source 1..20: mol_type = other DNA; note = Description of Artificial Sequence: Synthetic oligonucleotide; note = BM_tgt1_act.3'_d.17
  misc_feature 1..16: note = RNA
  misc_feature 17: note = DNA
  misc_feature 18..20: note = RNA
atataagtggaggcgtcgcg

SEQ ID NO: 292 - DNA, 20 bp, synthetic construct
  source 1..20: mol_type = other DNA; note = Description of Artificial Sequence: Synthetic oligonucleotide; note = BM_tgt1_act.3'_d.18
  misc_feature 1..17: note = RNA
  misc_feature 18: note = DNA
  misc_feature 19..20: note = RNA
atataagtggaggcgtcgcg

SEQ ID NO: 293 - DNA, 20 bp, synthetic construct
  source 1..20: mol_type = other DNA; note = Description of Artificial Sequence: Synthetic oligonucleotide; note = BM_tgt1_act.3'_d.19
  misc_feature 1..18: note = RNA
  misc_feature 19: note = DNA
  misc_feature 20: note = RNA
atataagtggaggcgtcgcg

SEQ ID NO: 294 - DNA, 20 bp, synthetic construct
  source 1..20: mol_type = other DNA; note = Description of Artificial Sequence: Synthetic oligonucleotide; note = BM_tgt1_act.3'_d.20
  misc_feature 1..19: note = RNA
  misc_feature 20: note = DNA
atataagtggaggcgtcgcg

SEQ ID NO: 295 - RNA, 20 nt, synthetic construct
  source 1..20: mol_type = other RNA; note = Description of Artificial Sequence: Synthetic oligonucleotide; note = BM.in_t12_act.3'_crRNA
tctggaggctctcaaggact

SEQ ID NO: 296 - DNA, 20 bp, synthetic construct
  source 1..20: mol_type = other DNA; note = Description of Artificial Sequence: Synthetic oligonucleotide; note = BM.in_t12_cr-3'_d.1
  misc_feature 1: note = DNA
  misc_feature 2..20: note = RNA
tctggaggctctcaaggact

SEQ ID NO: 297 - DNA, 20 bp, synthetic construct
  source 1..20: mol_type = other DNA; note = Description of Artificial Sequence: Synthetic oligonucleotide; note = BM.in_t12_cr-3'_d.2
  misc_feature 1: note = RNA
  misc_feature 2: note = DNA
  misc_feature 3..20: note = RNA
tctggaggctctcaaggact

SEQ ID NO: 298 - DNA, 20 bp, synthetic construct
  source 1..20: mol_type = other DNA; note = Description of Artificial Sequence: Synthetic oligonucleotide; note = BM.in_t12_cr-3'_d.3
  misc_feature 1..2: note = RNA
  misc_feature 3: note = DNA
  misc_feature 4..20: note = RNA
tctggaggctctcaaggact

SEQ ID NO: 299 - DNA, 20 bp, synthetic construct
  source 1..20: mol_type = other DNA; note = Description of Artificial Sequence: Synthetic oligonucleotide; note = BM.in_t12_cr-3'_d.4
  misc_feature 1..3: note = RNA
  misc_feature 4: note = DNA
  misc_feature 5..20: note = RNA
tctggaggctctcaaggact

SEQ ID NO: 300 - DNA, 20 bp, synthetic construct
  source 1..20: mol_type = other DNA; note = Description of Artificial Sequence: Synthetic oligonucleotide; note = BM.in_t12_cr-3'_d.5
  misc_feature 1..4: note = RNA
  misc_feature 5: note = DNA
  misc_feature 6..20: note = RNA
tctggaggctctcaaggact

SEQ ID NO: 301 - DNA, 20 bp, synthetic construct
  source 1..20: mol_type = other DNA; note = Description of Artificial Sequence: Synthetic oligonucleotide; note = BM.in_t12_cr-3'_d.6
  misc_feature 1..5: note = RNA
  misc_feature 6: note = DNA
  misc_feature 7..20: note = RNA
tctggaggctctcaaggact

SEQ ID NO: 302 - DNA, 20 bp, synthetic construct
  source 1..20: mol_type = other DNA; note = Description of Artificial Sequence: Synthetic oligonucleotide; note = BM.in_t12_cr-3'_d.7
  misc_feature 1..6: note = RNA
  misc_feature 7: note = DNA
  misc_feature 8..20: note = RNA
tctggaggctctcaaggact

SEQ ID NO: 303 - DNA, 20 bp, synthetic construct
  source 1..20: mol_type = other DNA; note = Description of Artificial Sequence: Synthetic oligonucleotide; note = BM.in_t12_cr-3'_d.8
  misc_feature 1..7: note = RNA
  misc_feature 8: note = DNA
  misc_feature 9..20: note = RNA
tctggaggctctcaaggact

SEQ ID NO: 304 - DNA, 20 bp, synthetic construct
  source 1..20: mol_type = other DNA; note = Description of Artificial Sequence: Synthetic oligonucleotide; note = BM.in_t12_cr-3'_d.9
  misc_feature 1..8: note = RNA
  misc_feature 9: note = DNA
  misc_feature 10..20: note = RNA
tctggaggctctcaaggact

SEQ ID NO: 305 - DNA, 20 bp, synthetic construct
  source 1..20: mol_type = other DNA; note = Description of Artificial Sequence: Synthetic oligonucleotide; note = BM.in_t12_cr-3'_d.10
  misc_feature 1..9: note = RNA
  misc_feature 10: note = DNA
  misc_feature 11..20: note = RNA
tctggaggctctcaaggact

SEQ ID NO: 306 - DNA, 20 bp, synthetic construct
  source 1..20: mol_type = other DNA; note = Description of Artificial Sequence: Synthetic oligonucleotide; note = BM.in_t12_cr-3'_d.11
  misc_feature 1..10: note = RNA
  misc_feature 11: note = DNA
  misc_feature 12..20: note = RNA
tctggaggctctcaaggact

SEQ ID NO: 307 - DNA, 20 bp, synthetic construct
  source 1..20: mol_type = other DNA; note = Description of Artificial Sequence: Synthetic oligonucleotide; note = BM.in_t12_cr-3'_d.12
  misc_feature 1..11: note = RNA
  misc_feature 12: note = DNA
  misc_feature 13..20: note = RNA
tctggaggctctcaaggact

SEQ ID NO: 308 - DNA, 20 bp, synthetic construct
  source 1..20: mol_type = other DNA; note = Description of Artificial Sequence: Synthetic oligonucleotide; note = BM.in_t12_cr-3'_d.13
  misc_feature 1..12: note = RNA
  misc_feature 13: note = DNA
  misc_feature 14..20: note = RNA
tctggaggctctcaaggact

SEQ ID NO: 309 - DNA, 20 bp, synthetic construct
  source 1..20: mol_type = other DNA; note = Description of Artificial Sequence: Synthetic oligonucleotide; note = BM.in_t12_cr-3'_d.14
  misc_feature 1..13: note = RNA
  misc_feature 14: note = DNA
  misc_feature 15..20: note = RNA
tctggaggctctcaaggact

SEQ ID NO: 310 - DNA, 20 bp, synthetic construct
  source 1..20: mol_type = other DNA; note = Description of Artificial Sequence: Synthetic oligonucleotide; note = BM.in_t12_cr-3'_d.15
  misc_feature 1..14: note = RNA
  misc_feature 15: note = DNA
  misc_feature 16..20: note = RNA
tctggaggctctcaaggact

SEQ ID NO: 311 - DNA, 20 bp, synthetic construct
  source 1..20: mol_type = other DNA; note = Description of Artificial Sequence: Synthetic oligonucleotide; note = BM.in_t12_cr-3'_d.16
  misc_feature 1..15: note = RNA
  misc_feature 16: note = DNA
  misc_feature 17..20: note = RNA
tctggaggctctcaaggact

SEQ ID NO: 312 - DNA, 20 bp, synthetic construct
  source 1..20: mol_type = other DNA; note = Description of Artificial Sequence: Synthetic oligonucleotide; note = BM.in_t12_cr-3'_d.17
  misc_feature 1..16: note = RNA
  misc_feature 17: note = DNA
  misc_feature 18..20: note = RNA
tctggaggctctcaaggact

SEQ ID NO: 313 - DNA, 20 bp, synthetic construct
  source 1..20: mol_type = other DNA; note = Description of Artificial Sequence: Synthetic oligonucleotide; note = BM.in_t12_cr-3'_d.18
  misc_feature 1..17: note = RNA
  misc_feature 18: note = DNA
  misc_feature 19..20: note = RNA
tctggaggctctcaaggact

SEQ ID NO: 314 - DNA, 20 bp, synthetic construct
  source 1..20: mol_type = other DNA; note = Description of Artificial Sequence: Synthetic oligonucleotide; note = BM.in_t12_cr-3'_d.19
  misc_feature 1..18: note = RNA
  misc_feature 19: note = DNA
  misc_feature 20: note = RNA
tctggaggctctcaaggact

SEQ ID NO: 315 - DNA, 20 bp, synthetic construct
  source 1..20: mol_type = other DNA; note = Description of Artificial Sequence: Synthetic oligonucleotide; note = BM.in_t12_cr-3'_d.20
  misc_feature 1..19: note = RNA
  misc_feature 20: note = DNA
tctggaggctctcaaggact

SEQ ID NO: 316 - RNA, 20 nt, synthetic construct
  source 1..20: mol_type = other RNA; note = Description of Artificial Sequence: Synthetic oligonucleotide; note = TR.t12_cr
gagtctctcagctggtacac

SEQ ID NO: 317 - DNA, 20 bp, synthetic construct
  source 1..20: mol_type = other DNA; note = Description of Artificial Sequence: Synthetic oligonucleotide; note = TR.t12_d20.19
  misc_feature 1..18: note = RNA
  misc_feature 19..20: note = DNA
gagtctctcagctggtacac

SEQ ID NO: 318 - DNA, 20 bp, synthetic construct
  source 1..20: mol_type = other DNA; note = Description of Artificial Sequence: Synthetic oligonucleotide; note = TR.t12_d20.17
  misc_feature 1..16: note = RNA
  misc_feature 17: note = DNA
  misc_feature 18..19: note = RNA
  misc_feature 20: note = DNA
gagtctctcagctggtacac

SEQ ID NO: 319 - DNA, 20 bp, synthetic construct
  source 1..20: mol_type = other DNA; note = Description of Artificial Sequence: Synthetic oligonucleotide; note = TR.t12_d19.15
  misc_feature 1..14: note = RNA
  misc_feature 15: note = DNA
  misc_feature 16..18: note = RNA
  misc_feature 19: note = DNA
  misc_feature 20: note = RNA
gagtctctcagctggtacac

SEQ ID NO: 320 - DNA, 20 bp, synthetic construct
  source 1..20: mol_type = other DNA; note = Description of Artificial Sequence: Synthetic oligonucleotide; note = TR.t12_d11.10
  misc_feature 1..9: note = RNA
  misc_feature 10..11: note = DNA
  misc_feature 12..20: note = RNA
gagtctctcagctggtacac

SEQ ID NO: 321 - DNA, 20 bp, synthetic construct
  source 1..20: mol_type = other DNA; note = Description of Artificial Sequence: Synthetic oligonucleotide; note = TR.t12_d20.11.1
  misc_feature 1: note = DNA
  misc_feature 2..10: note = RNA
  misc_feature 11: note = DNA
  misc_feature 12..19: note = RNA
  misc_feature 20: note = DNA
gagtctctcagctggtacac

SEQ ID NO: 322 - DNA, 20 bp, synthetic construct
  source 1..20: mol_type = other DNA; note = Description of Artificial Sequence: Synthetic oligonucleotide; note = TR.t12_d17.11
  misc_feature 1..10: note = RNA
  misc_feature 11: note = DNA
  misc_feature 12..16: note = RNA
  misc_feature 17: note = DNA
  misc_feature 18..20: note = RNA
gagtctctcagctggtacac

SEQ ID NO: 323 - DNA, 20 bp, synthetic construct
  source 1..20: mol_type = other DNA; note = Description of Artificial Sequence: Synthetic oligonucleotide; note = TR.t12_d2.1
  misc_feature 1..2: note = DNA
  misc_feature 3..20: note = RNA
gagtctctcagctggtacac

SEQ ID NO: 324 - DNA, 20 bp, synthetic construct
  source 1..20: mol_type = other DNA; note = Description of Artificial Sequence: Synthetic oligonucleotide; note = TR.t12_d15.10.11
  misc_feature 1..9: note = RNA
  misc_feature 10..11: note = DNA
  misc_feature 12..14: note = RNA
  misc_feature 15: note = DNA
  misc_feature 16..20: note = RNA
gagtctctcagctggtacac

SEQ ID NO: 325 - DNA, 20 bp, synthetic construct
  source 1..20: mol_type = other DNA; note = Description of Artificial Sequence: Synthetic oligonucleotide; note = TR.t12_d20.17.10.1
  misc_feature 1: note = DNA
  misc_feature 2..9: note = RNA
  misc_feature 10: note = DNA
  misc_feature 11..16: note = RNA
  misc_feature 17: note = DNA
  misc_feature 18..19: note = RNA
  misc_feature 20: note = DNA
gagtctctcagctggtacac

SEQ ID NO: 326 - DNA, 20 bp, synthetic construct
  source 1..20: mol_type = other DNA; note = Description of Artificial Sequence: Synthetic oligonucleotide; note = TR.t12_d18.15.2
  misc_feature 1: note = RNA
  misc_feature 2: note = DNA
  misc_feature 3..14: note = RNA
  misc_feature 15: note = DNA
  misc_feature 16..18: note = RNA
  misc_feature 19: note = DNA
  misc_feature 20: note = RNA
gagtctctcagctggtacac

SEQ ID NO: 327 - DNA, 20 bp, synthetic construct
  source 1..20: mol_type = other DNA; note = Description of Artificial Sequence: Synthetic oligonucleotide; note = TR.t12_d19.15.11.10.3
  misc_feature 1: note = RNA
  misc_feature 2: note = DNA
  misc_feature 3..9: note = RNA
  misc_feature 10..11: note = DNA
  misc_feature 12..14: note = RNA
  misc_feature 15: note = DNA
  misc_feature 16..18: note = RNA
  misc_feature 19: note = DNA
  misc_feature 20: note = RNA
gagtctctcagctggtacac

SEQ ID NO: 328 - DNA, 20 bp, synthetic construct
  source 1..20: mol_type = other DNA; note = Description of Artificial Sequence: Synthetic oligonucleotide; note = TR.t12_d20.17.15.10.1
  misc_feature 1: note = DNA
  misc_feature 2..9: note = RNA
  misc_feature 10: note = DNA
  misc_feature 11..14: note = RNA
  misc_feature 15: note = DNA
  misc_feature 16: note = RNA
  misc_feature 17: note = DNA
  misc_feature 18..19: note = RNA
  misc_feature 20: note = DNA
gagtctctcagctggtacac

SEQ ID NO: 329 - DNA, 20 bp, synthetic construct
  source 1..20: mol_type = other DNA; note = Description of Artificial Sequence: Synthetic oligonucleotide; note = TR.t12_d_7
  misc_feature 1: note = RNA
  misc_feature 2: note = DNA
  misc_feature 3..9: note = RNA
  misc_feature 10..11: note = DNA
  misc_feature 12..14: note = RNA
  misc_feature 15: note = DNA
  misc_feature 16: note = RNA
  misc_feature 17: note = DNA
  misc_feature 18: note = RNA
  misc_feature 19..20: note = DNA
gagtctctcagctggtacac

SEQ ID NO: 330 - RNA, 20 nt, synthetic construct
  source 1..20: mol_type = other RNA; note = Description of Artificial Sequence: Synthetic oligonucleotide; note = BM_tgt1_act.3'_crRNA
atataagtggaggcgtcgcg

SEQ ID NO: 331 - DNA, 20 bp, synthetic construct
  source 1..20: mol_type = other DNA; note = Description of Artificial Sequence: Synthetic oligonucleotide; note = BM.t12_d20.17
  misc_feature 1..16: note = RNA
  misc_feature 17: note = DNA
  misc_feature 18..19: note = RNA
  misc_feature 20: note = DNA
agtgggggtgaattcagtgt

SEQ ID NO: 332 - DNA, 20 bp, synthetic construct
  source 1..20: mol_type = other DNA; note = Description of Artificial Sequence: Synthetic oligonucleotide; note = BM.t12_d15.11
  misc_feature 1..10: note = RNA
  misc_feature 11: note = DNA
  misc_feature 12..14: note = RNA
  misc_feature 15: note = DNA
  misc_feature 16..20: note = RNA
agtgggggtgaattcagtgt

SEQ ID NO: 333 - DNA, 20 bp, synthetic construct
  source 1..20: mol_type = other DNA; note = Description of Artificial Sequence: Synthetic oligonucleotide; note = BM.t12_d15.14
  misc_feature 1..13: note = RNA
  misc_feature 14..15: note = DNA
  misc_feature 16..20: note = RNA
agtgggggtgaattcagtgt

SEQ ID NO: 334 - DNA, 20 bp, synthetic construct
  source 1..20: mol_type = other DNA; note = Description of Artificial Sequence: Synthetic oligonucleotide; note = BM.t12_d11.10
  misc_feature 1..9: note = RNA
  misc_feature 10..11: note = DNA
  misc_feature 12..20: note = RNA
agtgggggtgaattcagtgt

SEQ ID NO: 335 - DNA, 20 bp, synthetic construct
  source 1..20: mol_type = other DNA; note = Description of Artificial Sequence: Synthetic oligonucleotide; note = BM.t12_d20.11.1
  misc_feature 1: note = DNA
  misc_feature 2..10: note = RNA
  misc_feature 11: note = DNA
  misc_feature 12..19: note = RNA
  misc_feature 20: note = DNA
agtgggggtgaattcagtgt

SEQ ID NO: 336 - DNA, 20 bp, synthetic construct
  source 1..20: mol_type = other DNA; note = Description of Artificial Sequence: Synthetic oligonucleotide; note = BM.t12_d17.11
  misc_feature 1..10: note = RNA
  misc_feature 11: note = DNA
  misc_feature 12..16: note = RNA
  misc_feature 17: note = DNA
  misc_feature 18..20: note = RNA
agtgggggtgaattcagtgt

SEQ ID NO: 337 - DNA, 20 bp, synthetic construct
  source 1..20: mol_type = other DNA; note = Description of Artificial Sequence: Synthetic oligonucleotide; note = BM.t12_d2.1
  misc_feature 1..2: note = DNA
  misc_feature 3..20: note = RNA
agtgggggtgaattcagtgt

SEQ ID NO: 338 - DNA, 20 bp, synthetic construct
  source 1..20: mol_type = other DNA; note = Description of Artificial Sequence: Synthetic oligonucleotide; note = BM.t12_d20.17.15.2
  misc_feature 1: note = RNA
  misc_feature 2: note = DNA
  misc_feature 3..14: note = RNA
  misc_feature 15: note = DNA
  misc_feature 16: note = RNA
  misc_feature 17: note = DNA
  misc_feature 18..19: note = RNA
  misc_feature 20: note = DNA
agtgggggtgaattcagtgt

SEQ ID NO: 339 - DNA, 20 bp, synthetic construct
  source 1..20: mol_type = other DNA; note = Description of Artificial Sequence: Synthetic oligonucleotide; note = BM.t12_d15.14.11.10
  misc_feature 1..9: note = RNA
  misc_feature 10..11: note = DNA
  misc_feature 12..13: note = RNA
  misc_feature 14..15: note = DNA
  misc_feature 16..20: note = RNA
agtgggggtgaattcagtgt

SEQ ID NO: 340 - DNA, 20 bp, synthetic construct
  source 1..20: mol_type = other DNA; note = Description of Artificial Sequence: Synthetic oligonucleotide; note = BM.t12_d_6a
  misc_feature 1..2: note = DNA
  misc_feature 3..9: note = RNA
  misc_feature 10..11: note = DNA
  misc_feature 12..16: note = RNA
  misc_feature 17: note = DNA
  misc_feature 18..19: note = RNA
  misc_feature 20: note = DNA
agtgggggtgaattcagtgt

SEQ ID NO: 341 - DNA, 20 bp, synthetic construct
  source 1..20: mol_type = other DNA; note = Description of Artificial Sequence: Synthetic oligonucleotide; note = BM.t12_d_8
  misc_feature 1: note = DNA
  misc_feature 2: note = RNA
  misc_feature 3: note = DNA
  misc_feature 4..8: note = RNA
  misc_feature 9..11: note = DNA
  misc_feature 12..13: note = RNA
  misc_feature 14..15: note = DNA
  misc_feature 16: note = RNA
  misc_feature 17: note = DNA
  misc_feature 18..20: note = RNA
agtgggggtgaattcagtgt

SEQ ID NO: 342 - DNA, 20 bp, synthetic construct
  source 1..20: mol_type = other DNA; note = Description of Artificial Sequence: Synthetic oligonucleotide; note = BM.t12_d_6b
  misc_feature 1: note = DNA
  misc_feature 2..9: note = RNA
  misc_feature 10..11: note = DNA
  misc_feature 12: note = RNA
  misc_feature 13: note = DNA
  misc_feature 14..15: note = RNA
  misc_feature 16: note = DNA
  misc_feature 17..18: note = RNA
  misc_feature 19: note = DNA
  misc_feature 20: note = RNA
agtgggggtgaattcagtgt

SEQ ID NO: 343 - DNA, 20 bp, synthetic construct
  source 1..20: mol_type = other DNA; note = Description of Artificial Sequence: Synthetic oligonucleotide; note = BM.t12_d16.9.1-3
  misc_feature 1..3: note = DNA
  misc_feature 4..8: note = RNA
  misc_feature 9: note = DNA
  misc_feature 10..15: note = RNA
  misc_feature 16: note = DNA
  misc_feature 17..20: note = RNA
agtgggggtgaattcagtgt

SEQ ID NO: 344 - DNA, 20 bp, synthetic construct
  source 1..20: mol_type = other DNA; note = Description of Artificial Sequence: Synthetic oligonucleotide; note = BM.t12_d_10
  misc_feature 1..10: note = RNA
  misc_feature 11..20: note = DNA
agtgggggtgaattcagtgt

SEQ ID NO: 345 - RNA, 20 nt, synthetic construct
  source 1..20: mol_type = other RNA; note = Description of Artificial Sequence: Synthetic oligonucleotide; note = BM_tgt1_cr-3'
atataagtggaggcgtcgcg

SEQ ID NO: 346 - DNA, 20 bp, synthetic construct
  source 1..20: mol_type = other DNA; note = Description of Artificial Sequence: Synthetic oligonucleotide; note = BM_tgt1_cr-3'_d.20.11.1
  misc_feature 1: note = DNA
  misc_feature 2..10: note = RNA
  misc_feature 11: note = DNA
  misc_feature 12..19: note = RNA
  misc_feature 20: note = DNA
atataagtggaggcgtcgcg

SEQ ID NO: 347 - DNA, 20 bp, synthetic construct
  source 1..20: mol_type = other DNA; note = Description of Artificial Sequence: Synthetic oligonucleotide; note = BM_tgt1_cr-3'_d.19.14.8
  misc_feature 1..7: note = RNA
  misc_feature 8: note = DNA
  misc_feature 9..13: note = RNA
  misc_feature 14: note = DNA
  misc_feature 15..18: note = RNA
  misc_feature 19: note = DNA
  misc_feature 20: note = RNA
atataagtggaggcgtcgcg

SEQ ID NO: 348 - DNA, 20 bp, synthetic construct
  source 1..20: mol_type = other DNA; note = Description of Artificial Sequence: Synthetic oligonucleotide; note = BM_tgt1_cr-3'_d.19.17.14.8
  misc_feature 1..7: note = RNA
  misc_feature 8: note = DNA
  misc_feature 9..13: note = RNA
  misc_feature 14: note = DNA
  misc_feature 15..16: note = RNA
  misc_feature 17: note = DNA
  misc_feature 18: note = RNA
  misc_feature 19: note = DNA
  misc_feature 20: note = RNA
atataagtggaggcgtcgcg

SEQ ID NO: 349 - DNA, 20 bp, synthetic construct
  source 1..20: mol_type = other DNA; note = Description of Artificial Sequence: Synthetic oligonucleotide; note = BM_tgt1_cr-3'_d.12.11.8
  misc_feature 1..8: note = RNA
  misc_feature 9: note = DNA
  misc_feature 10: note = RNA
  misc_feature 11..12: note = DNA
  misc_feature 13..20: note = RNA
atataagtggaggcgtcgcg

SEQ ID NO: 350 - DNA, 20 bp, synthetic construct
  source 1..20: mol_type = other DNA; note = Description of Artificial Sequence: Synthetic oligonucleotide; note = BM_tgt1_cr-3'_d.18.16.10
  misc_feature 1..9: note = RNA
  misc_feature 10: note = DNA
  misc_feature 11..15: note = RNA
  misc_feature 16: note = DNA
  misc_feature 17: note = RNA
  misc_feature 18: note = DNA
  misc_feature 19..20: note = RNA
atataagtggaggcgtcgcg

SEQ ID NO: 351 - DNA, 20 bp, synthetic construct
  source 1..20: mol_type = other DNA; note = Description of Artificial Sequence: Synthetic oligonucleotide; note = BM_tgt1_cr-3'_d.19.17.15.1
  misc_feature 1: note = DNA
  misc_feature 2..14: note = RNA
  misc_feature 15: note = DNA
  misc_feature 16: note = RNA
  misc_feature 17: note = DNA
  misc_feature 18: note = RNA
  misc_feature 19: note = DNA
  misc_feature 20: note = RNA
atataagtggaggcgtcgcg

SEQ ID NO: 352 - DNA, 20 bp, synthetic construct
  source 1..20: mol_type = other DNA; note = Description of Artificial Sequence: Synthetic oligonucleotide; note = BM_tgt1_cr-3'_d.19-16
  misc_feature 1..15: note = RNA
  misc_feature 16..19: note = DNA
  misc_feature 20: note = RNA
atataagtggaggcgtcgcg

SEQ ID NO: 353 - RNA, 20 nt, synthetic construct
  source 1..20: mol_type = other RNA; note = Description of Artificial Sequence: Synthetic oligonucleotide; note = BM.in_t12_cr-3'
tctggaggctctcaaggact

SEQ ID NO: 354 - DNA, 20 bp, synthetic construct
  source 1..20: mol_type = other DNA; note = Description of Artificial Sequence: Synthetic oligonucleotide; note = BM.in_t12_cr-3'_d.01.11.20
  misc_feature 1: note = DNA
  misc_feature 2..10: note = RNA
  misc_feature 11: note = DNA
  misc_feature 12..19: note = RNA
  misc_feature 20: note = DNA
tctggaggctctcaaggact

SEQ ID NO: 355 - DNA, 20 bp, synthetic construct
  source 1..20: mol_type = other DNA; note = Description of Artificial Sequence: Synthetic oligonucleotide; note = BM.in_t12_cr-3'_d.11.17.20
  misc_feature 1..10: note = RNA
  misc_feature 11: note = DNA
  misc_feature 12..16: note = RNA
  misc_feature 17: note = DNA
  misc_feature 18..19: note = RNA
  misc_feature 20: note = DNA
tctggaggctctcaaggact

SEQ ID NO: 356 - DNA, 20 bp, synthetic construct
  source 1..20: mol_type = other DNA; note = Description of Artificial Sequence: Synthetic oligonucleotide; note = BM.in_t12_cr-3'_d.08.09.10.11
  misc_feature 1..7: note = RNA
  misc_feature 8..11: note = DNA
  misc_feature 12..20: note = RNA
tctggaggctctcaaggact

SEQ ID NO: 357 - DNA, 20 bp, synthetic construct
  source 1..20: mol_type = other DNA; note = Description of Artificial Sequence: Synthetic oligonucleotide; note = BM.in_t12_cr-3'_d.01.08.10.17
  misc_feature 1: note = DNA
  misc_feature 2..7: note = RNA
  misc_feature 8: note = DNA
  misc_feature 9: note = RNA
  misc_feature 10: note = DNA
  misc_feature 11..16: note = RNA
  misc_feature 17: note = DNA
  misc_feature 18..20: note = RNA
tctggaggctctcaaggact

SEQ ID NO: 358 - DNA, 20 bp, synthetic construct
  source 1..20: mol_type = other DNA; note = Description of Artificial Sequence: Synthetic oligonucleotide; note = BM.in_t12_cr-3'_d.17.19.20
  misc_feature 1..16: note = RNA
  misc_feature 17: note = DNA
  misc_feature 18: note = RNA
  misc_feature 19..20: note = DNA
tctggaggctctcaaggact

SEQ ID NO: 359 - DNA, 20 bp, synthetic construct
  source 1..20: mol_type = other DNA; note = Description of Artificial Sequence: Synthetic oligonucleotide; note = BM.in_t12_cr-3'_d.08.09.19.20
  misc_feature 1..7: note = RNA
  misc_feature 8..9: note = DNA
  misc_feature 10: note = RNA
  misc_feature 11: note = DNA
  misc_feature 12..19: note = RNA
  misc_feature 20: note = DNA
tctggaggctctcaaggact

SEQ ID NO: 360 - DNA, 20 bp, synthetic construct
  source 1..20: mol_type = other DNA; note = Description of Artificial Sequence: Synthetic oligonucleotide; note = BM.in_t12_cr-3'_d01.10.11.17.19.20
  misc_feature 1: note = DNA
  misc_feature 2..9: note = RNA
  misc_feature 10..11: note = DNA
  misc_feature 12..16: note = RNA
  misc_feature 17: note = DNA
  misc_feature 18: note = RNA
  misc_feature 19..20: note = DNA
tctggaggctctcaaggact

SEQ ID NO: 361 - RNA, 20 nt, synthetic construct
  source 1..20: mol_type = other RNA; note = Description of Artificial Sequence: Synthetic oligonucleotide; note = DNMt_crRNA
ctgatggtccatgtctgtta

SEQ ID NO: 362 - DNA, 20 bp, synthetic construct
  source 1..20: mol_type = other DNA; note = Description of Artificial Sequence: Synthetic oligonucleotide; note = DMT.t3_d1.20
  misc_feature 1: note = DNA
  misc_feature 2..19: note = RNA
  misc_feature 20: note = DNA
ctgatggtccatgtctgtta

SEQ ID NO: 363 - DNA, 20 bp, synthetic construct
  source 1..20: mol_type = other DNA; note = Description of Artificial Sequence: Synthetic oligonucleotide; note = DMT.t3_d14.20
  misc_feature 1..13: note = RNA
  misc_feature 14: note = DNA
  misc_feature 15..19: note = RNA
  misc_feature 20: note = DNA
ctgatggtccatgtctgtta

SEQ ID NO: 364 - DNA, 20 bp, synthetic construct
  source 1..20: mol_type = other DNA; note = Description of Artificial Sequence: Synthetic oligonucleotide; note = DMT.t3_d8.19
  misc_feature 1..7: note = RNA
  misc_feature 8: note = DNA
  misc_feature 9..18: note = RNA
  misc_feature 19: note = DNA
  misc_feature 20: note = RNA
ctgatggtccatgtctgtta

SEQ ID NO: 365 - DNA, 20 bp, synthetic construct
  source 1..20: mol_type = other DNA; note = Description of Artificial Sequence: Synthetic oligonucleotide; note = DMT.t3_d8.14
  misc_feature 1..7: note = RNA
  misc_feature 8: note = DNA
  misc_feature 9..13: note = RNA
  misc_feature 14: note = DNA
  misc_feature 15..20: note = RNA
ctgatggtccatgtctgtta

SEQ ID NO: 366 - DNA, 20 bp, synthetic construct
  source 1..20: mol_type = other DNA; note = Description of Artificial Sequence: Synthetic oligonucleotide; note = DMT.t3_d1.8.20
  misc_feature 1: note = DNA
  misc_feature 2..7: note = RNA
  misc_feature 8: note = DNA
  misc_feature 9..19: note = RNA
  misc_feature 20: note = DNA
ctgatggtccatgtctgtta

SEQ ID NO: 367 - DNA, 20 bp, synthetic construct
  source 1..20: mol_type = other DNA; note = Description of Artificial Sequence: Synthetic oligonucleotide; note = DMT.t3_d8-10
  misc_feature 1..7: note = RNA
  misc_feature 8..10: note = DNA
  misc_feature 11..20: note = RNA
ctgatggtccatgtctgtta

SEQ ID NO: 368 - DNA, 20 bp, synthetic construct
  source 1..20: mol_type = other DNA; note = Description of Artificial Sequence: Synthetic oligonucleotide; note = DMT.t3_d8.9.15
  misc_feature 1..7: note = RNA
  misc_feature 8..9: note = DNA
  misc_feature 10..14: note = RNA
  misc_feature 15: note = DNA
  misc_feature 16..20: note = RNA
ctgatggtccatgtctgtta

SEQ ID NO: 369 - DNA, 20 bp, synthetic construct
  source 1..20: mol_type = other DNA; note = Description of Artificial Sequence: Synthetic oligonucleotide; note = DMT.t3_d1.8.19
  misc_feature 1: note = DNA
  misc_feature 2..7: note = RNA
  misc_feature 8: note = DNA
  misc_feature 9..18: note = RNA
  misc_feature 19: note = DNA
  misc_feature 20: note = RNA
ctgatggtccatgtctgtta

SEQ ID NO: 370 - DNA, 24 bp, Homo sapiens
  source 1..24: mol_type = unassigned DNA; note = RPL32_on
tttggggtgatcagacccaacagc

SEQ ID NO: 371 - DNA, 24 bp, Homo sapiens
  source 1..24: mol_type = unassigned DNA; note = RPL_32-off.1
tttggggtgatcagacccaacacc

SEQ ID NO: 372 - DNA, 24 bp, Homo sapiens
  source 1..24: mol_type = unassigned DNA; note = RPL_32-off.2
tttggggtgatcagacccaacacc

SEQ ID NO: 373 - DNA, 24 bp, Homo sapiens
  source 1..24: mol_type = unassigned DNA; note = DNMT1_on
tttcctgatggtccatgtctgtta

SEQ ID NO: 374 - DNA, 24 bp, Homo sapiens
  source 1..24: mol_type = unassigned DNA; note = DNMT1_off-1
tttcctgatggtccatgtctgaat

SEQ ID NO: 375 - RNA, 20 nt, synthetic construct
  source 1..20: mol_type = other RNA; note = Description of Artificial Sequence: Synthetic oligonucleotide; note = RP_cr
gggtgatcagacccaacagc

SEQ ID NO: 376 - DNA, 20 bp, synthetic construct
  source 1..20: mol_type = other DNA; note = Description of Artificial Sequence: Synthetic oligonucleotide; note = RP_cr_2_d.1
  misc_feature 1: note = DNA
  misc_feature 2..20: note = RNA
gggtgatcagacccaacagc

SEQ ID NO: 377 - DNA, 20 bp, synthetic construct
  source 1..20: mol_type = other DNA; note = Description of Artificial Sequence: Synthetic oligonucleotide; note = RP_cr_2_d.8
  misc_feature 1..7: note = RNA
  misc_feature 8: note = DNA
  misc_feature 9..20: note = RNA
gggtgatcagacccaacagc

SEQ ID NO: 378 - DNA, 20 bp, synthetic construct
  source 1..20: mol_type = other DNA; note = Description of Artificial Sequence: Synthetic oligonucleotide; note = RP_cr_2_d.9
  misc_feature 1..8: note = RNA
  misc_feature 9: note = DNA
  misc_feature 10..20: note = RNA
gggtgatcagacccaacagc

SEQ ID NO: 379 - DNA, 20 bp, synthetic construct
  source 1..20: mol_type = other DNA; note = Description of Artificial Sequence: Synthetic oligonucleotide; note = RP_cr_2_d.10
  misc_feature 1..9: note = RNA
  misc_feature 10: note = DNA
  misc_feature 11..20: note = RNA
gggtgatcagacccaacagc

SEQ ID NO: 380 - DNA, 20 bp, synthetic construct
  source 1..20: mol_type = other DNA; note = Description of Artificial Sequence: Synthetic oligonucleotide; note = RP_cr_2_d.11
  misc_feature 1..10: note = RNA
  misc_feature 11: note = DNA
  misc_feature 12..20: note = RNA
gggtgatcagacccaacagc

SEQ ID NO: 381 - DNA, 20 bp, synthetic construct
  source 1..20: mol_type = other DNA; note = Description of Artificial Sequence: Synthetic oligonucleotide; note = RP_cr_2_d.12
  misc_feature 1..11: note = RNA
  misc_feature 12: note = DNA
  misc_feature 13..20: note = RNA
gggtgatcagacccaacagc

SEQ ID NO: 382 - DNA, 20 bp, synthetic construct
  source 1..20: mol_type = other DNA; note = Description of Artificial Sequence: Synthetic oligonucleotide; note = RP_cr_2_d.14
  misc_feature 1..13: note = RNA
  misc_feature 14: note = DNA
  misc_feature 15..20: note = RNA
gggtgatcagacccaacagc

SEQ ID NO: 383 - DNA, 20 bp, synthetic construct
  source 1..20: mol_type = other DNA; note = Description of Artificial Sequence: Synthetic oligonucleotide; note = RP_cr_2_d.15
  misc_feature 1..14: note = RNA
  misc_feature 15: note = DNA
  misc_feature 16..20: note = RNA
gggtgatcagacccaacagc

SEQ ID NO: 384 - DNA, 20 bp, synthetic construct
  source 1..20: mol_type = other DNA; note = Description of Artificial Sequence: Synthetic oligonucleotide; note = RP_cr_2_d.16
  misc_feature 1..15: note = RNA
  misc_feature 16: note = DNA
  misc_feature 17..20: note = RNA
gggtgatcagacccaacagc

SEQ ID NO: 385 - DNA, 20 bp, synthetic construct
  source 1..20: mol_type = other DNA; note = Description of Artificial Sequence: Synthetic oligonucleotide; note = RP_cr_2_d.17
  misc_feature 1..16: note = RNA
  misc_feature 17: note = DNA
  misc_feature 18..20: note = RNA
gggtgatcagacccaacagc

SEQ ID NO: 386 - DNA, 20 bp, synthetic construct
  source 1..20: mol_type = other DNA; note = Description of Artificial Sequence: Synthetic oligonucleotide; note = RP_cr_2_d.18
  misc_feature 1..17: note = RNA
  misc_feature 18: note = DNA
  misc_feature 19..20: note = RNA
gggtgatcagacccaacagc

SEQ ID NO: 387 - DNA, 20 bp, synthetic construct
  source 1..20: mol_type = other DNA; note = Description of Artificial Sequence: Synthetic oligonucleotide; note = RP_cr_2_d.19
  misc_feature 1..18: note = RNA
  misc_feature 19: note = DNA
  misc_feature 20: note = RNA
gggtgatcagacccaacagc

SEQ ID NO: 388 - DNA, 20 bp, synthetic construct
  source 1..20: mol_type = other DNA; note = Description of Artificial Sequence: Synthetic oligonucleotide; note = RP_cr_2_d.20
  misc_feature 1..19: note = RNA
  misc_feature 20: note = DNA
gggtgatcagacccaacagc

SEQ ID NO: 389 - DNA, 20 bp, synthetic construct
  source 1..20: mol_type = other DNA; note = Description of Artificial Sequence: Synthetic oligonucleotide; note = RP_cr_3'_d.13
  misc_feature 1..12: note = RNA
  misc_feature 13: note = DNA
  misc_feature 14..20: note = RNA
gggtgatcagacccaacagc

SEQ ID NO: 390 - DNA, 20 bp, synthetic construct
  source 1..20: mol_type = other DNA; note = Description of Artificial Sequence: Synthetic oligonucleotide; note = RP_cr_3'_d11.12
  misc_feature 1..10: note = RNA
  misc_feature 11..12: note = DNA
  misc_feature 13..20: note = RNA
gggtgatcagacccaacagc

SEQ ID NO: 391 - DNA, 20 bp, synthetic construct
  source 1..20: mol_type = other DNA; note = Description of Artificial Sequence: Synthetic oligonucleotide; note = RP_cr_3'_d12.14
  misc_feature 1..11: note = RNA
  misc_feature 12: note = DNA
  misc_feature 13: note = RNA
  misc_feature 14: note = DNA
  misc_feature 15..20: note = RNA
gggtgatcagacccaacagc

SEQ ID NO: 392 - DNA, 20 bp, synthetic construct
  source 1..20: mol_type = other DNA; note = Description of Artificial Sequence: Synthetic oligonucleotide; note = RP_cr_3'_d14.15
  misc_feature 1..13: note = RNA
  misc_feature 14..15: note = DNA
  misc_feature 16..20: note = RNA
gggtgatcagacccaacagc

SEQ ID NO: 393 - DNA, 20 bp, synthetic construct
  source 1..20: mol_type = other DNA; note = Description of Artificial Sequence: Synthetic oligonucleotide; note = RP_cr_3'_d11.15
  misc_feature 1..10: note = RNA
  misc_feature 11: note = DNA
  misc_feature 12..14: note = RNA
  misc_feature 15: note = DNA
  misc_feature 16..20: note = RNA
gggtgatcagacccaacagc

SEQ ID NO: 394 - DNA, 20 bp, synthetic construct
  source 1..20: mol_type = other DNA; note = Description of Artificial Sequence: Synthetic oligonucleotide; note = RP_cr_3'_d11.12.14
  misc_feature 1..10: note = RNA
  misc_feature 11..12: note = DNA
  misc_feature 13: note = RNA
  misc_feature 14: note = DNA
  misc_feature 15..20: note = RNA
gggtgatcagacccaacagc

SEQ ID NO: 395 - DNA, 20 bp, synthetic construct
  source 1..20: mol_type = other DNA; note = Description of Artificial Sequence: Synthetic oligonucleotide; note = RP_cr_3'_d12.14.15
  misc_feature 1..11: note = RNA
  misc_feature 12: note = DNA
  misc_feature 13: note = RNA
  misc_feature 14..15: note = DNA
  misc_feature 16..20: note = RNA
gggtgatcagacccaacagc

SEQ ID NO: 396 - DNA, 20 bp, synthetic construct
  source 1..20: mol_type = other DNA; note = Description of Artificial Sequence: Synthetic oligonucleotide; note = RP_cr_3'_d11.14.15
  misc_feature 1..10: note = RNA
  misc_feature 11: note = DNA
  misc_feature 12..13: note = RNA
  misc_feature 14..15: note = DNA
  misc_feature 16..20: note = RNA
gggtgatcagacccaacagc

SEQ ID NO: 397 - DNA, 20 bp, synthetic construct
  source 1..20: mol_type = other DNA; note = Description of Artificial Sequence: Synthetic oligonucleotide; note = RP_cr_3'_d11.12.14.15
  misc_feature 1..10: note = RNA
  misc_feature 11..12: note = DNA
  misc_feature 13: note = RNA
  misc_feature 14..15: note = DNA
  misc_feature 16..20: note = RNA
gggtgatcagacccaacagc

SEQ ID NO: 398 - DNA, 21 bp, synthetic construct
  source 1..21: mol_type = other DNA; note = Description of Artificial Sequence: Synthetic oligonucleotide; note = RP_cr_3'_sp.20
  misc_difference 20: note = modified_base - an abasic site that lacks 2'-hydroxyl
  misc_feature 1..19: note = RNA
  misc_feature 20..21: note = DNA
gggtgatcagacccaacagnt

SEQ ID NO: 399 - DNA, 21 bp, synthetic construct
  source 1..21: mol_type = other DNA; note = Description of Artificial Sequence: Synthetic oligonucleotide; note = RP_cr_3'_d14_sp20
  misc_difference 20: note = modified_base - an abasic site that lacks 2'-hydroxyl
  misc_feature 1..13: note = RNA
  misc_feature 14: note = DNA
  misc_feature 15..19: note = RNA
  misc_feature 20..21: note = DNA
gggtgatcagacccaacagnt

SEQ ID NO: 400 - DNA, 21 bp, synthetic construct
  source 1..21: mol_type = other DNA; note = Description of Artificial Sequence: Synthetic oligonucleotide; note = RP_cr_3'_d15_sp20
  misc_difference 20: note = modified_base - an abasic site that lacks 2'-hydroxyl
  misc_feature 1..14: note = RNA
  misc_feature 15: note = DNA
  misc_feature 16..19: note = RNA
  misc_feature 20..21: note = DNA
gggtgatcagacccaacagnt

SEQ ID NO: 401 - DNA, 21 bp, synthetic construct
  source 1..21: mol_type = other DNA; note = Description of Artificial Sequence: Synthetic oligonucleotide; note = RP_cr_3'_d12_sp20
  misc_difference 20: note = modified_base - an abasic site that lacks 2'-hydroxyl
  misc_feature 1..11: note = RNA
  misc_feature 12: note = DNA
  misc_feature 13..19: note = RNA
  misc_feature 20..21: note = DNA
gggtgatcagacccaacagnt

SEQ ID NO: 402 - DNA, 21 bp, synthetic construct
  source 1..21: mol_type = other DNA; note = Description of Artificial Sequence: Synthetic oligonucleotide; note = RP_cr_3'_d11_sp20
  misc_difference 20: note = modified_base - an abasic site that lacks 2'-hydroxyl
  misc_feature 1..10: note = RNA
  misc_feature 11: note = DNA
  misc_feature 12..19: note = RNA
  misc_feature 20..21: note = DNA
gggtgatcagacccaacagnt

SEQ ID NO: 403 - DNA, 21 bp, synthetic construct
  source 1..21: mol_type = other DNA; note = Description of Artificial Sequence: Synthetic oligonucleotide; note = RP_cr_3'_d14.15_sp20
  misc_difference 20: note = modified_base - an abasic site that lacks 2'-hydroxyl
  misc_feature 1..13: note = RNA
  misc_feature 14..15: note = DNA
  misc_feature 16..19: note = RNA
  misc_feature 20..21: note = DNA
gggtgatcagacccaacagnt

SEQ ID NO: 404 - RNA, 20 nt, synthetic construct
  source 1..20: mol_type = other RNA; note = Description of Artificial Sequence: Synthetic oligonucleotide; note = DNMT1-crRNA
ctgatggtccatgtctgtta

SEQ ID NO: 405 - DNA, 20 bp, synthetic construct
  source 1..20: mol_type = other DNA; note = Description of Artificial Sequence: Synthetic oligonucleotide; note = DNMT1-d1.20
  misc_feature 1: note = DNA
  misc_feature 2..19: note = RNA
  misc_feature 20: note = DNA
ctgatggtccatgtctgtta

SEQ ID NO: 406 - DNA, 20 bp, synthetic construct
  source 1..20: mol_type = other DNA; note = Description of Artificial Sequence: Synthetic oligonucleotide; note = DNMT1-d8.19
  misc_feature 1..7: note = RNA
  misc_feature 8: note = DNA
  misc_feature 9..18: note = RNA
  misc_feature 19: note = DNA
  misc_feature 20: note = RNA
ctgatggtccatgtctgtta

SEQ ID NO: 407 - DNA, 20 bp, synthetic construct
  source 1..20: mol_type = other DNA; note = Description of Artificial Sequence: Synthetic oligonucleotide; note = DNMT1-d9
  misc_feature 1..8: note = RNA
  misc_feature 9: note = DNA
  misc_feature 10..20: note = RNA
ctgatggtccatgtctgtta

SEQ ID NO: 408 - DNA, 20 bp, synthetic construct
  source 1..20: mol_type = other DNA; note = Description of Artificial Sequence: Synthetic oligonucleotide; note = DNMT1-d8.14
  misc_feature 1..7: note = RNA
  misc_feature 8: note = DNA
  misc_feature 9..13: note = RNA
  misc_feature 14: note = DNA
  misc_feature 15..20: note = RNA
ctgatggtccatgtctgtta

SEQ ID NO: 409 - DNA, 20 bp, synthetic construct
  source 1..20: mol_type = other DNA; note = Description of Artificial Sequence: Synthetic oligonucleotide; note = DNMT1-d10.17
  misc_feature 1..9: note = RNA
  misc_feature 10: note = DNA
  misc_feature 11..16: note = RNA
  misc_feature 17: note = DNA
  misc_feature 18..20: note = RNA
ctgatggtccatgtctgtta

SEQ ID NO: 410 - DNA, 20 bp, synthetic construct
  source 1..20: mol_type = other DNA; note = Description of Artificial Sequence: Synthetic oligonucleotide; note = DNMT1-d1.8.20
  misc_feature 1: note = DNA
  misc_feature 2..7: note = RNA
  misc_feature 8: note = DNA
  misc_feature 9..19: note = RNA
  misc_feature 20: note = DNA
ctgatggtccatgtctgtta

SEQ ID NO: 411 - DNA, 20 bp, synthetic construct
  source 1..20: mol_type = other DNA; note = Description of Artificial Sequence: Synthetic oligonucleotide; note = DNMT1-d1.8.19
  misc_feature 1: note = DNA
  misc_feature 2..7: note = RNA
  misc_feature 8: note = DNA
  misc_feature 9..18: note = RNA
  misc_feature 19: note = DNA
  misc_feature 20: note = RNA
ctgatggtccatgtctgtta

SEQ ID NO: 412 - DNA, 20 bp, synthetic construct
  source 1..20: mol_type = other DNA; note = Description of Artificial Sequence: Synthetic oligonucleotide; note = DNMT1-d8.9.15
  misc_feature 1..7: note = RNA
  misc_feature 8..9: note = DNA
  misc_feature 10..14: note = RNA
  misc_feature 15: note = DNA
  misc_feature 16..20: note = RNA
ctgatggtccatgtctgtta

SEQ ID NO: 413 - DNA, 3147 bp, synthetic construct
  source 1..3147: mol_type = other DNA; note = Description of Artificial Sequence: Synthetic polynucleotide; note = anti-BCMA CAR construct
cataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgtaccagcttaattaattagtccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagttattacctcggtggcagggcctgcatatggagggcatcgtatgtatctttcgtggccgtactcaggccttggtagagaccgtcgtggcctttccctctgcgtctctctcccttcatcccgatctcgctgtaagcctccgccatcttatctttctgcagttcattataaagcccctcttgcgggttttttctcctcggttttcctcccatctctgggtccctccctcggcgcttatccaatacatcatattcctctcgcctccccaggttaagctcattgtaaagttggttttgcccttgctgataggccggagcgtcagcagaccggctaaatttcactctcagttcgcaaccaccctcctcttcttcggggaatcggcagctacatccatcctcctcttgcgtagtttggaccgggcgcataaatggttgcttaaaaatatacagcaacttttttcttcctcttttgcagtaaagagtaataacgagggacagcagcaagaccccgcacgttccggcaagcggtgcccagatgtagatgtcgcaagcaaagtccagcccgcgagtatgaaccgcgcctcccgctgcggggcgacaagcctccggccgaagagagagtggctgggaagcgatagtgggtgcaggcgtgggtggccgaggggcaggcgtggttgtggcagcggctttaatttccactttggtgccgccgccaaaggtcggcggaaagctatggccgttctggcaataatacaccgcaaaatcttccggttccaggctgctaatggtcagggtaaaatcggtgccgctgccgctgccgctaaagcgcgccggaatgccggtaatgctctggctcgcataataaatcagcaggcgcggcgcctggcccggtttctgctgataccaatgcagataatcgctaatgctctggctcgcgcggcagctcagggtcgcgcgttcgcccgggctcaggctcagggtcgccgggctctgggtcagcacaatttcgctgccgccgccgccgctgccgccgccgccgctgccgccgccgccgctgctcacggtcaccagggtgccctggccccacacatcaaaatagccatcatagttatagcgcgcgcaataatacaccgcggtatcttcgctgcgcaggctgctcagttccatatacgcggtgctggtgcttttatccgcggtaatggtcacgcggcctttaaatttttcgttatatttggtcgcatcgttatacggaataatatagcccatccattccaggccctggcccggcgcctggcgcacccaatgcatcacatagctggtaaaggtatagccgctcgctttgcagctcactttcacgctgctgcccggttttttcacttccgcgccgctctgcaccagctgcacctgcggtcttgccgcgtggagcaggagtgccaatggaagcagaagtgcggtcacgggcaacgccatggtggctatgacgcgtcgcgccgagtgaggggttgtgggctcttttattgagctcggggagcagaagcgcgcgaacagaagcgagaagcgaactgattggttagttcaaataaggcacagggtcatttcaggtccttggggcaccctggaaacatctgatggttctctagaaactgctgagggcgggaccgcatctggggaccatctgttcttggccctgagccggggcaggaactgcttaccacagatatcctgtttggcccatattctgctgttccaactgttcttggccctgagccggggcaggaactgcttaccacagatatcctgtttggcccatattctgctgtctctctgttcctaaccttgatcctagcttgccaaacctacaggtggggtctttcattcccccctttttctggagactaaataaagtttaaactgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaactgagtt

SEQ ID NO: 414 - DNA, 3409 bp, synthetic construct
  source 1..3409: mol_type = other DNA; note = Description of Artificial Sequence: Synthetic polynucleotide; note = B2M-HLA-e construct
tgagtgctgagagggcatcagaagtccttgagagcctccagagaaaggctcttaaaaatgcagcgcaatctccagtgacagaagatactgctagaaatctgctagaaaaaaaacaaaaaaggcatgtatagaggaattatgagggaaagataccaagtcacggtttattcttcaaaatggaggtggcttgttgggaaggtggaagctcatttggccagagtggaaatggaattgggagaaatcgatgaccaaatgtaaacacttggtgcctgatatagcttgacaccaagttagccccaagtgaaataccctggcaatattaatgtgtcttttcccgatattcctcaggtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaatttttaattaattagtccatagagcccaccgcatccccagcatgcctgctattgtcttcccaatcctcccccttgctgtcctgccccaccccaccccccagaatagaatgacacctactcagacaatgcgatgcaatttcctcattttattaggaaaggacagtgggagtggcaccttccagggtcaaggaaggcacgggggaggggcaaacaacagatggctggcaactagaaggcacagttattacaagctgtgagactcagacccctgggcactgtcgctccactcagccttagagtagctccctccttttccacctgagctcttcttcctccatatcacagcagcaaccacagctccagagaccacagatccaaggagaaccaggccagcaatgatgcccacgatggggatggtgggctgggaagccggcttccatctcagggtgacgggctcgggtagcccctcatgctgcacatggcacgtgtatctctgctcctctccagaaggcaccaccacagctgcccacttctggaaggttccatcccctgcaggcctggtctccacgagctccgtgtcctgggtatggccctccccatcctgctgccaggtcagtgtgatctccgcagggtagaagcccagggcccagcacctcagggtggcctcatggtcagagatggggtggtgagtcacgtgtgtctttgggggctccaggtgaagcagcgtctccttccccttctccaggtatttgtggagccactccacgcatgtgtcttccaggtaggctctctggtgctccgcctcagaagcatcatttgacttttgctcggagatctgagccgccgtgtccaccgcggtccaggagcgcaggtcctcattcagggtgagataatccttgccgtcgtaggcgaactgttcatacccgcggaggaagcgcccgtcgggccccagctcgcagccatgcatccactgcagggtgtgagacccggcctcgctctgattgtagtagccgcgcagcgtccgcagattcactcggaaaatctgtgcggtgtccctggcgctccgtgtctcccggtcccaatactctgacccctcctgctccatccacggcgcccgcggcaccatcctcggactcgcggcgtcgttgtcgaagcgcacgaactgggtgtcgtccacgtagcccacagagatgaagcggggctccccgcggccgggccgggacacggaagtgtggaaatacttcaaggagtgggatccagaccctccgccaccagatccccctcctccagaaccgcctccgccagaccctccgccacccatgtctcgatcccatttcacgatcttgggctgtgacaaagtcacatggttcacacggcaggcatactcatctttctccgtaggtgtaaactctgtatagtacaagagatagaaagaccagtccttgctgaaagacaagtctgaatgctccactttttcaattctctctccattcttcagtaagtcaacttcaatgtcggatggatgaaacccagacacatagcaattcaggaaatttgactttccattctctgctggatgacgtgagtaaacctgaatctttggagtacgctggatggagccgccgcctccgctaccgcctcctccgctcccacctccacccagaaagagggtccgtggtgccattacagcctccaggccagaaagagagagtagcgcgagcacagctaaggccacggagcgagacatggtggctctagagtaggcgccggtcacagcttggatctgtaacggcgcagaacagaaaacgaaacaaagacgtagagttgagcaagcagggtcaggcaaagcgtggagagccggctgagtctaggtaggctccaagggagcgccggacaaaggcccggtctcgacctgagctttaaacttacctagacggcggacgcagttcaggaggcaccacaggcgggaggcggcagaacgcgactcaaccggcgtggatggcggcctcaggtagggcggcgggcgcgtgaaggagagatgcgagcccctcgaagcttcagctgtgttctggcggcaaacccgttgcgaaaaagaacgttcacggcgactactgcacttatatacggttctcccccaccctcgggaaaaaggcggagccagtacacgacatcactttcccagtttaccccgcgccaccttctctaggcacccgttcaattgccgacccctccccccaacttctcggggactgtgggcgatgtgcgctctgcccactgacgggcaccggagcgatcgcagatccttgtcgaccacccccactgaaaaagatgagtatgcctgccgtgtgaaccatgtgactttgtcacagcccaagatagttaagtggggtaagtcttacattcttttgtaagctgctgaaagttgtgtatgagtagtcatatcataaagctgctttgatataaaaaaggtctatggccatactaccctgaatgagtcccatcccatctgatataaacaatctgcatattgggattgtcagggaatgttcttaaagatcagattagtggcacctgctgagatactgatgcacagcatggtttctgaaccagtagtttccctgcagttgagcagggagcagcagcagcacttgcacaaatacatatacactcttaacacttcttacctactggcttcctctagcttttgtggcagcttcaggtatatttagcactgaacgaacatctcaagaaggtataggcctttgtttgtaagtcctgctgtcctagcatcctataatcctggacttctccagtactttctggctggattggtatctgaggctagtaggaagggcttgttc

SEQ ID NO: 415 - DNA, 40 bp, synthetic construct
  source 1..40: mol_type = other DNA; note = Description of Artificial Sequence: Synthetic oligonucleotide; note = TRAC_cr3'_tgt.12-d20.11.1
  misc_feature 1: note = DNA
  misc_feature 2: note = RNA
  misc_feature 3: note = DNA
  misc_feature 4..6: note = RNA
  misc_feature 7: note = DNA
  misc_feature 8..9: note = RNA
  misc_feature 10: note = DNA
  misc_feature 11: note = RNA
  misc_feature 12: note = DNA
  misc_feature 13: note = RNA
  misc_feature 14..15: note = DNA
  misc_feature 16..18: note = RNA
  misc_feature 19: note = DNA
  misc_feature 20: note = RNA
  misc_feature 21: note = DNA
  misc_feature 22..30: note = RNA
  misc_feature 31: note = DNA
  misc_feature 32..39: note = RNA
  misc_feature 40: note = DNA
taatttctactcttgtagatgagtctctcagctggtacac

SEQ ID NO: 416 - DNA, 40 bp, synthetic construct
  source 1..40: mol_type = other DNA; note = Description of Artificial Sequence: Synthetic oligonucleotide; note = B2M_cr3'_tgt.12-d20.11.1
  misc_feature 1: note = DNA
  misc_feature 2: note = RNA
  misc_feature 3: note = DNA
  misc_feature 4..6: note = RNA
  misc_feature 7: note = DNA
  misc_feature 8..9: note = RNA
  misc_feature 10: note = DNA
  misc_feature 11: note = RNA
  misc_feature 12: note = DNA
  misc_feature 13: note = RNA
  misc_feature 14..15: note = DNA
  misc_feature 16..18: note = RNA
  misc_feature 19: note = DNA
  misc_feature 20: note = RNA
  misc_feature 21: note = DNA
  misc_feature 22..30: note = RNA
  misc_feature 31: note = DNA
  misc_feature 32..39: note = RNA
  misc_feature 40: note = DNA
taatttctactcttgtagatagtgggggtgaattcagtgt

SEQ ID NO: 417 - RNA, 20 nt, synthetic construct
  source 1..20: mol_type = other RNA; note = Description of Artificial Sequence: Synthetic oligonucleotide; note = crRNA
taatttctactcttgtagat

SEQ ID NO: 418 - DNA, 20 bp, synthetic construct
  source 1..20: mol_type = other DNA; note = Description of Artificial Sequence: Synthetic oligonucleotide; note = CRISPR-d1
  misc_feature 1: note = DNA
  misc_feature 2..20: note = RNA
taatttctactcttgtagat

SEQ ID NO: 419 - DNA, 20 bp, synthetic construct
  source 1..20: mol_type = other DNA; note = Description of Artificial Sequence: Synthetic oligonucleotide; note = CRISPR-d2
  misc_feature 1: note = RNA
  misc_feature 2: note = DNA
  misc_feature 3..20: note = RNA
taatttctactcttgtagat

SEQ ID NO: 420 - DNA, 20 bp, synthetic construct
  source 1..20: mol_type = other DNA; note = Description of Artificial Sequence: Synthetic oligonucleotide; note = CRISPR-d3
  misc_feature 1..2: note = RNA
  misc_feature 3: note = DNA
  misc_feature 4..20: note = RNA
taatttctactcttgtagat

SEQ ID NO: 421 - DNA, 20 bp, synthetic construct
  source 1..20: mol_type = other DNA; note = Description of Artificial Sequence: Synthetic oligonucleotide; note = CRISPR-d4
  misc_feature 1..3: note = RNA
  misc_feature 4: note = DNA
  misc_feature 5..20: note = RNA
taatttctactcttgtagat

SEQ ID NO: 422 - DNA, 20 bp, synthetic construct
  source 1..20: mol_type = other DNA; note = Description of Artificial Sequence: Synthetic oligonucleotide; note = CRISPR-d5
  misc_feature 1..4: note = RNA
  misc_feature 5: note = DNA
  misc_feature 6..20: note = RNA
taatttctactcttgtagat

SEQ ID NO: 423 - DNA, 20 bp, synthetic construct
  source 1..20: mol_type = other DNA; note = Description of Artificial Sequence: Synthetic oligonucleotide; note = CRISPR-d6
  misc_feature 1..5: note = RNA
  misc_feature 6: note = DNA
  misc_feature 7..20: note = RNA
taatttctactcttgtagat

SEQ ID NO: 424 - DNA, 20 bp, synthetic construct
  source 1..20: mol_type = other DNA; note = Description of Artificial Sequence: Synthetic oligonucleotide; note = CRISPR-d7
  misc_feature 1..6: note = RNA
  misc_feature 7: note = DNA
  misc_feature 8..20: note = RNA
taatttctactcttgtagat

SEQ ID NO: 425 - DNA, 20 bp, synthetic construct
  source 1..20: mol_type = other DNA; note = Description of Artificial Sequence: Synthetic oligonucleotide; note = CRISPR-d8
  misc_feature 1..7: note = RNA
  misc_feature 8: note = DNA
  misc_feature 9..20: note = RNA
taatttctactcttgtagat

SEQ ID NO: 426 - DNA, 20 bp, synthetic construct
  source 1..20: mol_type = other DNA; note = Description of Artificial Sequence: Synthetic oligonucleotide; note = CRISPR-d9
  misc_feature 1..8: note = RNA
  misc_feature 9: note = DNA
  misc_feature 10..20: note = RNA
taatttctactcttgtagat

SEQ ID NO: 427 - DNA, 20 bp, synthetic construct
  source 1..20: mol_type = other DNA; note = Description of Artificial Sequence: Synthetic oligonucleotide; note = CRISPR-d10
  misc_feature 1..9: note = RNA
  misc_feature 10: note = DNA
  misc_feature 11..20: note = RNA
taatttctactcttgtagat

SEQ ID NO: 428 - DNA, 20 bp, synthetic construct
  source 1..20: mol_type = other DNA; note = Description of Artificial Sequence: Synthetic oligonucleotide; note = CRISPR-d11
  misc_feature 1..10: note = RNA
  misc_feature 11: note = DNA
  misc_feature 12..20: note = RNA
taatttctactcttgtagat

SEQ ID NO: 429 - DNA, 20 bp, synthetic construct
  source 1..20: mol_type = other DNA; note = Description of Artificial Sequence: Synthetic oligonucleotide; note = CRISPR-d12
  misc_feature 1..11: note = RNA
  misc_feature 12: note = DNA
  misc_feature 13..20: note = RNA
taatttctactcttgtagat

SEQ ID NO: 430 - DNA, 20 bp, synthetic construct
  source 1..20: mol_type = other DNA; note = Description of Artificial Sequence: Synthetic oligonucleotide; note = CRISPR-d13
  misc_feature 1..12: note = RNA
  misc_feature 13: note = DNA
  misc_feature 14..20: note = RNA
taatttctactcttgtagat

SEQ ID NO: 431 - DNA, 20 bp, synthetic construct
  source 1..20: mol_type = other DNA; note = Description of Artificial Sequence: Synthetic oligonucleotide; note = CRISPR-d14
  misc_feature 1..13: note = RNA
  misc_feature 14: note = DNA
  misc_feature 15..20: note = RNA
taatttctactcttgtagat

SEQ ID NO: 432 - DNA, 20 bp, synthetic construct
  source 1..20: mol_type = other DNA; note = Description of Artificial Sequence: Synthetic oligonucleotide; note = CRISPR-d15
  misc_feature 1..14: note = RNA
  misc_feature 15: note = DNA
  misc_feature 16..20: note = RNA
taatttctactcttgtagat

SEQ ID NO: 433 - DNA, 20 bp, synthetic construct
  source 1..20: mol_type = other DNA; note = Description of Artificial Sequence: Synthetic oligonucleotide; note = CRISPR-d16
  misc_feature 1..15: note = RNA
  misc_feature 16: note = DNA
  misc_feature 17..20: note = RNA
taatttctactcttgtagat

SEQ ID NO: 434 - DNA, 20 bp, synthetic construct
  source 1..20: mol_type = other DNA; note = Description of Artificial Sequence: Synthetic oligonucleotide; note = CRISPR-d17
  misc_feature 1..16: note = RNA
  misc_feature 17: note = DNA
  misc_feature 18..20: note = RNA
taatttctactcttgtagat

SEQ ID NO: 435 - DNA, 20 bp, synthetic construct
  source 1..20: mol_type = other DNA; note = Description of Artificial Sequence: Synthetic oligonucleotide; note = CRISPR-d18
  misc_feature 1..17: note = RNA
  misc_feature 18: note = DNA
  misc_feature 19..20: note = RNA
taatttctactcttgtagat

SEQ ID NO: 436 - DNA, 20 bp, synthetic construct
  source 1..20: mol_type = other DNA; note = Description of Artificial Sequence: Synthetic oligonucleotide; note = CRISPR-d19
  misc_feature 1..18: note = RNA
  misc_feature 19: note = DNA
  misc_feature 20: note = RNA
taatttctactcttgtagat

SEQ ID NO: 437 - DNA, 20 bp, synthetic construct
  source 1..20: mol_type = other DNA; note = Description of Artificial Sequence: Synthetic oligonucleotide; note = CRISPR-d20
  misc_feature 1..19: note = RNA
  misc_feature 20: note = DNA
taatttctactcttgtagat

SEQ ID NO: 438 - RNA, 40 nt, synthetic construct
  source 1..40: mol_type = other RNA; note = Description of Artificial Sequence: Synthetic oligonucleotide; note = DMT.t3_crRNA
taatttctactcttgtagatctgatggtccatgtctgtta

SEQ ID NO: 439 - DNA, 40 bp, synthetic construct
  source 1..40: mol_type = other DNA; note = Description of Artificial Sequence: Synthetic oligonucleotide; note = DMT.t3_cr-d1
  misc_feature 1: note = DNA
  misc_feature 2..40: note = RNA
taatttctactcttgtagatctgatggtccatgtctgtta

SEQ ID NO: 440 - DNA, 40 bp, synthetic construct; identical to SEQ ID NO: 439
  source 1..40: mol_type = other DNA; note = Description of Artificial Sequence: Synthetic oligonucleotide; note = DMT.t3_cr-d2
  misc_feature 1: note = RNA
  misc_feature 2: note = DNA
  misc_feature 3..40: note = RNA

SEQ ID NO: 441 - DNA, 40 bp, synthetic construct; identical to SEQ ID NO: 439
  source 1..40: mol_type = other DNA; note = Description of Artificial Sequence: Synthetic oligonucleotide; note = DMT.t3_cr-d3
  misc_feature 1..2: note = RNA
  misc_feature 3: note = DNA
  misc_feature 4..40: note = RNA

SEQ ID NO: 442 - DNA, 40 bp, synthetic construct; identical to SEQ ID NO: 439
  source 1..40: mol_type = other DNA; note = Description of Artificial Sequence: Synthetic oligonucleotide; note = DMT.t3_cr-d4
  misc_feature 1..3: note = RNA
  misc_feature 4: note = DNA
  misc_feature 5..40: note = RNA

SEQ ID NO: 443 - DNA, 40 bp, synthetic construct; identical to SEQ ID NO: 439
  source 1..40: mol_type = other DNA; note = Description of Artificial Sequence: Synthetic oligonucleotide; note = DMT.t3_cr-d5
  misc_feature 1..4: note = RNA
  misc_feature 5: note = DNA
  misc_feature 6..40: note = RNA

SEQ ID NO: 444 - DNA, 40 bp, synthetic construct; identical to SEQ ID NO: 439
  source 1..40: mol_type = other DNA; note = Description of Artificial Sequence: Synthetic oligonucleotide; note = DMT.t3_cr-d6
  misc_feature 1..5: note = RNA
  misc_feature 6: note = DNA
  misc_feature 7..40: note = RNA

SEQ ID NO: 445 - DNA, 40 bp, synthetic construct; identical to SEQ ID NO: 439
  source 1..40: mol_type = other DNA; note = Description of Artificial Sequence: Synthetic oligonucleotide; note = DMT.t3_cr-d7
  misc_feature 1..6: note = RNA
  misc_feature 7: note = DNA
  misc_feature 8..40: note = RNA

SEQ ID NO: 446 - DNA, 40 bp, synthetic construct; identical to SEQ ID NO: 439
  source 1..40: mol_type = other DNA; note = Description of Artificial Sequence: Synthetic oligonucleotide; note = DMT.t3_cr-d8
  misc_feature 1..7: note = RNA
  misc_feature 8: note = DNA
  misc_feature 9..40: note = RNA

SEQ ID NO: 447 - DNA, 40 bp, synthetic construct; identical to SEQ ID NO: 439
  source 1..40: mol_type = other DNA; note = Description of Artificial Sequence: Synthetic oligonucleotide; note = DMT.t3_cr-d9
  misc_feature 1..8: note = RNA
  misc_feature 9: note = DNA
  misc_feature 10..40: note = RNA

SEQ ID NO: 448 - DNA, 40 bp, synthetic construct; identical to SEQ ID NO: 439
  source 1..40: mol_type = other DNA; note = Description of Artificial Sequence: Synthetic oligonucleotide; note = DMT.t3_cr-d10
  misc_feature 1..9: note = RNA
  misc_feature 10: note = DNA
  misc_feature 11..40: note = RNA

SEQ ID NO: 449 - DNA, 40 bp, synthetic construct; identical to SEQ ID NO: 439
  source 1..40: mol_type = other DNA; note = Description of Artificial Sequence: Synthetic oligonucleotide; note = DMT.t3_cr-d11
  misc_feature 1..10: note = RNA
  misc_feature 11: note = DNA
  misc_feature 12..40: note = RNA

SEQ ID NO: 450 - DNA, 40 bp, synthetic construct; identical to SEQ ID NO: 439
  source 1..40: mol_type = other DNA; note = Description of Artificial Sequence: Synthetic oligonucleotide; note = DMT.t3_cr-d12
  misc_feature 1..11: note = RNA
  misc_feature 12: note = DNA
  misc_feature 13..40: note = RNA

SEQ ID NO: 451 - DNA, 40 bp, synthetic construct; identical to SEQ ID NO: 439
  source 1..40: mol_type = other DNA; note = Description of Artificial Sequence: Synthetic oligonucleotide; note = DMT.t3_cr-d13
  misc_feature 1..12: note = RNA
  misc_feature 13: note = DNA
  misc_feature 14..40: note = RNA

SEQ ID NO: 452 - DNA, 40 bp, synthetic construct; identical to SEQ ID NO: 439
  source 1..40: mol_type = other DNA; note = Description of Artificial Sequence: Synthetic oligonucleotide; note = DMT.t3_cr-d14
  misc_feature 1..13: note = RNA
  misc_feature 14: note = DNA
  misc_feature 15..40: note = RNA

SEQ ID NO: 453 - DNA, 40 bp, synthetic construct; identical to SEQ ID NO: 439
  source 1..40: mol_type = other DNA; note = Description of Artificial Sequence: Synthetic oligonucleotide; note = DMT.t3_cr-d15
  misc_feature 1..14: note = RNA
  misc_feature 15: note = DNA
  misc_feature 16..40: note = RNA

SEQ ID NO: 454 - DNA, 40 bp, synthetic construct; identical to SEQ ID NO: 439
  source 1..40: mol_type = other DNA; note = Description of Artificial Sequence: Synthetic oligonucleotide; note = DMT.t3_cr-d16
  misc_feature 1..15: note = RNA
  misc_feature 16: note = DNA
  misc_feature 17..40: note = RNA

SEQ ID NO: 455 - DNA, 40 bp, synthetic construct; identical to SEQ ID NO: 439
  source 1..40: mol_type = other DNA; note = Description of Artificial Sequence: Synthetic oligonucleotide; note = DMT.t3_cr-d17
  misc_feature 1..16: note = RNA
  misc_feature 17: note = DNA
  misc_feature 18..40: note = RNA

SEQ ID NO: 456 - DNA, 40 bp, synthetic construct; identical to SEQ ID NO: 439
  source 1..40: mol_type = other DNA; note = Description of Artificial Sequence: Synthetic oligonucleotide; note = DMT.t3_cr-d18
  misc_feature 1..17: note = RNA
  misc_feature 18: note = DNA
  misc_feature 19..40: note = RNA

SEQ ID NO: 457 - DNA, 40 bp, synthetic construct; identical to SEQ ID NO: 439
  source 1..40: mol_type = other DNA; note = Description of Artificial Sequence: Synthetic oligonucleotide; note = DMT.t3_cr-d19
  misc_feature 1..18: note = RNA
  misc_feature 19: note = DNA
  misc_feature 20..40: note = RNA

SEQ ID NO: 458 - DNA, 40 bp, synthetic construct; identical to SEQ ID NO: 439
  source 1..40: mol_type = other DNA; note = Description of Artificial Sequence: Synthetic oligonucleotide; note = DMT.t3_cr-d20
  misc_feature 1..19: note = RNA
  misc_feature 20: note = DNA
  misc_feature 21..40: note = RNA

SEQ ID NO: 459 - DNA, 20 bp, synthetic construct
  source 1..20: mol_type = other DNA; note = Description of Artificial Sequence: Synthetic oligonucleotide; note = CRISRP-3'
  misc_feature 1: note = DNA
  misc_feature 2: note = RNA
  misc_feature 3: note = DNA
  misc_feature 4..6: note = RNA
  misc_feature 7: note = DNA
  misc_feature 8..9: note = RNA
  misc_feature 10: note = DNA
  misc_feature 11: note = RNA
  misc_feature 12: note = DNA
  misc_feature 13: note = RNA
  misc_feature 14..15: note = DNA
  misc_feature 16..18: note = RNA
  misc_feature 19: note = DNA
  misc_feature 20: note = RNA
taatttctactcttgtagat

SEQ ID NO: 460 - DNA, 20 bp, synthetic construct
  source 1..20: mol_type = other DNA; note = Description of Artificial Sequence: Synthetic oligonucleotide; note = CRISRP-2
  misc_feature 1: note = DNA
  misc_feature 2..14: note = RNA
  misc_feature 15..19: note = DNA
  misc_feature 20: note = RNA
taatttctactcttgtagat

SEQ ID NO: 461 - DNA, 20 bp, synthetic construct
  source 1..20: mol_type = other DNA; note = Description of Artificial Sequence: Synthetic oligonucleotide; note = CRISRP-6
  misc_feature 1: note = DNA
  misc_feature 2..12: note = RNA
  misc_feature 13: note = DNA
  misc_feature 14..15: note = RNA
  misc_feature 16..17: note = DNA
  misc_feature 18..20: note = RNA
taatttctactcttgtagat

SEQ ID NO: 462 - DNA, 20 bp, synthetic construct
  source 1..20: mol_type = other DNA; note = Description of Artificial Sequence: Synthetic oligonucleotide; note = CRISRP-401
  misc_feature 1..6: note = RNA
  misc_feature 7: note = DNA
  misc_feature 8..9: note = RNA
  misc_feature 10: note = DNA
  misc_feature 11: note = RNA
  misc_feature 12: note = DNA
  misc_feature 13: note = RNA
  misc_feature 14..15: note = DNA
  misc_feature 16: note = RNA
  misc_feature 17: note = DNA
  misc_feature 18: note = RNA
  misc_feature 19: note = DNA
  misc_feature 20: note = RNA
taatttctactcttgtagat

SEQ ID NO: 463 - DNA, 20 bp, synthetic construct
  source 1..20: mol_type = other DNA; note = Description of Artificial Sequence: Synthetic oligonucleotide; note = CRISRP-402
  misc_feature 1: note = DNA
  misc_feature 2: note = RNA
  misc_feature 3: note = DNA
  misc_feature 4..11: note = RNA
  misc_feature 12: note = DNA
  misc_feature 13: note = RNA
  misc_feature 14: note = DNA
  misc_feature 15: note = RNA
  misc_feature 16..17: note = DNA
  misc_feature 18: note = RNA
  misc_feature 19: note = DNA
  misc_feature 20: note = RNA
taatttctactcttgtagat

SEQ ID NO: 464 - DNA, 20 bp, synthetic construct
  source 1..20: mol_type = other DNA; note = Description of Artificial Sequence: Synthetic oligonucleotide; note = CRISRP-403
  misc_feature 1..6: note = RNA
  misc_feature 7: note = DNA
  misc_feature 8..9: note = RNA
  misc_feature 10: note = DNA
  misc_feature 11: note = RNA
  misc_feature 12: note = DNA
  misc_feature 13: note = RNA
  misc_feature 14: note = DNA
  misc_feature 15..16: note = RNA
  misc_feature 17: note = DNA
  misc_feature 18: note = RNA
  misc_feature 19: note = DNA
  misc_feature 20: note = RNA
taatttctactcttgtagat

SEQ ID NO: 465 - DNA, 20 bp, synthetic construct
  source 1..20: mol_type = other DNA; note = Description of Artificial Sequence: Synthetic oligonucleotide; note = CRISRP-404
  misc_feature 1: note = DNA
  misc_feature 2: note = RNA
  misc_feature 3: note = DNA
  misc_feature 4..6: note = RNA
  misc_feature 7: note = DNA
  misc_feature 8..9: note = RNA
  misc_feature 10: note = DNA
  misc_feature 11: note = RNA
  misc_feature 12: note = DNA
  misc_feature 13: note = RNA
  misc_feature 14: note = DNA
  misc_feature 15..16: note = RNA
  misc_feature 17: note = DNA
  misc_feature 18: note = RNA
  misc_feature 19: note = DNA
  misc_feature 20: note = RNA
taatttctactcttgtagat

SEQ ID NO: 466 - DNA, 40 bp, synthetic construct; identical to SEQ ID NO: 415
  source 1..40: mol_type = other DNA; note = Description of Artificial Sequence: Synthetic oligonucleotide; note = TR_cr_tgt.12-d20.11.1
  misc_feature 1..20: note = RNA
  misc_feature 21: note = DNA
  misc_feature 22..30: note = RNA
  misc_feature 31: note = DNA
  misc_feature 32..39: note = RNA
  misc_feature 40: note = DNA

SEQ ID NO: 467 - DNA, 40 bp, synthetic construct; identical to SEQ ID NO: 415
  source 1..40: mol_type = other DNA; note = Description of Artificial Sequence: Synthetic oligonucleotide; note = TR_cr3'_tgt.12-d20.11.1
  misc_feature 1: note = DNA
  misc_feature 2: note = RNA
  misc_feature 3: note = DNA
  misc_feature 4..6: note = RNA
  misc_feature 7: note = DNA
  misc_feature 8..9: note = RNA
  misc_feature 10: note = DNA
  misc_feature 11: note = RNA
  misc_feature 12: note = DNA
  misc_feature 13: note = RNA
  misc_feature 14..15: note = DNA
  misc_feature 16..18: note = RNA
  misc_feature 19: note = DNA
  misc_feature 20: note = RNA
  misc_feature 21: note = DNA
  misc_feature 22..30: note = RNA
  misc_feature 31: note = DNA
  misc_feature 32..39: note = RNA
  misc_feature 40: note = DNA

SEQ ID NO: 468 - DNA, 40 bp, synthetic construct; identical to SEQ ID NO: 415
  source 1..40: mol_type = other DNA; note = Description of Artificial Sequence: Synthetic oligonucleotide; note = TR_cr-2_tgt.12-d20.11.1
  misc_feature 1: note = DNA
  misc_feature 2..14: note = RNA
  misc_feature 15..19: note = DNA
  misc_feature 20: note = RNA
  misc_feature 21: note = DNA
  misc_feature 22..30: note = RNA
  misc_feature 31: note = DNA
  misc_feature 32..39: note = RNA
  misc_feature 40: note = DNA

SEQ ID NO: 469 - DNA, 40 bp, synthetic construct; identical to SEQ ID NO: 415
  source 1..40: mol_type = other DNA; note = Description of Artificial Sequence: Synthetic oligonucleotide; note = TR_cr-6_tgt.12-d20.11.1
  misc_feature 1: note = DNA
  misc_feature 2..12: note = RNA
  misc_feature 13: note = DNA
  misc_feature 14..15: note = RNA
  misc_feature 16..17: note = DNA
  misc_feature 18..20: note = RNA
  misc_feature 21: note = DNA
  misc_feature 22..30: note = RNA
  misc_feature 31: note = DNA
  misc_feature 32..39: note = RNA
  misc_feature 40: note = DNA

SEQ ID NO: 470 - DNA, 40 bp, synthetic construct; identical to SEQ ID NO: 415
  source 1..40: mol_type = other DNA; note = Description of Artificial Sequence: Synthetic oligonucleotide; note = TR_cr-401_tgt.12-d20.11.1
  misc_feature 1..6: note = RNA
  misc_feature 7: note = DNA
  misc_feature 8..9: note = RNA
  misc_feature 10: note = DNA
  misc_feature 11: note = RNA
  misc_feature 12: note = DNA
  misc_feature 13: note = RNA
  misc_feature 14..15: note = DNA
  misc_feature 16: note = RNA
  misc_feature 17: note = DNA
  misc_feature 18: note = RNA
  misc_feature 19: note = DNA
  misc_feature 20: note = RNA
  misc_feature 21: note = DNA
  misc_feature 22..30: note = RNA
  misc_feature 31: note = DNA
  misc_feature 32..39: note = RNA
  misc_feature 40: note = DNA

SEQ ID NO: 471 - DNA, 40 bp, synthetic construct; identical to SEQ ID NO: 415
  source 1..40: mol_type = other DNA; note = Description of Artificial Sequence: Synthetic oligonucleotide; note = TR_cr-403_tgt.12-d20.11.1
  misc_feature 1: note = DNA
  misc_feature 2: note = RNA
  misc_feature 3: note = DNA
  misc_feature 4..11: note = RNA
  misc_feature 12: note = DNA
  misc_feature 13: note = RNA
  misc_feature 14: note = DNA
  misc_feature 15: note = RNA
  misc_feature 16..17: note = DNA
  misc_feature 18: note = RNA
  misc_feature 19: note = DNA
  misc_feature 20: note = RNA
  misc_feature 21: note = DNA
  misc_feature 22..30: note = RNA
  misc_feature 31: note = DNA
  misc_feature 32..39: note = RNA
  misc_feature 40: note = DNA

SEQ ID NO: 472 - DNA, 40 bp, synthetic construct; identical to SEQ ID NO: 415
  source 1..40: mol_type = other DNA; note = Description of Artificial Sequence: Synthetic oligonucleotide; note = TR_cr-404_tgt.12-d20.11.1
  misc_feature 1..6: note = RNA
  misc_feature 7: note = DNA
  misc_feature 8..9: note = RNA
  misc_feature 10: note = DNA
  misc_feature 11: note = RNA
  misc_feature 12: note = DNA
  misc_feature 13: note = RNA
  misc_feature 14: note = DNA
  misc_feature 15..16: note = RNA
  misc_feature 17: note = DNA
  misc_feature 18: note = RNA
  misc_feature 19: note = DNA
  misc_feature 20: note = RNA
  misc_feature 21: note = DNA
  misc_feature 22..30: note = RNA
  misc_feature 31: note = DNA
  misc_feature 32..39: note = RNA
  misc_feature 40: note = DNA

SEQ ID NO: 473 - DNA, 40 bp, synthetic construct; identical to SEQ ID NO: 415
  source 1..40: mol_type = other DNA; note = Description of Artificial Sequence: Synthetic oligonucleotide; note = TR_cr-405_tgt.12-d20.11.1
  misc_feature 1: note = DNA
  misc_feature 2: note = RNA
  misc_feature 3: note = DNA
  misc_feature 4..6: note = RNA
  misc_feature 7: note = DNA
  misc_feature 8..9: note = RNA
  misc_feature 10: note = DNA
  misc_feature 11: note = RNA
  misc_feature 12: note = DNA
  misc_feature 13: note = RNA
  misc_feature 14: note = DNA
  misc_feature 15..16: note = RNA
  misc_feature 17: note = DNA
  misc_feature 18: note = RNA
  misc_feature 19: note = DNA
  misc_feature 20: note = RNA
  misc_feature 21: note = DNA
  misc_feature 22..30: note = RNA
  misc_feature 31: note = DNA
  misc_feature 32..39: note = RNA
  misc_feature 40: note = DNA

SEQ ID NO: 474 - protein, 117 aa, synthetic construct
  source 1..117: note = Description of Artificial Sequence: Synthetic polypeptide; note = BCMA scFv chain 1
QVQLVQSGAEVKKPGSSVKVSCKASGYTFTSYVMHWVRQAPGQGLEWMGYIIPYNDATKYNEKFKGRVTITADKSTSTAYMELSSLRSEDTAVYYCARYNYDGYFDVWGQGTLVTVS

SEQ ID NO: 475 - protein, 107 aa, synthetic construct
  source 1..107: note = Description of Artificial Sequence: Synthetic polypeptide; note = BCMA scFv chain 2
EIVLTQSPATLSLSPGERATLSCRASQSISDYLHWYQQKPGQAPRLLIYYASQSITGIPARFSGSGSGTDFTLTISSLEPEDFAVYYCQNGHSFPPTFGGGTKVEIK

SEQ ID NO: 476 - protein, 15 aa, synthetic construct
  source 1..15: note = Description of Artificial Sequence: Synthetic peptide; note = BCMA scFv linker
GGGGSGGGGSGGGGS

SEQ ID NO: 477 - protein, 240 aa, synthetic construct
  source 1..240: note = Description of Artificial Sequence: Synthetic polypeptide; note = BCMA scFv
QVQLVQSGAEVKKPGSSVKVSCKASGYTFTSYVMHWVRQAPGQGLEWMGYIIPYNDATKYNEKFKGRVTITADKSTSTAYMELSSLRSEDTAVYYCARYNYDGYFDVWGQGTLVTVSSGGGGSGGGGSGGGGSEIVLTQSPATLSLSPGERATLSCRASQSISDYLHWYQQKPGQAPRLLIYYASQSITGIPARFSGSGSGTDFTLTISSLEPEDFAVYYCQNGHSFPPTFGGGTKVEIK

SEQ ID NO: 478 - DNA, 63 bp, Homo sapiens
  source 1..63: mol_type = unassigned DNA; note = CD8-alpha signal sequence
atggcgttgcccgtgaccgcacttctgcttccattggcactcctgctccacgcggcaagaccg

SEQ ID NO: 479 - protein, 9 aa, synthetic construct
  source 1..9: note = Description of Artificial Sequence: Synthetic peptide; note = GS_SV40-NLS
GSPKKKRKV

SEQ ID NO: 480 - protein, 18 aa, synthetic construct
  source 1..18: note = Description of Artificial Sequence: Synthetic peptide; note = GS_SV40-NLS_GS_SV40-NLS
GSPKKKRKVGSPKKKRKV

SEQ ID NO: 481 - protein, 18 aa, synthetic construct
  source 1..18: note = Description of Artificial Sequence: Synthetic peptide; note = GS_NPL-NLS
GSKRPAATKKAGQAKKKK

SEQ ID NO: 482 - protein, 36 aa, synthetic construct
  source 1..36: note = Description of Artificial Sequence: Synthetic polypeptide; note = GS_NPL-NLS_GS_NPL-NLS
GSKRPAATKKAGQAKKKKGSKRPAATKKAGQAKKKK

SEQ ID NO: 483 - protein, 27 aa, synthetic construct
  source 1..27: note = Description of Artificial Sequence: Synthetic peptide; note = GS_SV40-NLS_GS_NPL-NLS
GSPKKKRKVGSKRPAATKKAGQAKKKK

SEQ ID NO: 484 - protein, 27 aa, synthetic construct
  source 1..27: note = Description of Artificial Sequence: Synthetic peptide; note = GS_NPL-NLS_GS_SV40-NLS
GSKRPAATKKAGQAKKKKGSPKKKRKV

SEQ ID NO: 485 - protein, 35 aa, synthetic construct
  source 1..35: note = Description of Artificial Sequence: Synthetic polypeptide; note = (GGGGS)2_SV40-NLS_GS_NPL-NLS
GGGGSGGGGSPKKKRKVGSKRPAATKKAGQAKKKK

SEQ ID NO: 486 - protein, 35 aa, synthetic construct
  source 1..35: note = Description of Artificial Sequence: Synthetic polypeptide; note = (GGGGS)2_NPL-NLS_GS_SV40-NLS
GGGGSGGGGSKRPAATKKAGQAKKKKGSPKKKRKV

SEQ ID NO: 487 - protein, 17 aa, synthetic construct
  source 1..17: note = Description of Artificial Sequence: Synthetic peptide; note = (GGGGS)2_SV40-NLS
GGGGSGGGGSPKKKRKV

SEQ ID NO: 488 - protein, 26 aa, synthetic construct
  source 1..26: note = Description of Artificial Sequence: Synthetic peptide; note = (GGGGS)2_SV40-NLS_GS_SV40-NLS
GGGGSGGGGSPKKKRKVGSPKKKRKV

SEQ ID NO: 489 - protein, 26 aa, synthetic construct
  source 1..26: note = Description of Artificial Sequence: Synthetic peptide; note = (GGGGS)2_NPL-NLS
GGGGSGGGGSKRPAATKKAGQAKKKK

SEQ ID NO: 490 - protein, 44 aa, synthetic construct
  source 1..44: note = Description of Artificial Sequence: Synthetic polypeptide; note = (GGGGS)2_NPL-NLS_GS_NPL-NLS
GGGGSGGGGSKRPAATKKAGQAKKKKGSKRPAATKKAGQAKKKK

SEQ ID NO: 491 - RNA, 20 nt, synthetic construct
  source 1..20: mol_type = other RNA; note = Description of Artificial Sequence: Synthetic oligonucleotide; note = TRAC-tgt25
cagatacgaacctaaacttt

SEQ ID NO: 492 - RNA, 20 nt, synthetic construct
  source 1..20: mol_type = other RNA; note = Description of Artificial Sequence: Synthetic oligonucleotide; note = TRAC-tgt28
aggaggaggattcggaaccc

SEQ ID NO: 493 - protein, 21 aa, synthetic construct
  source 1..21: note = Description of Artificial Sequence: Synthetic peptide; note = 53BP1
GKRKLITSEEERSPAKRGRKS

SEQ ID NO: 494 - protein, 6 aa, synthetic construct
  source 1..6: note = Description of Artificial Sequence: Synthetic peptide; note = VACM-1/CUL5
PKLKRQ

SEQ ID NO: 495 - protein, 4 aa, synthetic construct
  source 1..4: note = Description of Artificial Sequence: Synthetic peptide; note = CXCR4
RPRK

SEQ ID NO: 496 - protein, 8 aa, synthetic construct
  source 1..8: note = Description of Artificial Sequence: Synthetic peptide; note = VP1
RRARRPRG

SEQ ID NO: 497 - protein, 22 aa, synthetic construct
  source 1..22: note = Description of Artificial Sequence: Synthetic peptide; note = ING4
KGKKGRTQKEKKAARARSKGKN

SEQ ID NO: 498 - protein, 28 aa, synthetic construct
  source 1..28: note = Description of Artificial Sequence: Synthetic peptide; note = IER5
RKRCAAGVGGGPAGCPAPGSTPLKKPRR

SEQ ID NO: 499 - protein, 36 aa, synthetic construct
  source 1..36: note = Description of Artificial Sequence: Synthetic polypeptide; note = ERK5
RKPVTAQERQREREEKRRRRQERAKEREKRRQERER

SEQ ID NO: 500 - protein, 27 aa, synthetic construct
  source 1..27: note = Description of Artificial Sequence: Synthetic peptide; note = UL79
TLLLRETMNNLGVSDHAVLSRKTPQPY

SEQ ID NO: 501 - protein, 18 aa, synthetic construct
  source 1..18: note = Description of Artificial Sequence: Synthetic peptide; note = EWS
PGKMDKGEHRQERRDRPY

SEQ ID NO: 502 - protein, 27 aa, synthetic construct
  source 1..27: note = Description of Artificial Sequence: Synthetic peptide; note = Hrp1
RSGGNHRRNGRGGRGGYNRRNNGYHPY

SEQ ID NO: 503 - protein, 9 aa, synthetic construct
  source 1..9: note = Description of Artificial Sequence: Synthetic peptide; note = cMyc (1)
PAAKRCKLD

SEQ ID NO: 504 - protein, 11 aa, synthetic construct
  source 1..11: note = Description of Artificial Sequence: Synthetic peptide; note = cMyc (2)
RQRRNELKRSP

SEQ ID NO: 505 - protein, 12 aa, synthetic construct
  source 1..12: note = Description of Artificial Sequence: Synthetic peptide; note = mouse c-able IV
SALIKKKKKNAP

SEQ ID NO: 506 - protein, 5 aa, synthetic construct
  source 1..5: note = Description of Artificial Sequence: Synthetic peptide; note = Matalpha2
KIPIK

SEQ ID NO: 507 - protein, 6 aa, synthetic construct
  source 1..6: note = Description of Artificial Sequence: Synthetic peptide; note = MINIYO
KSGSRK

SEQ ID NO: 508 - DNA, 40 bp, synthetic construct
  source 1..40: mol_type = other DNA; note = Description of Artificial Sequence: Synthetic oligonucleotide; note = TRAC-tgt25 chRDNA
  misc_feature 1: note = DNA
  misc_feature 2: note = RNA
  misc_feature 3: note = DNA
  misc_feature 4..6: note = RNA
  misc_feature 7: note = DNA
  misc_feature 8..9: note = RNA
  misc_feature 10: note = DNA
  misc_feature 11: note = RNA
  misc_feature 12: note = DNA
  misc_feature 13: note = RNA
  misc_feature 14..15: note = DNA
  misc_feature 16..18: note = RNA
  misc_feature 19: note = DNA
  misc_feature 20..30: note = RNA
  misc_feature 31: note = DNA
  misc_feature 32..36: note = RNA
  misc_feature 37: note = DNA
  misc_feature 38..39: note = RNA
  misc_feature 40: note = DNA
taatttctactcttgtagatcagatacgaacctaaacttt

SEQ ID NO: 509 - DNA, 40 bp, synthetic construct
  source 1..40: mol_type = other DNA; note = Description of Artificial Sequence: Synthetic oligonucleotide; note = CISH-tgt3 chRDNA
  misc_feature 1: note = DNA
  misc_feature 2: note = RNA
  misc_feature 3: note = DNA
  misc_feature 4..6: note = RNA
  misc_feature 7: note = DNA
  misc_feature 8..9: note = RNA
  misc_feature 10: note = DNA
  misc_feature 11: note = RNA
  misc_feature 12: note = DNA
  misc_feature 13: note = RNA
  misc_feature 14..15: note = DNA
  misc_feature 16..18: note = RNA
  misc_feature 19: note = DNA
  misc_feature 20..40: note = RNA
taatttctactcttgtagatggtgtacagcagtggctggt

SEQ ID NO: 510 - DNA, 40 bp, synthetic construct
  source 1..40: mol_type = other DNA; note = Description of Artificial Sequence: Synthetic oligonucleotide; note = CBLB-tgt3 chRDNA
  misc_feature 1: note = DNA
  misc_feature 2: note = RNA
  misc_feature 3: note = DNA
  misc_feature 4..6: note = RNA
  misc_feature 7: note = DNA
  misc_feature 8..9: note = RNA
  misc_feature 10: note = DNA
  misc_feature 11: note = RNA
  misc_feature 12: note = DNA
  misc_feature 13: note = RNA
  misc_feature 14..15: note = DNA
  misc_feature 16..18: note = RNA
  misc_feature 19: note = DNA
  misc_feature 20..40: note = RNA
taatttctactcttgtagatcctgatacatatcagcattt

SEQ ID NO: 511 - RNA, 40 nt, synthetic construct
  source 1..40: mol_type = other RNA; note = Description of Artificial Sequence: Synthetic oligonucleotide; note = TRAC-tgt12 crRNA
taatttctactcttgtagatgagtctctcagctggtacac

SEQ ID NO: 512 - RNA, 40 nt, synthetic construct; identical to SEQ ID NO: 511
  source 1..40: mol_type = other RNA; note = Description of Artificial Sequence: Synthetic oligonucleotide; note = TRAC-tgt12 ps-r1.2_s19.20
  modified_base 1: mod_base = OTHER; note = Nucleotide with phosphorothioate linkage
  modified_base 2: mod_base = OTHER; note = Nucleotide with phosphorothioate linkage
  modified_base 38: mod_base = OTHER; note = Nucleotide with phosphorothioate linkage
  modified_base 39: mod_base = OTHER; note = Nucleotide with phosphorothioate linkage

SEQ ID NO: 513 - DNA, 40 bp, synthetic construct; identical to SEQ ID NO: 415
  source 1..40: mol_type = other DNA; note = Description of Artificial Sequence: Synthetic oligonucleotide; note = TRAC-tgt12 ps-r2_r-d1
  modified_base 1: mod_base = OTHER; note = Nucleotide with phosphorothioate linkage
  modified_base 2: mod_base = OTHER; note = Nucleotide with phosphorothioate linkage
  modified_base 38: mod_base = OTHER; note = Nucleotide with phosphorothioate linkage
  modified_base 39: mod_base = OTHER; note = Nucleotide with phosphorothioate linkage
  misc_feature 1: note = DNA
  misc_feature 2..40: note = RNA

SEQ ID NO: 514 - DNA, 40 bp, synthetic construct; identical to SEQ ID NO: 415
  source 1..40: mol_type = other DNA; note = Description of Artificial Sequence: Synthetic oligonucleotide; note = TRAC-tgt12 ps-r1.2_r-d7
  modified_base 1: mod_base = OTHER; note = Nucleotide with phosphorothioate linkage
  modified_base 2: mod_base = OTHER; note = Nucleotide with phosphorothioate linkage
  modified_base 7: mod_base = OTHER; note = Nucleotide with phosphorothioate linkage
  modified_base 38: mod_base = OTHER; note = Nucleotide with phosphorothioate linkage
  modified_base 39: mod_base = OTHER; note = Nucleotide with phosphorothioate linkage
  misc_feature 1..6: note = RNA
  misc_feature 7: note = DNA
  misc_feature 8..40: note = RNA

SEQ ID NO: 515 - DNA, 40 bp, synthetic construct; identical to SEQ ID NO: 415
  source 1..40: mol_type = other DNA; note = Description of Artificial Sequence: Synthetic oligonucleotide; note = TRAC-tgt12 ps-r1.2_r-d10
  modified_base 1: mod_base = OTHER; note = Nucleotide with phosphorothioate linkage
  modified_base 2: mod_base = OTHER; note = Nucleotide with phosphorothioate linkage
  modified_base 10: mod_base = OTHER; note = Nucleotide with phosphorothioate linkage
  modified_base 38: mod_base = OTHER; note = Nucleotide with phosphorothioate linkage
  modified_base 39: mod_base = OTHER; note = Nucleotide with phosphorothioate linkage
  misc_feature 1..9: note = RNA
  misc_feature 10: note = DNA
  misc_feature 11..40: note = RNA

SEQ ID NO: 516 - DNA, 40 bp, synthetic construct; identical to SEQ ID NO: 415
  source 1..40: mol_type = other DNA; note = Description of Artificial Sequence: Synthetic oligonucleotide; note = TRAC-tgt12 ps-r1.2_r-d12
  modified_base 1: mod_base = OTHER; note = Nucleotide with phosphorothioate linkage
  modified_base 2: mod_base = OTHER; note = Nucleotide with phosphorothioate linkage
  modified_base 12: mod_base = OTHER; note = Nucleotide with phosphorothioate linkage
  modified_base 38: mod_base = OTHER; note = Nucleotide with phosphorothioate linkage
  modified_base 39: mod_base = OTHER; note = Nucleotide with phosphorothioate linkage
  misc_feature 1..11: note = RNA
  misc_feature 12: note = DNA
  misc_feature 13..40: note = RNA

SEQ ID NO: 517 - DNA, 40 bp, synthetic construct; identical to SEQ ID NO: 415
  source 1..40: mol_type = other DNA; note = Description of Artificial Sequence: Synthetic oligonucleotide; note = TRAC-tgt12 ps-r1.2_r-d14
  modified_base 1: mod_base = OTHER; note = Nucleotide with phosphorothioate linkage
  modified_base 2: mod_base = OTHER; note = Nucleotide with phosphorothioate linkage
  modified_base 14: mod_base = OTHER; note = Nucleotide with phosphorothioate linkage
  modified_base 38: mod_base = OTHER; note = Nucleotide with phosphorothioate linkage
  modified_base 39: mod_base = OTHER; note = Nucleotide with phosphorothioate linkage
  misc_feature 1..13: note = RNA
  misc_feature 14: note = DNA
  misc_feature 15..40: note = RNA

SEQ ID NO: 518 - DNA, 40 bp, synthetic construct; identical to SEQ ID NO: 509
  source 1..40: mol_type = other DNA; note = Description of Artificial Sequence: Synthetic oligonucleotide; note = CSH-tgt3_3'_d1
  misc_feature 1: note = DNA
  misc_feature 2: note = RNA
  misc_feature 3: note = DNA
  misc_feature 4..6: note = RNA
  misc_feature 7: note = DNA
  misc_feature 8..9: note = RNA
  misc_feature 10: note = DNA
  misc_feature 11: note = RNA
  misc_feature 12: note = DNA
  misc_feature 13: note = RNA
  misc_feature 14..15: note = DNA
  misc_feature 16..18: note = RNA
  misc_feature 19: note = DNA
  misc_feature 20: note = RNA
  misc_feature 21: note = DNA
  misc_feature 22..40: note = RNA

SEQ ID NO: 519 - DNA, 40 bp, synthetic construct; identical to SEQ ID NO: 509
  source 1..40: mol_type = other DNA; note = Description of Artificial Sequence: Synthetic oligonucleotide; note = CSH-tgt3_3'_d8
  misc_feature 1: note = DNA
  misc_feature 2: note = RNA
  misc_feature 3: note = DNA
  misc_feature 4..6: note = RNA
  misc_feature 7: note = DNA
  misc_feature 8..9: note = RNA
  misc_feature 10: note = DNA
  misc_feature 11: note = RNA
  misc_feature 12: note = DNA
  misc_feature 13: note = RNA
  misc_feature 14..15: note = DNA
  misc_feature 16..18: note = RNA
  misc_feature 19: note = DNA
  misc_feature 20..27: note = RNA
  misc_feature 28: note = DNA
  misc_feature 29..40: note = RNA

SEQ ID NO: 520 - DNA, 40 bp, synthetic construct; identical to SEQ ID NO: 509
  source 1..40: mol_type = other DNA; note = Description of Artificial Sequence: Synthetic oligonucleotide; note = CSH-tgt3_3'_d9
  misc_feature 1: note = DNA
  misc_feature 2: note = RNA
  misc_feature 3: note = DNA
  misc_feature 4..6: note = RNA
  misc_feature 7: note = DNA
  misc_feature 8..9: note = RNA
  misc_feature 10: note = DNA
  misc_feature 11: note = RNA
  misc_feature 12: note = DNA
  misc_feature 13: note = RNA
  misc_feature 14..15: note = DNA
  misc_feature 16..18: note = RNA
  misc_feature 19: note = DNA
  misc_feature 20..28: note = RNA
  misc_feature 29: note = DNA
  misc_feature 30..40: note = RNA

SEQ ID NO: 521 - DNA, 40 bp, synthetic construct; identical to SEQ ID NO: 509
  source 1..40: mol_type = other DNA; note = Description of Artificial Sequence: Synthetic oligonucleotide; note = CSH-tgt3_3'_d10
  misc_feature 1: note = DNA
  misc_feature 2: note = RNA
  misc_feature 3: note = DNA
  misc_feature 4..6: note = RNA
  misc_feature 7: note = DNA
  misc_feature 8..9: note = RNA
  misc_feature 10: note = DNA
  misc_feature 11: note = RNA
  misc_feature 12: note = DNA
  misc_feature 13: note = RNA
  misc_feature 14..15: note = DNA
  misc_feature 16..18: note = RNA
  misc_feature 19: note = DNA
  misc_feature 20..29: note = RNA
  misc_feature 30: note = DNA
  misc_feature 31..40: note = RNA

SEQ ID NO: 522 - DNA, 40 bp, synthetic construct; identical to SEQ ID NO: 509
  source 1..40: mol_type = other DNA; note = Description of Artificial Sequence: Synthetic oligonucleotide; note = CSH-tgt3_3'_d11
  misc_feature 1: note = DNA
  misc_feature 2: note = RNA
  misc_feature 3: note = DNA
  misc_feature 4..6: note = RNA
  misc_feature 7: note = DNA
  misc_feature 8..9: note = RNA
  misc_feature 10: note = DNA
  misc_feature 11: note = RNA
  misc_feature 12: note = DNA
  misc_feature 13: note = RNA
  misc_feature 14..15: note = DNA
  misc_feature 16..18: note = RNA
  misc_feature 19: note = DNA
  misc_feature 20..30: note = RNA
  misc_feature 31: note = DNA
  misc_feature 32..40: note = RNA

SEQ ID NO: 523 - DNA, 40 bp, synthetic construct; identical to SEQ ID NO: 509
  source 1..40: mol_type = other DNA; note = Description of Artificial Sequence: Synthetic oligonucleotide; note = CSH-tgt3_3'_d12
  misc_feature 1: note = DNA
  misc_feature 2: note = RNA
  misc_feature 3: note = DNA
  misc_feature 4..6: note = RNA
  misc_feature 7: note = DNA
  misc_feature 8..9: note = RNA
  misc_feature 10: note = DNA
  misc_feature 11: note = RNA
  misc_feature 12: note = DNA
  misc_feature 13: note = RNA
  misc_feature 14..15: note = DNA
  misc_feature 16..18: note = RNA
  misc_feature 19: note = DNA
  misc_feature 20..31: note = RNA
  misc_feature 32: note = DNA
  misc_feature 33..40: note = RNA

SEQ ID NO: 524 - DNA, 40 bp, synthetic construct; identical to SEQ ID NO: 509
  source 1..40: mol_type = other DNA; note = Description of Artificial Sequence: Synthetic oligonucleotide; note = CSH-tgt3_3'_d14
  misc_feature 1: note = DNA
  misc_feature 2: note = RNA
  misc_feature 3: note = DNA
  misc_feature 4..6: note = RNA
  misc_feature 7: note = DNA
  misc_feature 8..9: note = RNA
  misc_feature 10: note = DNA
  misc_feature 11: note = RNA
  misc_feature 12: note = DNA
  misc_feature 13: note = RNA
  misc_feature 14..15: note = DNA
  misc_feature 16..18: note = RNA
  misc_feature 19: note = DNA
  misc_feature 20..33: note = RNA
  misc_feature 34: note = DNA
  misc_feature 35..40: note = RNA

SEQ ID NO: 525 - DNA, 40 bp, synthetic construct; identical to SEQ ID NO: 509
  source 1..40: mol_type = other DNA; note = Description of Artificial Sequence: Synthetic oligonucleotide; note = CSH-tgt3_3'_d15
  misc_feature 1: note = DNA
  misc_feature 2: note = RNA
  misc_feature 3: note = DNA
  misc_feature 4..6: note = RNA
  misc_feature 7: note = DNA
  misc_feature 8..9: note = RNA
  misc_feature 10: note = DNA
  misc_feature 11: note = RNA
  misc_feature 12: note = DNA
  misc_feature 13: note = RNA
  misc_feature 14..15: note = DNA
  misc_feature 16..18: note = RNA
  misc_feature 19: note = DNA
  misc_feature 20..34: note = RNA
  misc_feature 35: note = DNA
  misc_feature 36..40: note = RNA

SEQ ID NO: 526 - DNA, 40 bp, synthetic construct; identical to SEQ ID NO: 509
  source 1..40: mol_type = other DNA; note = Description of Artificial Sequence: Synthetic oligonucleotide; note = CSH-tgt3_3'_d17
  misc_feature 1: note = DNA
  misc_feature 2: note = RNA
  misc_feature 3: note = DNA
  misc_feature 4..6: note = RNA
  misc_feature 7: note = DNA
  misc_feature 8..9: note = RNA
  misc_feature 10: note = DNA
  misc_feature 11: note = RNA
  misc_feature 12: note = DNA
  misc_feature 13: note = RNA
  misc_feature 14..15: note = DNA
  misc_feature 16..18: note = RNA
  misc_feature 19: note = DNA
  misc_feature 20..36: note = RNA
  misc_feature 37: note = DNA
  misc_feature 38..40: note = RNA

SEQ ID NO: 527 - DNA, 40 bp, synthetic construct; identical to SEQ ID NO: 509
  source 1..40: mol_type = other DNA; note = Description of Artificial Sequence: Synthetic oligonucleotide; note = CSH-tgt3_3'_d18
  misc_feature 1: note = DNA
  misc_feature 2: note = RNA
  misc_feature 3: note = DNA
  misc_feature 4..6: note = RNA
  misc_feature 7: note = DNA
  misc_feature 8..9: note = RNA
  misc_feature 10: note = DNA
  misc_feature 11: note = RNA
  misc_feature 12: note = DNA
  misc_feature 13: note = RNA
  misc_feature 14..15: note = DNA
  misc_feature 16..18: note = RNA
  misc_feature 19: note = DNA
  misc_feature 20..37: note = RNA
  misc_feature 38: note = DNA
  misc_feature 39..40: note = RNA

SEQ ID NO: 528 - DNA, 40 bp, synthetic construct; identical to SEQ ID NO: 509
  source 1..40: mol_type = other DNA; note = Description of Artificial Sequence: Synthetic oligonucleotide; note = CSH-tgt3_3'_d19
  misc_feature 1: note = DNA
  misc_feature 2: note = RNA
  misc_feature 3: note = DNA
  misc_feature 4..6: note = RNA
  misc_feature 7: note = DNA
  misc_feature 8..9: note = RNA
  misc_feature 10: note = DNA
  misc_feature 11: note = RNA
  misc_feature 12: note = DNA
  misc_feature 13: note = RNA
  misc_feature 14..15: note = DNA
  misc_feature 16..18: note = RNA
  misc_feature 19: note = DNA
  misc_feature 20..38: note = RNA
  misc_feature 39: note = DNA
  misc_feature 40: note = RNA

SEQ ID NO: 529 - DNA, 40 bp, synthetic construct; identical to SEQ ID NO: 509
  source 1..40: mol_type = other DNA; note = Description of Artificial Sequence: Synthetic oligonucleotide; note = CSH-tgt3_3'_d20
  misc_feature 1: note = DNA
  misc_feature 2: note = RNA
  misc_feature 3: note = DNA
  misc_feature 4..6: note = RNA
  misc_feature 7: note = DNA
  misc_feature 8..9: note = RNA
  misc_feature 10: note = DNA
  misc_feature 11: note = RNA
  misc_feature 12: note = DNA
  misc_feature 13: note = RNA
  misc_feature 14..15: note = DNA
  misc_feature 16..18: note = RNA
  misc_feature 19: note = DNA
  misc_feature 20..39: note = RNA
  misc_feature 40: note = DNA

SEQ ID NO: 530 - RNA, 40 nt, synthetic construct; identical to SEQ ID NO: 511
  source 1..40: mol_type = other RNA

SEQ ID NO: 531 - RNA, 40 nt, synthetic construct
  source 1..40: mol_type = other RNA
taatttctactcttgtagatagtgggggtgaattcagtgt

SEQ ID NO: 532 - RNA, 20 nt, synthetic construct
  source 1..20: mol_type = other RNA
gagtctctcagctggtacac

SEQ ID NO: 533 - RNA, 20 nt, synthetic construct
  source 1..20: mol_type = other RNA
agtgggggtgaattcagtgt